(19) United States
(12) Patent Application Publication
(10) Pub. No.: US 2025/0026727 A1
(43) Pub. Date: Jan. 23, 2025

(54) COMPOUND FOR ORGANIC ELECTRICAL ELEMENT, ORGANIC ELECTRICAL ELEMENT USING SAME, AND ELECTRONIC DEVICE THEREOF

(71) Applicant: DUK SAN NEOLUX CO., LTD., Cheonan-si, Chungcheongnam-do (KR)

(72) Inventors: Ki Ho SO, Cheonan-si, Chungcheongnam-do (KR); Jong Gwang PARK, Cheonan-si, Chungcheongnam-do (KR); Yun Suk LEE, Cheonan-si, Chungcheongnam-do (KR); Sun Hee LEE, Cheonan-si, Chungcheongnam-do (KR); Soung Yun MUN, Cheonan-si, Chungcheongnam-do (KR); In Goo LEE, Cheonan-si, Chungcheongnam-do (KR); Won Joan CHEONG, Cheonan-si, Chungcheongnam-do (KR)

(73) Assignee: DUK SAN NEOLUX CO., LTD., Cheonan-si, Chungcheongnam-do (KR)

(21) Appl. No.: 18/712,751

(22) PCT Filed: Dec. 8, 2022
(86) PCT No.: PCT/KR2022/019929

§ 371 Date: May 23, 2024

(30) Foreign Application Priority Data
Dec. 13, 2021 (KR) 10-2021-0177804

(51) Int. Cl. C07D 251/24; C07B 59/00; C09K 11/02; H10K 50/12; H10K 85/40; H10K 85/60; H10K 101/00

(52) U.S. Cl. CPC C07D 251/24; C07B 59/004; C09K 11/02; H10K 50/12; H10K 85/40; H10K 85/615; H10K 85/622; H10K 85/633; H10K 85/654; H10K 85/6572; H10K 85/6574; H10K 85/6576; C07B 2200/05; H10K 2101/90

(57) ABSTRACT

Provided are a novel compound which can improve the luminous efficiency, stability, and service life of an element; an organic electronic element using same, and an electronic device thereof.

### BACKGROUND

### Technical Field

The present invention relates to compound for organic electronic element, organic electronic element using same, and an electronic device thereof.

### Background Art

In general, organic light emitting phenomenon refers to a phenomenon that converts electric energy into light energy by using an organic material. An organic electronic element using an organic light emitting phenomenon usually has a structure including an anode, a cathode, and an organic material layer interposed therebetween. Wherein, the organic material layer is often composed of a multi-layered structure composed of different materials in order to increase the efficiency and stability of the organic electronic element, and for example, may include a hole injection layer, a hole transport layer, an emitting layer, an electron transport layer, an electron injection layer and the like.

A material used as an organic material layer in an organic electronic element may be classified into a light emitting material and a charge transport material, such as a hole injection material, a hole transport material, an electron transport material, an electron injection material and the like depending on its function. And the light emitting material can be classified into a high molecular weight type and a low molecular weight type according to the molecular weight, and it can be classified into a fluorescent material derived from a singlet excited state of an electron and a phosphorescent material derived from a triplet excited state of an electron according to the light emission mechanism. Also, the light emitting material may be divided into blue, green, and red light emitting materials and yellow and orange light emitting materials necessary for realizing a better natural color according to the emission color.

However, when only one material is used as a light emitting material, due to intermolecular interaction, the maximum emission wavelength shifts to a longer wavelength, and there are problems in that the color purity is lowered or the device efficiency is reduced due to the emission attenuation effect, therefore in order to increase color purity and increase luminous efficiency through energy transfer, a host/dopant system may be used as a light emitting material. The principle is that when a small amount of a dopant having a smaller energy band gap than that of the host forming the emitting layer is mixed in the emitting layer, excitons generated in the emitting layer are transported to the dopant to emit light with high efficiency. Here, since the wavelength of the host moves to the wavelength band of the dopant, light having a desired wavelength can be obtained according to the type of dopant used.

The biggest issues with organic light emitting devices are lifespan and efficiency, and as displays become larger, these efficiency and lifespan issues must be resolved. Efficiency, lifespan and driving voltage are related to each other, and when the efficiency is increased, the driving voltage is relatively decreased, and as the driving voltage is decreased, crystallization of organic materials due to Joule heating generated during driving decreases, and consequently the lifespan tends to increase.

Short-wavelength dopants are used as a method to increase maximum luminous efficiency. The short-wavelength dopants have higher maximum luminous efficiency than existing long-wavelength dopants, so can be used to increase the overall device efficiency. However, the x color coordinate is very low, which is a disadvantage in terms of color purity. To solve the disadvantages, a host whose maximum emission wavelength is shifted to a longer wavelength is needed, and such a host affects not only the maximum emission efficiency but also the lifespan.

However, the efficiency cannot be maximized simply by improving the organic material layer. This is because, when the energy level and T1 value between each organic material layer, and the intrinsic properties (mobility, interfacial properties, etc.) of materials are optimally combined, long lifespan and high efficiency can be achieved at the same time. Therefore, there is a need to develop an emitting material that has high thermal stability and can efficiently achieve charge balance within the emitting layer. That is, in order to fully exhibit the excellent characteristics of an organic electronic element, it should be preceded that the material constituting the organic material layer in the device, such as a hole injection material, a hole transport material, a light emitting material, an electron transport material, an electron injection material, etc., is supported by a stable and efficient material. But the development of a stable and efficient organic material layer material for an organic electronic element has not yet been sufficiently made, and in particular, the development of materials for the emitting layer is urgently needed.

### BRIEF DESCRIPTION OF THE INVENTION

### Summary

In order to solve the problems of the above-mentioned background technology, the present invention calculates the energy level of heterogeneous compounds mixed to develop a long-wavelength host, and aims to design an optimal compound based on this. Also, it was discovered that when the compound is applied to organic electric elements, the luminous efficiency and stability of the elements can be greatly improved.

Accordingly, the purpose of the present invention is to provide a novel compound, an organic electronic element using the same, and an electronic device thereof.

### Technical Solution

The present invention provides a compound represented by Formula (1).

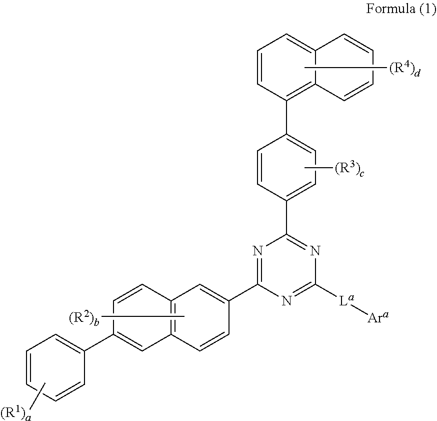

In another aspect, the present invention provides a composition for an organic electronic element comprising a compound represented by Formula 1 and an electronic device thereof.

### Effects of the Invention

By using the long-wavelength compound according to the present invention, high luminous efficiency, low driving voltage and high heat resistance of the element can be achieved, and color purity and lifespan of the element can be greatly improved.

### BRIEF DESCRIPTION OF THE DRAWINGS

FIG. 1 to FIG. 3 are exemplary views of an organic electroluminescent device according to the present invention.

| | |
|---|---|
| 100, 200, 300: organic electronic element | 110: the first electrode |
| 120: hole injection layer | 130: hole transport layer |
| 140: emitting layer | 150: electron transport layer |
| 160: electron injection layer | 170: second electrode |
| 180: light efficiency enhancing Layer | 210: buffer layer |
| 220: emitting auxiliary layer | 320: first hole injection layer |
| 330: first hole transport layer | 340: first emitting layer |
| 350: first electron transport layer | 360: first charge generation layer |
| 361: second charge generation layer | 420: second hole injection layer |
| 430: second hole transport layer | 440: second emitting layer |
| 450: second electron transport layer | CGL: charge generation layer |
| ST1: first stack | ST2: second stack |

### DETAILED DESCRIPTION OF THE INVENTION

Hereinafter, some embodiments of the present invention will be described in detail. Further, in the following description of the present invention, a detailed description of known functions and configurations incorporated herein will be omitted when it may make the subject matter of the present invention rather unclear.

In addition, terms, such as first, second, A, B, (a), (b) or the like may be used herein when describing components of the present invention. Each of these terminologies is not used to define an essence, order or sequence of a corresponding component but used merely to distinguish the corresponding component from other component(s). It should be noted that if a component is described as being “connected”, “coupled”, or “connected” to another component, the component may be directly connected or connected to the other component, but another component may be “connected”, “coupled” or “connected” between each component.

As used in the specification and the accompanying claims, unless otherwise stated, the following is the meaning of the term as follows.

Unless otherwise stated, the term “halo” or “halogen”, as used herein, includes fluorine, bromine, chlorine, or iodine.

Unless otherwise stated, the term “alkyl” or “alkyl group”, as used herein, has a single bond of 1 to 60 carbon atoms, and means saturated aliphatic functional radicals including a linear alkyl group, a branched chain alkyl group, a cycloalkyl group (alicyclic), an cycloalkyl group substituted with a alkyl or an alkyl group substituted with a cycloalkyl.

Unless otherwise stated, the term “alkenyl” or “alkynyl”, as used herein, has double or triple bonds of 2 to 60 carbon atoms, but is not limited thereto, and includes a linear or a branched chain group.

Unless otherwise stated, the term “cycloalkyl”, as used herein, means alkyl forming a ring having 3 to 60 carbon atoms, but is not limited thereto.

Unless otherwise stated, the term “alkoxyl group”, “alkoxy group” or “alkyloxy group”, as used herein, means an alkyl group bonded to oxygen radical, but is not limited thereto, and has 1 to 60 carbon atoms.

Unless otherwise stated, the term “aryloxyl group” or “aryloxy group”, as used herein, means an aryl group bonded to oxygen radical, but is not limited thereto, and has 6 to 60 carbon atoms.

The terms “aryl group” and “arylene group” used in the present invention have 6 to 60 carbon atoms, respectively, unless otherwise specified, but are not limited thereto. In the present invention, an aryl group or arylene group refers to an aromatic group of a single ring or multiple rings, and includes an aromatic ring formed by combining adjacent substituents or participating in a reaction. For example, the aryl group may be a phenyl group, a biphenyl group, a fluorene group, or a spirofluorene group.

The prefix “aryl” or “ar” means a radical substituted with an aryl group. For example, an arylalkyl may be an alkyl substituted with an aryl, and an arylalkenyl may be an alkenyl substituted with aryl, and a radical substituted with an aryl has a number of carbon atoms as defined herein.

Also, when prefixes are named subsequently, it means that substituents are listed in the order described first. For example, an arylalkoxy means an alkoxy substituted with an aryl, an alkoxylcarbonyl means a carbonyl substituted with an alkoxyl, and an arylcarbonylalkenyl also means an alkenyl substituted with an arylcarbonyl, wherein the arylcarbonyl may be a carbonyl substituted with an aryl.

Unless otherwise stated, the term “heterocyclic group”, as used herein, contains one or more heteroatoms, but is not limited thereto, has 2 to 60 carbon atoms, includes any one of a single ring or multiple ring, and may include heteroaliphadic ring and heteroaromatic ring. Also, the heterocyclic group may also be formed in conjunction with an adjacent functional group.

Unless otherwise stated, the term “heteroatom”, as used herein, represents at least one of N, O, S, P, or Si.

Also, the term “heterocyclic group” may include a ring including SO₂ instead of carbon consisting of cycle. For example, “heterocyclic group” includes the following compound.

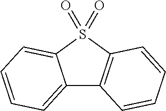 [SMILES: O=S1(=O)c2ccccc2-c2ccccc21]

Unless otherwise stated, the term “fluorenyl group” or “fluorenylene group”, as used herein, means a monovalent or divalent functional group, in which R, R′ and R″ are all hydrogen in the following structures, and the term “substituted fluorenyl group” or “substituted fluorenylene group” means that at least one of the substituents R, R′, R″ is a substituent other than hydrogen, and include those in which R and R′ are bonded to each other to form a spiro compound together with the carbon to which they are bonded.

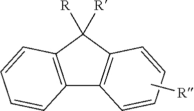

The term “spiro compound”, as used herein, has a ‘spiro union’, and the spiro union means a connection in which two rings share only one atom. Here, atoms shared in the two rings are called ‘spiro atoms’, and these compounds are called ‘monospiro-’, ‘di-spiro-’ and ‘tri-spiro-’, respectively, depending on the number of spiro atoms in a compound.

Unless otherwise stated, the term “aliphatic”, as used herein, means an aliphatic hydrocarbon having 1 to 60 carbon atoms, and the term “aliphatic ring”, as used herein, means an aliphatic hydrocarbon ring having 3 to 60 carbon atoms.

Unless otherwise stated, the term “ring”, as used herein, means an aliphatic ring having 3 to 60 carbon atoms, or an aromatic ring having 6 to 60 carbon atoms, or a hetero ring having 2 to 60 carbon atoms, or a fused ring formed by the combination of them, and includes a saturated or unsaturated ring.

Other hetero compounds or hetero radicals other than the above-mentioned hetero compounds include, but are not limited thereto, one or more heteroatoms.

Additionally, unless explicitly stated otherwise, “substituted” in the term “substituted or unsubstituted” used in the present invention means substituted with one or more substituents selected from the group consisting of deuterium, halogen, an amino group, a nitrile group, a nitro group, a C₁-C₂₀ alkyl group, a C₁-C₂₀ alkoxyl group, a C₁-C₂₀ alkylamine group, a C₁-C₂₀ alkylthiopen group, a C₆-C₂₀ arylthiopen group, a C₂-C₂₀ alkenyl group, a C₂-C₂₀ alkynyl group, a C₃-C₂₀ cycloalkyl group, a C₆-C₂₀ aryl group, a C₆-C₂₀ aryl group substituted by deuterium, a C₈-C₂₀ arylalkenyl group, a silane group, a boron group, a germanium group, and a C₂-C₂₀ heterocyclic group, but is not limited to these substituents.

Also, unless explicitly stated otherwise, the formula used in the present invention is the same as the definition of the substituent by the exponent definition of the following formula.

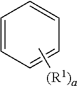

Here, when a is an integer of 0, the substituent R¹ is absent, when a is an integer of 1, the sole substituent R¹ is linked to any one of the carbon constituting the benzene ring, when a is an integer of 2 or 3, each is combined as follows, where R¹ may be the same or different from each other, when a is an integer of 4 to 6, it is bonded to the carbon of the benzene ring in a similar manner, while the indication of the hydrogen bonded to the carbon forming the benzene ring is omitted.

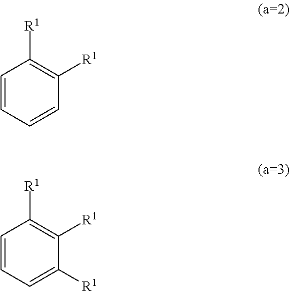

Hereinafter, a compound according to an aspect of the present invention, and an organic electronic element comprising the same will be described.

The present invention provides a compound represented by Formula 1.

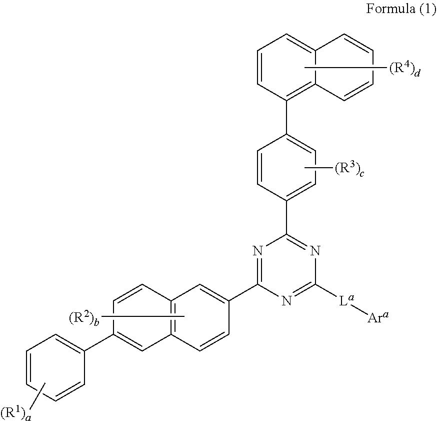

Wherein:
1) L^{a} is a direct bond; phenylene group; naphthylene group; biphenylene group; or a phenanthrene group;
2) Ar^{a} is a phenyl group; or a phenanthrenyl group;
3) R¹, R², R³ and R⁴ are each the same or different, and each independently hydrogen; or deuterium;
4) a is an integer from 0 to 5, b is an integer from 0 to 6, c is an integer from 0 to 4, d is an integer from 0 to 7,

Wherein, the phenyl group, phenanthrenyl group, phenylene group, naphthylene group and biphenylene group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxy group; C₆-C₂₀ aryloxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₁-C₂₀ heteroalkyl group; C₇-C₂₀ arylalkyl group; and C₈-C₂₀ arylalkenyl group; and also the substituents may be bonded to each other to form a saturated or unsaturated ring, wherein the term ‘ring’ means a C₃-C₂₀ aliphatic ring or a C₆-C₂₀ aromatic ring or a C₂-C₂₀ heterocyclic group or a fused ring formed by the combination thereof.

Additionally, Ar^{a} is represented by any one of the following formulas (A-1) to (A-6):

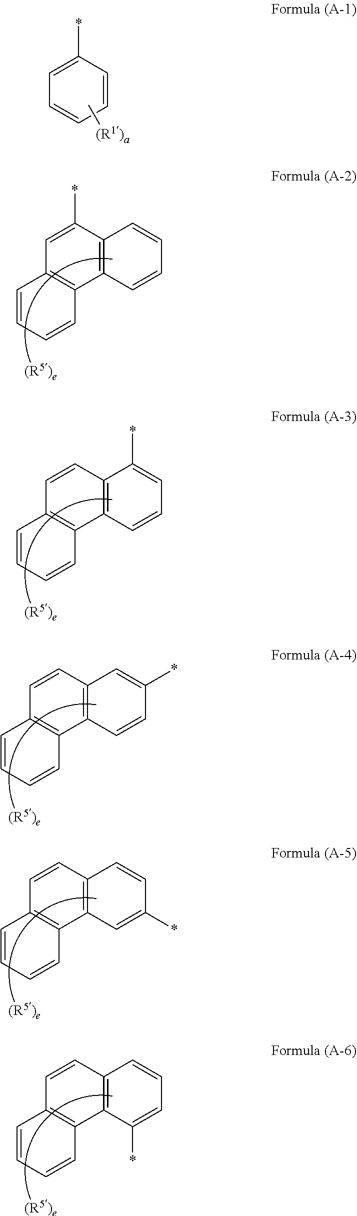

Wherein:
1) * means the position to be bonded,
2) R^{1′} and R^{5′} are each independently same or different, and each independently selected from the group consisting of a hydrogen; deuterium; halogen; silane group; siloxane group; boron group; germanium group; cyano group; nitro group; C₁-C₂₀ alkylthio group; C₁-C₂₀ alkoxy group; C₆-C₂₀ aryloxy group; C₁-C₂₀ alkyl group; C₂-C₂₀ alkenyl group; C₂-C₂₀ alkynyl group; C₆-C₂₀ aryl group; C₆-C₂₀ aryl group substituted with deuterium; a fluorenyl group; C₂-C₂₀ heterocyclic group; C₃-C₂₀ cycloalkyl group; C₁-C₂₀ heteroalkyl; C₇-C₂₀ arylalkyl group; and C₈-C₂₀ arylalkenyl group; or an adjacent plurality of R^{1′} or plurality of R^{5′} may be bonded to each other to form a ring, 3) a is an integer from 0 to 5, e is an integer from 0 to 9,

Also, L^{a} is represented by any of the following formulas (L-1) to (L-14): Formula (L-1) Formula (L-2) Formula (L-3) Formula (L-4) Formula (L-5)

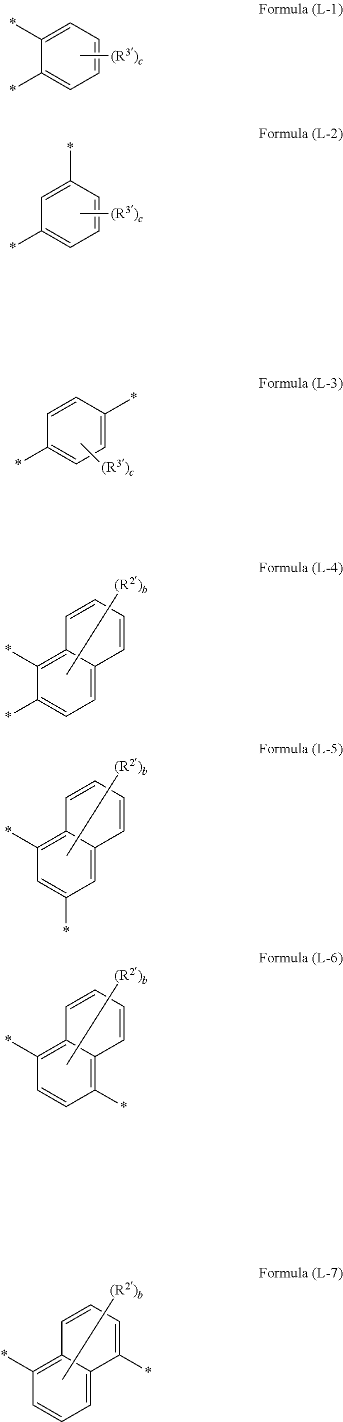

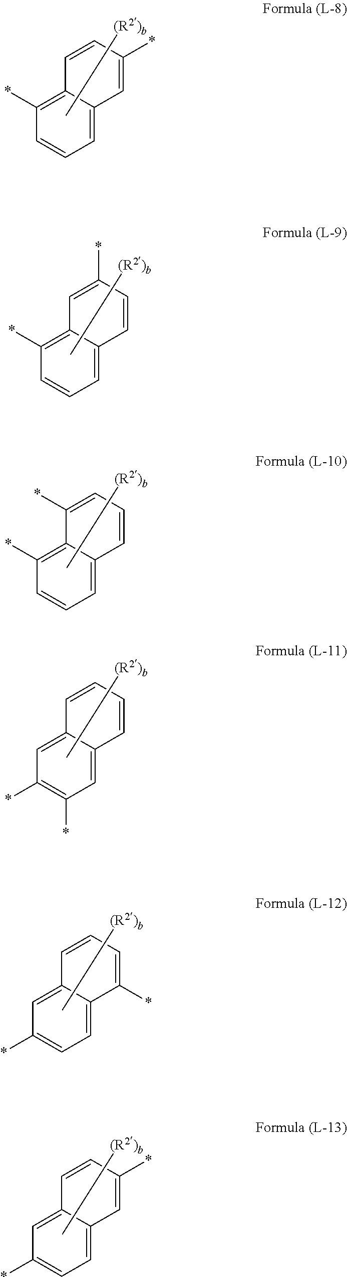

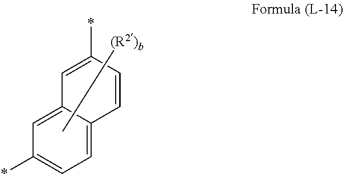

Wherein:
1) * means the position to be bonded,
2) R^{2′} and R^{3′} are the same as the definition of R^{1′},
3) b is an integer from 0 to 6, c is an integer from 0 to 4.

Additionally, the present invention provides a compound represented by Formula (1) having a Reorganization Energy value higher than 0.23.

Additionally, the present invention provides a compound represented by Formula (1) having a Reorganization Energy value higher than 0.23 and lower than 0.29.

Formula (1) may be represented by any one of the following compounds P-1 to P-40.

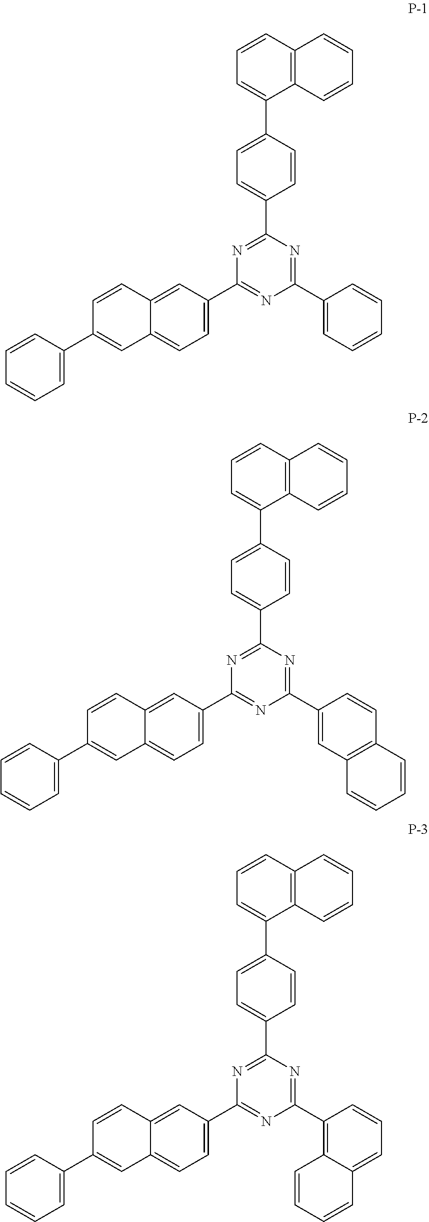 [SMILES: c1ccc(-c2ccc3cc(-c4nc(-c5ccccc5)nc(-c5ccc(-c6cccc7ccccc67)cc5)n4)ccc3c2)cc1 ; c1ccc(-c2ccc3cc(-c4nc(-c5ccc(-c6cccc7ccccc67)cc5)nc(-c5ccc6ccccc6c5)n4)ccc3c2)cc1 ; c1ccc(-c2ccc3cc(-c4nc(-c5ccc(-c6cccc7ccccc67)cc5)nc(-c5cccc6ccccc56)n4)ccc3c2)cc1]

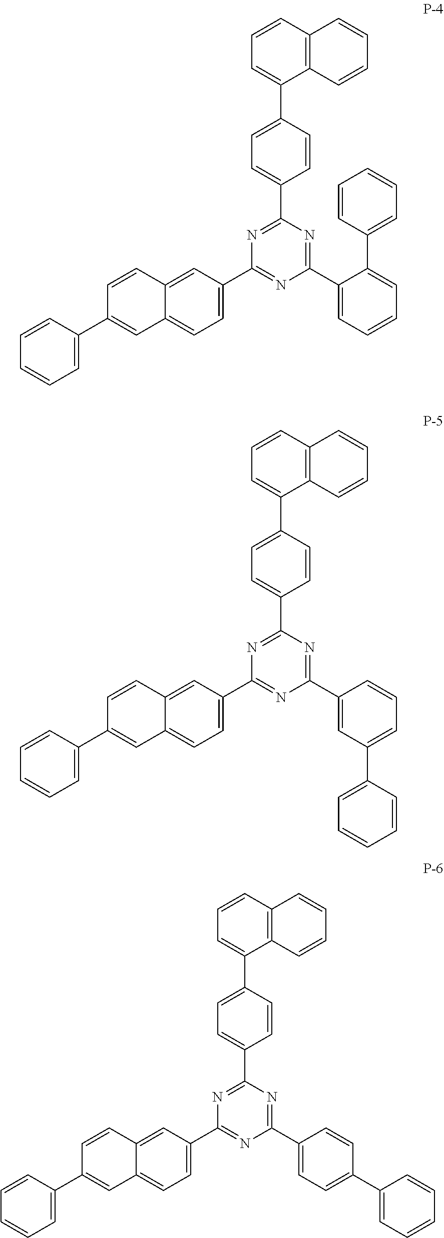 [SMILES: c1ccc(-c2ccc3cc(-c4nc(-c5ccc(-c6cccc7ccccc67)cc5)nc(-c5ccccc5-c5ccccc5)n4)ccc3c2)cc1 ; c1ccc(-c2cccc(-c3nc(-c4ccc(-c5cccc6ccccc56)cc4)nc(-c4ccc5cc(-c6ccccc6)ccc5c4)n3)c2)cc1 ; c1ccc(-c2ccc(-c3nc(-c4ccc(-c5cccc6ccccc56)cc4)nc(-c4ccc5cc(-c6ccccc6)ccc5c4)n3)cc2)cc1]

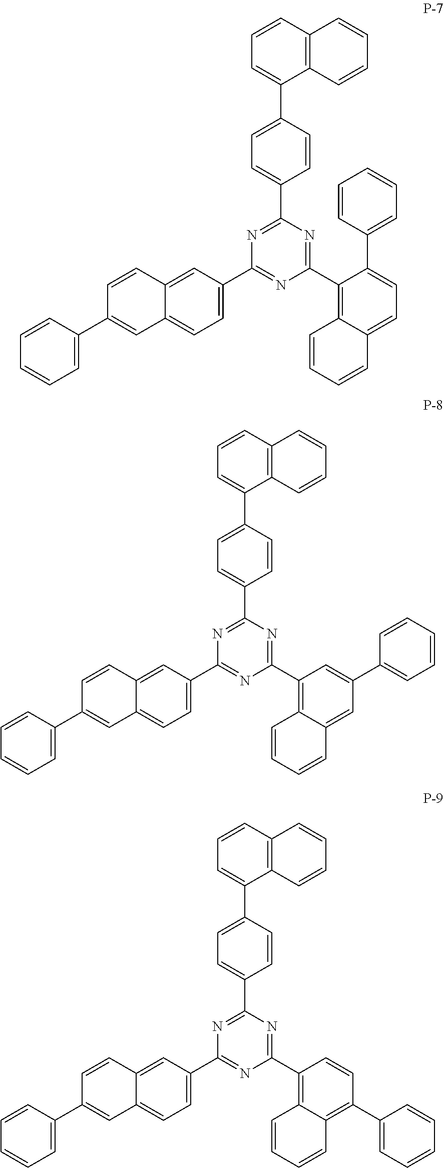 [SMILES: c1ccc(-c2ccc3cc(-c4nc(-c5ccc(-c6cccc7ccccc67)cc5)nc(-c5c(-c6ccccc6)ccc6ccccc56)n4)ccc3c2)cc1 ; c1ccc(-c2ccc3cc(-c4nc(-c5ccc(-c6cccc7ccccc67)cc5)nc(-c5cc(-c6ccccc6)cc6ccccc56)n4)ccc3c2)cc1 ; c1ccc(-c2ccc3cc(-c4nc(-c5ccc(-c6cccc7ccccc67)cc5)nc(-c5ccc(-c6ccccc6)c6ccccc56)n4)ccc3c2)cc1]

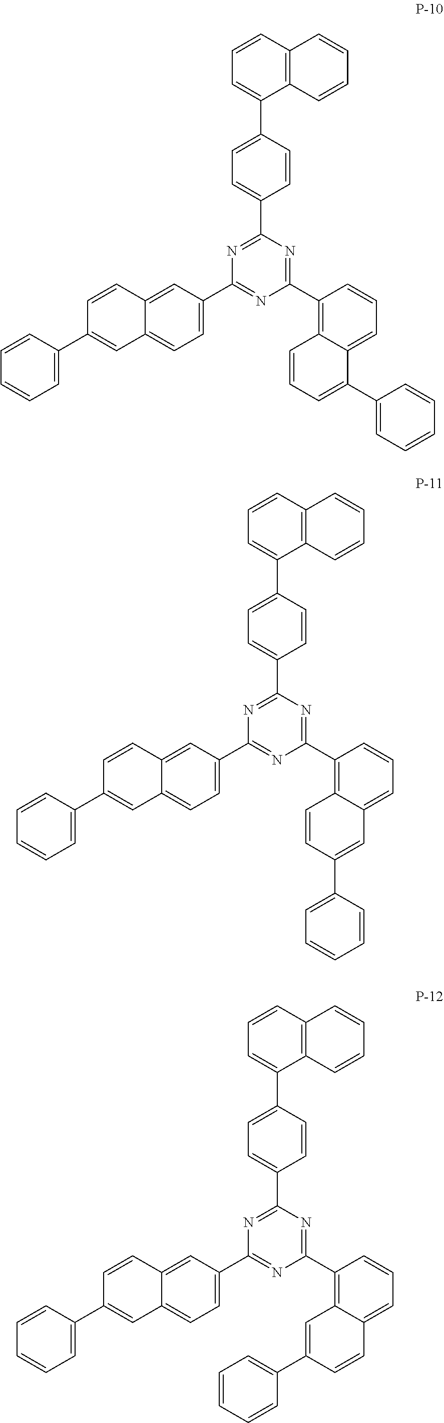

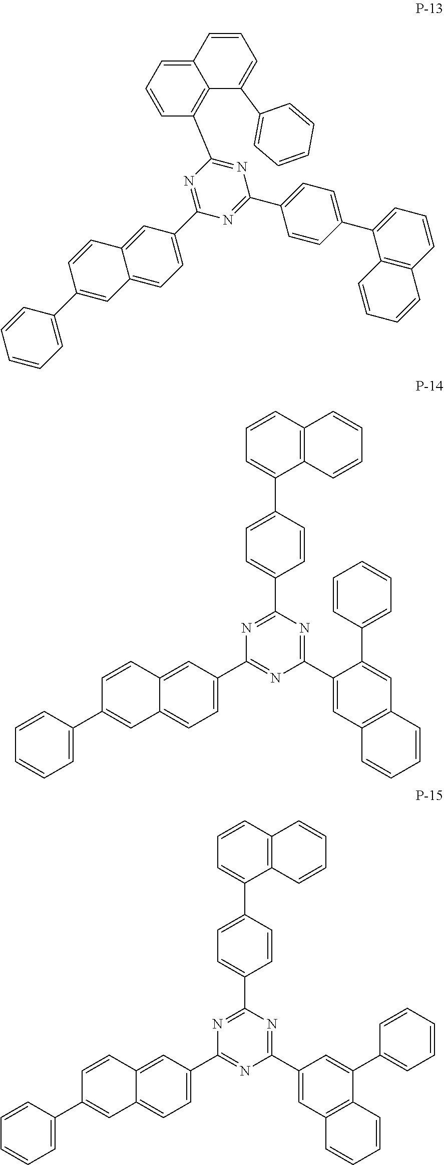 [SMILES: c1ccc(-c2ccc3cc(-c4nc(-c5ccc(-c6cccc7ccccc67)cc5)nc(-c5cccc6cccc(-c7ccccc7)c56)n4)ccc3c2)cc1 ; c1ccc(-c2ccc3cc(-c4nc(-c5ccc(-c6cccc7ccccc67)cc5)nc(-c5cc6ccccc6cc5-c5ccccc5)n4)ccc3c2)cc1 ; c1ccc(-c2ccc3cc(-c4nc(-c5ccc(-c6cccc7ccccc67)cc5)nc(-c5cc(-c6ccccc6)c6ccccc6c5)n4)ccc3c2)cc1]

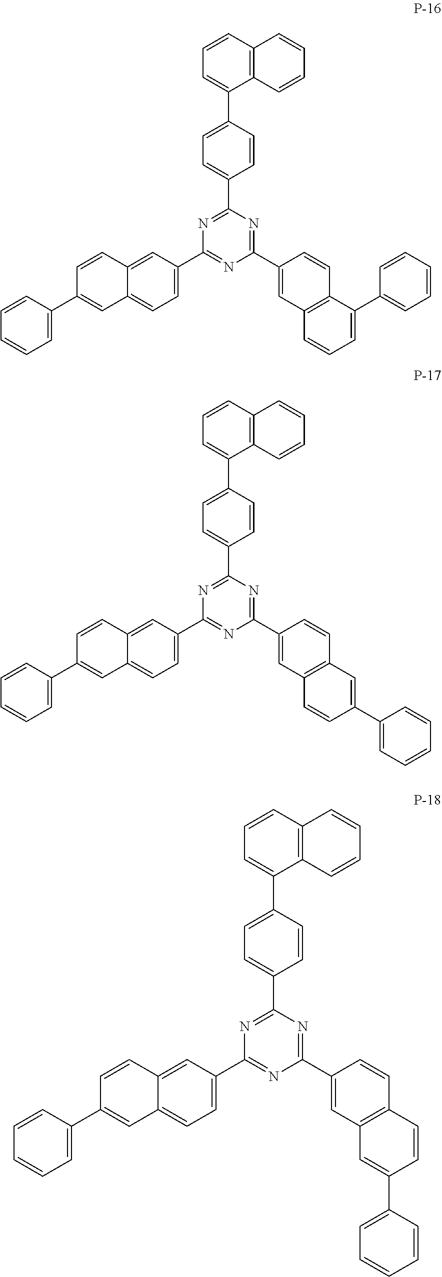 [SMILES: c1ccc(-c2ccc3cc(-c4nc(-c5ccc(-c6cccc7ccccc67)cc5)nc(-c5ccc6c(-c7ccccc7)cccc6c5)n4)ccc3c2)cc1 ; c1ccc(-c2ccc3cc(-c4nc(-c5ccc(-c6cccc7ccccc67)cc5)nc(-c5ccc6cc(-c7ccccc7)ccc6c5)n4)ccc3c2)cc1 ; c1ccc(-c2ccc3cc(-c4nc(-c5ccc(-c6cccc7ccccc67)cc5)nc(-c5ccc6ccc(-c7ccccc7)cc6c5)n4)ccc3c2)cc1]

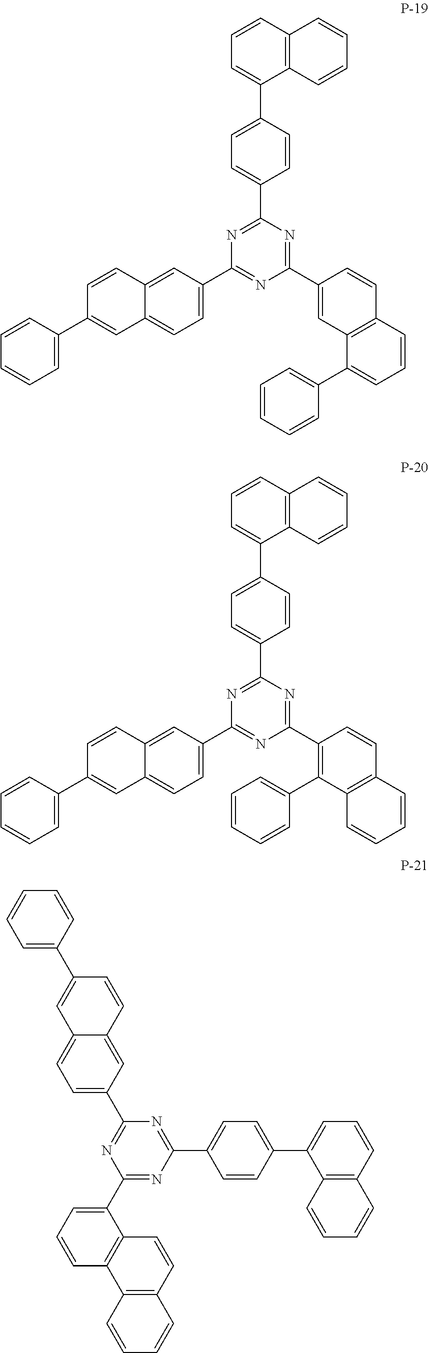 [SMILES: c1ccc(-c2ccc3cc(-c4nc(-c5ccc(-c6cccc7ccccc67)cc5)nc(-c5ccc6cccc(-c7ccccc7)c6c5)n4)ccc3c2)cc1 ; c1ccc(-c2ccc3cc(-c4nc(-c5ccc(-c6cccc7ccccc67)cc5)nc(-c5ccc6ccccc6c5-c5ccccc5)n4)ccc3c2)cc1 ; c1ccc(-c2ccc3cc(-c4nc(-c5ccc(-c6cccc7ccccc67)cc5)nc(-c5cccc6c5ccc5ccccc56)n4)ccc3c2)cc1]

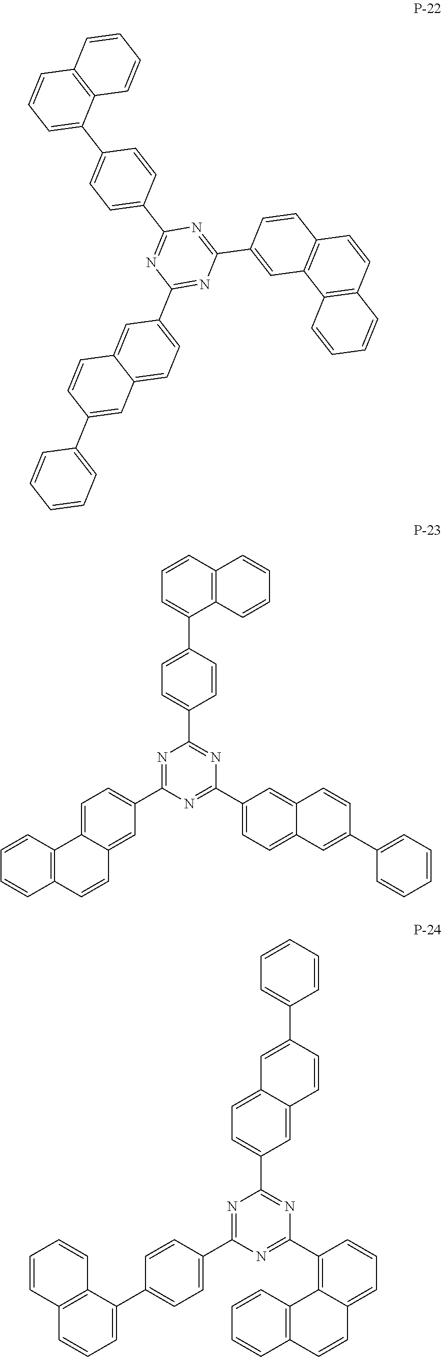 [SMILES: c1ccc(-c2ccc3cc(-c4nc(-c5ccc(-c6cccc7ccccc67)cc5)nc(-c5ccc6ccc7ccccc7c6c5)n4)ccc3c2)cc1 ; c1ccc(-c2ccc3cc(-c4nc(-c5ccc(-c6cccc7ccccc67)cc5)nc(-c5ccc6c(ccc7ccccc76)c5)n4)ccc3c2)cc1 ; c1ccc(-c2ccc3cc(-c4nc(-c5ccc(-c6cccc7ccccc67)cc5)nc(-c5cccc6ccc7ccccc7c56)n4)ccc3c2)cc1]

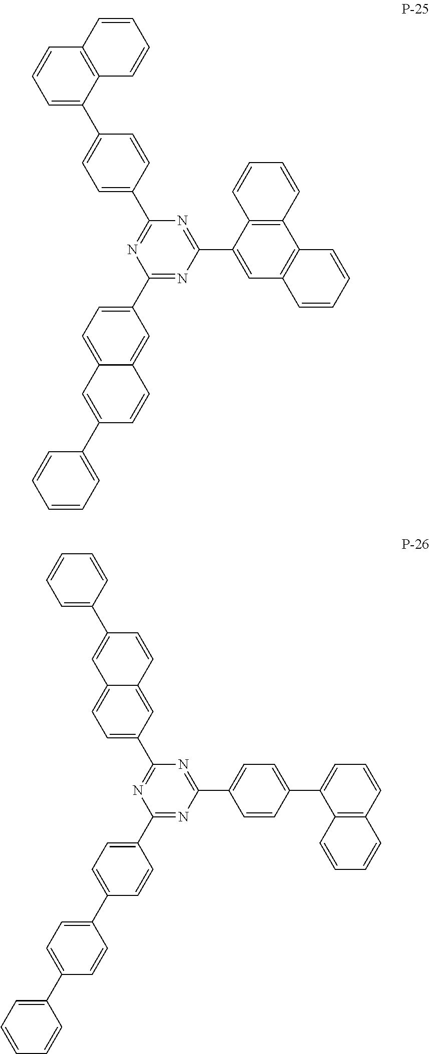 [SMILES: c1ccc(-c2ccc3cc(-c4nc(-c5ccc(-c6cccc7ccccc67)cc5)nc(-c5cc6ccccc6c6ccccc56)n4)ccc3c2)cc1 ; c1ccc(-c2ccc(-c3ccc(-c4nc(-c5ccc(-c6cccc7ccccc67)cc5)nc(-c5ccc6cc(-c7ccccc7)ccc6c5)n4)cc3)cc2)cc1]

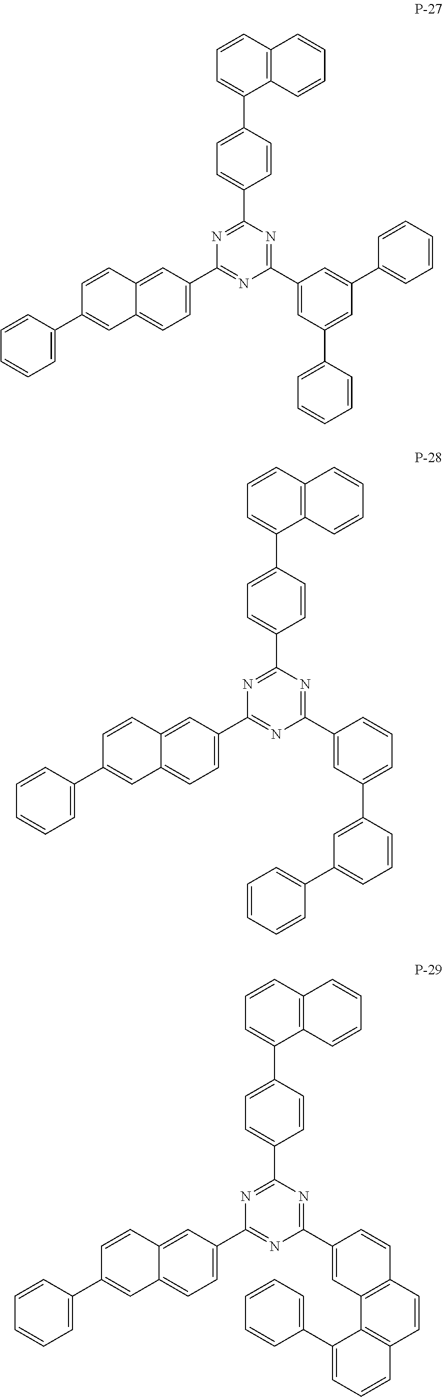

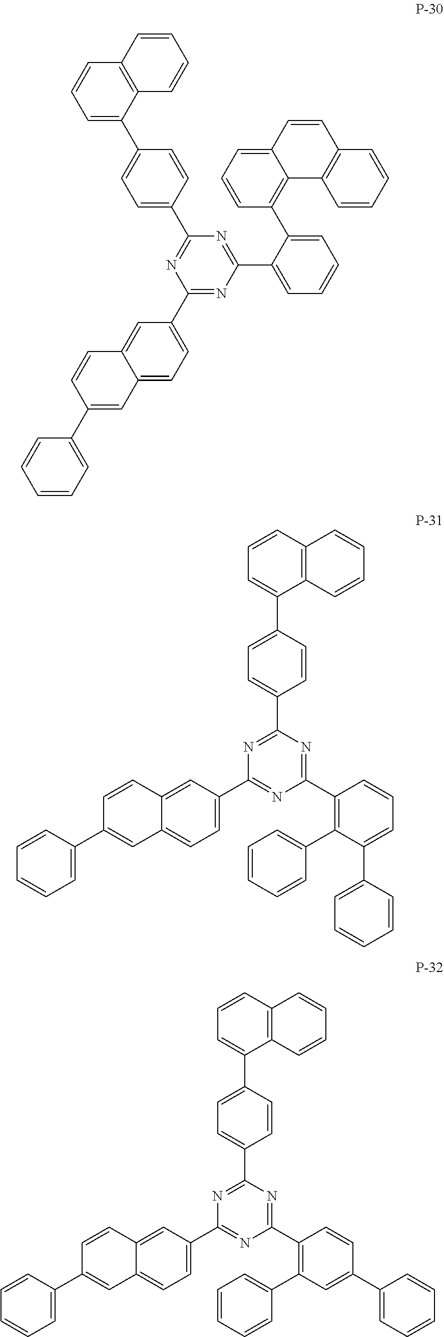 [SMILES: c1ccc(-c2ccc3cc(-c4nc(-c5ccc(-c6cccc7ccccc67)cc5)nc(-c5ccccc5-c5cccc6ccc7ccccc7c56)n4)ccc3c2)cc1 ; c1ccc(-c2ccc3cc(-c4nc(-c5ccc(-c6cccc7ccccc67)cc5)nc(-c5cccc(-c6ccccc6)c5-c5ccccc5)n4)ccc3c2)cc1 ; c1ccc(-c2ccc(-c3nc(-c4ccc(-c5cccc6ccccc56)cc4)nc(-c4ccc5cc(-c6ccccc6)ccc5c4)n3)c(-c3ccccc3)c2)cc1]

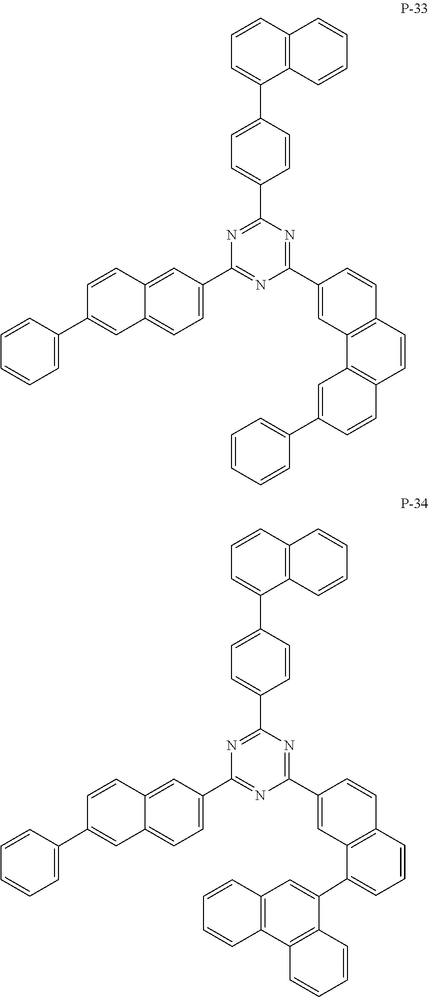

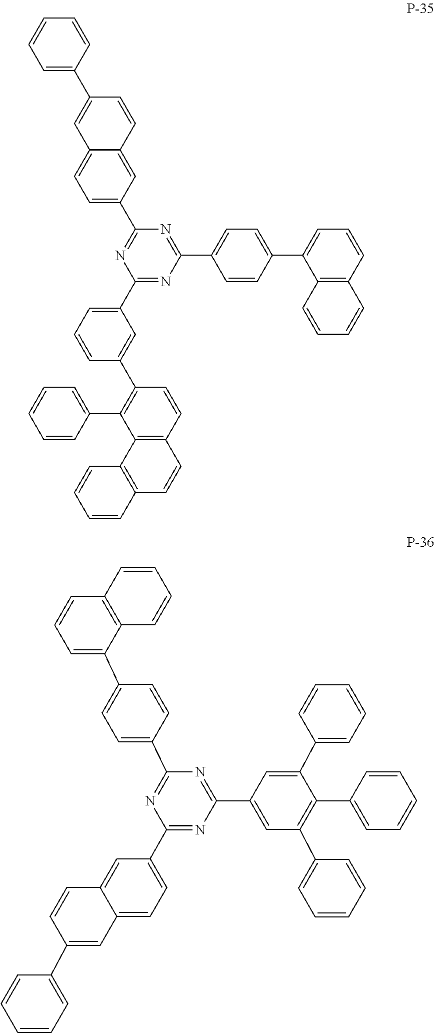 [SMILES: c1ccc(-c2ccc3cc(-c4nc(-c5ccc(-c6cccc7ccccc67)cc5)nc(-c5cccc(-c6ccc7ccc8ccccc8c7c6-c6ccccc6)c5)n4)ccc3c2)cc1 ; c1ccc(-c2ccc3cc(-c4nc(-c5ccc(-c6cccc7ccccc67)cc5)nc(-c5cc(-c6ccccc6)c(-c6ccccc6)c(-c6ccccc6)c5)n4)ccc3c2)cc1]

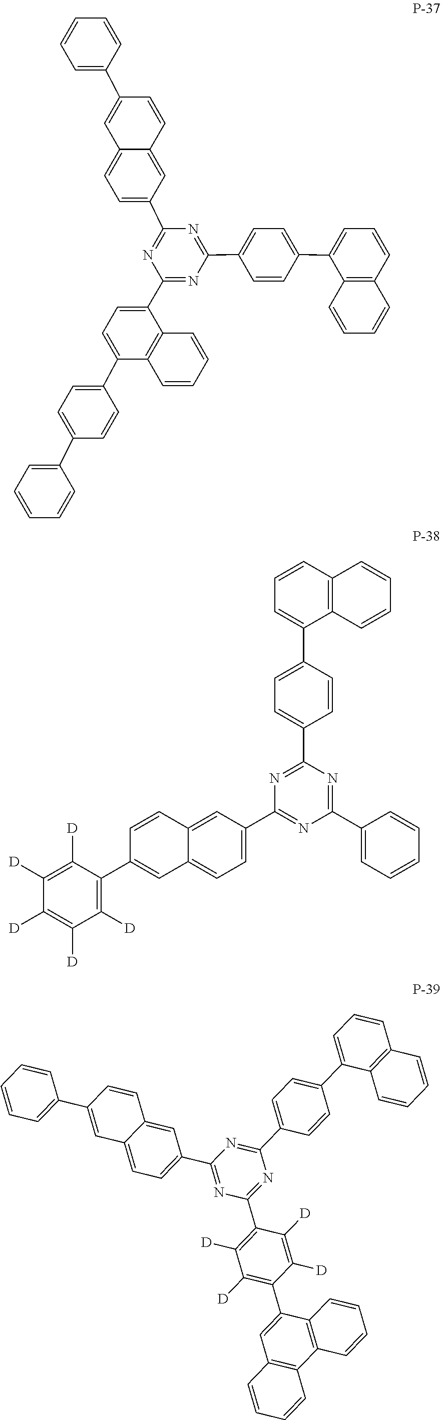 [SMILES: c1ccc(-c2ccc(-c3ccc(-c4nc(-c5ccc(-c6cccc7ccccc67)cc5)nc(-c5ccc6cc(-c7ccccc7)ccc6c5)n4)c4ccccc34)cc2)cc1 ; [2H]c1c([2H])c([2H])c(-c2ccc3cc(-c4nc(-c5ccccc5)nc(-c5ccc(-c6cccc7ccccc67)cc5)n4)ccc3c2)c([2H])c1[2H] ; [2H]c1c([2H])c(-c2cc3ccccc3c3ccccc23)c([2H])c([2H])c1-c1nc(-c2ccc(-c3cccc4ccccc34)cc2)nc(-c2ccc3cc(-c4ccccc4)ccc3c2)n1]

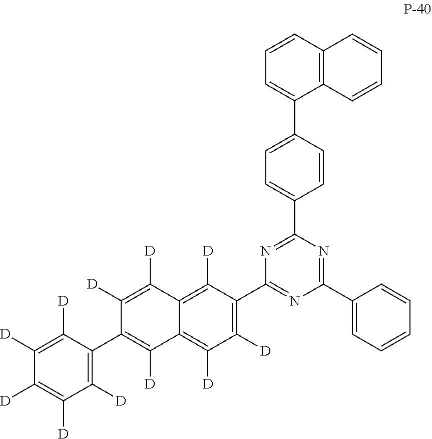 [SMILES: [2H]c1c([2H])c([2H])c(-c2c([2H])c([2H])c3c([2H])c(-c4nc(-c5ccccc5)nc(-c5ccc(-c6cccc7ccccc67)cc5)n4)c([2H])c([2H])c3c2[2H])c([2H])c1[2H]]

Also, in another aspect, the present invention relates to an organic electronic element comprising an anode, a cathode, and an organic material layer formed between the anode and the cathode, wherein the organic material layer comprises a single compound or 2 or more compounds represented by Formula (1).

The organic material layer comprises at least one of a hole injection layer, a hole transport layer, an emitting auxiliary layer, an emitting layer, an electron transport auxiliary layer, an electron transport layer and an electron injection layer.

Also, the present invention provides an organic electric device comprising the compound represented by Formula (1) as a host material of the emitting layer, wherein the dopant material of the emitting layer has a maximum emission wavelength of 610 nm to 620 nm.

Also, the present invention provides an organic electric device comprising the compound represented by Formula (1) as a host material of the emitting layer, wherein the dopant material of the emitting layer has a maximum emission wavelength of 610 nm to 615 nm.

Also, the present invention provides an organic electronic element comprising a compound represented by Formula (1) as a host material of the emitting layer, and a compound represented by Formula (2) or (3) as another heterogeneous host material.

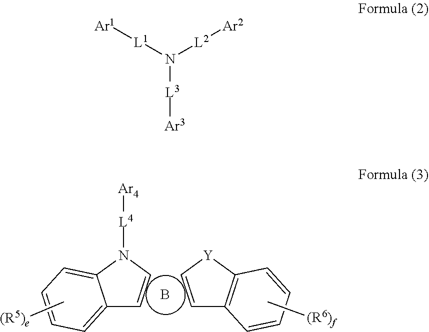

Wherein:
1) Y is O, S, CR′R″ or NR^{a},
2) R′ and R″ are each independently selected from the group consisting of a C₆-C₆₀ aryl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; and a C₆-C₃₀ aryloxy group; or R′ and R″ may be bonded to each other to form a ring, When R and R″ are an aryl group, it is preferably an C₆-C₃₀ aryl group, more preferably an C₆-C₂₅ aryl group, for example, it may be phenylene, biphenyl, naphthalene, terphenyl, and the like.

When R′ and R″ are a heterocyclic group, it is preferably a C₂-C₃₀ heterocyclic group, more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

When R′ and R″ are a fused ring group, it is preferably a fused ring group of a C₃-C₃₀ aliphatic ring and an C₆-C₃₀ aromatic ring, more preferably a fused ring group of an C₃-C₂₄ aliphatic ring and an C₆-C₂₄ aromatic ring.

When R′ and R″ are an alkyl group, it is preferably a C₁-C₃₀ alkyl group, more preferably a C₁-C₂₄ alkyl group.

When R′ and R″ are an alkoxy group, it is preferably a C₁-C₂₄ alkoxy group.

When R′ and R″ are an aryloxy group, it is preferably a C₆-C₂₄ aryloxy group. 3) L¹, L², L³ and L⁴ are each independently selected from the group consisting of single bond; a C₆-C₆₀ arylene group; and a C₂-C₆₀ heteroarylene group;

When L¹, L², L³ and L⁴ are an arylene group, it is preferably an C₆-C₃₀ arylene group, more preferably an C₆-C₂₄ arylene group, for example, it may be phenylene, biphenyl, naphthalene, terphenyl, and the like.

When L¹, L², L³ and L⁴ are an heteroarylene group, it is preferably an C₂-C₃₀ heteroarylene group, more preferably an C₂-C₂₄ heteroarylene group,
4) R^{a}, Ar¹, Ar² and Ar³ are each independently a C₆-C₆₀ aryl group; or a C₂-C₆₀ heteroaryl group;

When R^{a}, Ar¹, Ar² and Ar³ are an aryl group, it is preferably an C₆-C₃₀ aryl group, more preferably an C₆-C₂₅ aryl group, for example, it may be phenylene, biphenyl, naphthalene, terphenyl, and the like.

When R^{a}, Ar¹, Ar² and Ar³ are an heteroaryl group, it is preferably an C₂-C₃₀ heteroaryl group, more preferably an C₂-C₂₄ heteroaryl group,
5) Ar⁴ is selected from the group consisting of a C₆-C₆₀ aryl group; or a C₂-C₆₀ heteroaryl group; and -L′-NR^{b}R^{c};

When Ar⁴ is an aryl group, it is preferably an C₆-C₃₀ aryl group, more preferably an C₆-C₂₅ aryl group, for example, it may be phenylene, biphenyl, naphthalene, terphenyl, and the like.

When Ar⁴ is an heteroaryl group, it is preferably an C₂-C₃₀ heteroaryl group, more preferably an C₂-C₂₄ heteroaryl group,
6) wherein L′ is selected from the group consisting of single bond; a C₆-C₆₀ arylene group; a fluorenylene group; a C₂-C₆₀ heterocyclic group including at least one heteroatom of O, N, S, Si or P; and a C₃-C₆₀ aliphatic ring; wherein R^{b} and R^{c} are each independently selected from the group consisting of an C₆-C₆₀ aryl group; a fluorenyl group; a C₂-C₆₀ heterocyclic group including at least one heteroatom of O, N, S, Si or P; and a fused ring group of a C₃-C₂₀ aliphatic ring and a C₆-C₆₀ aromatic ring;

When L′ is an arylene group, it is preferably an C₆-C₃₀ arylene group, more preferably an C₆-C₂₄ arylene group, for example, it may be phenylene, biphenyl, naphthalene, terphenyl, and the like.

When L′ is a heterocyclic group, it is preferably a C₂-C₃₀ heterocyclic group, more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

When L′ is an aliphatic ring group, preferably a C₃-C₃₀ aliphatic ring group, more preferably a C₃-C₂₄ aliphatic ring group.

When R^{b} and R^{c} are an aryl group, they are preferably an C₆-C₃₀ aryl group, more preferably an C₆-C₂₅ aryl group, for example, it may be phenylene, biphenyl, naphthalene, terphenyl, and the like.

When R^{b} and R^{c} are a heterocyclic group, they are preferably a C₂-C₃₀ heterocyclic group, more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

When R^{b} and R^{c} are a fused ring group, it is preferably a fused ring group of a C₃-C₃₀ aliphatic ring and an C₆-C₃₀ aromatic ring, more preferably a fused ring group of an C₃-C₂₄ aliphatic ring and an C₆-C₂₄ aromatic ring.
7) Ring B is an C₆-C₂₀ aryl group;
8) R⁵ and R⁶ are each independently same or different, and each independently selected from the group consisting of a hydrogen; deuterium; halogen; cyano group; nitro group; C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; and a C₆-C₃₀ aryloxy group; or adjacent plurality of R⁵ or plurality of R⁶ may be bonded to each other to form a ring,
9) e and f are each independently an integer of 0 to 4,
10) wherein the aryl group, arylene group, heterocyclic group, fluorenyl group, fluorenylene group, fused ring group, alkyl group, alkenyl group, alkoxy group and aryloxy group may be substituted with one or more substituents selected from the group consisting of deuterium; halogen; silane group; siloxane group; boron group; germanium group; cyano group; nitro group; C₁-C₂₀ alkylthio group; C₁-C₂₀ alkoxy group; C₁-C₂₀ alkyl group; C₂-C₂₀ alkenyl group; C₂-C₂₀ alkynyl group; C₅-C₂₀ aryl group; C₆-C₂₀ aryl group substituted with deuterium; a fluorenyl group; C₂-C₂₀ heterocyclic group; C₃-C₂₀ cycloalkyl group; C₇-C₂₀ arylalkyl group; C₈-C₂₀ arylalkenyl group; and -L′-NR^{b}R^{c}; and also the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

Also, Formula (2) is represented by any one of the following formulas (2-1) to (2-3):

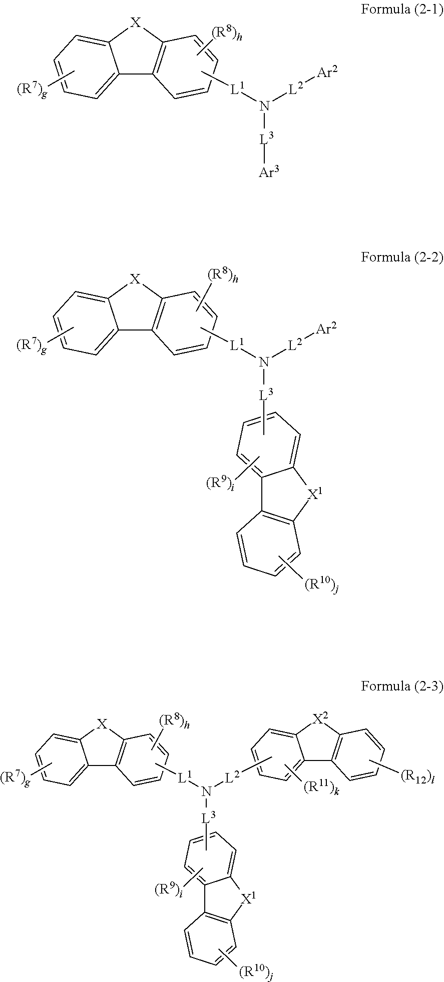

Wherein:
1) X, X¹ and X² are the same as definition of Y,
2) R⁷, R⁸, R⁹, R¹⁰, R¹¹ and R¹² are each the same or different, and each independently selected from the group consisting of hydrogen; deuterium; halogen; cyano group; nitro group; a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; and a C₆-C₆₀ aryloxy group; and -L′-NR^{b}R^{c}; and or an adjacent plurality of R⁷ or plurality of R⁸ or plurality of R⁹ or plurality of R¹⁰ or plurality of R¹¹ or plurality of R¹² may be bonded to each other to form a ring, When R⁷, R⁸, R⁹, R¹⁰, R¹¹ and R¹² are an aryl group, it is preferably an C₆-C₃₀ aryl group, more preferably an C₆-C₂₅ aryl group, for example, it may be phenylene, biphenyl, naphthalene, terphenyl, and the like.

When R⁷, R⁸, R⁹, R¹⁰, R¹¹ and R¹² are a heterocyclic group, it is preferably a C₂-C₃₀ heterocyclic group, more preferably a C₂-C₂₄ heterocyclic group, for example, it may be pyrazine, thiophene, pyridine, pyrimidoindole, 5-phenyl-5H-pyrimido[5,4-b]indole, quinazoline, benzoquinazoline, carbazole, dibenzoquinazoline, dibenzofuran, dibenzothiophene, benzothienopyrimidine, benzofuropyrimidine, phenothiazine, phenylphenothiazine, etc.

When R⁷, R⁸, R⁹, R¹⁰, R¹¹ and R¹² are a fused ring group, it is preferably a fused ring group of a C₃-C₃₀ aliphatic ring and an C₆-C₃₀ aromatic ring, more preferably a fused ring group of an C₃-C₂₄ aliphatic ring and an C₆-C₂₄ aromatic ring.

When R⁷, R⁸, R⁹, R¹⁰, R¹¹ and R¹² are an alkyl group, it is preferably a C₁-C₃₀ alkyl group, more preferably a C₁-C₂₄ alkyl group.

When R⁷, R⁸, R⁹, R¹⁰, R¹¹ and R¹² are an alkenyl group, it is preferably a C₂-C₃₀ alkenyl group, more preferably a C₂-C₂₄ alkenyl group.

When R⁷, R⁸, R⁹, R¹⁰, R¹¹ and R¹² are an alkynyl group, it is preferably a C₂-C₃₀ alkynyl group, more preferably a C₂-C₂₄ alkynyl group.

When R⁷, R⁸, R⁹, R¹⁰, R¹¹ and R¹² are an alkoxy group, it is preferably a C₁-C₃₀ alkoxy group, more preferably a C₁-C₂₄ alkoxy group.

When R⁷, R⁸, R⁹, R¹⁰, R¹¹ and R¹² are an aryloxy group, it is preferably a C₆-C₃₀ aryloxy group, more preferably a C₆-C₂₄ aryloxy group.
3) L¹, L², L³, Ar², Ar³, L′, R^{b} and R^{c} are the same as defined above,
4) g, j and l are each independently an integer of 0 to 4, h, i and k are each independently integer of 0 to 3.

Also, Formula (3) is represented by any one of the following formulas (3-1) to (3-6):

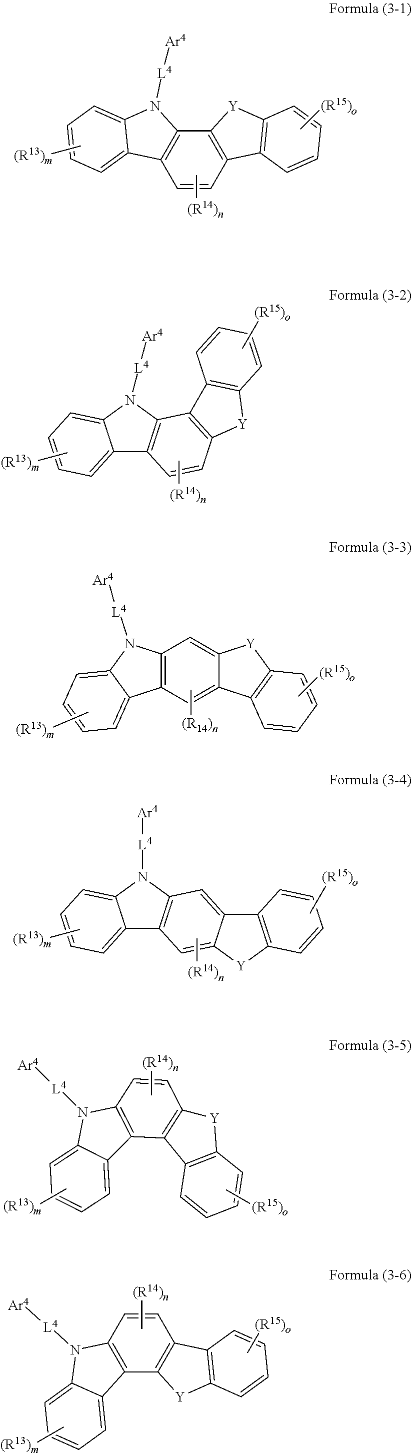

Wherein:
1) Y, L⁴ and Ar⁴ are the same as defined above,
2) R¹³, R¹⁴ and R¹⁵ are the same as definition of R⁵,
3) m and o are each independently an integer of 0 to 4, n is an integer of 0 to 2.

Also, Formula (3) is represented by any one of the following formulas (3-7) to (3-9):

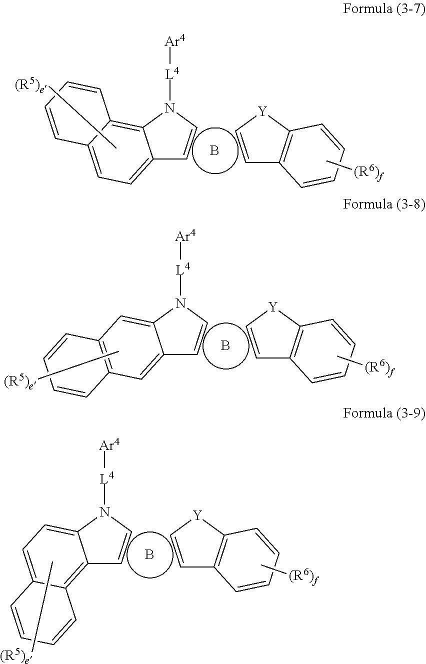

Wherein:
1) Ring B, R⁵, R⁶, f, Y, L⁴ and Ar⁴ are the same as defined above,
2) e′ is an integer of 0 to 6.

Also, Formula (3) is represented by any one of the following formulas (3-10) to (3-12):

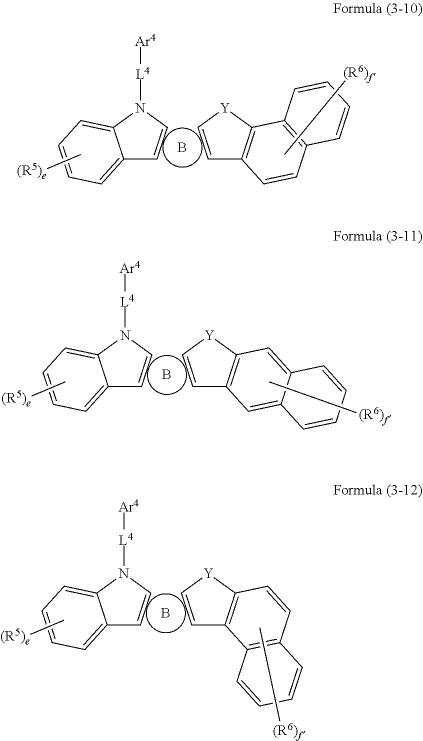

1) Ring B, R⁵, R⁶, e, Y, L⁴ and Ar⁴ are the same as defined above,
2) f′ is an integer of 0 to 6.

Also, Formula (3) is represented by any one of the following formulas (3-13) to (3-18):

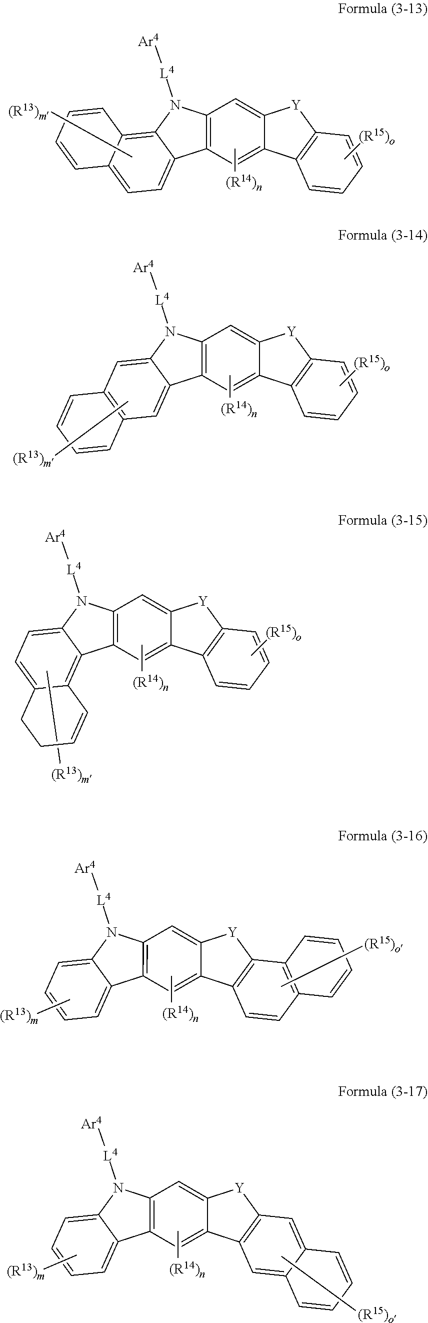

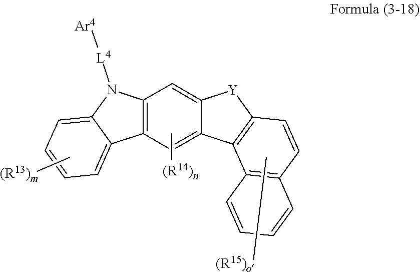

Wherein:
1) Y, L⁴, Ar⁴, R¹³, R¹⁴, R¹⁵, m, n and o are the same as defined above,
2) m′ and o′ are each independently an integer of 0 to 6.

Also, Formula (3) is represented by Formula (3-19):

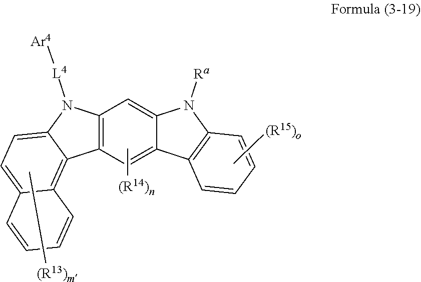

Wherein:
1) Ar⁴ and R^{a} are each independently a C₆-C₁₈ aryl group;
2) L⁴ is a single bond; or a C₆-C₆₀ arylene group.

When L⁴ is an arylene group, it is preferably an C₆-C₃₀ arylene group, more preferably an C₆-C₂₄ arylene group, for example, it may be phenylene, biphenyl, naphthalene, terphenyl, and the like.
3) R¹³, R¹⁴, R¹⁵, n and o are the same as defined above,
4) m′ is an integer of 0 to 6.

Also, the compound represented by Formula (2) is represented by any one of the following compounds N-1 to N-100.

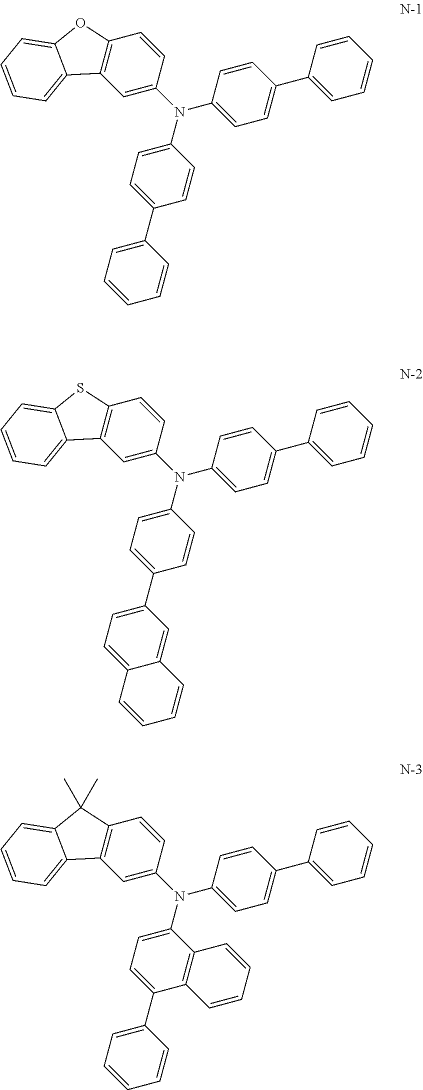 [SMILES: c1ccc(-c2ccc(N(c3ccc(-c4ccccc4)cc3)c3ccc4oc5ccccc5c4c3)cc2)cc1 ; c1ccc(-c2ccc(N(c3ccc(-c4ccc5ccccc5c4)cc3)c3ccc4sc5ccccc5c4c3)cc2)cc1 ; CC1(C)c2ccccc2-c2cc(N(c3ccc(-c4ccccc4)cc3)c3ccc(-c4ccccc4)c4ccccc34)ccc21]

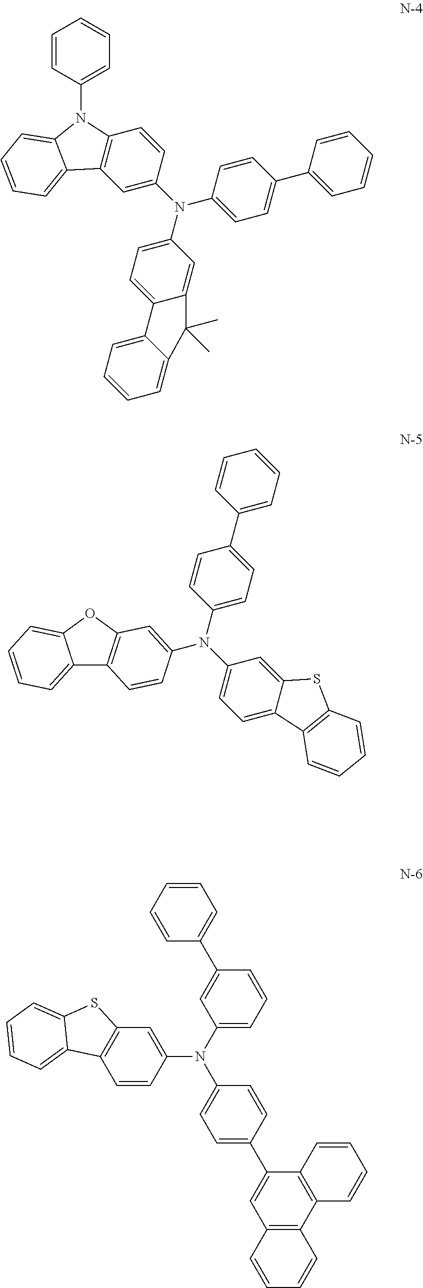 [SMILES: CC1(C)c2ccccc2-c2ccc(N(c3ccc(-c4ccccc4)cc3)c3ccc4c(c3)c3ccccc3n4-c3ccccc3)cc21 ; c1ccc(-c2ccc(N(c3ccc4c(c3)oc3ccccc34)c3ccc4c(c3)sc3ccccc34)cc2)cc1 ; c1ccc(-c2cccc(N(c3ccc(-c4cc5ccccc5c5ccccc45)cc3)c3ccc4c(c3)sc3ccccc34)c2)cc1]

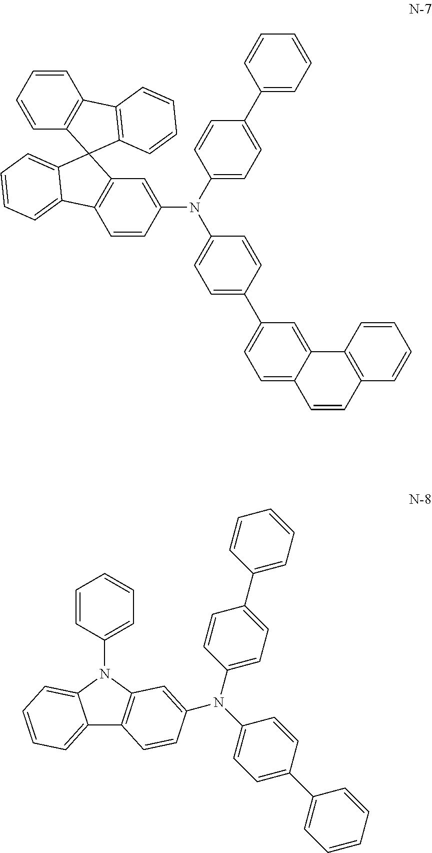 [SMILES: c1ccc(-c2ccc(N(c3ccc(-c4ccc5ccc6ccccc6c5c4)cc3)c3ccc4c(c3)C3(c5ccccc5-c5ccccc53)c3ccccc3-4)cc2)cc1 ; c1ccc(-c2ccc(N(c3ccc(-c4ccccc4)cc3)c3ccc4c5ccccc5n(-c5ccccc5)c4c3)cc2)cc1]

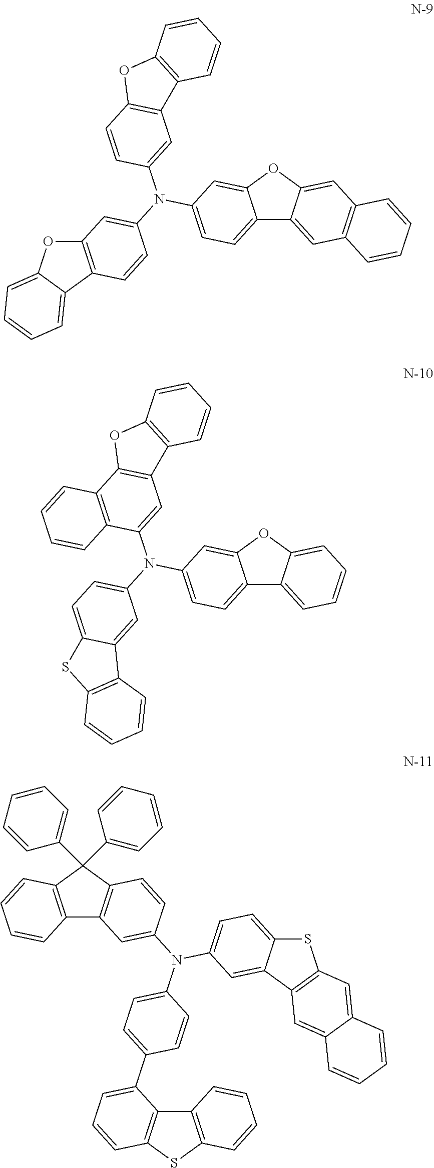

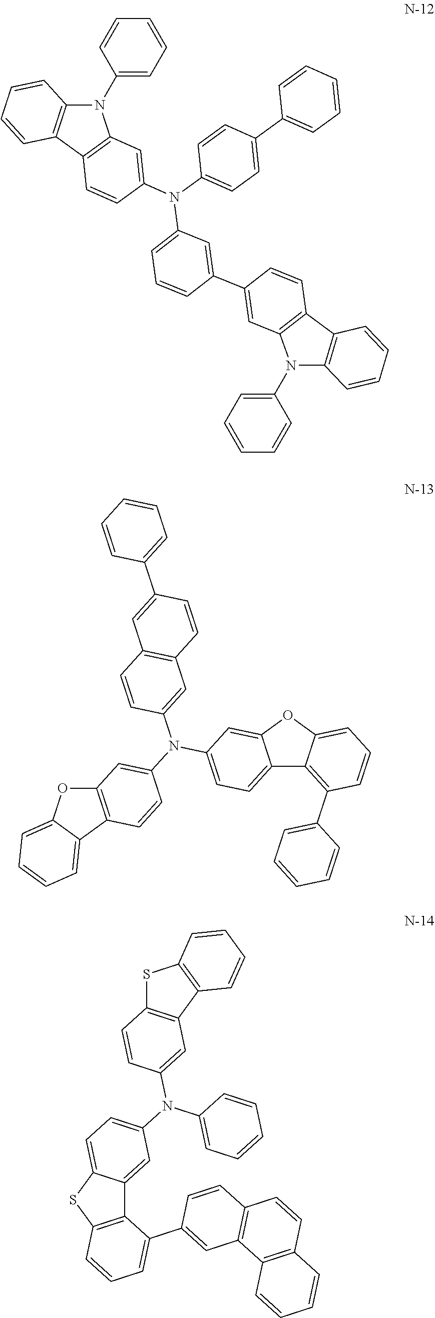 [SMILES: c1ccc(-c2ccc(N(c3cccc(-c4ccc5c6ccccc6n(-c6ccccc6)c5c4)c3)c3ccc4c5ccccc5n(-c5ccccc5)c4c3)cc2)cc1 ; c1ccc(-c2ccc3cc(N(c4ccc5c(c4)oc4ccccc45)c4ccc5c(c4)oc4cccc(-c6ccccc6)c45)ccc3c2)cc1 ; c1ccc(N(c2ccc3sc4ccccc4c3c2)c2ccc3sc4cccc(-c5ccc6ccc7ccccc7c6c5)c4c3c2)cc1]

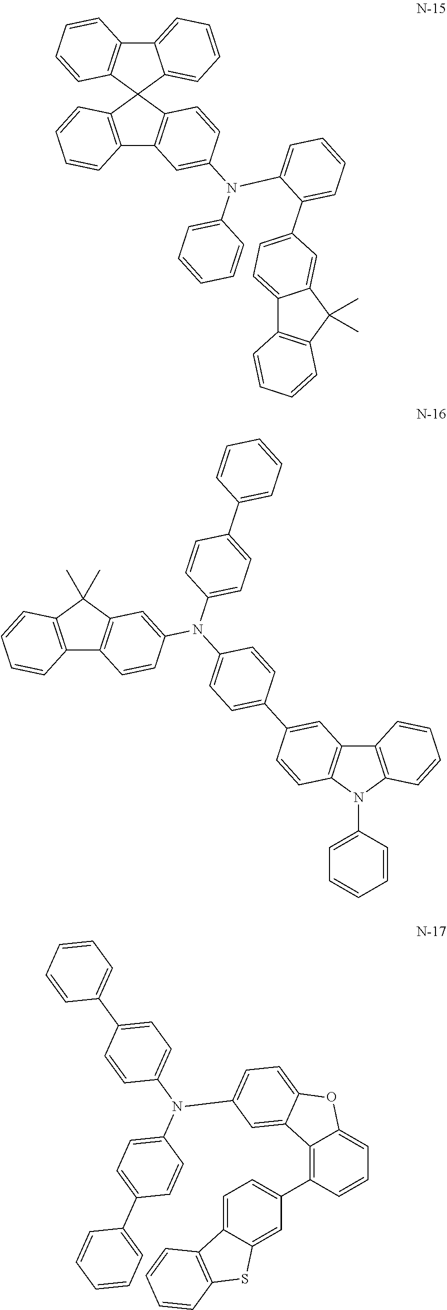 [SMILES: CC1(C)c2ccccc2-c2ccc(-c3ccccc3N(c3ccccc3)c3ccc4c(c3)-c3ccccc3C43c4ccccc4-c4ccccc43)cc21 ; CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccccc4)cc3)c3ccc4oc5cccc(-c6ccc7c(c6)sc6ccccc67)c5c4c3)cc2)cc1]

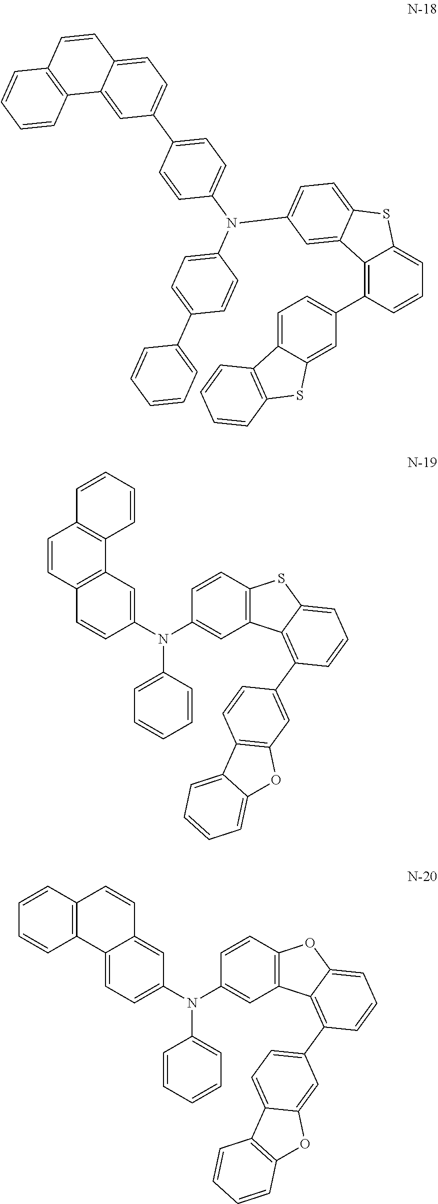 [SMILES: c1ccc(-c2ccc(N(c3ccc(-c4ccc5ccc6ccccc6c5c4)cc3)c3ccc4sc5cccc(-c6ccc7c(c6)sc6ccccc67)c5c4c3)cc2)cc1 ; c1ccc(N(c2ccc3ccc4ccccc4c3c2)c2ccc3sc4cccc(-c5ccc6c(c5)oc5ccccc56)c4c3c2)cc1 ; c1ccc(N(c2ccc3c(ccc4ccccc43)c2)c2ccc3oc4cccc(-c5ccc6c(c5)oc5ccccc56)c4c3c2)cc1]

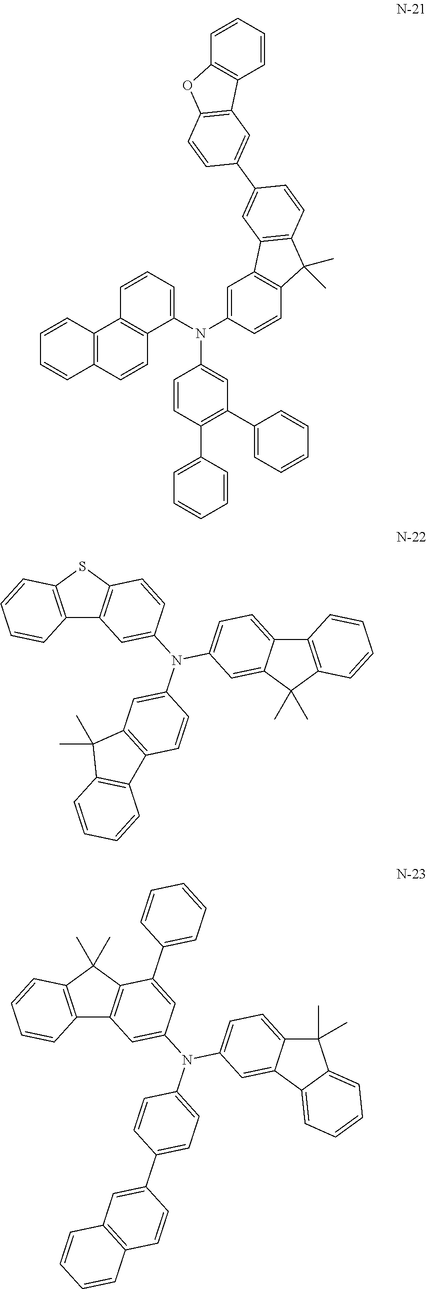 [SMILES: CC1(C)c2ccc(-c3ccc4oc5ccccc5c4c3)cc2-c2cc(N(c3ccc(-c4ccccc4)c(-c4ccccc4)c3)c3cccc4c3ccc3ccccc34)ccc21 ; CC1(C)c2ccccc2-c2ccc(N(c3ccc4c(c3)C(C)(C)c3ccccc3-4)c3ccc4sc5ccccc5c4c3)cc21 ; CC1(C)c2ccccc2-c2cc(N(c3ccc(-c4ccc5ccccc5c4)cc3)c3cc(-c4ccccc4)c4c(c3)-c3ccccc3C4(C)C)ccc21]

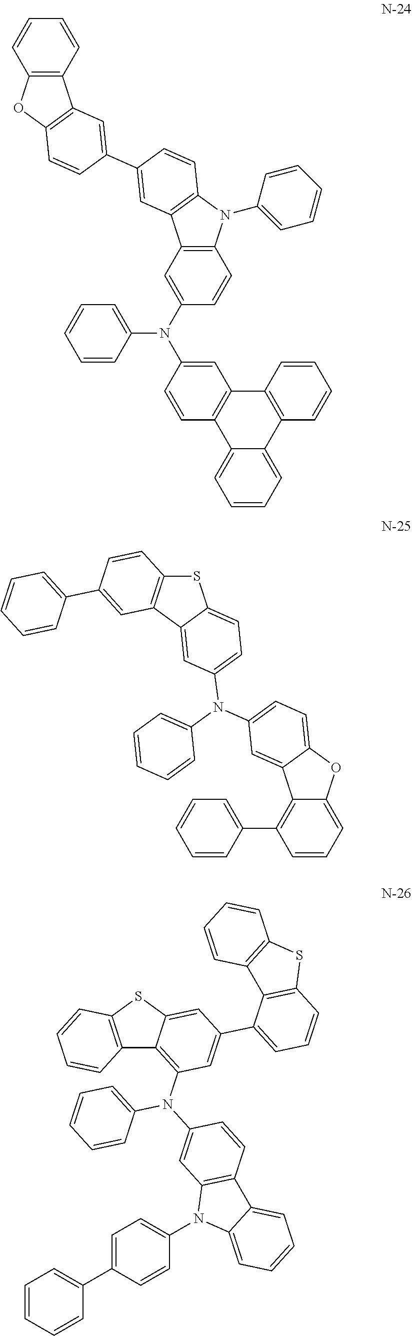

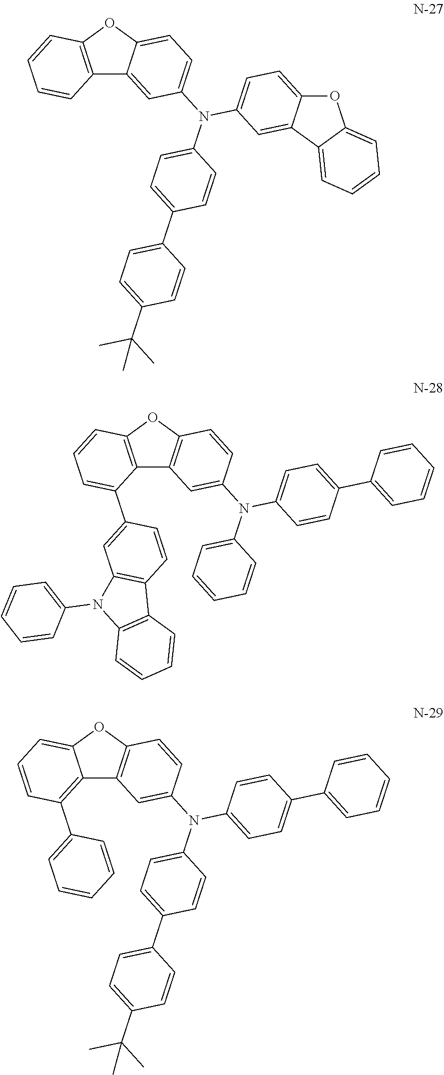 [SMILES: CC(C)(C)c1ccc(-c2ccc(N(c3ccc4oc5ccccc5c4c3)c3ccc4oc5ccccc5c4c3)cc2)cc1 ; c1ccc(-c2ccc(N(c3ccccc3)c3ccc4oc5cccc(-c6ccc7c8ccccc8n(-c8ccccc8)c7c6)c5c4c3)cc2)cc1 ; CC(C)(C)c1ccc(-c2ccc(N(c3ccc(-c4ccccc4)cc3)c3ccc4oc5cccc(-c6ccccc6)c5c4c3)cc2)cc1]

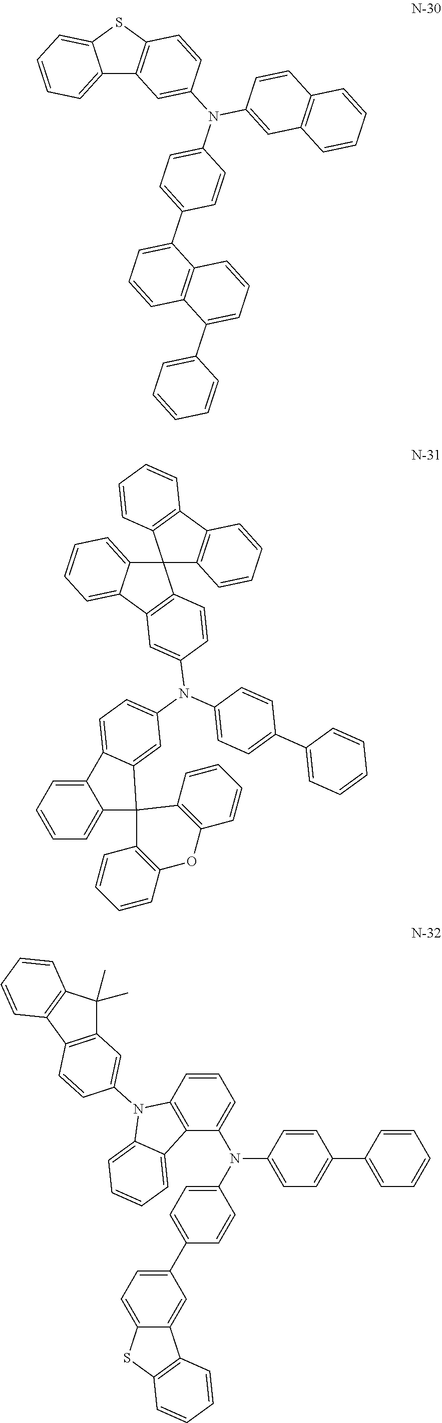 [SMILES: c1ccc(-c2cccc3c(-c4ccc(N(c5ccc6ccccc6c5)c5ccc6sc7ccccc7c6c5)cc4)cccc23)cc1 ; c1ccc(-c2ccc(N(c3ccc4c(c3)-c3ccccc3C43c4ccccc4-c4ccccc43)c3ccc4c(c3)C3(c5ccccc5Oc5ccccc53)c3ccccc3-4)cc2)cc1 ; CC1(C)c2ccccc2-c2ccc(-n3c4ccccc4c4c(N(c5ccc(-c6ccccc6)cc5)c5ccc(-c6ccc7sc8ccccc8c7c6)cc5)cccc43)cc21]

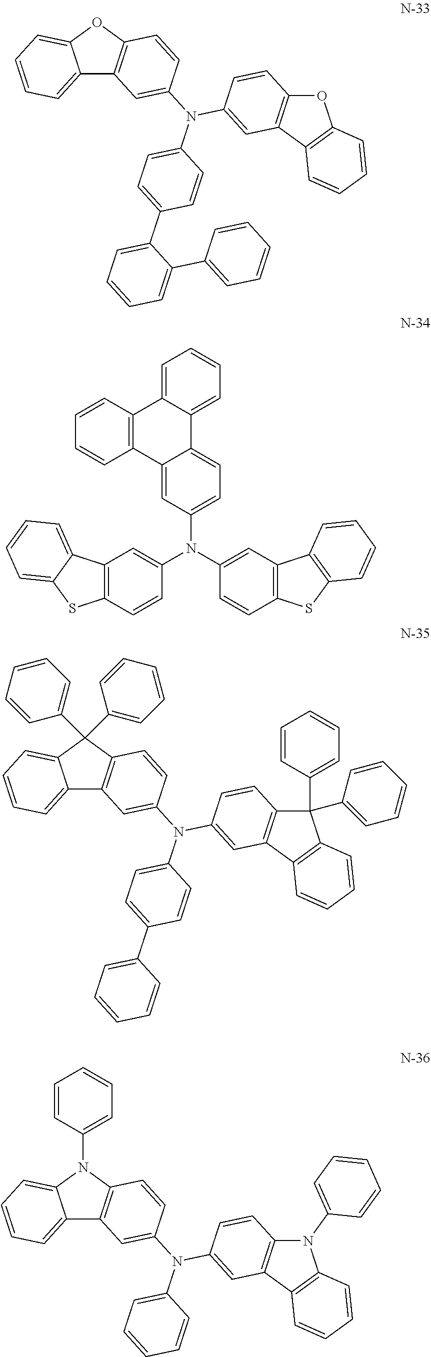

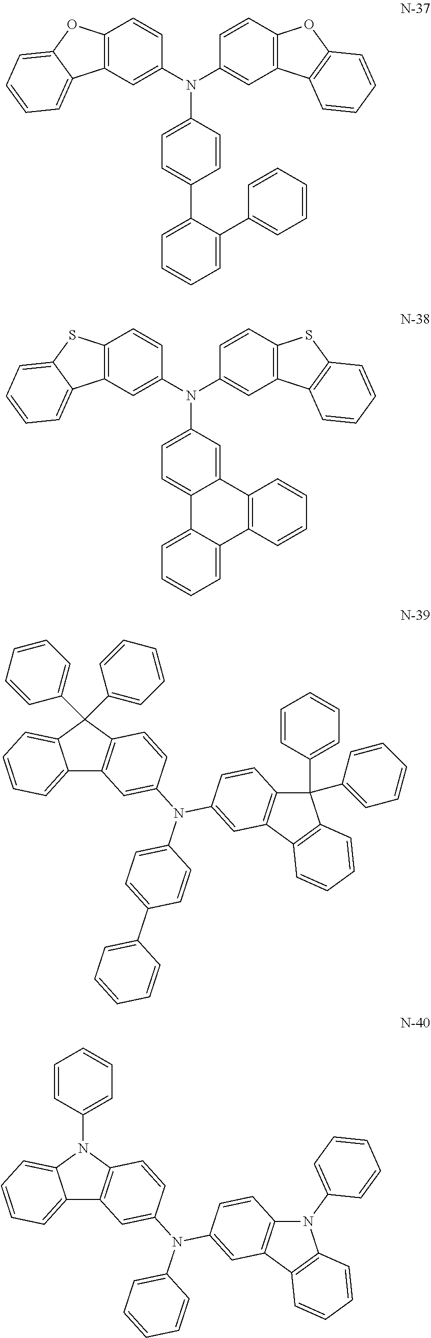 [SMILES: c1ccc(-c2ccccc2-c2ccc(N(c3ccc4oc5ccccc5c4c3)c3ccc4oc5ccccc5c4c3)cc2)cc1 ; c1ccc2c(c1)sc1ccc(N(c3ccc4sc5ccccc5c4c3)c3ccc4c5ccccc5c5ccccc5c4c3)cc12 ; c1ccc(-c2ccc(N(c3ccc4c(c3)-c3ccccc3C4(c3ccccc3)c3ccccc3)c3ccc4c(c3)-c3ccccc3C4(c3ccccc3)c3ccccc3)cc2)cc1 ; c1ccc(N(c2ccc3c(c2)c2ccccc2n3-c2ccccc2)c2ccc3c(c2)c2ccccc2n3-c2ccccc2)cc1]

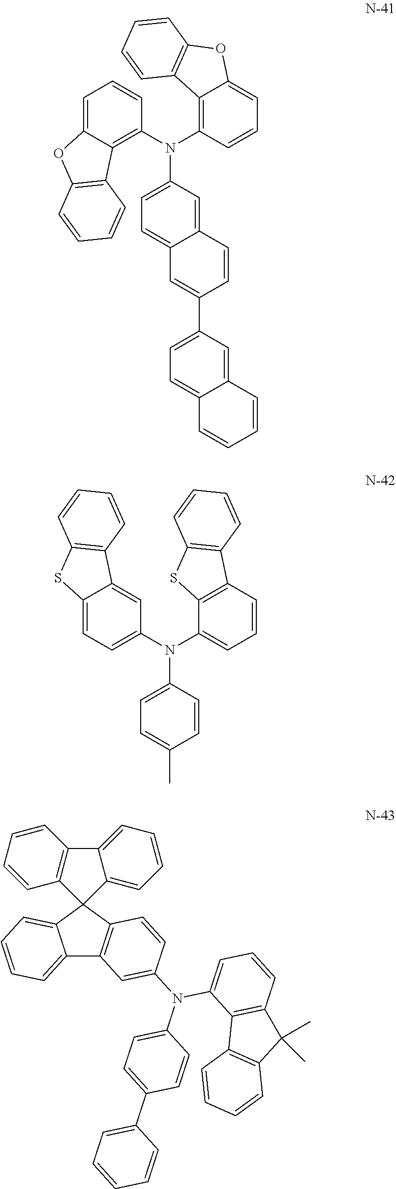

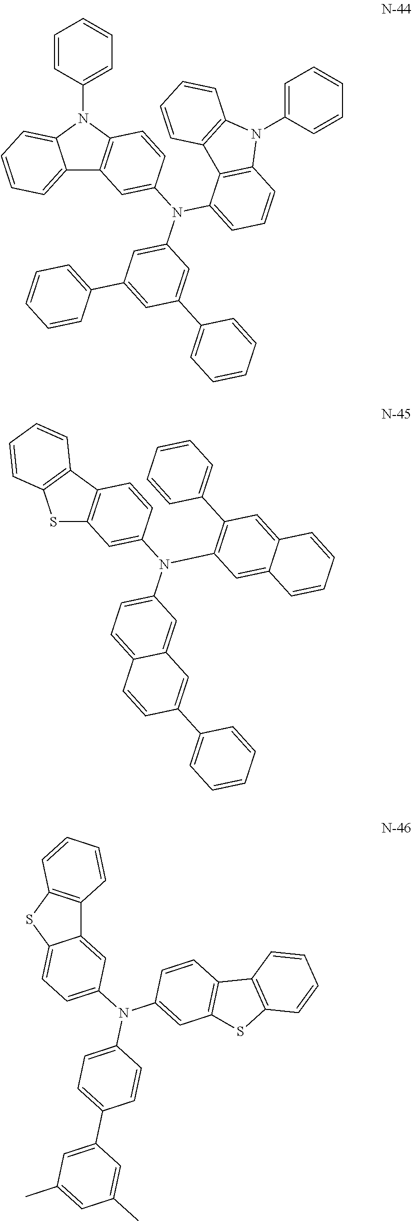 [SMILES: c1ccc(-c2cc(-c3ccccc3)cc(N(c3ccc4c(c3)c3ccccc3n4-c3ccccc3)c3cccc4c3c3ccccc3n4-c3ccccc3)c2)cc1 ; c1ccc(-c2ccc3ccc(N(c4ccc5c(c4)sc4ccccc45)c4cc5ccccc5cc4-c4ccccc4)cc3c2)cc1 ; Cc1cc(C)cc(-c2ccc(N(c3ccc4c(c3)sc3ccccc34)c3ccc4sc5ccccc5c4c3)cc2)c1]

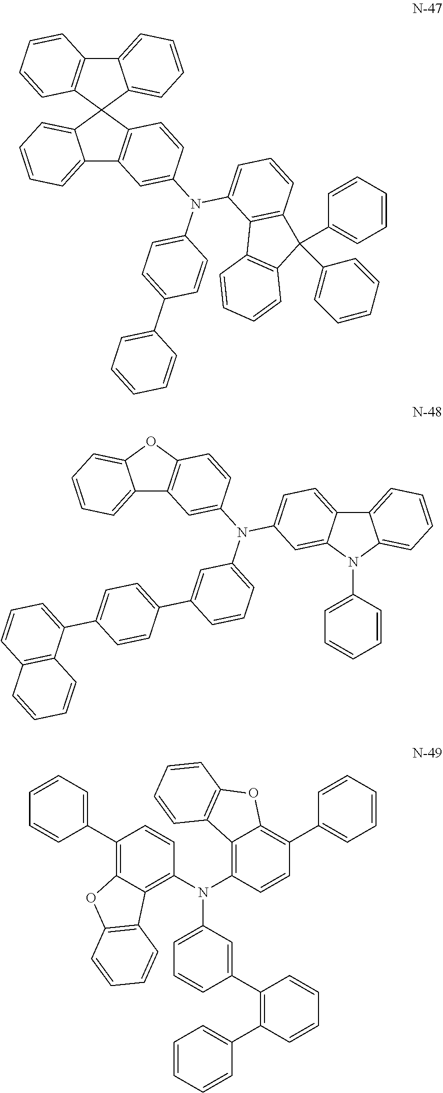 [SMILES: c1ccc(-c2ccc(N(c3ccc4c(c3)-c3ccccc3C43c4ccccc4-c4ccccc43)c3cccc4c3-c3ccccc3C4(c3ccccc3)c3ccccc3)cc2)cc1 ; c1ccc(-n2c3ccccc3c3ccc(N(c4cccc(-c5ccc(-c6cccc7ccccc67)cc5)c4)c4ccc5oc6ccccc6c5c4)cc32)cc1 ; c1ccc(-c2ccccc2-c2cccc(N(c3ccc(-c4ccccc4)c4oc5ccccc5c34)c3ccc(-c4ccccc4)c4oc5ccccc5c34)c2)cc1]

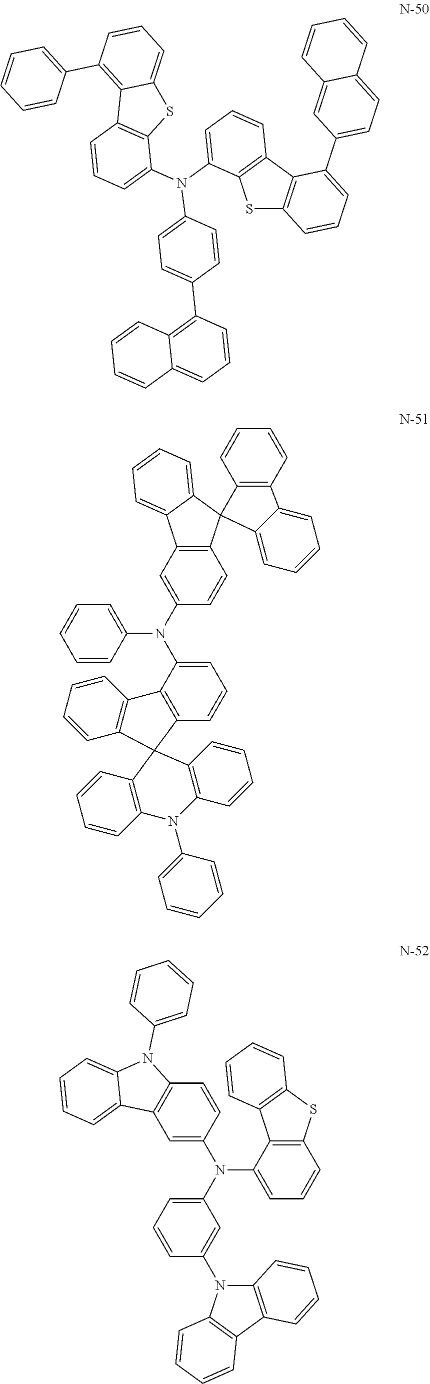 [SMILES: c1ccc(-c2cccc3sc4c(N(c5ccc(-c6cccc7ccccc67)cc5)c5cccc6c5sc5cccc(-c7ccc8ccccc8c7)c56)cccc4c23)cc1 ; c1ccc(N(c2ccc3c(c2)-c2ccccc2C32c3ccccc3-c3ccccc32)c2cccc3c2-c2ccccc2C32c3ccccc3N(c3ccccc3)c3ccccc32)cc1 ; c1ccc(-n2c3ccccc3c3cc(N(c4cccc(-n5c6ccccc6c6ccccc65)c4)c4cccc5sc6ccccc6c45)ccc32)cc1]

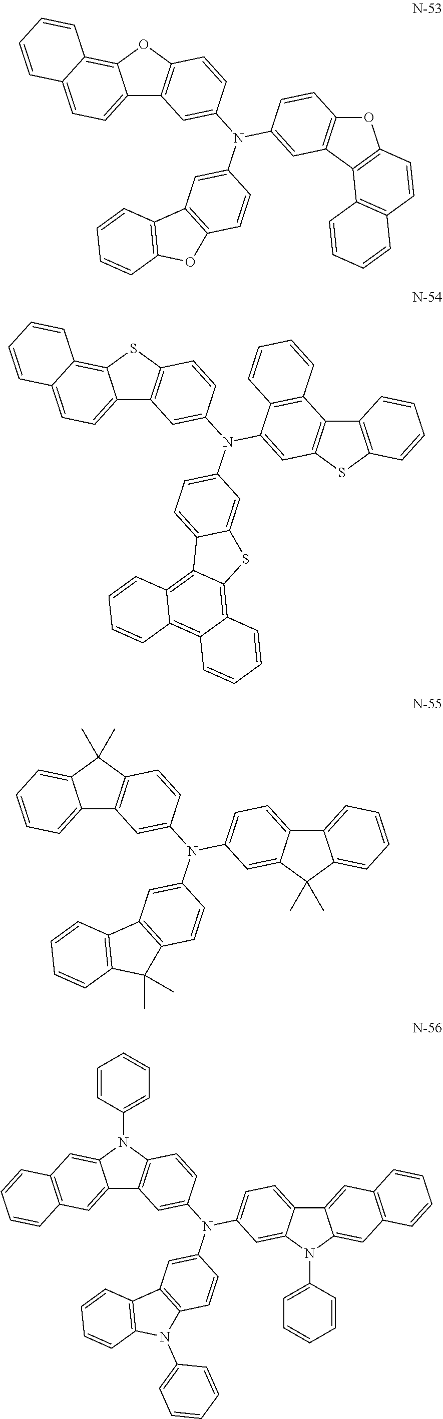 [SMILES: c1ccc2c(c1)ccc1c3cc(N(c4ccc5oc6ccccc6c5c4)c4ccc5oc6ccc7ccccc7c6c5c4)ccc3oc21 ; c1ccc2c(c1)ccc1c3cc(N(c4ccc5c(c4)sc4c6ccccc6c6ccccc6c54)c4cc5sc6ccccc6c5c5ccccc45)ccc3sc21 ; CC1(C)c2ccccc2-c2cc(N(c3ccc4c(c3)-c3ccccc3C4(C)C)c3ccc4c(c3)C(C)(C)c3ccccc3-4)ccc21 ; c1ccc(-n2c3ccccc3c3cc(N(c4ccc5c(c4)c4cc6ccccc6cc4n5-c4ccccc4)c4ccc5c6cc7ccccc7cc6n(-c6ccccc6)c5c4)ccc32)cc1]

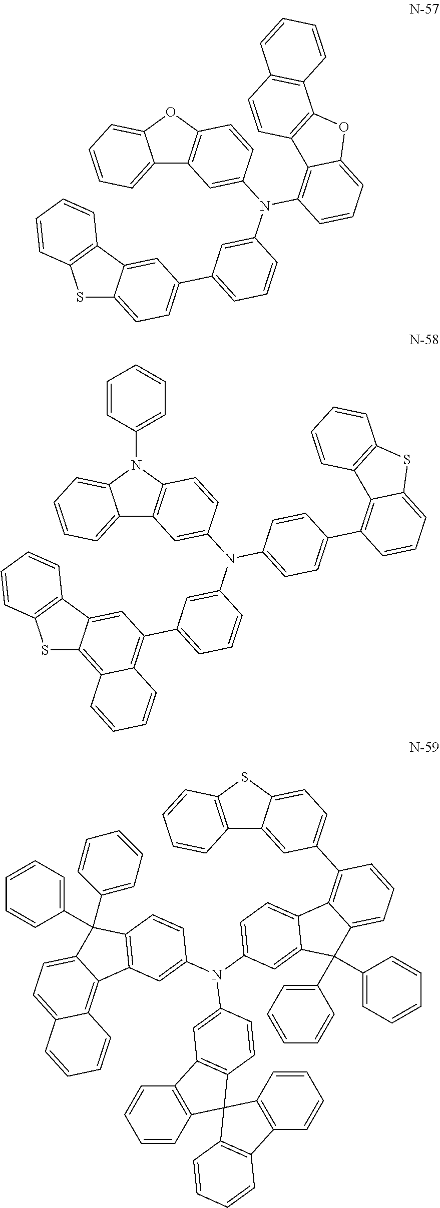

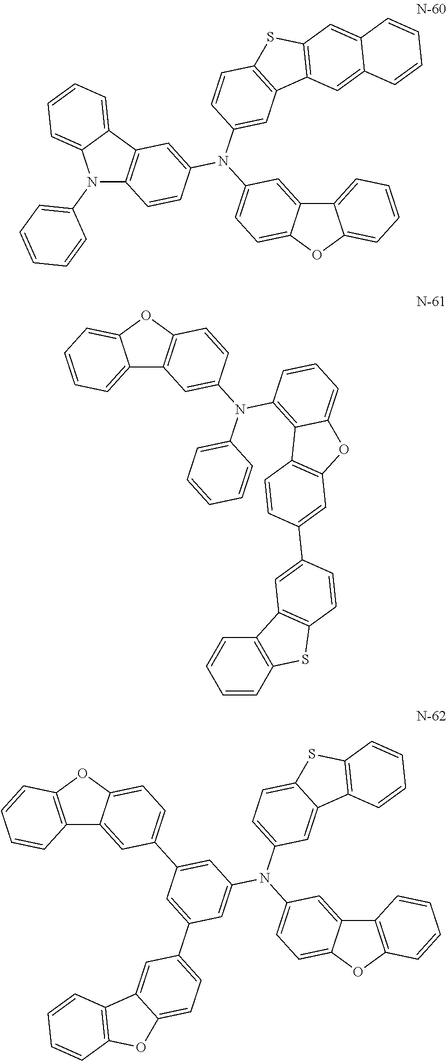 [SMILES: c1ccc(-n2c3ccccc3c3cc(N(c4ccc5oc6ccccc6c5c4)c4ccc5sc6cc7ccccc7cc6c5c4)ccc32)cc1 ; c1ccc(N(c2ccc3oc4ccccc4c3c2)c2cccc3oc4cc(-c5ccc6sc7ccccc7c6c5)ccc4c23)cc1 ; c1ccc2c(c1)oc1ccc(-c3cc(-c4ccc5oc6ccccc6c5c4)cc(N(c4ccc5oc6ccccc6c5c4)c4ccc5sc6ccccc6c5c4)c3)cc12]

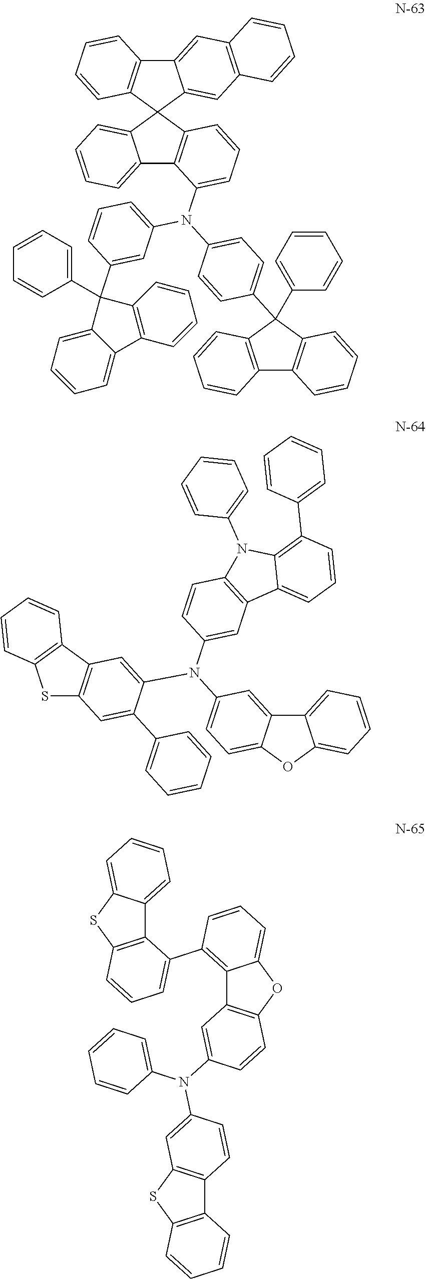 [SMILES: c1ccc(C2(c3ccc(N(c4cccc(C5(c6ccccc6)c6ccccc6-c6ccccc65)c4)c4cccc5c4-c4ccccc4C54c5ccccc5-c5cc6ccccc6cc54)cc3)c3ccccc3-c3ccccc32)cc1 ; c1ccc(-c2cc3sc4ccccc4c3cc2N(c2ccc3oc4ccccc4c3c2)c2ccc3c(c2)c2cccc(-c4ccccc4)c2n3-c2ccccc2)cc1 ; c1ccc(N(c2ccc3c(c2)sc2ccccc23)c2ccc3oc4cccc(-c5cccc6sc7ccccc7c56)c4c3c2)cc1]

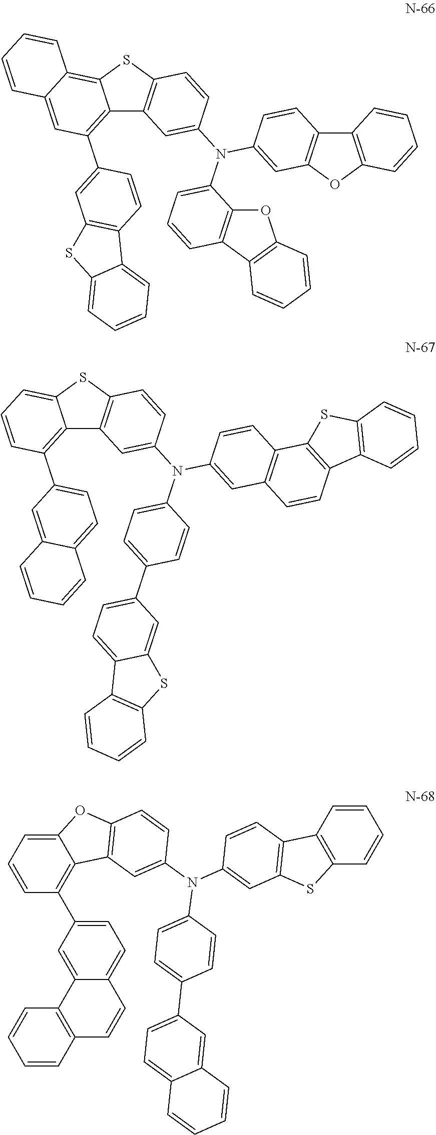 [SMILES: c1ccc2c(c1)cc(-c1ccc3c(c1)sc1ccccc13)c1c3cc(N(c4ccc5c(c4)oc4ccccc45)c4cccc5c4oc4ccccc45)ccc3sc21 ; c1ccc2cc(-c3cccc4sc5ccc(N(c6ccc(-c7ccc8c(c7)sc7ccccc78)cc6)c6ccc7c(ccc8c9ccccc9sc78)c6)cc5c34)ccc2c1 ; c1ccc2cc(-c3ccc(N(c4ccc5c(c4)sc4ccccc45)c4ccc5oc6cccc(-c7ccc8ccc9ccccc9c8c7)c6c5c4)cc3)ccc2c1]

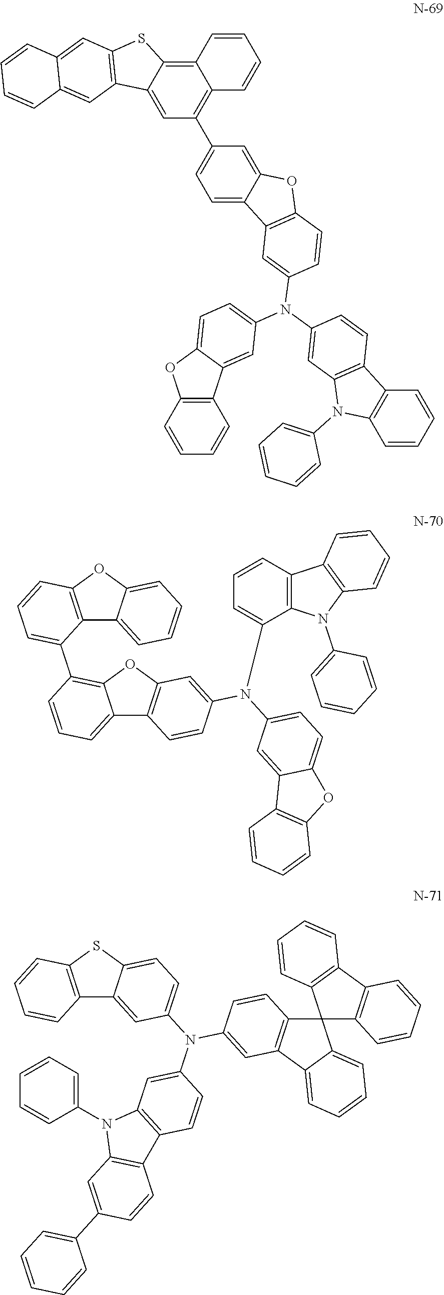 [SMILES: c1ccc(-n2c3ccccc3c3ccc(N(c4ccc5oc6ccccc6c5c4)c4ccc5oc6cc(-c7cc8c9cc%10ccccc%10cc9sc8c8ccccc78)ccc6c5c4)cc32)cc1 ; c1ccc(-n2c3ccccc3c3cccc(N(c4ccc5c(c4)oc4c(-c6cccc7oc8ccccc8c67)cccc45)c4ccc5oc6ccccc6c5c4)c32)cc1 ; c1ccc(-c2ccc3c4ccc(N(c5ccc6c(c5)-c5ccccc5C65c6ccccc6-c6ccccc65)c5ccc6sc7ccccc7c6c5)cc4n(-c4ccccc4)c3c2)cc1]

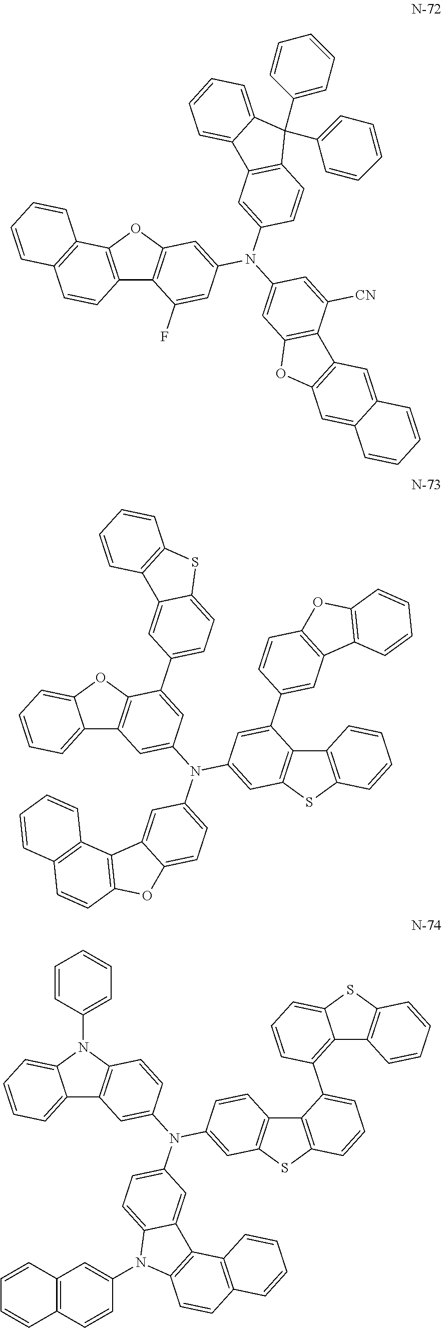

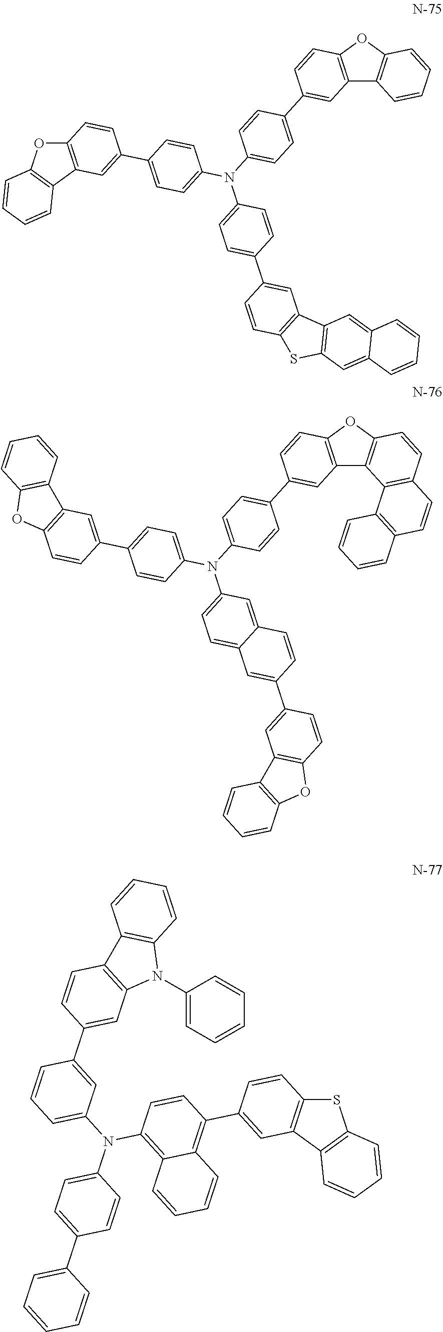 [SMILES: c1ccc2cc3c(cc2c1)sc1ccc(-c2ccc(N(c4ccc(-c5ccc6oc7ccccc7c6c5)cc4)c4ccc(-c5ccc6oc7ccccc7c6c5)cc4)cc2)cc13 ; c1ccc2c(c1)ccc1ccc3oc4ccc(-c5ccc(N(c6ccc(-c7ccc8oc9ccccc9c8c7)cc6)c6ccc7cc(-c8ccc9oc%10ccccc%10c9c8)ccc7c6)cc5)cc4c3c12 ; c1ccc(-c2ccc(N(c3cccc(-c4ccc5c6ccccc6n(-c6ccccc6)c5c4)c3)c3ccc(-c4ccc5sc6ccccc6c5c4)c4ccccc34)cc2)cc1]

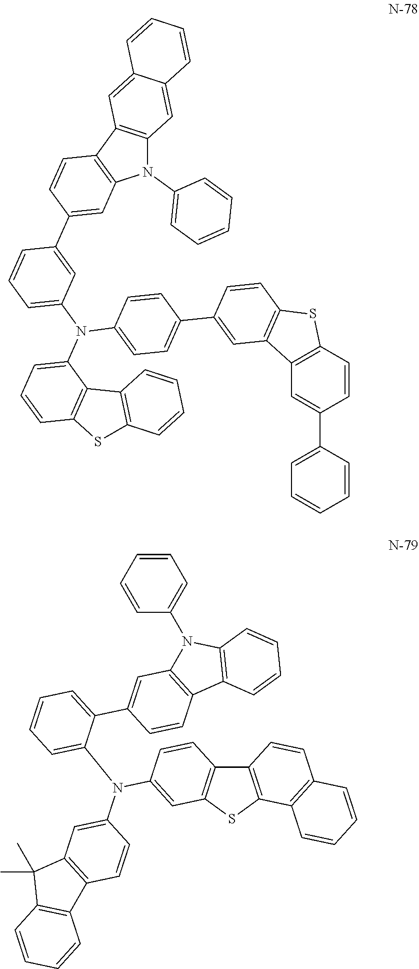 [SMILES: c1ccc(-c2ccc3sc4ccc(-c5ccc(N(c6cccc(-c7ccc8c9cc%10ccccc%10cc9n(-c9ccccc9)c8c7)c6)c6cccc7sc8ccccc8c67)cc5)cc4c3c2)cc1 ; CC1(C)c2ccccc2-c2ccc(N(c3ccc4c(c3)sc3c5ccccc5ccc43)c3ccccc3-c3ccc4c5ccccc5n(-c5ccccc5)c4c3)cc21]

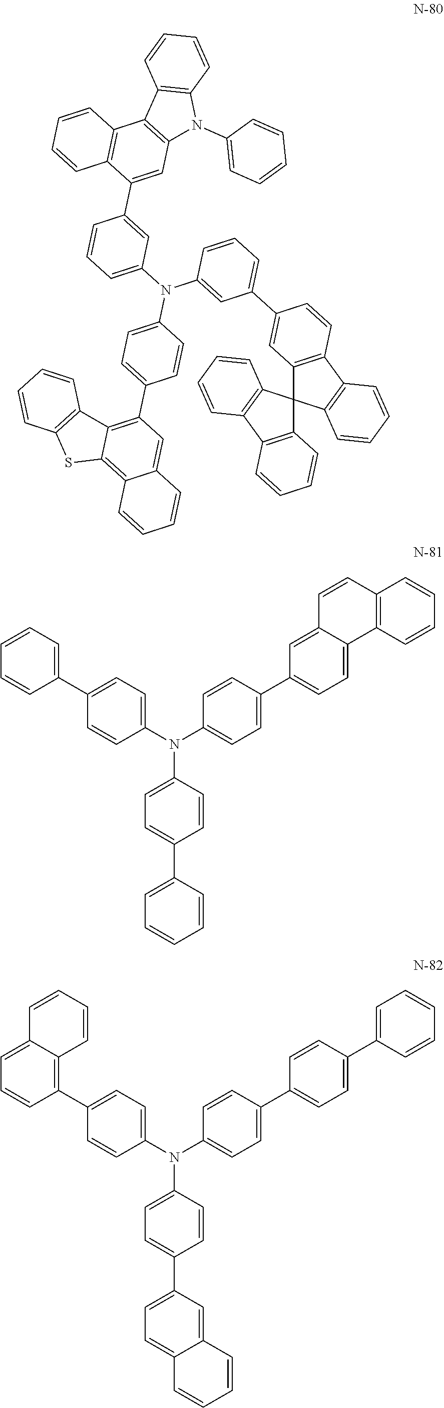 [SMILES: c1ccc(-n2c3ccccc3c3c4ccccc4c(-c4cccc(N(c5ccc(-c6cc7ccccc7c7sc8ccccc8c67)cc5)c5cccc(-c6ccc7c(c6)C6(c8ccccc8-c8ccccc86)c6ccccc6-7)c5)c4)cc32)cc1 ; c1ccc(-c2ccc(N(c3ccc(-c4ccccc4)cc3)c3ccc(-c4ccc5c(ccc6ccccc65)c4)cc3)cc2)cc1 ; c1ccc(-c2ccc(-c3ccc(N(c4ccc(-c5ccc6ccccc6c5)cc4)c4ccc(-c5cccc6ccccc56)cc4)cc3)cc2)cc1]

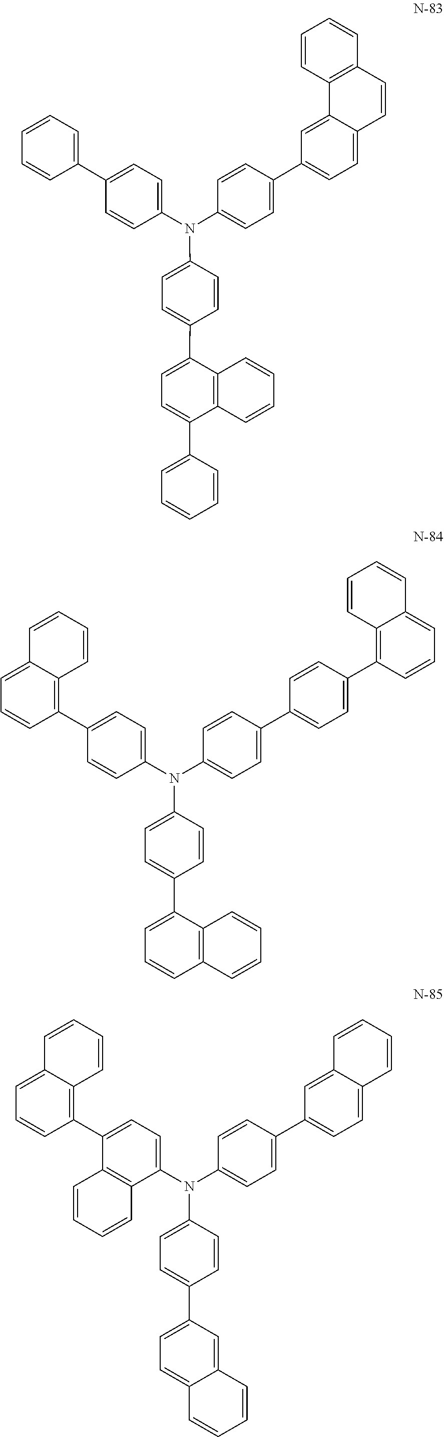 [SMILES: c1ccc(-c2ccc(N(c3ccc(-c4ccc5ccc6ccccc6c5c4)cc3)c3ccc(-c4ccc(-c5ccccc5)c5ccccc45)cc3)cc2)cc1 ; c1ccc2c(-c3ccc(-c4ccc(N(c5ccc(-c6cccc7ccccc67)cc5)c5ccc(-c6cccc7ccccc67)cc5)cc4)cc3)cccc2c1 ; c1ccc2cc(-c3ccc(N(c4ccc(-c5ccc6ccccc6c5)cc4)c4ccc(-c5cccc6ccccc56)c5ccccc45)cc3)ccc2c1]

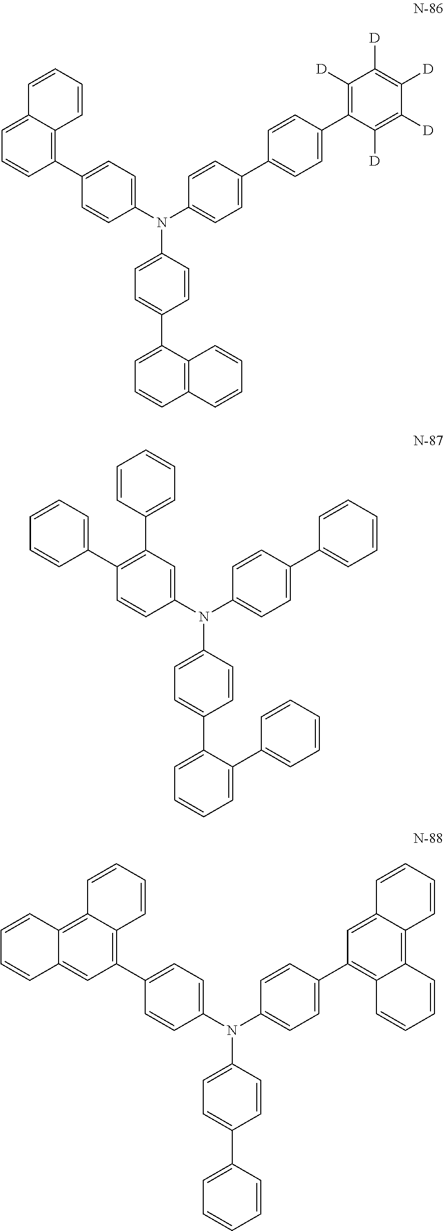

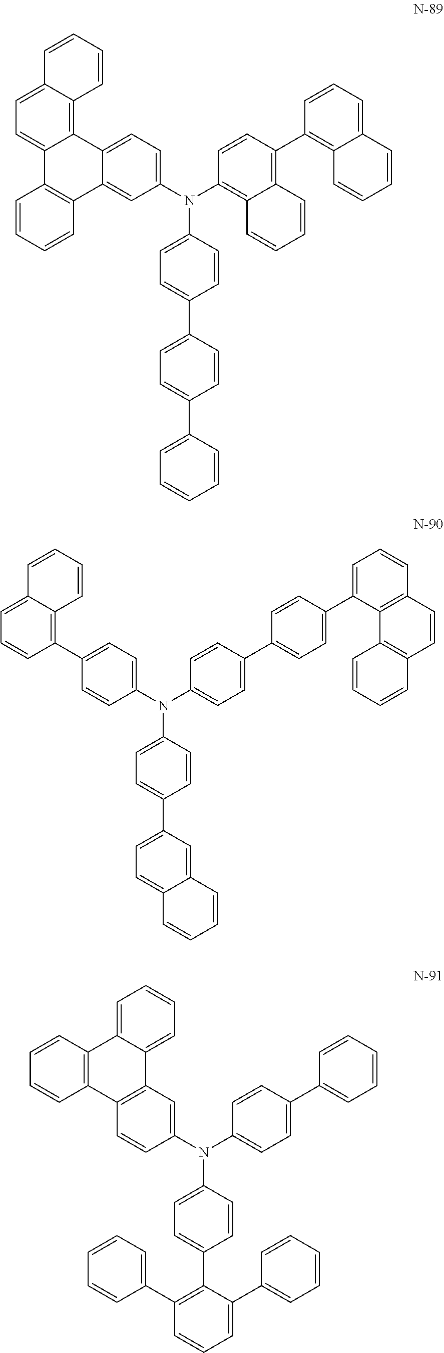 [SMILES: c1ccc(-c2ccc(-c3ccc(N(c4ccc5c(c4)c4ccccc4c4ccc6ccccc6c45)c4ccc(-c5cccc6ccccc56)c5ccccc45)cc3)cc2)cc1 ; c1ccc2cc(-c3ccc(N(c4ccc(-c5ccc(-c6cccc7ccc8ccccc8c67)cc5)cc4)c4ccc(-c5cccc6ccccc56)cc4)cc3)ccc2c1 ; c1ccc(-c2ccc(N(c3ccc(-c4c(-c5ccccc5)cccc4-c4ccccc4)cc3)c3ccc4c5ccccc5c5ccccc5c4c3)cc2)cc1]

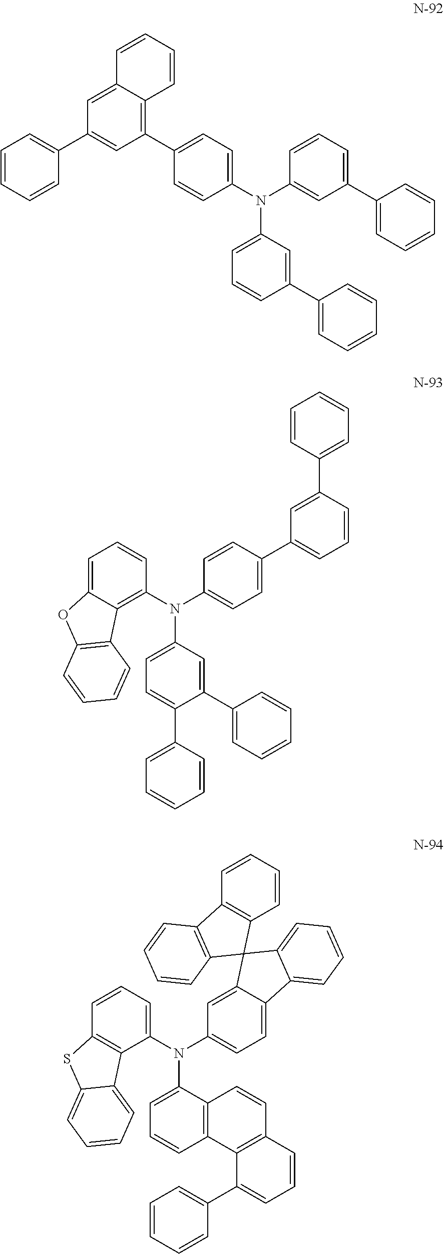 [SMILES: c1ccc(-c2cccc(N(c3ccc(-c4cc(-c5ccccc5)cc5ccccc45)cc3)c3cccc(-c4ccccc4)c3)c2)cc1 ; c1ccc(-c2cccc(-c3ccc(N(c4ccc(-c5ccccc5)c(-c5ccccc5)c4)c4cccc5oc6ccccc6c45)cc3)c2)cc1 ; c1ccc(-c2cccc3ccc4c(N(c5ccc6c(c5)C5(c7ccccc7-c7ccccc75)c5ccccc5-6)c5cccc6sc7ccccc7c56)cccc4c23)cc1]

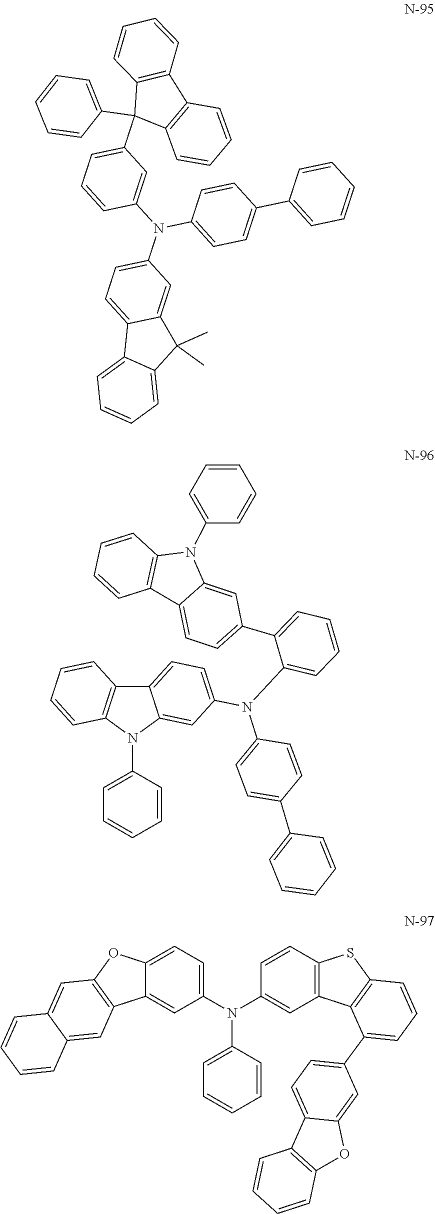 [SMILES: CC1(C)c2ccccc2-c2ccc(N(c3ccc(-c4ccccc4)cc3)c3cccc(C4(c5ccccc5)c5ccccc5-c5ccccc54)c3)cc21 ; c1ccc(-c2ccc(N(c3ccc4c5ccccc5n(-c5ccccc5)c4c3)c3ccccc3-c3ccc4c5ccccc5n(-c5ccccc5)c4c3)cc2)cc1 ; c1ccc(N(c2ccc3oc4cc5ccccc5cc4c3c2)c2ccc3sc4cccc(-c5ccc6c(c5)oc5ccccc56)c4c3c2)cc1]

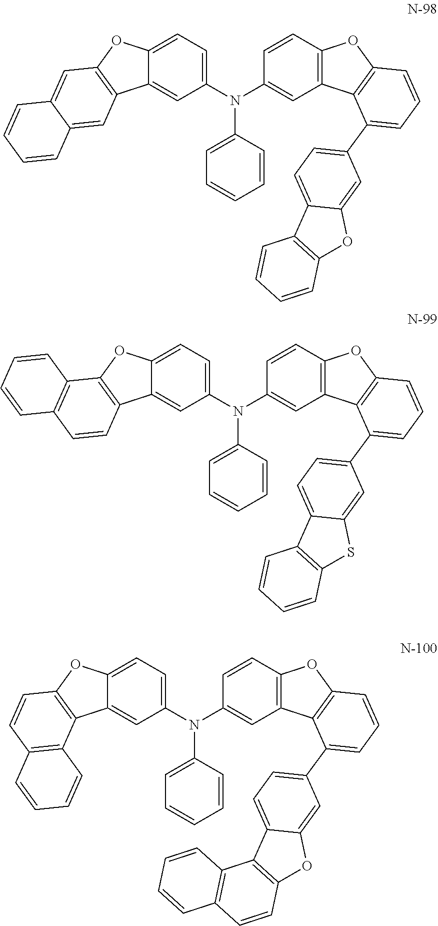

Also, the compound represented by Formula (3) is represented by any one of the following compounds S-1 to S-112.

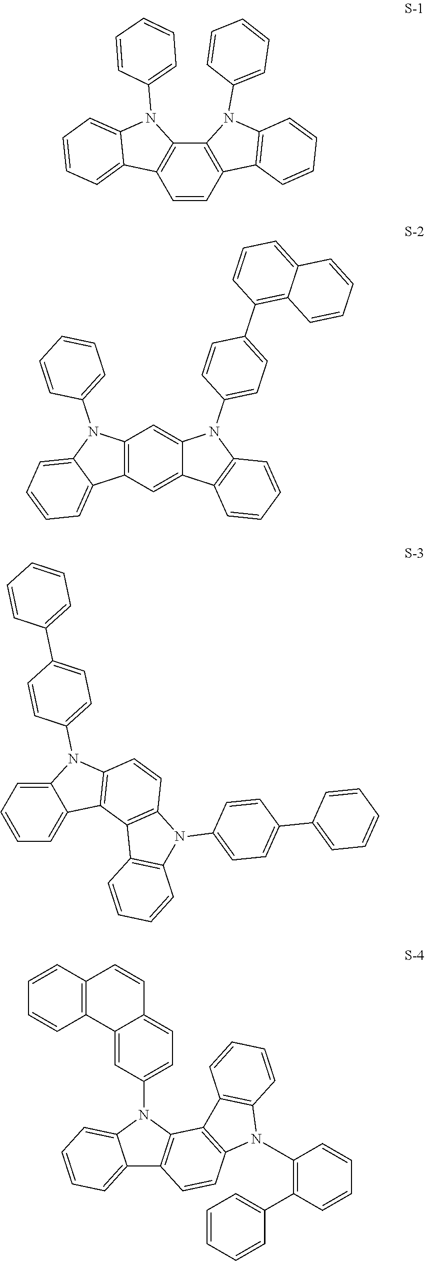

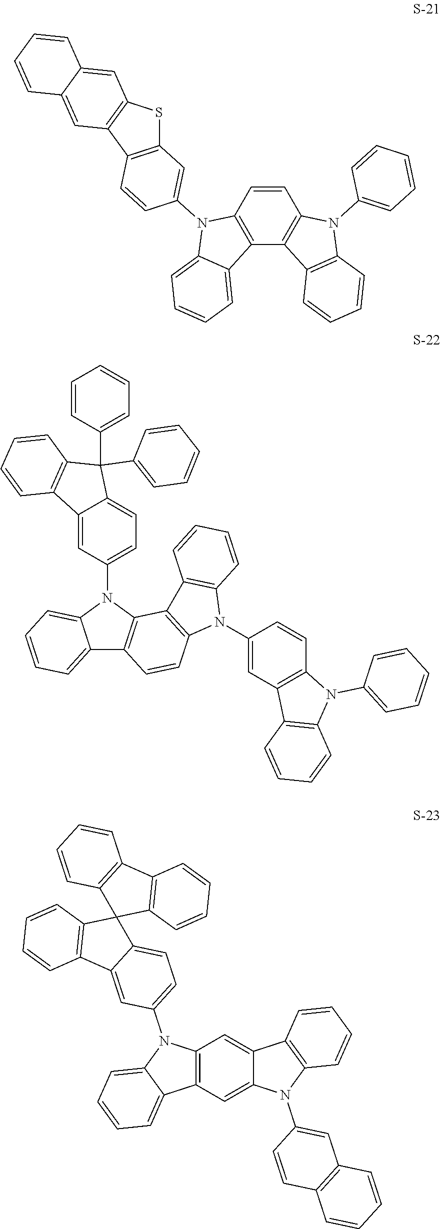 [SMILES: c1ccc(-n2c3ccccc3c3c4c5ccccc5n(-c5ccc6c(c5)sc5cc7ccccc7cc56)c4ccc32)cc1 ; c1ccc(-n2c3ccccc3c3cc(-n4c5ccccc5c5c4ccc4c6ccccc6n(-c6ccc7c(c6)-c6ccccc6C7(c6ccccc6)c6ccccc6)c45)ccc32)cc1 ; c1ccc2c(c1)-c1ccccc1C21c2ccccc2-c2cc(-n3c4ccccc4c4cc5c(cc43)c3ccccc3n5-c3ccc4ccccc4c3)ccc21]

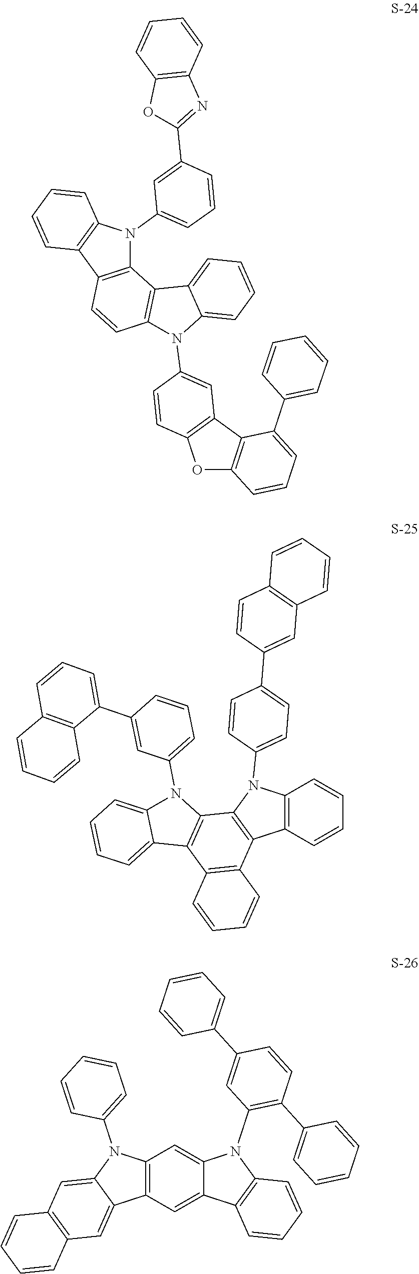

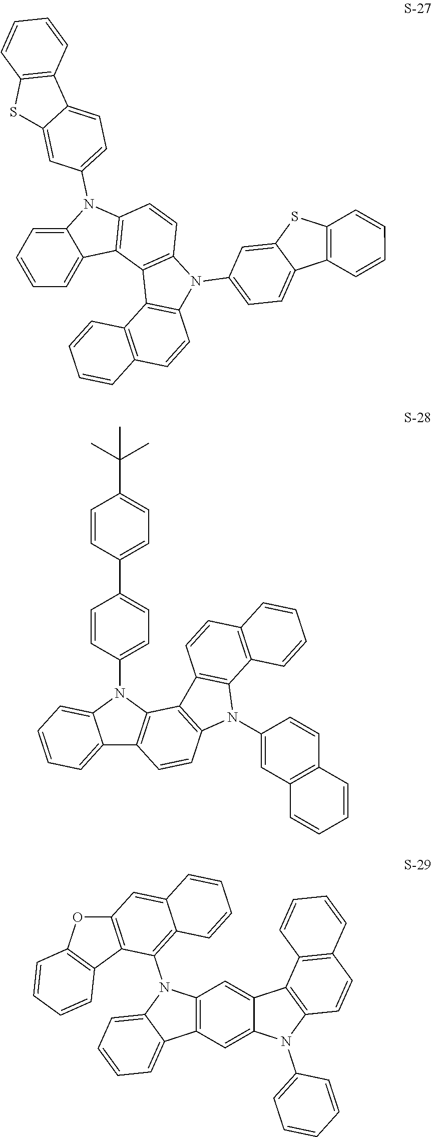

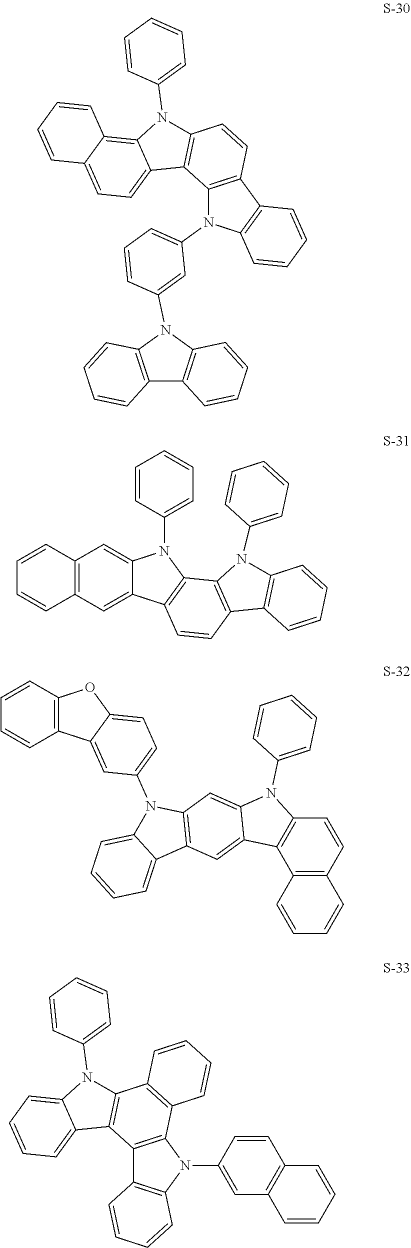 [SMILES: c1ccc(-n2c3ccc4c5ccccc5n(-c5cccc(-n6c7ccccc7c7ccccc76)c5)c4c3c3ccc4ccccc4c32)cc1 ; c1ccc(-n2c3ccccc3c3ccc4c5cc6ccccc6cc5n(-c5ccccc5)c4c32)cc1 ; c1ccc(-n2c3cc4c(cc3c3c5ccccc5ccc32)c2ccccc2n4-c2ccc3oc4ccccc4c3c2)cc1 ; c1ccc(-n2c3ccccc3c3c4c5ccccc5n(-c5ccc6ccccc6c5)c4c4ccccc4c32)cc1]

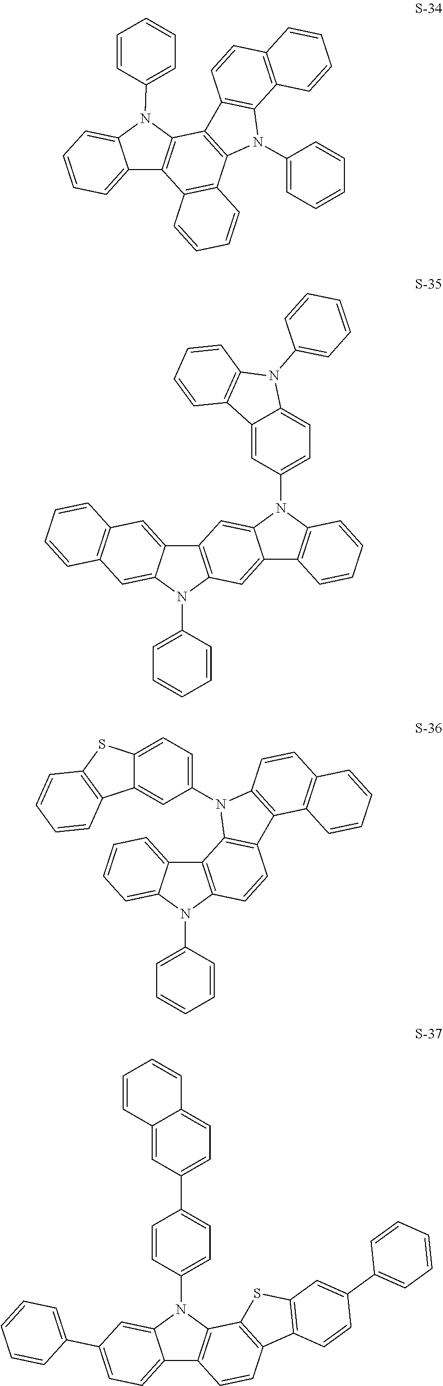

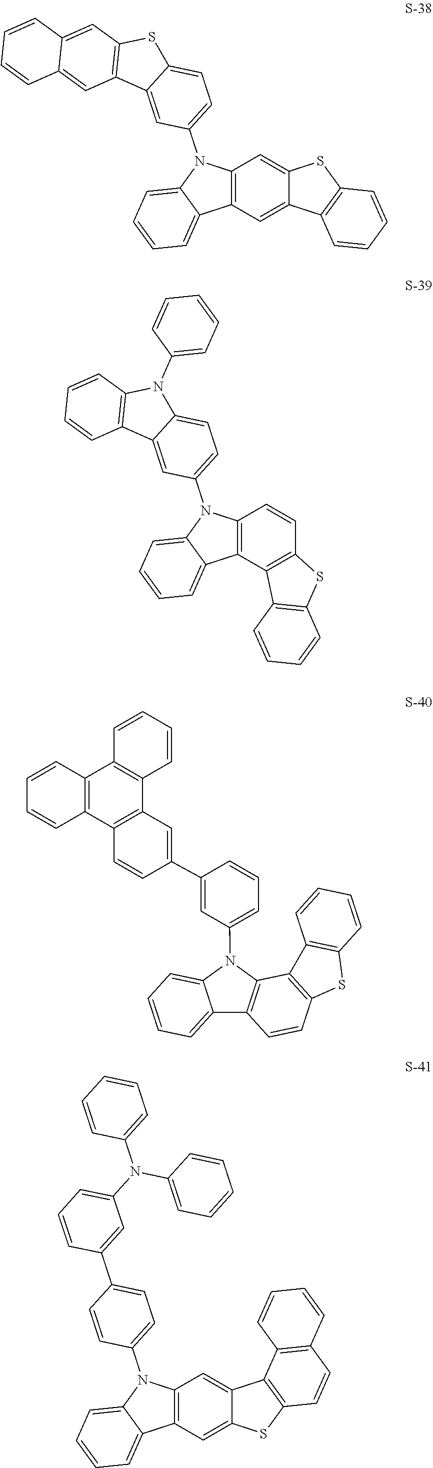 [SMILES: c1ccc2cc3c(cc2c1)sc1ccc(-n2c4ccccc4c4cc5c(cc42)sc2ccccc25)cc13 ; c1ccc(-n2c3ccccc3c3cc(-n4c5ccccc5c5c6c(ccc54)sc4ccccc46)ccc32)cc1 ; c1cc(-c2ccc3c4ccccc4c4ccccc4c3c2)cc(-n2c3ccccc3c3ccc4sc5ccccc5c4c32)c1 ; c1ccc(N(c2ccccc2)c2cccc(-c3ccc(-n4c5ccccc5c5cc6sc7ccc8ccccc8c7c6cc54)cc3)c2)cc1]

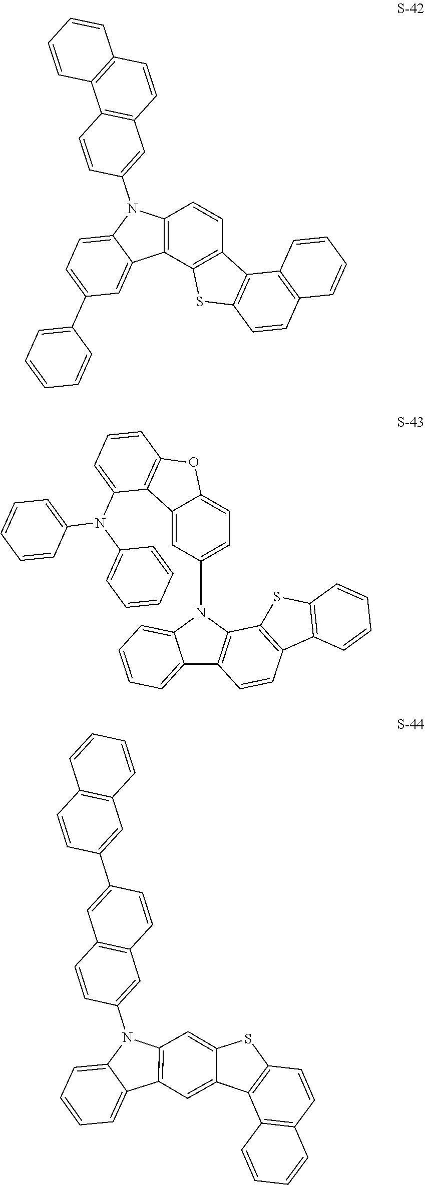 [SMILES: c1ccc(-c2ccc3c(c2)c2c4sc5ccc6ccccc6c5c4ccc2n3-c2ccc3c(ccc4ccccc43)c2)cc1 ; c1ccc(N(c2ccccc2)c2cccc3oc4ccc(-n5c6ccccc6c6ccc7c8ccccc8sc7c65)cc4c23)cc1 ; c1ccc2cc(-c3ccc4cc(-n5c6ccccc6c6cc7c(cc65)sc5ccc6ccccc6c57)ccc4c3)ccc2c1]

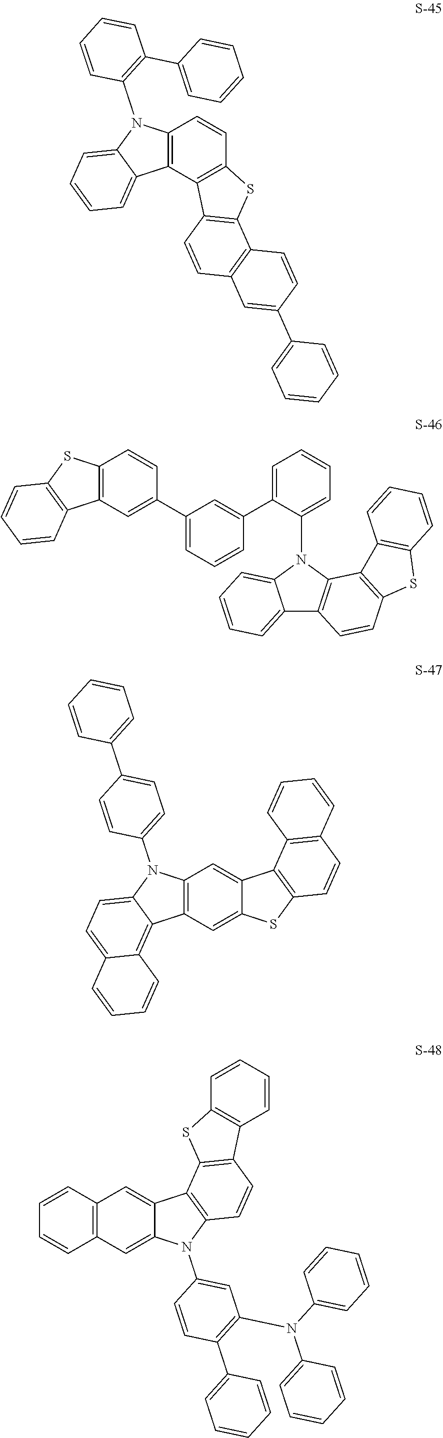 [SMILES: c1ccc(-c2ccc3c(ccc4c3sc3ccc5c(c6ccccc6n5-c5ccccc5-c5ccccc5)c34)c2)cc1 ; c1cc(-c2ccc3sc4ccccc4c3c2)cc(-c2ccccc2-n2c3ccccc3c3ccc4sc5ccccc5c4c32)c1 ; c1ccc(-c2ccc(-n3c4cc5c(cc4c4c6ccccc6ccc43)sc3ccc4ccccc4c35)cc2)cc1 ; c1ccc(-c2ccc(-n3c4cc5ccccc5cc4c4c5sc6ccccc6c5ccc43)cc2N(c2ccccc2)c2ccccc2)cc1]

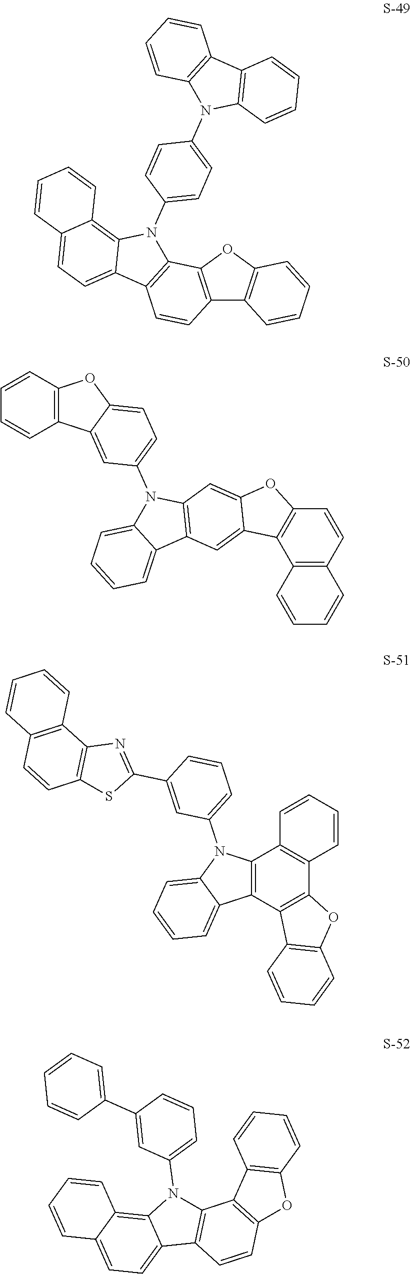 [SMILES: c1ccc2c(c1)ccc1c3ccc4c5ccccc5oc4c3n(-c3ccc(-n4c5ccccc5c5ccccc54)cc3)c21 ; c1ccc2c(c1)ccc1oc3cc4c(cc3c12)c1ccccc1n4-c1ccc2oc3ccccc3c2c1 ; c1cc(-c2nc3c(ccc4ccccc43)s2)cc(-n2c3ccccc3c3c4c5ccccc5oc4c4ccccc4c32)c1 ; c1ccc(-c2cccc(-n3c4c5ccccc5ccc4c4ccc5oc6ccccc6c5c43)c2)cc1]

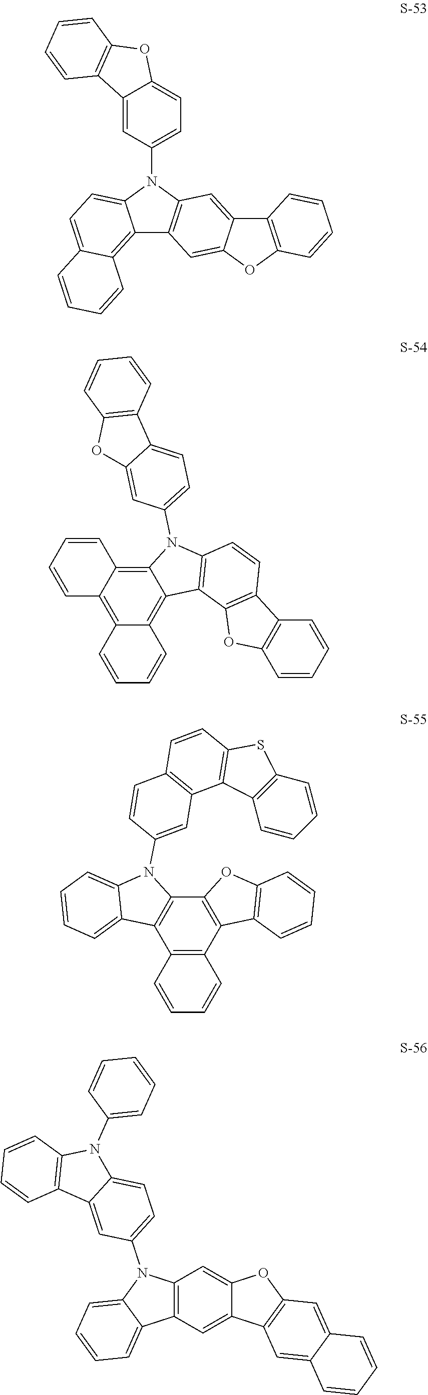

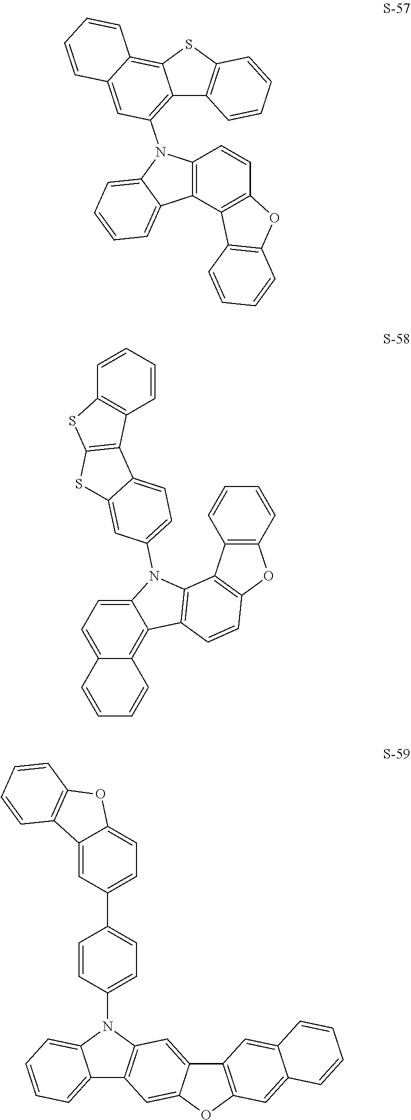 [SMILES: c1ccc2c(c1)cc(-n1c3ccccc3c3c4c(ccc31)oc1ccccc14)c1c3ccccc3sc21 ; c1ccc2c(c1)ccc1c2c2ccc3oc4ccccc4c3c2n1-c1ccc2c(c1)sc1sc3ccccc3c12 ; c1ccc2cc3c(cc2c1)oc1cc2c4ccccc4n(-c4ccc(-c5ccc6oc7ccccc7c6c5)cc4)c2cc13]

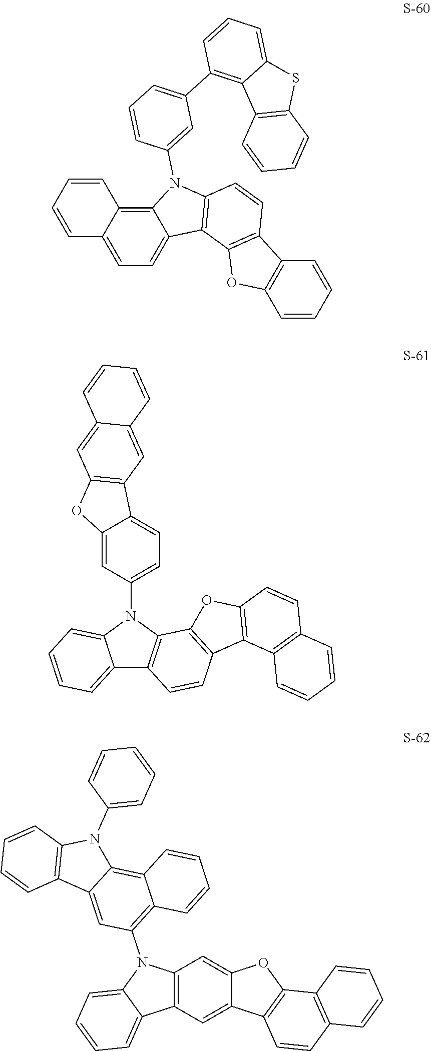 [SMILES: c1cc(-c2cccc3sc4ccccc4c23)cc(-n2c3ccc4c5ccccc5oc4c3c3ccc4ccccc4c32)c1 ; c1ccc2cc3c(cc2c1)oc1cc(-n2c4ccccc4c4ccc5c(oc6ccc7ccccc7c65)c42)ccc13 ; c1ccc(-n2c3ccccc3c3cc(-n4c5ccccc5c5cc6c(cc54)oc4c5ccccc5ccc64)c4ccccc4c32)cc1]

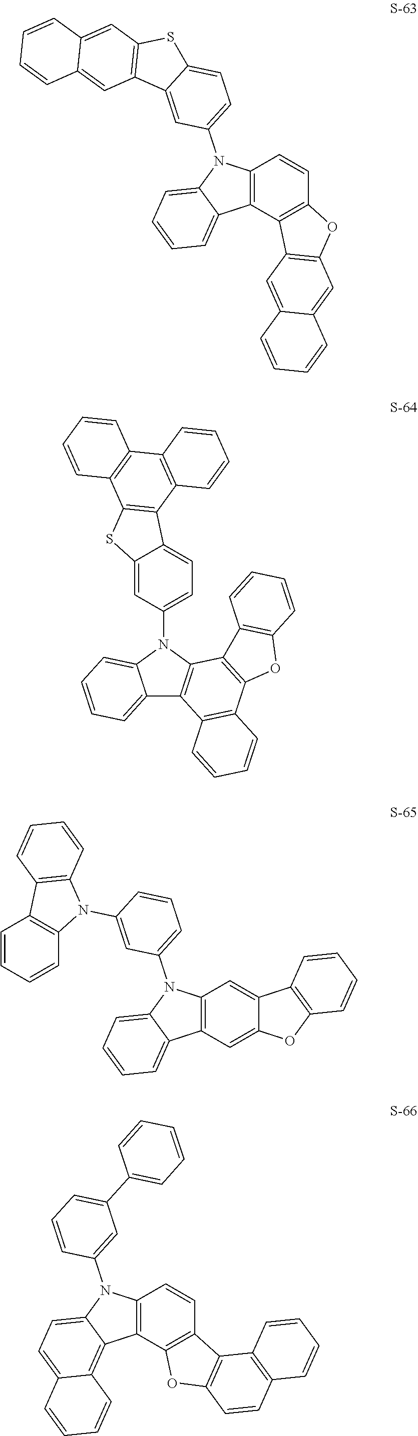 [SMILES: c1ccc2cc3c(cc2c1)oc1ccc2c(c4ccccc4n2-c2ccc4sc5cc6ccccc6cc5c4c2)c13 ; c1ccc2c(c1)oc1c3ccccc3c3c4ccccc4n(-c4ccc5c(c4)sc4c6ccccc6c6ccccc6c54)c3c21 ; c1cc(-n2c3ccccc3c3ccccc32)cc(-n2c3ccccc3c3cc4oc5ccccc5c4cc32)c1 ; c1ccc(-c2cccc(-n3c4ccc5ccccc5c4c4c5oc6ccc7ccccc7c6c5ccc43)c2)cc1]

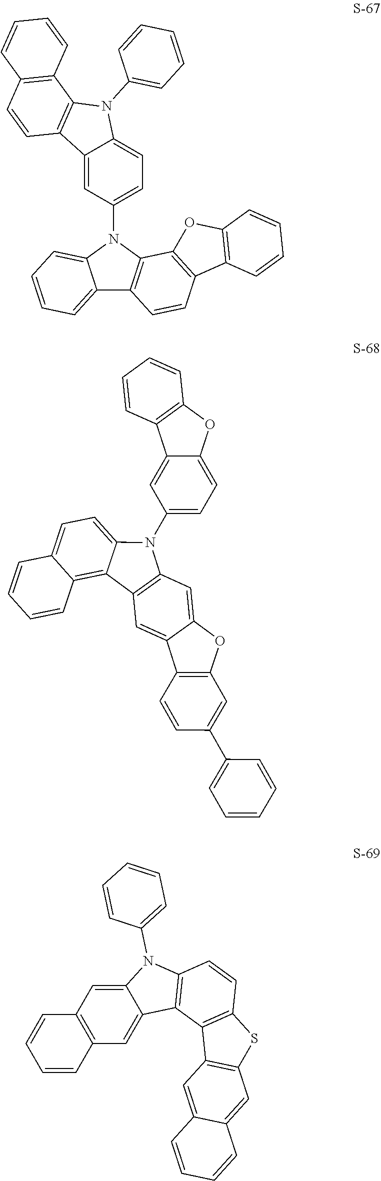

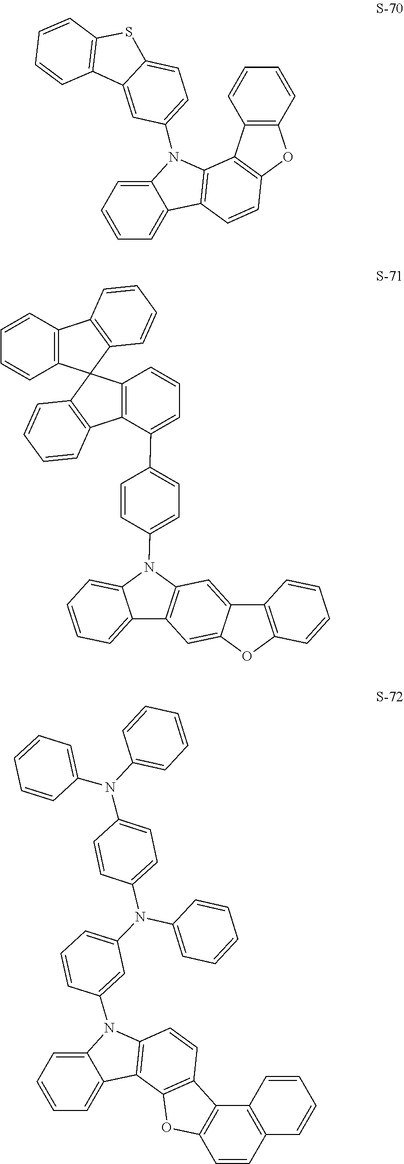 [SMILES: c1ccc2c(c1)oc1ccc3c4ccccc4n(-c4ccc5sc6ccccc6c5c4)c3c12 ; c1ccc2c(c1)-c1ccccc1C21c2ccccc2-c2c(-c3ccc(-n4c5ccccc5c5cc6oc7ccccc7c6cc54)cc3)cccc21 ; c1ccc(N(c2ccccc2)c2ccc(N(c3ccccc3)c3cccc(-n4c5ccccc5c5c6oc7ccc8ccccc8c7c6ccc54)c3)cc2)cc1]

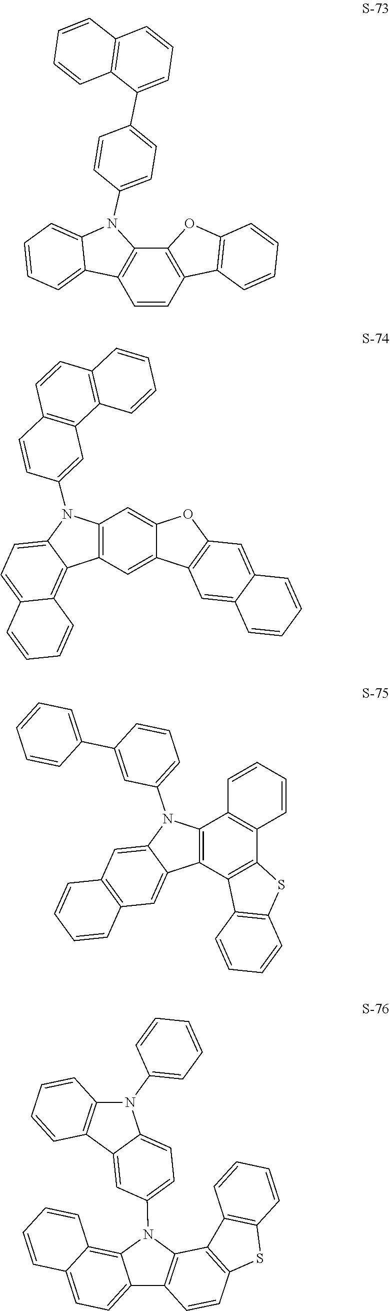

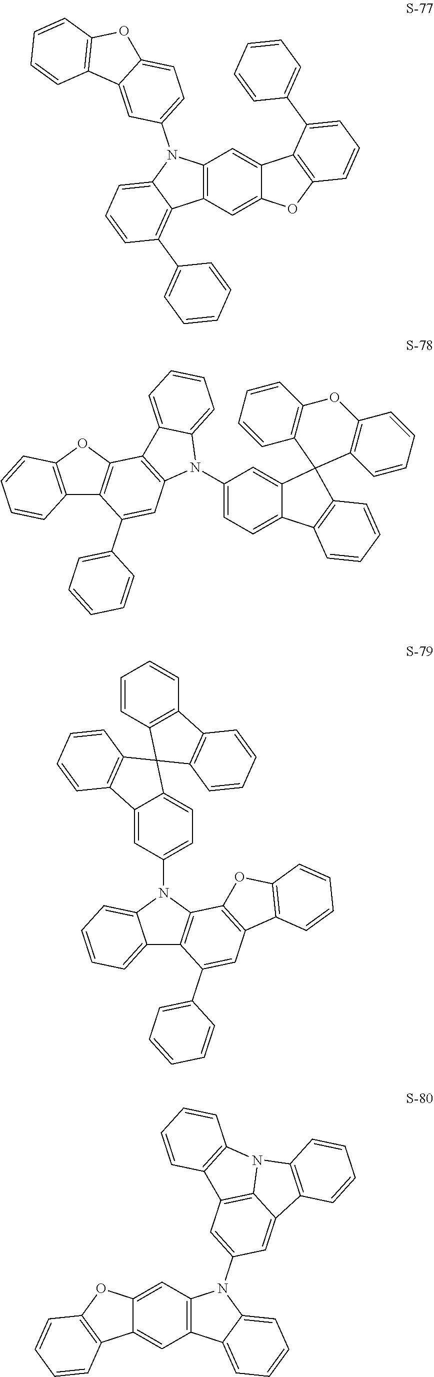

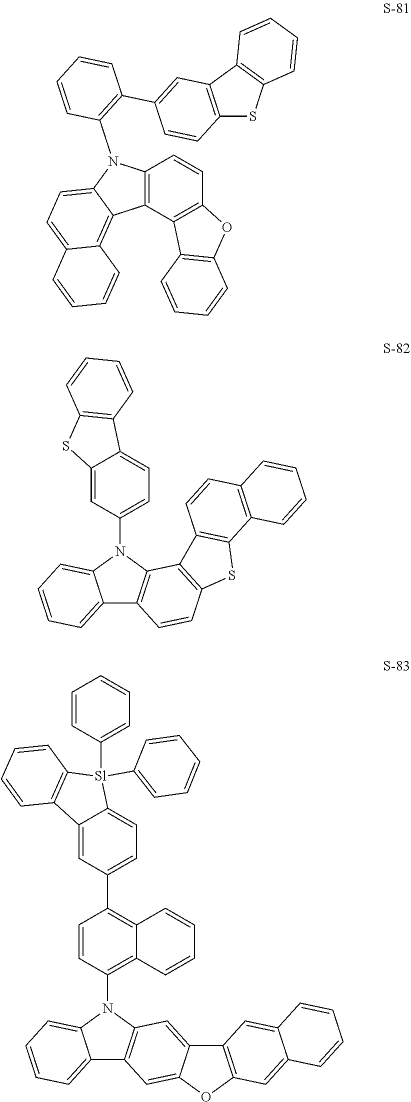

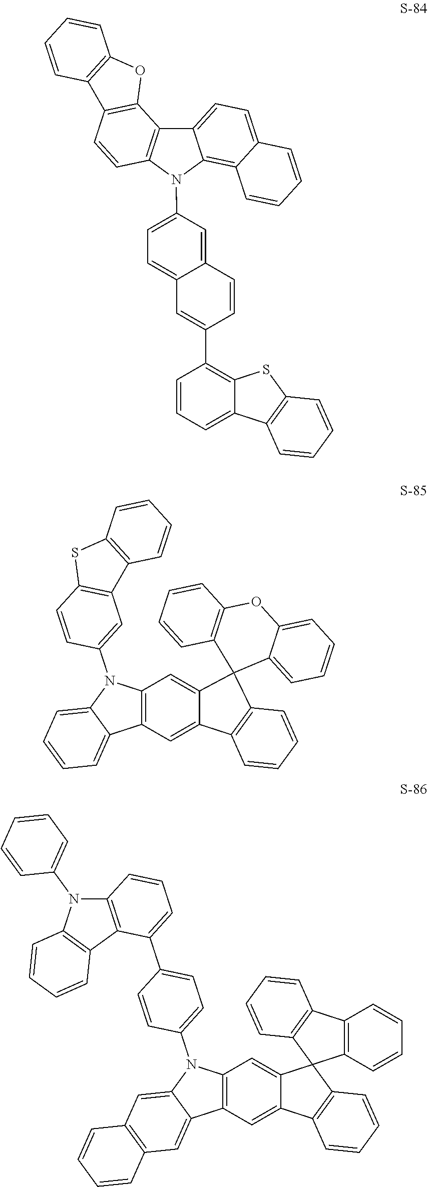 [SMILES: c1ccc2c(c1)ccc1c3c4oc5ccccc5c4ccc3n(-c3ccc4cc(-c5cccc6c5sc5ccccc56)ccc4c3)c21 ; c1ccc2c(c1)Oc1ccccc1C21c2ccccc2-c2cc3c4ccccc4n(-c4ccc5sc6ccccc6c5c4)c3cc21 ; c1ccc(-n2c3ccccc3c3c(-c4ccc(-n5c6cc7c(cc6c6cc8ccccc8cc65)-c5ccccc5C75c6ccccc6-c6ccccc65)cc4)cccc32)cc1]

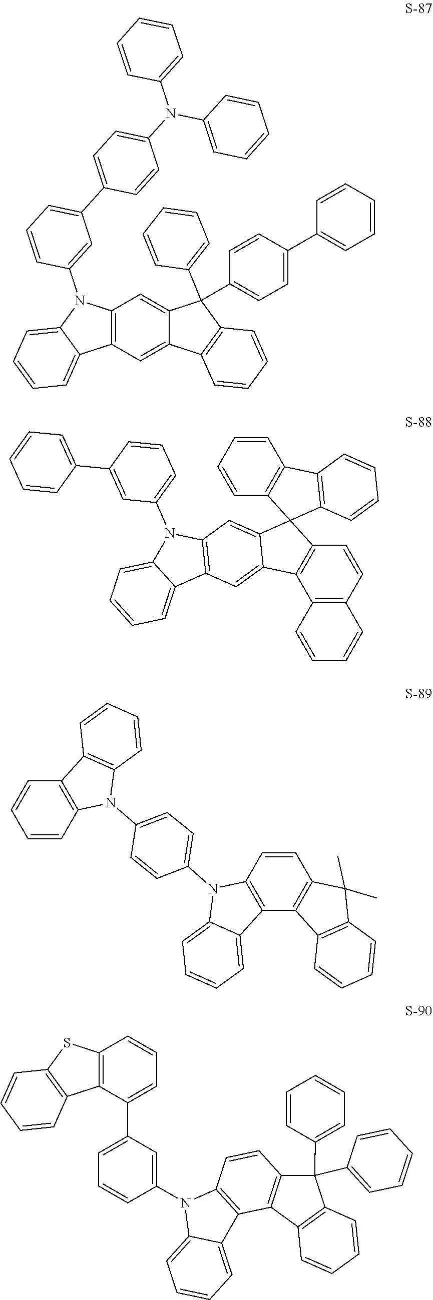

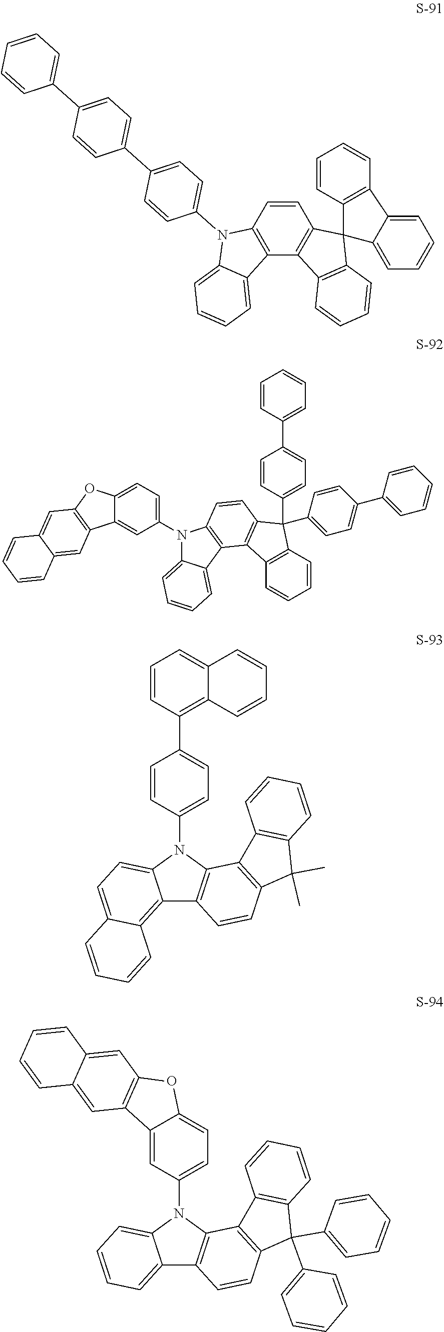

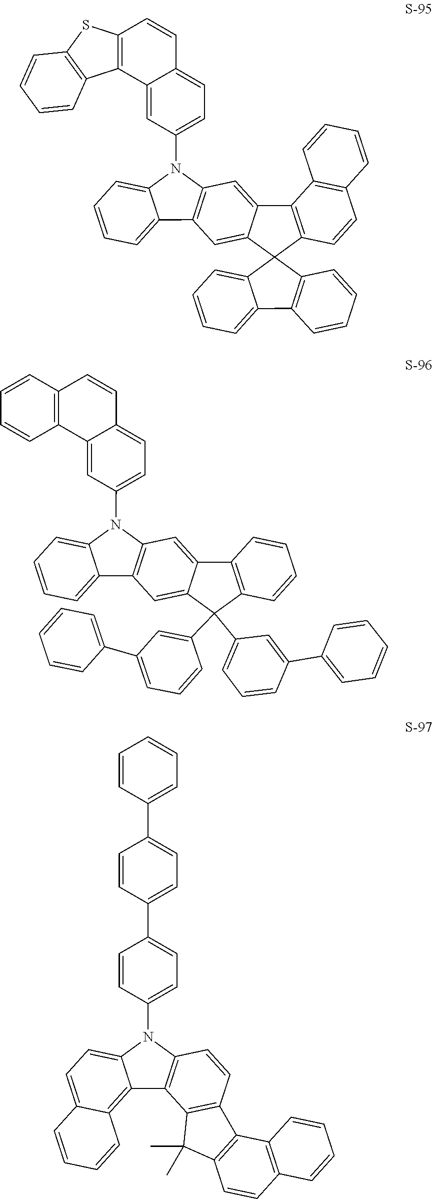 [SMILES: c1ccc2c(c1)-c1ccccc1C21c2cc3c4ccccc4n(-c4ccc5ccc6sc7ccccc7c6c5c4)c3cc2-c2c1ccc1ccccc21 ; c1ccc(-c2cccc(C3(c4cccc(-c5ccccc5)c4)c4ccccc4-c4cc5c(cc43)c3ccccc3n5-c3ccc4ccc5ccccc5c4c3)c2)cc1 ; CC1(C)c2ccc3ccccc3c2-c2ccc3c(c21)c1c2ccccc2ccc1n3-c1ccc(-c2ccc(-c3ccccc3)cc2)cc1]

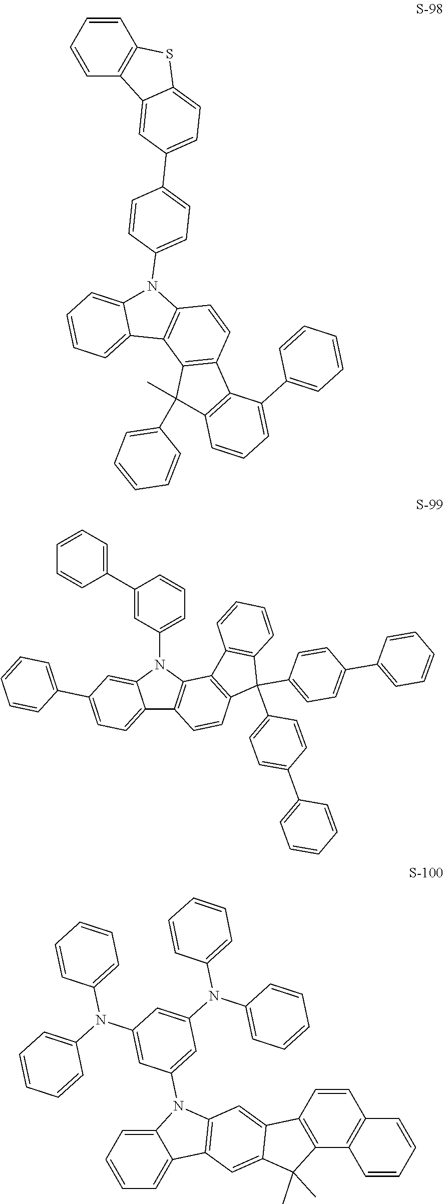 [SMILES: CC1(c2ccccc2)c2cccc(-c3ccccc3)c2-c2ccc3c(c21)c1ccccc1n3-c1ccc(-c2ccc3sc4ccccc4c3c2)cc1 ; c1ccc(-c2ccc(C3(c4ccc(-c5ccccc5)cc4)c4ccccc4-c4c3ccc3c5ccc(-c6ccccc6)cc5n(-c5cccc(-c6ccccc6)c5)c43)cc2)cc1 ; CC1(C)c2cc3c4ccccc4n(-c4cc(N(c5ccccc5)c5ccccc5)cc(N(c5ccccc5)c5ccccc5)c4)c3cc2-c2ccc3ccccc3c21]

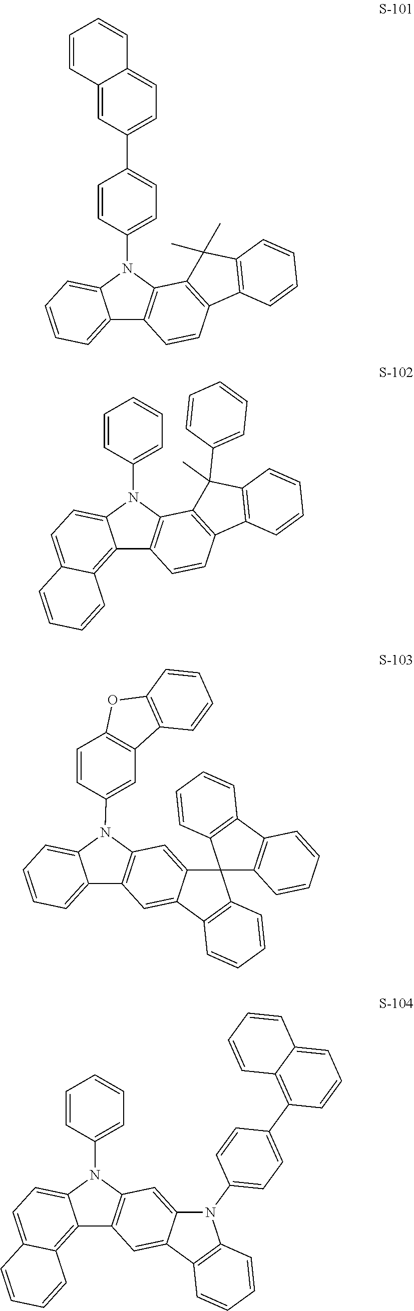

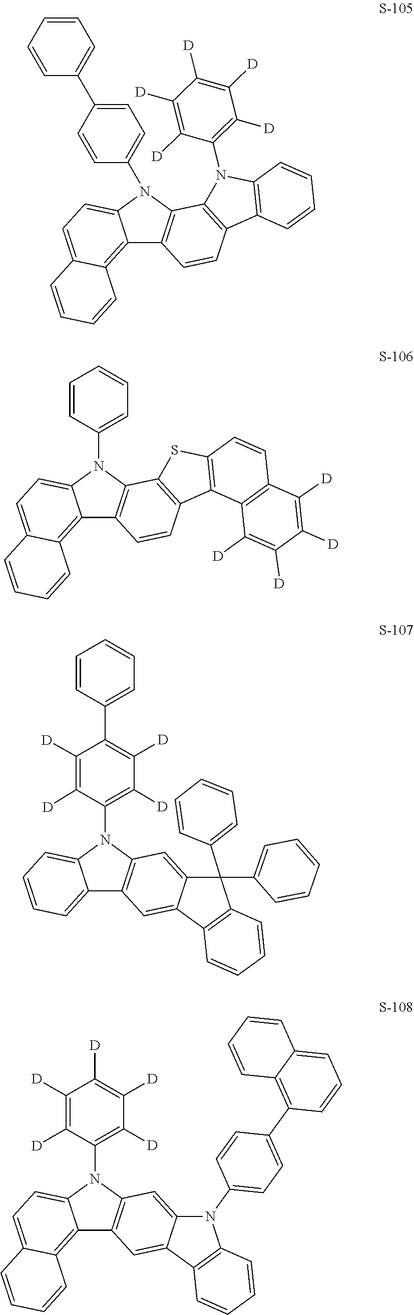

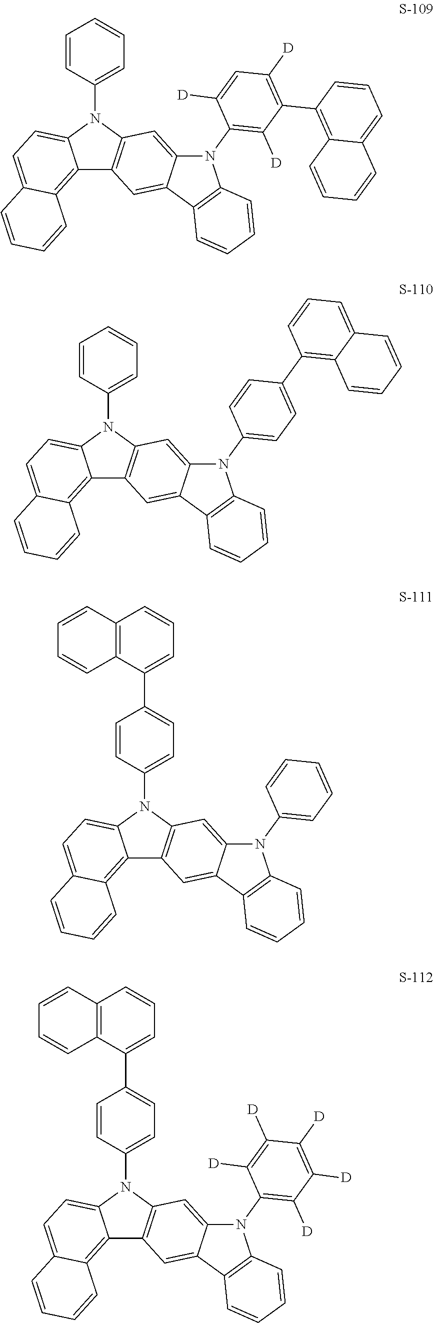 [SMILES: [2H]c1cc([2H])c(-n2c3ccccc3c3cc4c5c6ccccc6ccc5n(-c5ccccc5)c4cc32)c([2H])c1-c1cccc2ccccc12 ; c1ccc(-n2c3cc4c(cc3c3c5ccccc5ccc32)c2ccccc2n4-c2ccc(-c3cccc4ccccc34)cc2)cc1 ; c1ccc(-n2c3ccccc3c3cc4c5c6ccccc6ccc5n(-c5ccc(-c6cccc7ccccc67)cc5)c4cc32)cc1 ; [2H]c1c([2H])c([2H])c(-n2c3ccccc3c3cc4c5c6ccccc6ccc5n(-c5ccc(-c6cccc7ccccc67)cc5)c4cc32)c([2H])c1[2H]]

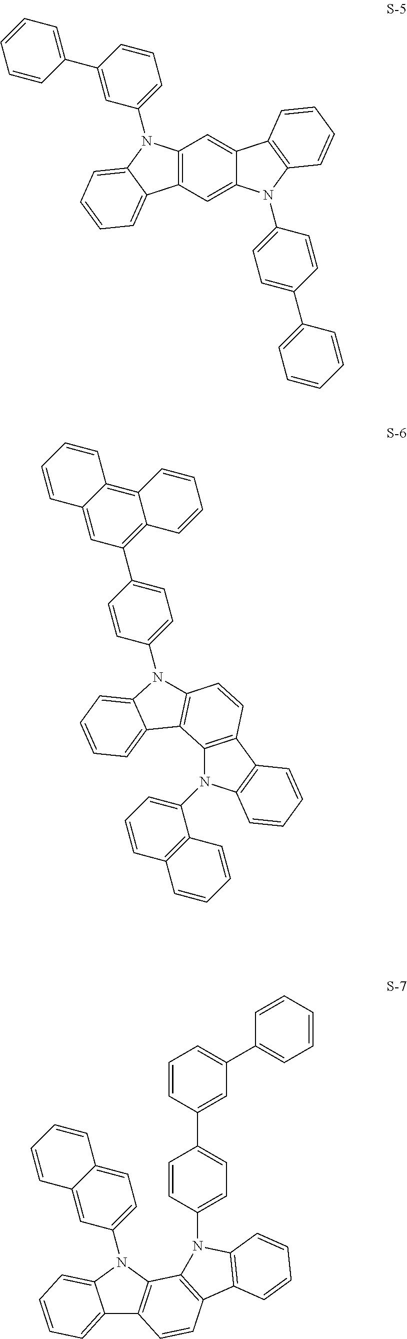 [SMILES: c1ccc(-c2ccc(-n3c4ccccc4c4cc5c(cc43)c3ccccc3n5-c3cccc(-c4ccccc4)c3)cc2)cc1 ; c1ccc2c(-n3c4ccccc4c4ccc5c(c6ccccc6n5-c5ccc(-c6cc7ccccc7c7ccccc67)cc5)c43)cccc2c1 ; c1ccc(-c2cccc(-c3ccc(-n4c5ccccc5c5ccc6c7ccccc7n(-c7ccc8ccccc8c7)c6c54)cc3)c2)cc1]

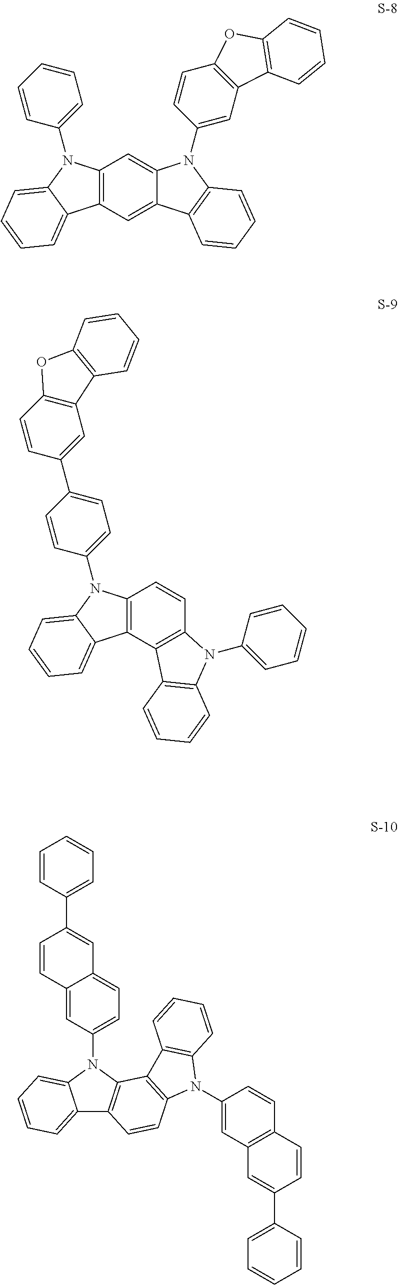 [SMILES: c1ccc(-n2c3ccccc3c3cc4c5ccccc5n(-c5ccc6oc7ccccc7c6c5)c4cc32)cc1 ; c1ccc(-n2c3ccccc3c3c4c5ccccc5n(-c5ccc(-c6ccc7oc8ccccc8c7c6)cc5)c4ccc32)cc1 ; c1ccc(-c2ccc3cc(-n4c5ccccc5c5ccc6c(c7ccccc7n6-c6ccc7ccc(-c8ccccc8)cc7c6)c54)ccc3c2)cc1]

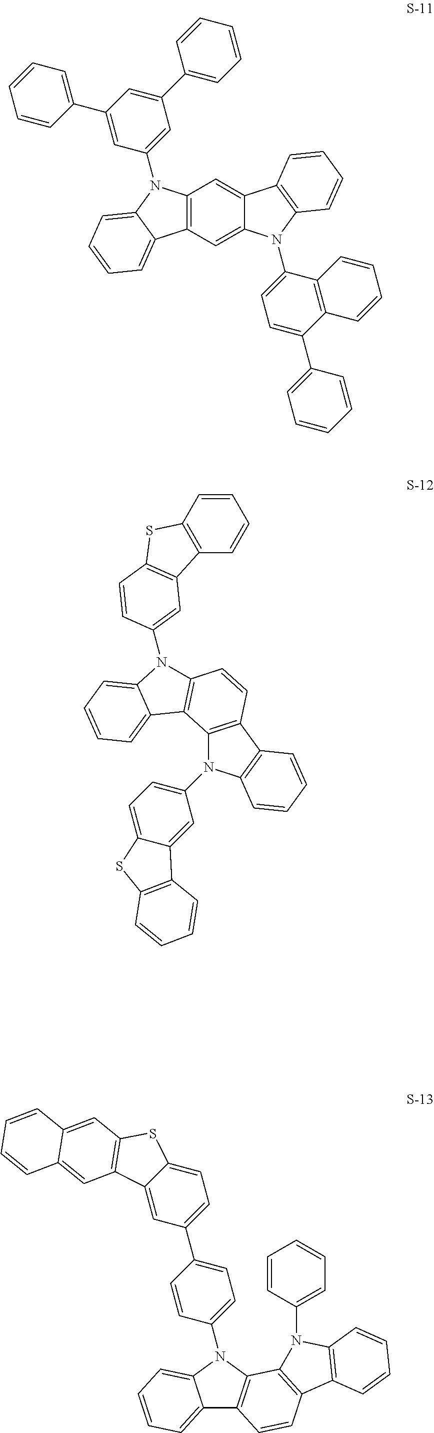 [SMILES: c1ccc(-c2cc(-c3ccccc3)cc(-n3c4ccccc4c4cc5c(cc43)c3ccccc3n5-c3ccc(-c4ccccc4)c4ccccc34)c2)cc1 ; c1ccc2c(c1)sc1ccc(-n3c4ccccc4c4c3ccc3c5ccccc5n(-c5ccc6sc7ccccc7c6c5)c34)cc12 ; c1ccc(-n2c3ccccc3c3ccc4c5ccccc5n(-c5ccc(-c6ccc7sc8cc9ccccc9cc8c7c6)cc5)c4c32)cc1]

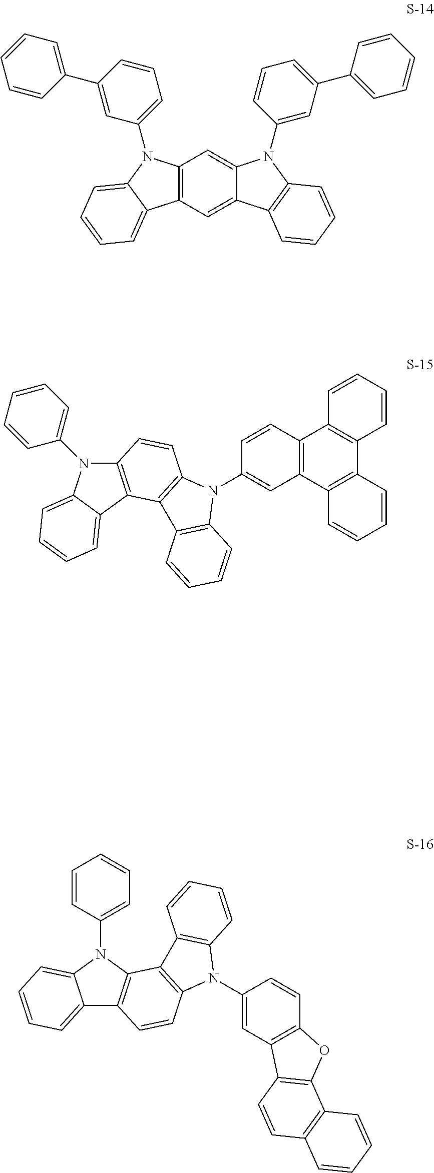 [SMILES: c1ccc(-c2cccc(-n3c4ccccc4c4cc5c6ccccc6n(-c6cccc(-c7ccccc7)c6)c5cc43)c2)cc1 ; c1ccc(-n2c3ccccc3c3c4c5ccccc5n(-c5ccc6c7ccccc7c7ccccc7c6c5)c4ccc32)cc1 ; c1ccc(-n2c3ccccc3c3ccc4c(c5ccccc5n4-c4ccc5oc6c7ccccc7ccc6c5c4)c32)cc1]

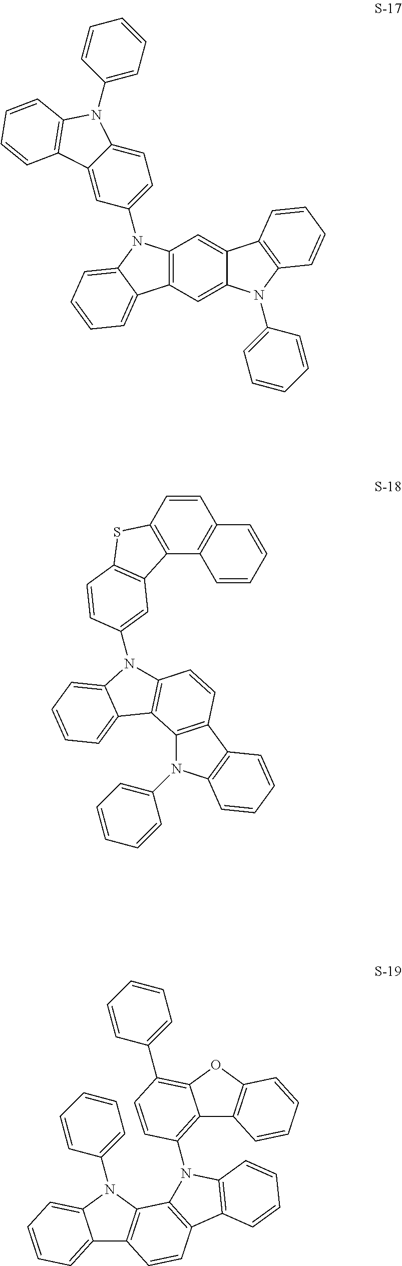 [SMILES: c1ccc(-n2c3ccccc3c3cc(-n4c5ccccc5c5cc6c(cc54)c4ccccc4n6-c4ccccc4)ccc32)cc1 ; c1ccc(-n2c3ccccc3c3ccc4c(c5ccccc5n4-c4ccc5sc6ccc7ccccc7c6c5c4)c32)cc1 ; c1ccc(-c2ccc(-n3c4ccccc4c4ccc5c6ccccc6n(-c6ccccc6)c5c43)c3c2oc2ccccc23)cc1]

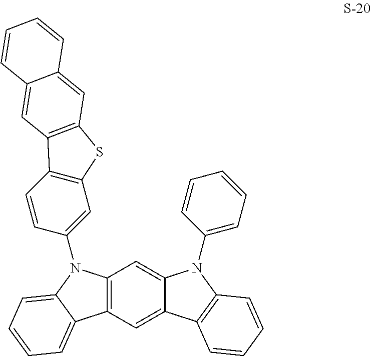 [SMILES: c1ccc(-n2c3ccccc3c3cc4c5ccccc5n(-c5ccc6c(c5)sc5cc7ccccc7cc56)c4cc32)cc1]

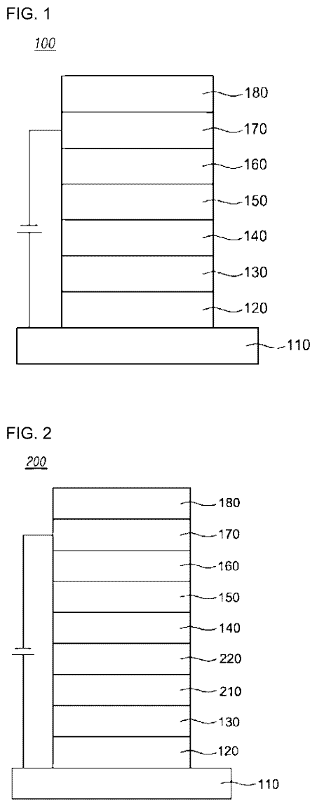

Referring to FIG. 1, the organic electronic element (100) according to the present invention comprises a first electrode (110), a second electrode (170), an organic material layer comprising single compound or 2 or more compounds represented by Formula 1 between the first electrode (110) and the second electrode (170). Wherein, the first electrode (110) may be an anode or a positive electrode, and the second electrode (170) may be a cathode or a negative electrode. In the case of an inverted organic electronic element, the first electrode may be a cathode, and the second electrode may be an anode.

The organic material layer may sequentially comprise a hole injection layer (120), a hole transport layer (130), an emitting layer (140), an electron transport layer (150), and an electron injection layer (160) formed in sequence on the first electrode (110). Here, the remaining layers except the emitting layer (140) may not be formed. The organic material layer may further comprise a hole blocking layer, an electron blocking layer, an emitting-auxiliary layer (220), a buffer layer (210), etc., and the electron transport layer (150) and the like may serve as a hole blocking layer (see FIG. 2).

Also, the organic electronic element according to an embodiment of the present invention may further include a protective layer or a light efficiency enhancing layer (180). The light efficiency enhancing layer may be formed on the surface not in contact with the organic material layer among both surfaces of the first electrode or on the surface not in contact with the organic material layer among both surfaces of the second electrode. The compound according to an embodiment of the present invention applied to the organic material layer may be used as a material for a hole injection layer (120), a hole transport layer (130), an emitting-auxiliary layer (220), an electron transport auxiliary layer, an electron transport layer (150), an electron injection layer (160), a host or dopant of an emitting layer (140), or the light efficiency enhancing layer. Preferably, for example, the compound according to Formula 1 of the present invention can be used as a host material for the emitting layer.

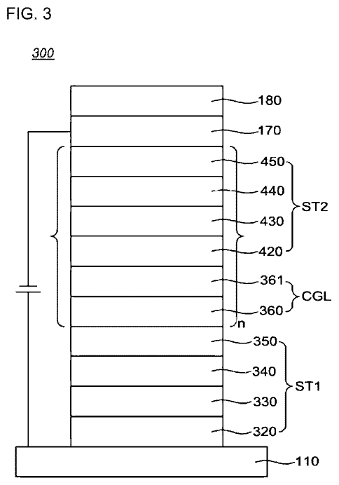

The organic material layer may include 2 or more stacks comprising a hole transport layer, an emitting layer and an electron transport layer sequentially formed on the anode, and may further comprise a charge generation layer formed between the 2 or more stacks (see FIG. 3).

Otherwise, even if the same core is used, the band gap, the electrical characteristics, the interface characteristics, and the like may vary depending on which substituent is bonded at which position, therefore the choice of core and the combination of sub-substituents associated therewith is also very important, and in particular, when the optimal combination of energy levels and T1 values, and unique properties of materials (mobility, interfacial characteristics, etc.) of each organic material layer is achieved, a long life span and high efficiency can be achieved at the same time.

The organic electroluminescent device according to an embodiment of the present invention may be manufactured using a PVD (physical vapor deposition) method. For example, a metal or a metal oxide having conductivity or an alloy thereof is deposited on a substrate to form a cathode, and the organic material layer including the hole injection layer (120), the hole transport layer (130), the emitting layer (140), the electron transport layer (150), and the electron injection layer (160) is formed thereon, and then depositing a material usable as a cathode thereon can manufacture an organic electroluminescent device according to an embodiment of the present invention.

Also, the present invention provides the organic electronic element wherein the organic material layer is formed by one of a spin coating process, a nozzle printing process, an inkjet printing process, a slot coating process, a dip coating process or a roll-to-roll process, and the organic material layer provides an organic electronic element comprising the compound or a composition for an organic electronic element as an electron transport material.

As another specific example, the present invention provides an organic electronic element used by mixing the same or different compounds of the compound represented by Formula (1) to the organic material layer.

Additionally, the present invention provides an emitting layer composition comprising the compound represented by Formula (1), and provides an organic electronic element comprising the emitting layer.

Also, the present invention also provides an electronic device comprising a display device comprising the organic electronic element; and a control unit for driving the display device.

According to another aspect, the present invention provides a display device wherein the organic electronic element is at least one of an OLED, an organic solar cell, an organic photo conductor, an organic transistor (organic TFT) and an element for monochromic or white illumination. Here, the electronic device may be a wired/wireless communication terminal which is currently used or will be used in the future, and covers all kinds of electronic devices including a mobile communication terminal such as a cellular phone, a personal digital assistant (PDA), an electronic dictionary, a point-to-multipoint (PMP), a remote controller, a navigation unit, a game player, various kinds of TVs, and various kinds of computers.

Hereinafter, Synthesis examples of the compound represented by Formula (1) and preparation examples of the organic electronic element of the present invention will be described in detail by way of example, but are not limited to the following examples.

### Synthesis Example

The compound (final products) represented by Formula (1) according to the present invention is synthesized by reacting Sub 1 and Sub 2 as shown in Reaction Scheme 1, but is not limited thereto.

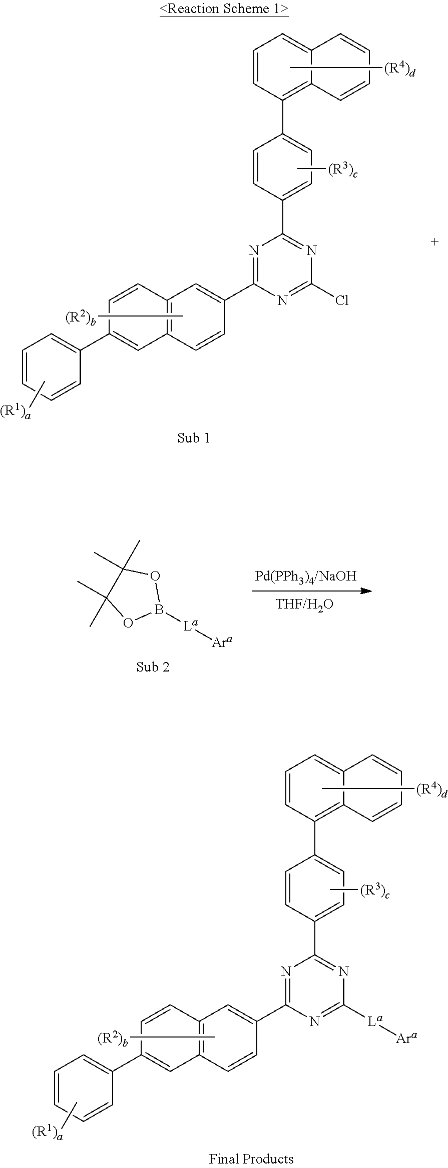

### 1. Synthesis of Sub 1

Sub 1 of Reaction Scheme 1 may be synthesized through the reaction route of Reaction Scheme 2, but is not limited thereto.

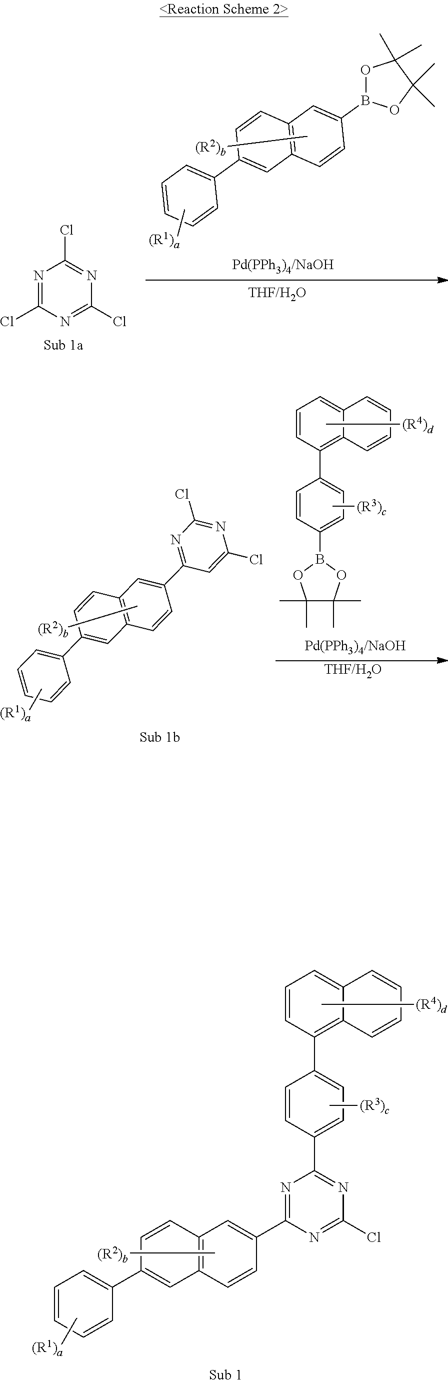

Synthesis examples of specific compounds belonging to Sub 1 are as follows.

### 1. Synthesis Example of Sub1-1

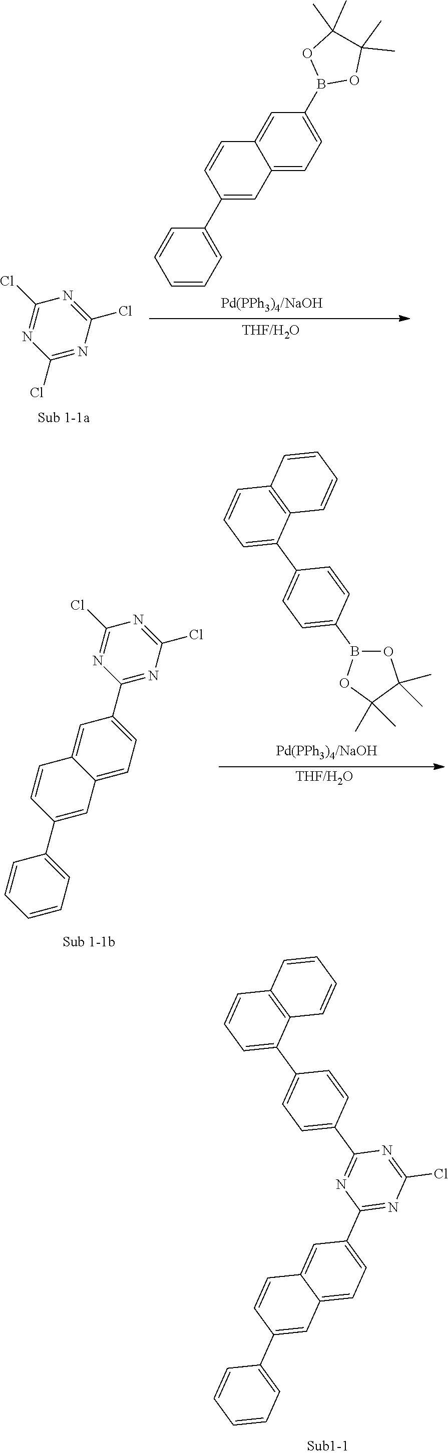 [SMILES: Clc1nc(Cl)nc(Cl)n1 ; CC1(C)OB(c2ccc3cc(-c4ccccc4)ccc3c2)OC1(C)C ; Clc1nc(Cl)nc(-c2ccc3cc(-c4ccccc4)ccc3c2)n1 ; CC1(C)OB(c2ccc(-c3cccc4ccccc34)cc2)OC1(C)C ; Clc1nc(-c2ccc(-c3cccc4ccccc34)cc2)nc(-c2ccc3cc(-c4ccccc4)ccc3c2)n1]

### (1) Synthesis of Sub1-1b

4,4,5,5-tetramethyl-2-(6-phenylnaphthalen-2-yl)-1,3,2-dioxaborolane (89.7 g, 0.27 mol), Pd(PPh₃)₄ (9.4 g, 0.008 mol), NaOH (32.6 g, 0.82 mol), THE (543 mL) and water (150 mL) were added to Sub1-1a (50 g, 0.27 mol), and reacted at 70° C. for 6 hours. When the reaction is completed, the temperature of the reactant is cooled to room temperature and the reaction solvent is removed. Afterwards, the concentrated reactant was separated using a silica gel column or recrystallization method to obtain 72 g (75.3%) of product Sub1-1b.

### (2) Synthesis of Sub1-1

4,4,5,5-tetramethyl-2-(4-(naphthalen-1-yl)phenyl)-1,3,2-dioxaborolane (46.9 g, 0.14 mol), Pd(PPh₃)₄ (4.9 g, 0.003 mol), NaOH (17 g, 0.43 mol), THE (285 mL) and water (100 mL) were added to Sub1-1b (50 g, 0.14 mol), and reacted at 70° C. for 6 hours. When the reaction is completed, the temperature of the reactant is cooled to room temperature and the reaction solvent is removed. Afterwards, the concentrated reactant was separated using a silica gel column or recrystallization method to obtain 68 g (92.1%) of product Sub1-1.

### 2. Synthesis Example of Sub1-3

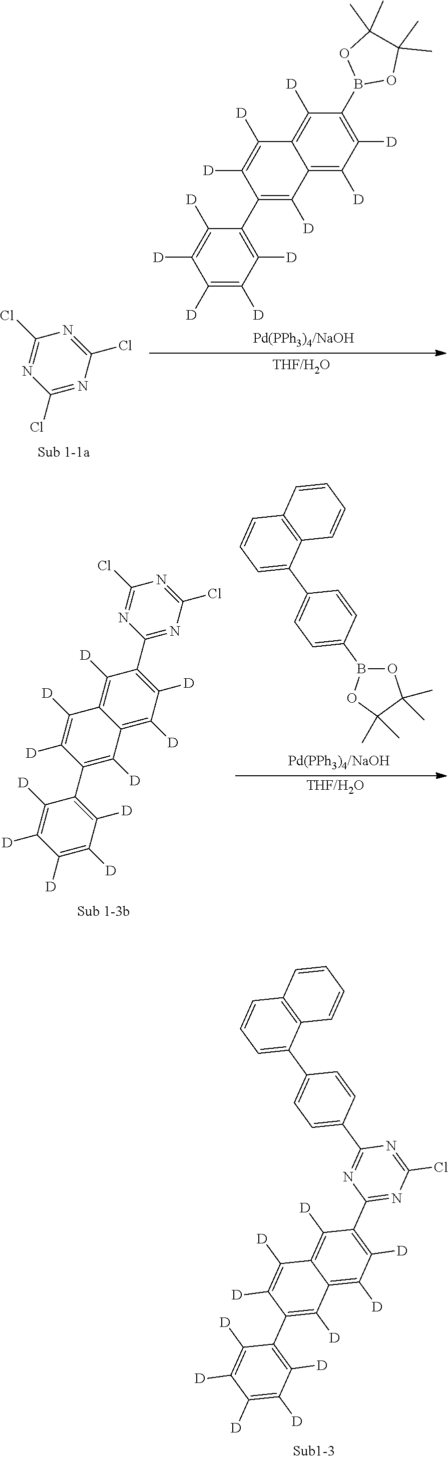 [SMILES: [2H]c1c([2H])c([2H])c(-c2c([2H])c([2H])c3c([2H])c(B4OC(C)(C)C(C)(C)O4)c([2H])c([2H])c3c2[2H])c([2H])c1[2H] ; Clc1nc(Cl)nc(Cl)n1 ; [2H]c1c([2H])c([2H])c(-c2c([2H])c([2H])c3c([2H])c(-c4nc(Cl)nc(Cl)n4)c([2H])c([2H])c3c2[2H])c([2H])c1[2H] ; CC1(C)OB(c2ccc(-c3cccc4ccccc34)cc2)OC1(C)C ; [2H]c1c([2H])c([2H])c(-c2c([2H])c([2H])c3c([2H])c(-c4nc(Cl)nc(-c5ccc(-c6cccc7ccccc67)cc5)n4)c([2H])c([2H])c3c2[2H])c([2H])c1[2H]]

### (1) Synthesis of Sub1-3b

4,4,5,5-tetramethyl-2-(6-(phenyl-d5)naphthalen-2-yl-1,3,4,5,7,8-d6)-1,3,2-dioxaborolane (92.7 g, 0.27 mol), Pd(PPh₃)₄ (9.4 g, 0.008 mol), NaOH (32.6 g, 0.82 mol), THE (550 mL) and water (180 mL) were added to Sub1-1a (50 g, 0.27 mol), and reacted at 70° C. for 6 hours. When the reaction is completed, 78 g (79.1%) of product Sub1-3b was obtained using the separation method of Sub1-1b.

### (2) Synthesis of Sub1-3

4,4,5,5-tetramethyl-2-(4-(naphthalen-1-yl)phenyl)-1,3,2-dioxaborolane (45.5 g, 0.14 mol), Pd(PPh₃)₄ (4.8 g, 0.004 mol), NaOH (16.5 g, 0.41 mol), THE (275 mL) and water (90 mL) were added to Sub1-3b (50 g, 0.14 mol), and reacted at 70° C. for 6 hours. When the reaction is completed, 66 g (90.2%) of product Sub1-3 was obtained using the separation method of Sub1-1.

### 3. Synthesis Example of Sub1-5

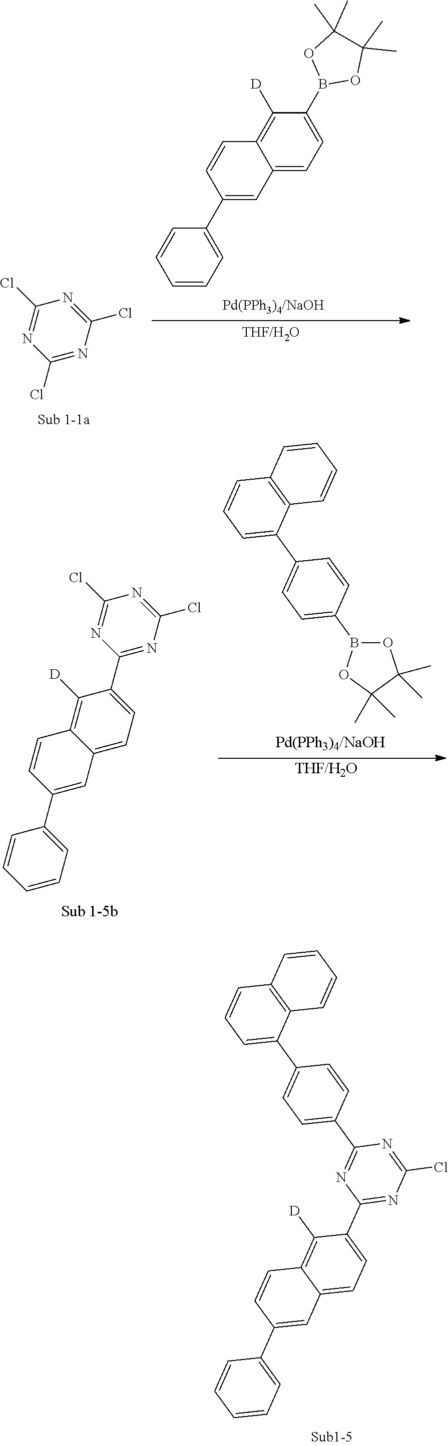 [SMILES: Clc1nc(Cl)nc(Cl)n1 ; [2H]c1c(B2OC(C)(C)C(C)(C)O2)ccc2cc(-c3ccccc3)ccc12 ; [2H]c1c(-c2nc(Cl)nc(Cl)n2)ccc2cc(-c3ccccc3)ccc12 ; CC1(C)OB(c2ccc(-c3cccc4ccccc34)cc2)OC1(C)C ; [2H]c1c(-c2nc(Cl)nc(-c3ccc(-c4cccc5ccccc45)cc3)n2)ccc2cc(-c3ccccc3)ccc12]

### (1) Synthesis of Sub1-5b

4,4,5,5-tetramethyl-2-(6-phenylnaphthalen-2-yl-1-d)-1,3,2-dioxaborolane (54 g, 0.16 mol), Pd(PPh₃)₄ (5.7 g, 0.005 mol), NaOH (19.6 g, 0.49 mol), THE (330 mL) and water (110 mL) were added to Sub1-1a (30 g, 0.16 mol), and reacted at 70° C. for 6 hours. When the reaction is completed, 42 g (73%) of product Sub1-7b was obtained using the separation method of Sub1-1b.

### (2) Synthesis of Sub1-5

4,4,5,5-tetramethyl-2-(4-(naphthalen-1-yl)phenyl)-1,3,2-dioxaborolane (28 g, 0.08 mol), Pd(PPh₃)₄ (2.9 g, 0.003 mol), NaOH (10.2 g, 0.25 mol), THE (170 mL) and water (60 mL) were added to Sub1-5b (30 g, 0.08 mol), and reacted at 70° C. for 6 hours. When the reaction is completed, 40 g (90%) of product Sub1-5 was obtained using the separation method of Sub1-1.

Meanwhile, the compounds belonging to Sub 1 may be the following compounds, but are not limited thereto, and Table 1 shows the FD-MS (Field Desorption-Mass Spectrometry) values of the compounds belonging to Sub 1.

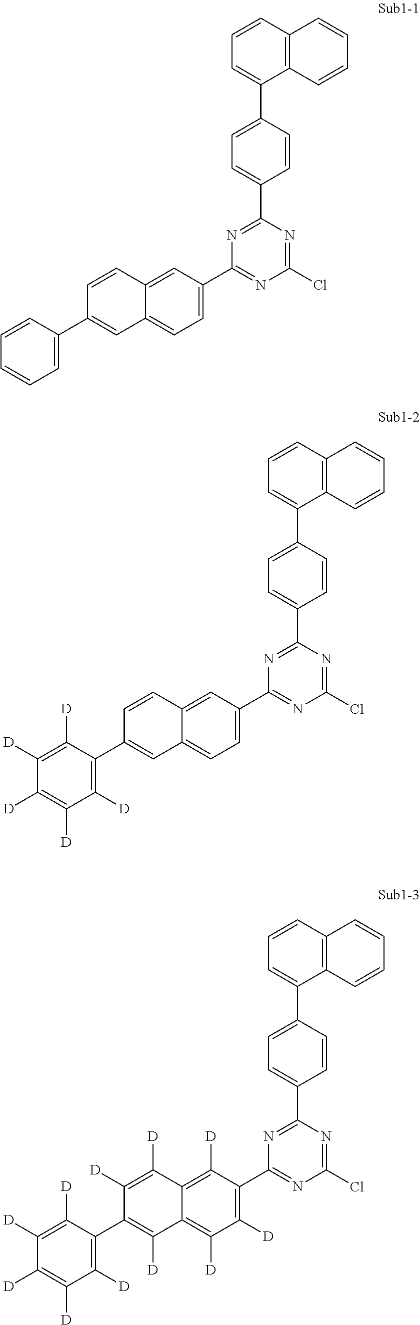 [SMILES: Clc1nc(-c2ccc(-c3cccc4ccccc34)cc2)nc(-c2ccc3cc(-c4ccccc4)ccc3c2)n1 ; [2H]c1c([2H])c([2H])c(-c2ccc3cc(-c4nc(Cl)nc(-c5ccc(-c6cccc7ccccc67)cc5)n4)ccc3c2)c([2H])c1[2H] ; [2H]c1c([2H])c([2H])c(-c2c([2H])c([2H])c3c([2H])c(-c4nc(Cl)nc(-c5ccc(-c6cccc7ccccc67)cc5)n4)c([2H])c([2H])c3c2[2H])c([2H])c1[2H]]

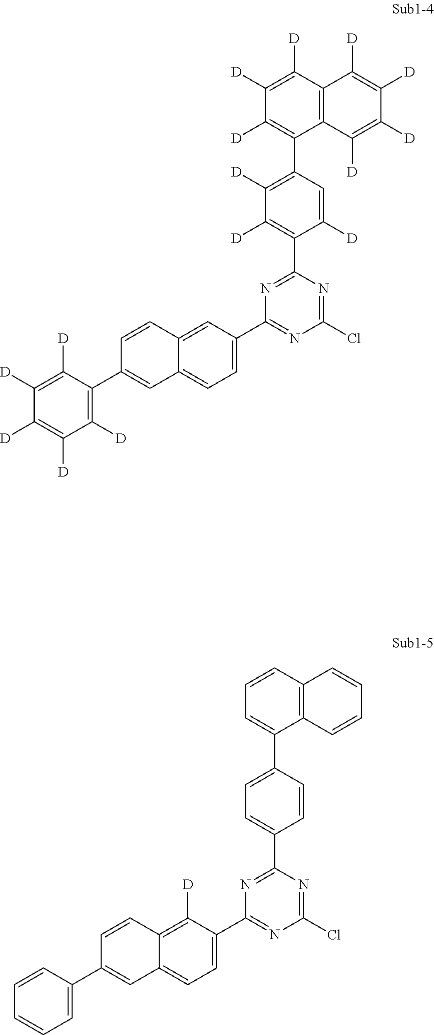 [SMILES: [2H]c1cc(-c2c([2H])c([2H])c([2H])c3c([2H])c([2H])c([2H])c([2H])c23)c([2H])c([2H])c1-c1nc(Cl)nc(-c2ccc3cc(-c4c([2H])c([2H])c([2H])c([2H])c4[2H])ccc3c2)n1 ; [2H]c1c(-c2nc(Cl)nc(-c3ccc(-c4cccc5ccccc45)cc3)n2)ccc2cc(-c3ccccc3)ccc12]

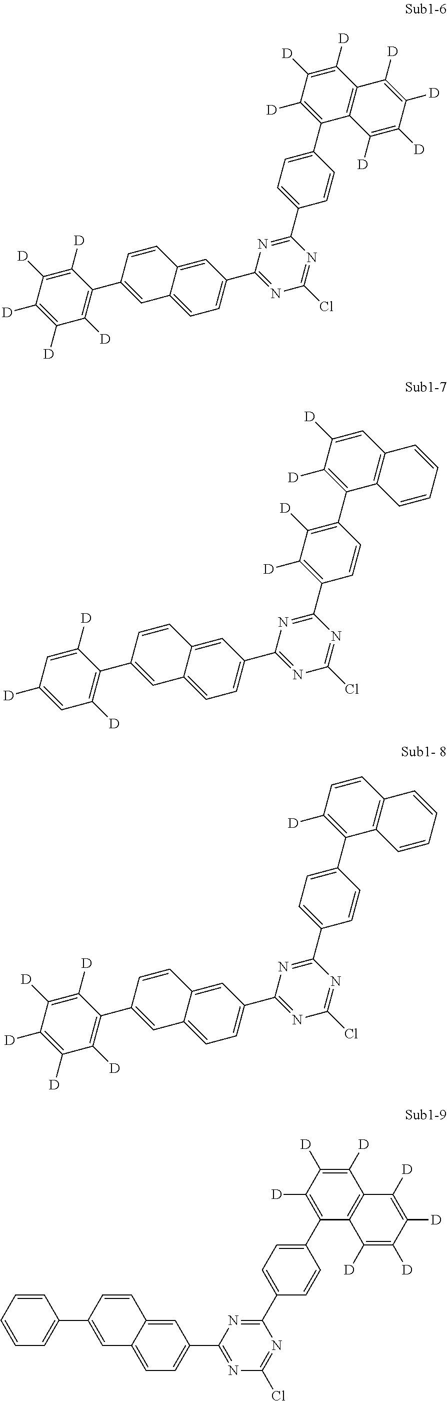 [SMILES: [2H]c1c([2H])c([2H])c(-c2ccc3cc(-c4nc(Cl)nc(-c5ccc(-c6c([2H])c([2H])c([2H])c7c([2H])c([2H])c([2H])c([2H])c67)cc5)n4)ccc3c2)c([2H])c1[2H] ; [2H]c1cc([2H])c(-c2ccc3cc(-c4nc(Cl)nc(-c5ccc(-c6c([2H])c([2H])cc7ccccc67)c([2H])c5[2H])n4)ccc3c2)c([2H])c1 ; [2H]c1ccc2ccccc2c1-c1ccc(-c2nc(Cl)nc(-c3ccc4cc(-c5c([2H])c([2H])c([2H])c([2H])c5[2H])ccc4c3)n2)cc1 ; [2H]c1c([2H])c([2H])c2c(-c3ccc(-c4nc(Cl)nc(-c5ccc6cc(-c7ccccc7)ccc6c5)n4)cc3)c([2H])c([2H])c([2H])c2c1[2H]]

TABLE 1

| Com- | |
|---|---|
| pound | FD-MS |
| Sub1-1 | m/z = 519.15(C₃₅H₂₂CIN₃ = 520.03) |
| Sub1-2 | m/z = 524.18(C₃₅H₁₇D₅CIN₃ = 525.06) |
| Sub1-3 | m/z = 530.22(C₃₅H₁₁D₁₁CIN₃ = 531.1) |
| Sub1-4 | m/z = 534.24(C₃₅H₇D₁₅CIN₃ = 535.12) |
| Sub1-5 | m/z = 520.16(C₃₅H₂₁DCIN₃ = 521.04) |
| Sub1-6 | m/z = 531.23(C₃₅H₁₀D₁₂CIN₃ = 532.11) |
| Sub1-7 | m/z = 526.19(C₃₅H₁₅D₇CIN₃ = 527.07) |
| Sub1-8 | m/z = 525.19(C₃₅H₁₆D₆CIN₃ = 526.07) |
| Sub1-9 | m/z = 526.19(C₃₅H₁₅D₇CIN₃ = 527.07) |

Meanwhile, the compounds belonging to Sub 2 may be the following compounds, but are not limited thereto, and Table 2 shows the FD-MS values of the compounds belonging to Sub 2.

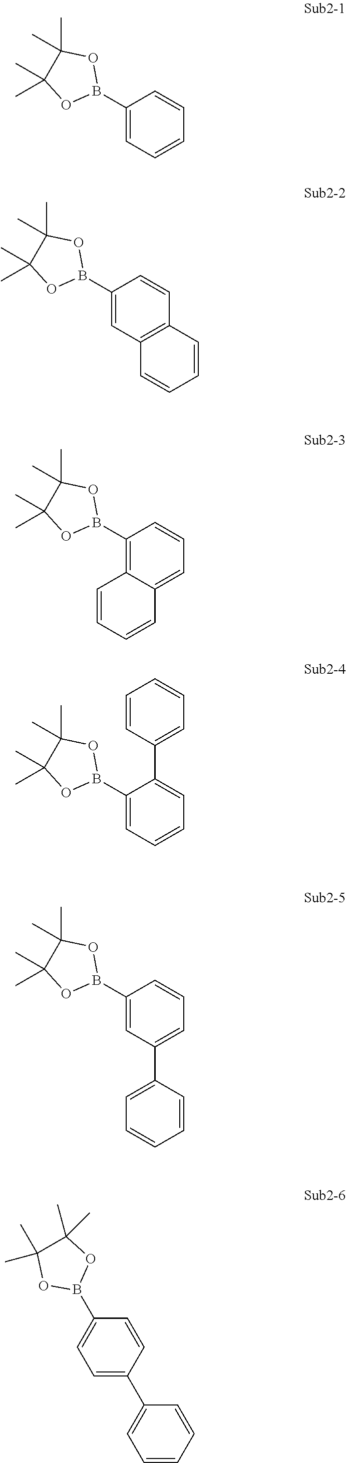 [SMILES: CC1(C)OB(c2ccccc2)OC1(C)C ; CC1(C)OB(c2ccc3ccccc3c2)OC1(C)C ; CC1(C)OB(c2cccc3ccccc23)OC1(C)C ; CC1(C)OB(c2ccccc2-c2ccccc2)OC1(C)C ; CC1(C)OB(c2cccc(-c3ccccc3)c2)OC1(C)C ; CC1(C)OB(c2ccc(-c3ccccc3)cc2)OC1(C)C]

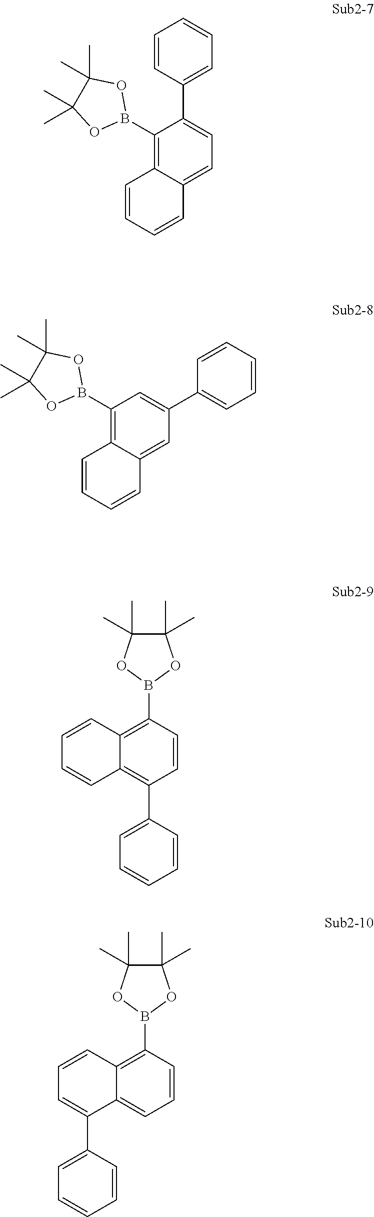 [SMILES: CC1(C)OB(c2c(-c3ccccc3)ccc3ccccc23)OC1(C)C ; CC1(C)OB(c2cc(-c3ccccc3)cc3ccccc23)OC1(C)C ; CC1(C)OB(c2ccc(-c3ccccc3)c3ccccc23)OC1(C)C ; CC1(C)OB(c2cccc3c(-c4ccccc4)cccc23)OC1(C)C]

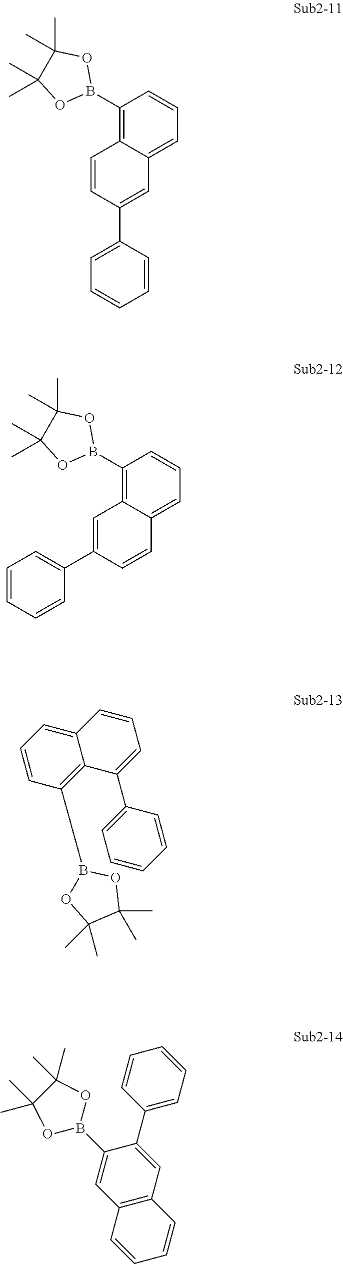 [SMILES: CC1(C)OB(c2cccc3cc(-c4ccccc4)ccc23)OC1(C)C ; CC1(C)OB(c2cccc3ccc(-c4ccccc4)cc23)OC1(C)C ; CC1(C)OB(c2cccc3cccc(-c4ccccc4)c23)OC1(C)C ; CC1(C)OB(c2cc3ccccc3cc2-c2ccccc2)OC1(C)C]

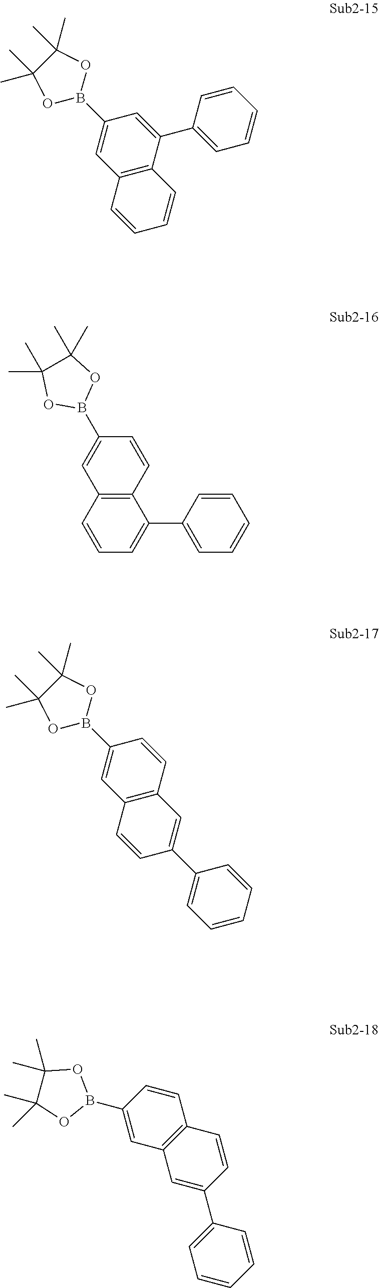 [SMILES: CC1(C)OB(c2cc(-c3ccccc3)c3ccccc3c2)OC1(C)C ; CC1(C)OB(c2ccc3c(-c4ccccc4)cccc3c2)OC1(C)C ; CC1(C)OB(c2ccc3cc(-c4ccccc4)ccc3c2)OC1(C)C ; CC1(C)OB(c2ccc3ccc(-c4ccccc4)cc3c2)OC1(C)C]

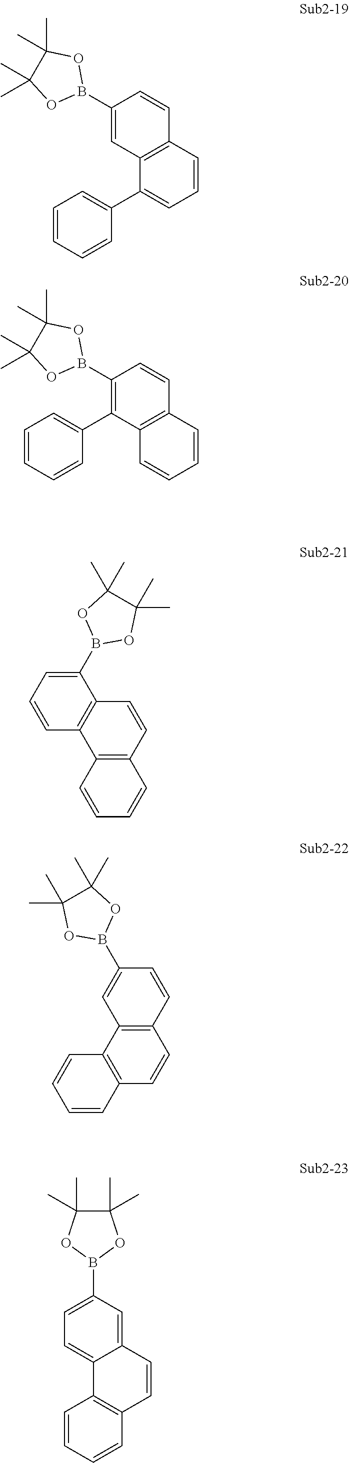

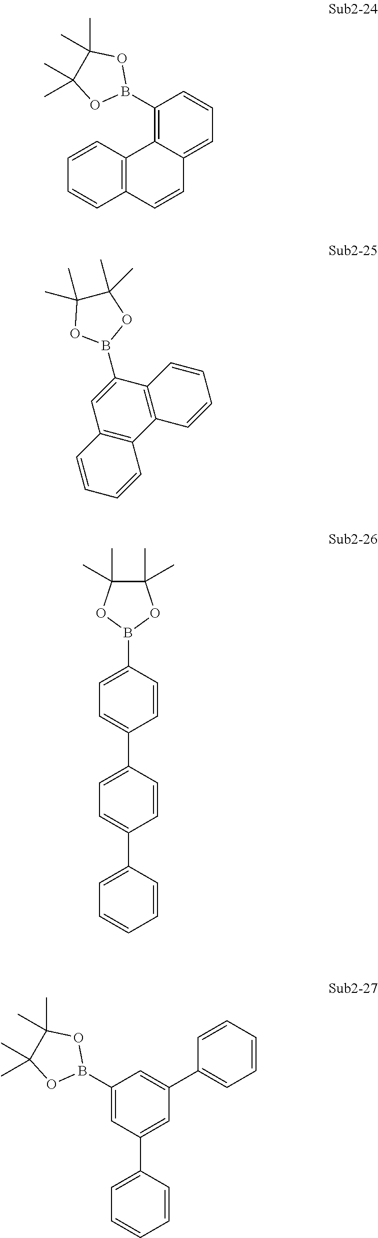

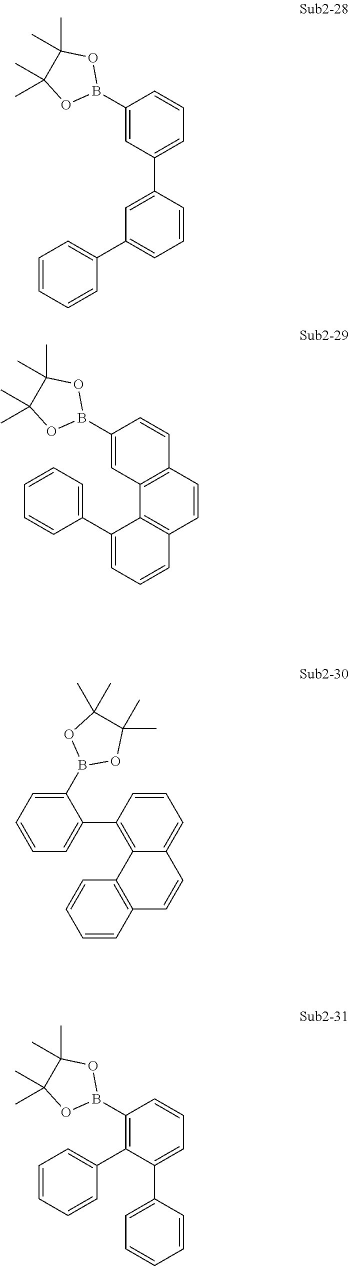

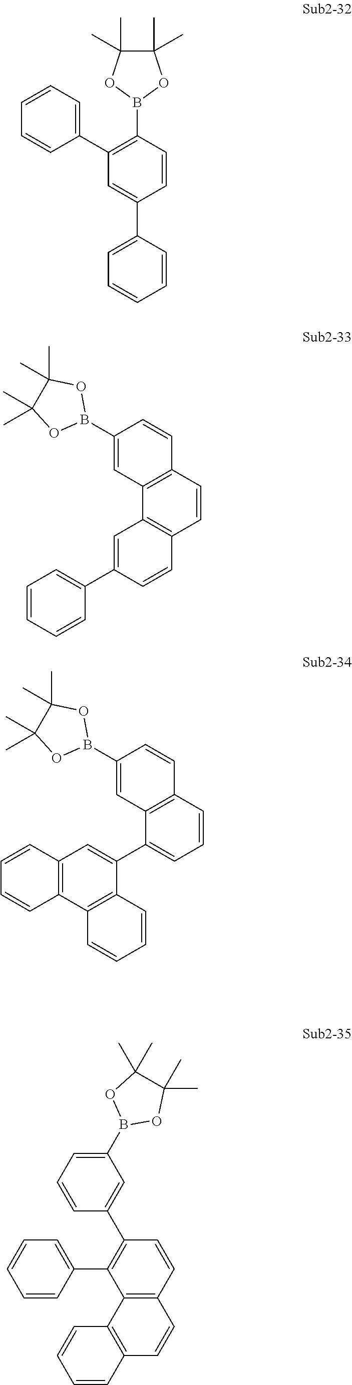

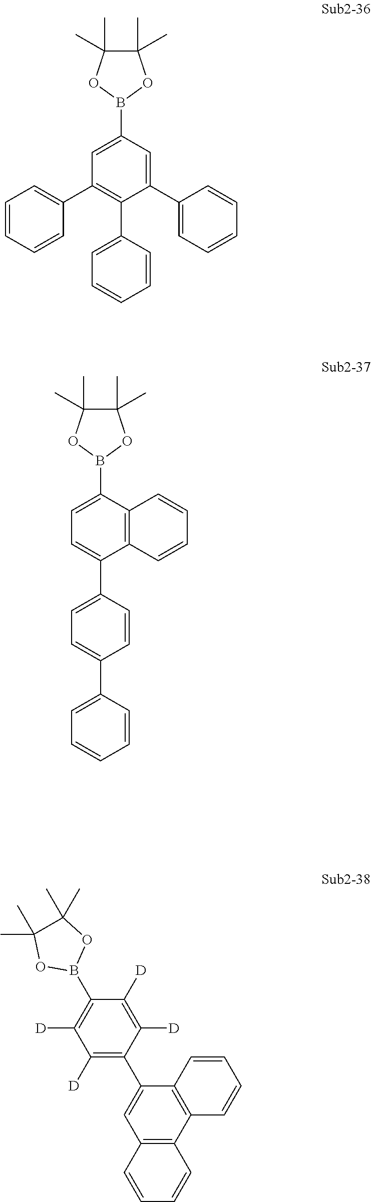 [SMILES: CC1(C)OB(c2cc(-c3ccccc3)c(-c3ccccc3)c(-c3ccccc3)c2)OC1(C)C ; CC1(C)OB(c2ccc(-c3ccc(-c4ccccc4)cc3)c3ccccc23)OC1(C)C ; [2H]c1c([2H])c(-c2cc3ccccc3c3ccccc23)c([2H])c([2H])c1B1OC(C)(C)C(C)(C)O1]

TABLE 2

| Compound | FD-MS |
|---|---|
| Sub2-1 | m/z = 204.13(C₁₂H₁₇BO₂ = 204.08) |
| Sub2-2 | m/z = 254.15(C₁₆H₁₉BO₂ = 254.14) |
| Sub2-3 | m/z = 254.15(C₁₆H₁₉BO₂ = 254.14) |
| Sub2-4 | m/z = 280.16(C₁₈H₂₁BO₂ = 280.17) |
| Sub2-5 | m/z = 280.16(C₁₈H₂₁BO₂ = 280.17) |
| Sub2-6 | m/z = 280.16(C₁₈H₂₁BO₂ = 280.17) |
| Sub2-7 | m/z = 330.18(C₂₂H₂₃BO₂ = 330.23) |
| Sub2-8 | m/z = 330.18(C₂₂H₂₃BO₂ = 330.23) |
| Sub2-9 | m/z = 330.18(C₂₂H₂₃BO₂ = 330.23) |
| Sub2-10 | m/z = 330.18(C₂₂H₂₃BO₂ = 330.23) |
| Sub2-11 | m/z = 330.18(C₂₂H₂₃BO₂ = 330.23) |
| Sub2-12 | m/z = 330.18(C₂₂H₂₃BO₂ = 330.23) |
| Sub2-13 | m/z = 330.18(C₂₂H₂₃BO₂ = 330.23) |
| Sub2-14 | m/z = 330.18(C₂₂H₂₃BO₂ = 330.23) |
| Sub2-15 | m/z = 330.18(C₂₂H₂₃BO₂ = 330.23) |
| Sub2-16 | m/z = 330.18(C₂₂H₂₃BO₂ = 330.23) |
| Sub2-17 | m/z = 330.18(C₂₂H₂₃BO₂ = 330.23) |
| Sub2-18 | m/z = 330.18(C₂₂H₂₃BO₂ = 330.23) |
| Sub2-19 | m/z = 330.18(C₂₂H₂₃BO₂ = 330.23) |
| Sub2-20 | m/z = 330.18(C₂₂H₂₃BO₂ = 330.23) |
| Sub2-21 | m/z = 304.16(C₂₀H₂₁BO₂ = 304.2) |
| Sub2-22 | m/z = 304.16(C₂₀H₂₁BO₂ = 304.2) |
| Sub2-23 | m/z = 304.16(C₂₀H₂₁BO₂ = 304.2) |
| Sub2-24 | m/z = 304.16(C₂₀H₂₁BO₂ = 304.2) |
| Sub2-25 | m/z = 304.16(C₂₀H₂₁BO₂ = 304.2) |
| Sub2-26 | m/z = 356.19(C₂₄H₂₅BO₂ = 356.27) |
| Sub2-27 | m/z = 356.19(C₂₄H₂₅BO₂ = 356.27) |
| Sub2-28 | m/z = 356.19(C₂₄H₂₅BO₂ = 356.27) |
| Sub2-29 | m/z = 380.19(C₂₆H₂₅BO₂ = 380.29) |
| Sub2-30 | m/z = 380.19(C₂₆H₂₅BO₂ = 380.29) |
| Sub2-31 | m/z = 356.19(C₂₄H₂₅BO₂ = 356.27) |
| Sub2-32 | m/z = 380.19(C₂₆H₂₅BO₂ = 380.29) |
| Sub2-33 | m/z = 356.19(C₂₄H₂₅BO₂ = 356.27) |
| Sub2-34 | m/z = 430.21(C₃₀H₂₇BO₂ = 430.35) |
| Sub2-35 | m/z = 456.23(C₃₂H₂₉BO₂ = 456.39) |
| Sub2-36 | m/z = 432.23(C₃₀H₂₉BO₂ = 432.37) |
| Sub2-37 | m/z = 406.21(C₂₈H₂₇BO₂ = 406.33) |
| Sub2-38 | m/z = 384.22(C₂₆H₂₁D₄BO₂ = 384.32) |

### II. Synthesis of Final Product

### 1. Synthesis Example of P-1

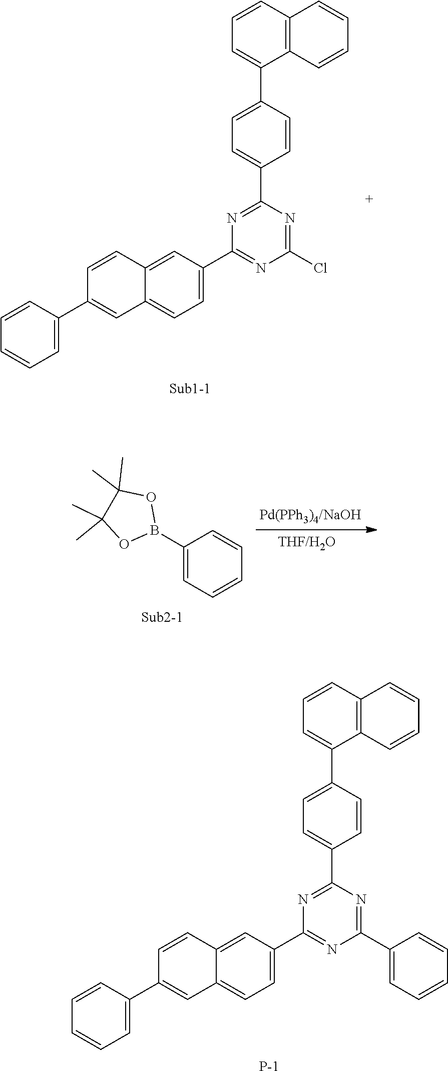

Sub1-1 (20 g, 0.04 mol), Sub2-1 (7.8 g, 0.04 mol), Pd(PPh₃)₄ (1.3 g, 0.001 mol), NaOH (4.6 g, 0.12 mol), THE (80 mL) and water (20 mL) were added, and reacted at 70° C. for 6 hours. When the reaction is completed, the temperature of the reactant is cooled to room temperature and the reaction solvent is removed. Afterwards, the concentrated reactant was separated using a silica gel column or recrystallization method to obtain 19 g (88.1%) of product P-1.

### 2. Synthesis Example of P-6

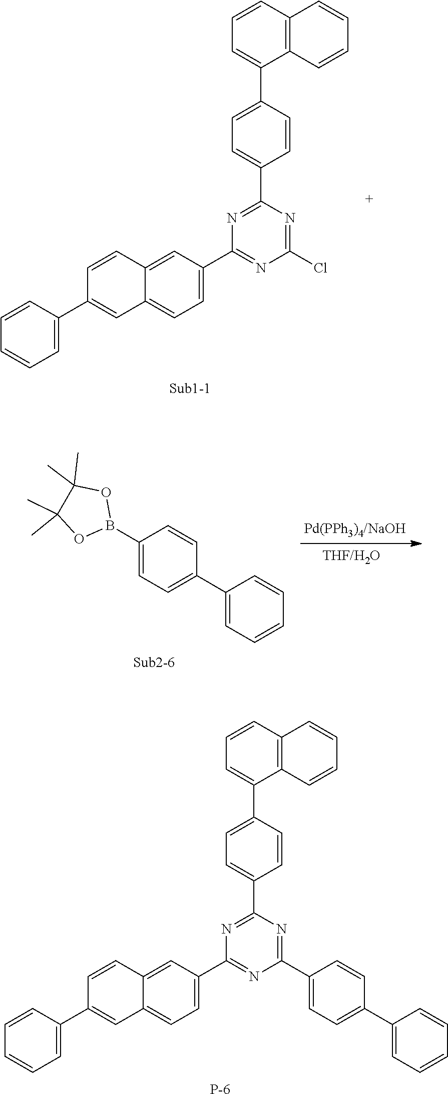

Sub1-1 (20 g, 0.04 mol), Sub2-6 (10.8 g, 0.04 mol), Pd(PPh₃)₄ (1.3 g, 0.001 mol), NaOH (4.6 g, 0.12 mol), THE (80 mL) and water (20 mL) were added, and reacted at 70° C. for 6 hours. When the reaction is completed, 22 g (89.8%) of product P-6 was obtained using the separation method of P-1.

### 3. Synthesis Example of P-17

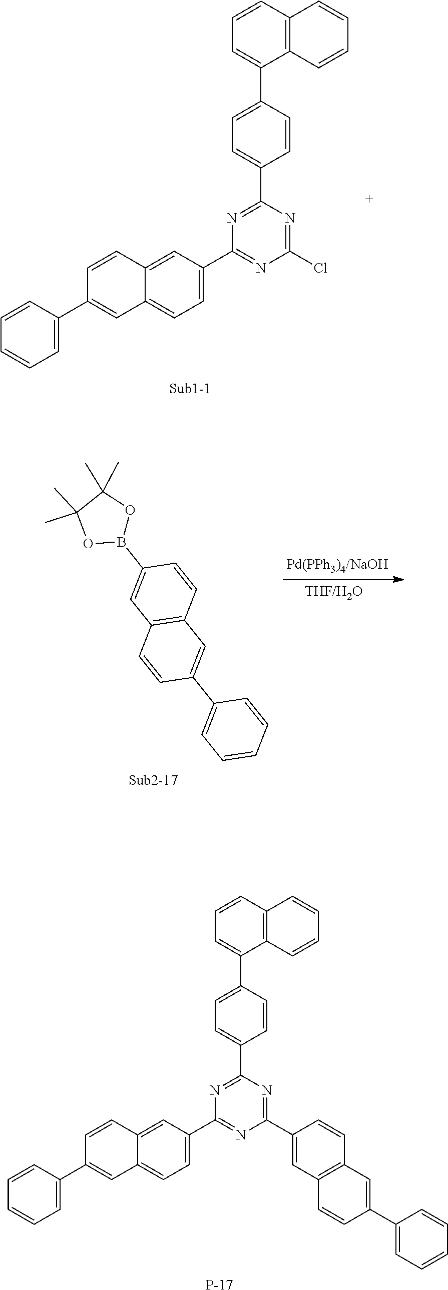

Sub1-1 (20 g, 0.04 mol), Sub2-17 (12.7 g, 0.04 mol), Pd(PPh₃)₄ (1.3 g, 0.001 mol), NaOH (4.6 g, 0.12 mol), THE (80 mL) and water (20 mL) were added, and reaction was performed at 70° C. for 6 hours. When the reaction was completed, 23 g (87%) of product P-17 was obtained using the separation method of P-1.

### 4. Synthesis Example of P-24

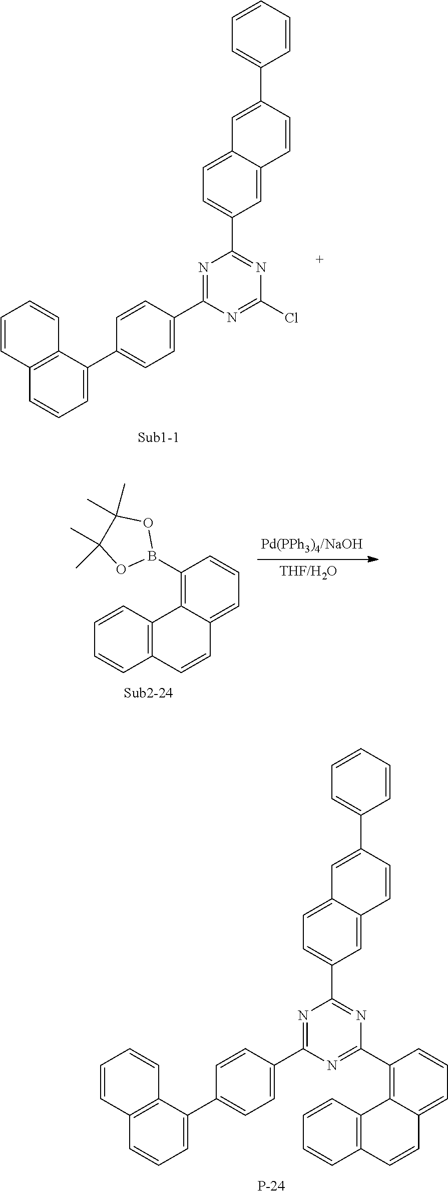

Sub1-1 (30 g, 0.06 mol), Sub2-24 (17.5 g, 0.06 mol), Pd(PPh₃)₄ (2 g, 0.002 mol), NaOH (6.9 g, 0.17 mol), THE (120 mL) and water (40 mL) were added, and reaction was performed at 70° C. for 6 hours. When the reaction was completed, 35 g (91.8%) of product P-24 was obtained using the separation method of P-1.

### 5. Synthesis Example of P-38

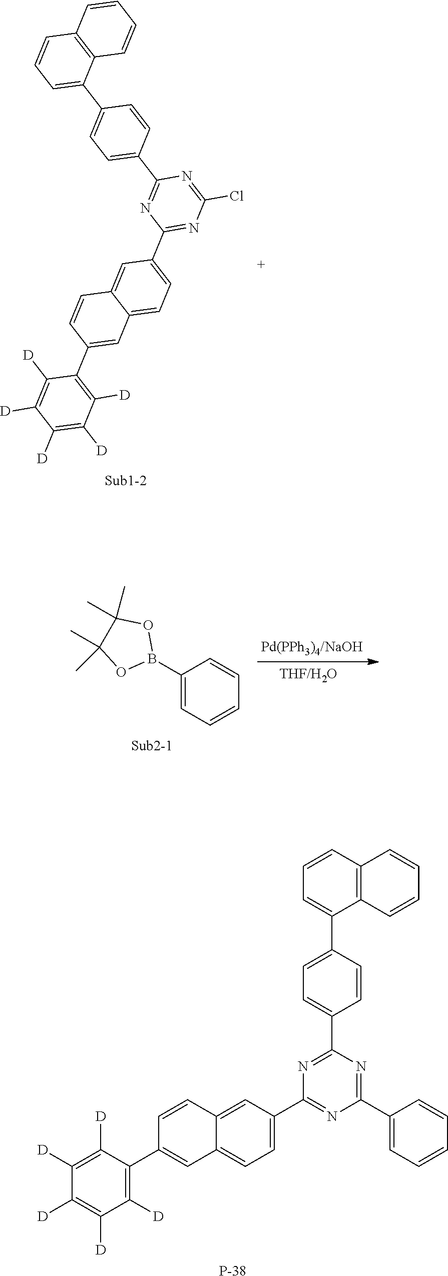

Sub1-2 (30 g, 0.06 mol), Sub2-1 (11.7 g, 0.06 mol), Pd(PPh₃)₄ (2 g, 0.002 mol), NaOH (6.9 g, 0.17 mol), THE (120 mL) and water (40 mL) were added, and reaction was performed at 70° C. for 6 hours. When the reaction was completed, 30 g (92.8%) of product P-38 was obtained using the separation method of P-1.

Meanwhile, the FD-MS values of compounds P-1 to P-40, N-1 to N-100, and S-1 to S-112 of the present invention prepared according to the above synthesis examples are shown in Table 3.

TABLE 3

| Com- | |
|---|---|
| pound | FD-MS |
| P-1 | m/z = 561.22(C₄₁H₂₇N₃ = 561.69) |
| P-2 | m/z = 611.24(C₄₅H₂₉N₃ = 611.75) |
| P-3 | m/z = 611.24(C₄₅H₂₉N₃ = 611.75) |
| P-4 | m/z = 637.25(C₄₇H₃₁N₃ = 637.79) |
| P-5 | m/z = 637.25(C₄₇H₃₁N₃ = 637.79) |
| P-6 | m/z = 637.25(C₄₇H₃₁N₃ = 637.79) |
| P-7 | m/z = 687.27(C₅₁H₃₃N₃ = 687.85) |
| P-8 | m/z = 687.27(C₅₁H₃₃N₃ = 687.85) |
| P-9 | m/z = 687.27(C₅₁H₃₃N₃ = 687.85) |
| P-10 | m/z = 687.27(C₅₁H₃₃N₃ = 687.85) |
| P-11 | m/z = 687.27(C₅₁H₃₃N₃ = 687.85) |
| P-12 | m/z = 687.27(C₅₁H₃₃N₃ = 687.85) |
| P-13 | m/z = 687.27(C₅₁H₃₃N₃ = 687.85) |
| P-14 | m/z = 687.27(C₅₁H₃₃N₃ = 687.85) |
| P-15 | m/z = 687.27(C₅₁H₃₃N₃ = 687.85) |
| P-16 | m/z = 687.27(C₅₁H₃₃N₃ = 687.85) |
| P-17 | m/z = 687.27(C₅₁H₃₃N₃ = 687.85) |
| P-18 | m/z = 687.27(C₅₁H₃₃N₃ = 687.85) |
| P-19 | m/z = 687.27(C₅₁H₃₃N₃ = 687.85) |
| P-20 | m/z = 687.27(C₅₁H₃₃N₃ = 687.85) |
| P-21 | m/z = 661.25(C₄₉H₃₁N₃ = 661.81) |
| P-22 | m/z = 661.25(C₄₉H₃₁N₃ = 661.81) |
| P-23 | m/z = 661.25(C₄₉H₃₁N₃ = 661.81) |
| P-24 | m/z = 661.25(C₄₉H₃₁N₃ = 661.81) |
| P-25 | m/z = 661.25(C₄₉H₃₁N₃ = 661.81) |
| P-26 | m/z = 713.28(C₅₃H₃₅N₃ = 713.88) |
| P-27 | m/z = 713.28(C₅₃H₃₅N₃ = 713.88) |
| P-28 | m/z = 713.28(C₅₃H₃₅N₃ = 713.88) |
| P-29 | m/z = 737.28(C₅₅H₃₅N₃ = 737.91) |
| P-30 | m/z = 737.28(C₅₅H₃₅N₃ = 737.91) |
| P-31 | m/z = 713.28(C₅₃H₃₅N₃ = 713.88) |
| P-32 | m/z = 713.28(C₅₃H₃₅N₃ = 713.88) |
| P-33 | m/z = 737.28(C₅₃H₃₅N₃ = 737.91) |
| P-34 | m/z = 787.3(C₅₉H₃₇N₃ = 787.97) |
| P-35 | m/z = 813.31(C₆₁H₃₉N₃ = 814) |
| P-36 | m/z = 789.31(C₅₉H₃₉N₃ = 789.98) |
| P-37 | m/z = 763.3(C₅₇H₃₇N₃ = 763.94) |
| P-38 | m/z = 566.25(C₄₁H₂₂D₅N₃ = 566.72) |
| P-39 | m/z = 741.31(C₅₅H₃₁D₄N₃ = 741.93) |
| P-40 | m/z = 572.29(C₄₁H₁₆D₁₁N₃ = 572.76) |
| N-1 | m/z = 487.19(C₃₆H₂₅NO = 487.6) |
| N-2 | m/z = 553.19(C₄₀H₂₇NS = 553.72) |
| N-3 | m/z = 563.26(C₄₃H₃₃N = 563.74) |
| N-4 | m/z = 602.27(C₄₅H₃₄N₂ = 602.78) |
| N-5 | m/z = 517.15(C₃₆H₂₃NOS = 517.65) |
| N-6 | m/z = 603.2(C₄₄H₂₉NS = 603.78) |
| N-7 | m/z = 735.29(C₅₇H₃₇N = 735.93) |
| N-8 | m/z = 562.24(C₄₂H₃₀N₂ = 562.72) |
| N-9 | m/z = 565.17(C₄₀H₂₃NO₃ = 565.63) |
| N-10 | m/z = 581.14(C₄₀H₂₃NO₂S = 581.69) |
| N-11 | m/z = 823.24(C₅₉H₃₇NS₂ = 824.07) |
| N-12 | m/z = 727.3(C₅₄H₃₇N₃ = 727.91) |
| N-13 | m/z = 627.22(C₄₆H₂₉NO₂ = 627.74) |
| N-14 | m/z = 633.16(C₄₄H₂₇NS₂ = 633.83) |
| N-15 | m/z = 675.29(C₅₂H₃₇N = 675.88) |
| N-16 | m/z = 678.3(C₅₁H₃₈N₂ = 678.88) |
| N-17 | m/z = 669.21(C₄₈H₃₁NOS = 669.84) |
| N-18 | m/z = 785.22(C₅₆H₃₅NS₂ = 786.02) |
| N-19 | m/z = 617.18(C₄₄H₂₇NOS = 617.77) |
| N-20 | m/z = 601.2(C₄₄H₂₇NO₂ = 601.71) |
| N-21 | m/z = 779.32(C₅₉H₄₁NO = 779.98) |
| N-22 | m/z = 583.23(C₄₂H₃₃NS = 583.79) |
| N-23 | m/z = 679.32(C₅₂H₄₁N = 679.91) |
| N-24 | m/z = 726.27(C₅₄H₃₄N₂O = 726.88) |
| N-25 | m/z = 593.18(C₄₂H₂₇NOS = 593.74) |
| N-26 | m/z = 774.22(C₅₄H₃₄N₂S₂ = 775) |
| N-27 | m/z = 557.24(C₄₀H₃₁NO₂ = 557.69) |
| N-28 | m/z = 652.25(C₄₈H₃₂N₂O = 652.8) |
| N-29 | m/z = 619.29(C₄₆H₃₇NO = 619.81) |
| N-30 | m/z = 603.2(C₄₄H₂₉NS = 603.78) |
| N-31 | m/z = 813.3(C₆₂H₃₉NO = 814) |
| N-32 | m/z = 784.29(C₅₇H₄₀N₂S = 785.02) |
| N-33 | m/z = 577.2(C₄₂H₂₇NO₂ = 577.68) |
| N-34 | m/z = 607.14(C₄₂H₂₅NS₂ = 607.79) |
| N-35 | m/z = 801.34(C₆₂H₄₃N = 802.03) |
| N-36 | m/z = 575.24(C₄₂H₂₉N₃ = 575.72) |
| N-37 | m/z = 577.2(C₄₂H₂₇NO₂ = 577.68) |
| N-38 | m/z = 607.14(C₄₂H₂₅NS₂ = 607.79) |
| N-39 | m/z = 801.34(C₆₂H₄₃N = 802.03) |
| N-40 | m/z = 575.24(C₄₂H₂₉N₃ = 575.72) |
| N-41 | m/z = 601.2(C₄₄H₂₇NO₂ = 601.71) |
| N-42 | m/z = 471.11(C₃₁H₂₁NS₂ = 471.64) |
| N-43 | m/z = 675.29(C₅₂H₃₇N = 675.88) |
| N-44 | m/z = 727.3(C₅₄H₃₇N₃ = 727.91) |
| N-45 | m/z = 603.2(C₄₄H₂₉NS = 603.78) |
| N-46 | m/z = 561.16(C₃₈H₂₇NS₂ = 561.76) |
| N-47 | m/z = 799.32(C₆₂H₄₁N = 800.02) |
| N-48 | m/z = 702.27(C₅₂H₃₄N₂O = 702.86) |
| N-49 | m/z = 729.27(C₅₄H₃₅NO₂ = 729.88) |
| N-50 | m/z = 785.22(C₅₆H₃₅NS₂ = 786.02) |
| N-51 | m/z = 812.32(C₆₂H₄₀N₂ = 813.02) |
| N-52 | m/z = 681.22(C₄₈H₃₁N₃S = 681.86) |
| N-53 | m/z = 615.18(C₄₄H₂₅NO₃ = 615.69) |
| N-54 | m/z = 763.15(C₅₂H₂₉NS₃ = 763.99) |
| N-55 | m/z = 593.31(C₄₅H₃₉N = 593.81) |
| N-56 | m/z = 840.33(C₆₂H₄₀N₄ = 841.03) |
| N-57 | m/z = 657.18(C₄₆H₂₇NO₂S = 657.79) |
| N-58 | m/z = 824.23(C₅₈H₃₆N₂S₂ = 825.06) |
| N-59 | m/z = 1195.42(C₉₁H₅₇NS = 1196.52) |
| N-60 | m/z = 656.19(C₄₆H₂₈N₂OS = 656.8) |
| N-61 | m/z = 607.16(C₄₂H₂₅NO₂S = 607.73) |
| N-62 | m/z = 773.2(C₅₄H₃₁NO₃S = 773.91) |
| N-63 | m/z = 1013.4(C₇₉H₅₁N = 1014.28) |
| N-64 | m/z = 758.24(C₅₄H₃₄N₂OS = 758.94) |
| N-65 | m/z = 623.14(C₄₂H₂₅NOS₂ = 623.79) |
| N-66 | m/z = 763.16(C₅₂H₂₉NO₂S₂ = 763.93) |
| N-67 | m/z = 799.2(C₅₆H₃₃NOS₂ = 800.01) |
| N-68 | m/z = 743.23(C₅₄H₃₃NOS = 743.92) |
| N-69 | m/z = 872.25(C₆₂H₃₆N₂O₂S = 873.04) |
| N-70 | m/z = 772.22(C₅₄H₃₂N₂O₂S = 772.92) |
| N-71 | m/z = 830.28(C₆₁H₃₈N₂S = 831.05) |
| N-72 | m/z = 808.25(C₅₈H₃₃FN₂O₂ = 808.91) |
| N-73 | m/z = 929.21(C₆₄H₃₅NO₃S₂ = 930.11) |
| N-74 | m/z = 963.27(C₆₈H₄₁N₃S₂ = 964.22) |
| N-75 | m/z = 809.24(C₅₈H₃₅NO₂S = 809.98) |
| N-76 | m/z = 893.29(C₆₆H₃₉NO₃ = 894.04) |
| N-77 | m/z = 794.28(C₅₈H₃₈N₂S = 795.02) |
| N-78 | m/z = 900.26(C₆₄H₄₀N₂S₂ = 901.16) |
| N-79 | m/z = 758.28(C₅₅H₃₈N₂S = 758.98) |
| N-80 | m/z = 1082.37(C₈₁H₅₀N₂S = 1083.37) |
| N-81 | m/z = 573.25(C₄₄H₃₁N = 573.74) |
| N-82 | m/z = 649.28(C₅₀H₃₅N = 649.84) |
| N-83 | m/z = 699.29(C₅₄H₃₇N = 699.9) |
| N-84 | m/z = 699.29(C₅₄H₃₇N = 699.9) |
| N-85 | m/z = 673.28(C₅₂H₃₅N = 673.86) |
| N-86 | m/z = 649.28(C₅₀H₃₅N = 649.84) |
| N-87 | m/z = 625.28(C₄₈H₃₅N = 625.82) |
| N-88 | m/z = 673.28(C₅₂H₃₅N = 673.86) |
| N-89 | m/z = 773.31(C₆₀H₃₉N = 773.98) |
| N-90 | m/z = 749.31(C₅₈H₃₉N = 749.96) |
| N-91 | m/z = 699.29(C₅₄H₃₇N = 699.9) |
| N-92 | m/z = 599.26(C₄₆H₃₃N = 599.78) |
| N-93 | m/z = 639.26(C₄₈H₃₃NO = 639.8) |
| N-94 | m/z = 765.25(C₅₇H₃₅NS = 765.97) |
| N-95 | m/z = 677.31(C₅₂H₃₉N = 677.89) |
| N-96 | m/z = 727.3(C₅₄H₃₇N₃ = 727.91) |
| N-97 | m/z = 657.18(C₄₆H₂₇NO₂S = 657.79) |
| N-98 | m/z = 641.20(C₄₆H₂₇NO₃ = 641.73) |
| N-99 | m/z = 657.18(C₄₆H₂₇NO₂S = 657.79) |
| N-100 | m/z = 691.21(C₅₀H₂₉NO₃ = 691.79) |
| S-1 | m/z = 408.16(C₃₀H₂₀N₂ = 408.5) |
| S-2 | m/z = 534.21(C₄₀H₂₆N₂ = 534.66) |
| S-3 | m/z = 560.23(C₄₂H₂₈N₂ = 560.7) |
| S-4 | m/z = 584.23(C₄₄H₂₈N₂ = 584.72) |
| S-5 | m/z = 560.23(C₄₂H₂₈N₂ = 560.7) |
| S-6 | m/z = 634.24(C₄₈H₃₀N₂ = 634.78) |
| S-7 | m/z = 610.24(C₄₆H₃₀N₂ = 610.76) |
| S-8 | m/z = 498.17(C₃₆H₂₂N₂O = 498.59) |
| S-9 | m/z = 574.2(C₄₂H₂₆N₂O = 574.68) |
| S-10 | m/z = 660.26(C₅₀H₃₂N₂ = 660.82) |
| S-11 | m/z = 686.27(C₅₂H₃₄N₂ = 686.86) |
| S-12 | m/z = 620.14(C₄₂H₂₄N₂S₂ = 620.79) |
| S-13 | m/z = 640.2(C₄₆H₂₈N₂S = 640.8) |
| S-14 | m/z = 560.23(C₄₂H₂₈N₂ = 560.7) |
| S-15 | m/z = 558.21(C₄₂H₂₆N₂ = 558.68) |
| S-16 | m/z = 548.19(C₄₀H₂₄N₂O = 548.65) |
| S-17 | m/z = 573.22(C₄₂H₂₇N₃ = 573.7) |
| S-18 | m/z = 564.17(C₄₀H₂₄N₂S = 564.71) |
| S-19 | m/z = 574.2(C₄₂H₂₆N₂O = 574.68) |
| S-20 | m/z = 564.17(C₄₀H₂₄N₂S = 564.71) |
| S-21 | m/z = 564.17(C₄₀H₂₄N₂S = 564.71) |
| S-22 | m/z = 813.31(C₆₁H₃₉N₃ = 814) |
| S-23 | m/z = 696.26(C₅₃H₃₂N₂ = 696.85) |
| S-24 | m/z = 691.23(C₄₉H₂₉N₃O₂ = 691.79) |
| S-25 | m/z = 710.27(C₅₄H₃₄N₂ = 710.88) |
| S-26 | m/z = 610.24(C₄₆H₃₀N₂ = 610.76) |
| S-27 | m/z = 670.15(C₄₆H₂₆N₂S₂ = 670.85) |
| S-28 | m/z = 640.29(C₄₈H₃₆N₂ = 640.83) |
| S-29 | m/z = 598.2(C₄₄H₂₆N₂O = 598.71) |
| S-30 | m/z = 623.24(C₄₆H₂₉N₃ = 623.76) |
| S-31 | m/z = 458.18(C₃₄H₂₂N₂ = 458.56) |
| S-32 | m/z = 548.19(C₄₀H₂₄N₂O = 548.65) |
| S-33 | m/z = 508.19(C₃₈H₂₄N₂ = 508.62) |
| S-34 | m/z = 508.19(C₃₈H₂₄N₂ = 508.62) |
| S-35 | m/z = 623.24(C₄₆H₂₉N₃ = 623.76) |
| S-36 | m/z = 564.17(C₄₀H₂₄N₂S = 564.71) |
| S-37 | m/z = 627.2(C₄₆H₂₉NS = 627.81) |
| S-38 | m/z = 505.1(C₃₄H₁₉NS₂ = 505.65) |
| S-39 | m/z = 514.15(C₃₆H₂₂N₂S = 514.65) |
| S-40 | m/z = 575.17(C₄₂H₂₅NS = 575.73) |
| S-41 | m/z = 642.21(C₄₆H₃₀N₂S = 642.82) |
| S-42 | m/z = 575.17(C₄₂H₂₅NS = 575.73) |
| S-43 | m/z = 606.18(C₄₂H₂₆N₂OS = 606.74) |
| S-44 | m/z = 575.17(C₄₂H₂₅NS = 575.73) |
| S-45 | m/z = 551.17(C₄₀H₂₅NS = 551.71) |
| S-46 | m/z = 607.14(C₄₂H₂₅NS₂ = 607.79) |
| S-47 | m/z = 525.16(C₃₈H₂₃NS = 525.67) |
| S-48 | m/z = 642.21(C₄₆H₃₀N₂S = 642.82) |
| S-49 | m/z = 548.19(C₄₀H₂₄N₂O = 548.65) |
| S-50 | m/z = 473.14(C₃₄H₁₉NO₂ = 473.53) |
| S-51 | m/z = 566.15(C₃₉H₂₂N₂OS = 566.68) |
| S-52 | m/z = 459.16(C₃₄H₂₁NO = 459.55) |
| S-53 | m/z = 473.14(C₃₄H₁₉NO₂ = 473.53) |
| S-54 | m/z = 523.16(C₃₈H₂₁NO₂ = 523.59) |
| S-55 | m/z = 539.13(C₃₈H₂₁NOS = 539.65) |
| S-56 | m/z = 548.19(C₄₀H₂₄N₂O = 548.65) |
| S-57 | m/z = 489.12(C₃₄H₁₉NOS = 489.59) |
| S-58 | m/z = 545.09(C₃₆H₁₉NOS₂ = 545.67) |
| S-59 | m/z = 549.17(C₄₀H₂₃NO₂ = 549.63) |
| S-60 | m/z = 565.15(C₄₀H₂₃NOS = 565.69) |
| S-61 | m/z = 523.16(C₃₈H₂₁NO₂ = 523.59) |
| S-62 | m/z = 598.2(C₄₄H₂₆N₂O = 598.71) |
| S-63 | m/z = 539.13(C₃₈H₂₁NOS = 539.65) |
| S-64 | m/z = 589.15(C₄₂H₂₃NOS = 589.71) |
| S-65 | m/z = 498.17(C₃₆H₂₂N₂O = 498.59) |
| S-66 | m/z = 509.18(C₃₈H₂₃NO = 509.61) |
| S-67 | m/z = 548.19(C₄₀H₂₄N₂O = 548.65) |
| S-68 | m/z = 549.17(C₄₀H₂₃NO₂ = 549.63) |
| S-69 | m/z = 449.12(C₃₂H₁₉NS = 449.57) |
| S-70 | m/z = 439.1(C₃₀H₁₇NOS = 439.53) |
| S-71 | m/z = 647.22(C₄₉H₂₉NO = 647.78) |
| S-72 | m/z = 717.28(C₅₂H₃₅N₃O = 717.87) |
| S-73 | m/z = 459.16(C₃₄H₂₁NO = 459.55) |
| S-74 | m/z = 533.18(C₄₀H₂₃NO = 533.63) |
| S-75 | m/z = 525.16(C₃₈H₂₃NS = 525.67) |
| S-76 | m/z = 564.17(C₄₀H₂₄N₂S = 564.71) |
| S-77 | m/z = 575.19(C₄₂H₂₅NO₂ = 575.67) |
| S-78 | m/z = 663.22(C₄₉H₂₉NO₂ = 663.78) |
| S-79 | m/z = 647.22(C₄₉H₂₉NO = 647.78) |
| S-80 | m/z = 496.16(C₃₆H₂₀N₂O = 496.57) |
| S-81 | m/z = 565.15(C₄₀H₂₃NOS = 565.69) |
| S-82 | m/z = 505.1(C₃₄H₁₉NS₂ = 505.65) |
| S-83 | m/z = 765.25(C₅₆H₃₅NOSi = 765.99) |
| S-84 | m/z = 615.17(C₄₄H₂₅NOS = 615.75) |
| S-85 | m/z = 603.17(C₄₃H₂₅NOS = 603.74) |
| S-86 | m/z = 772.29(C₅₉H₃₆N₂ = 772.95) |
| S-87 | m/z = 802.33(C₆₁H₄₂N₂ = 803.02) |
| S-88 | m/z = 607.23(C₄₇H₂₉N = 607.76) |
| S-89 | m/z = 524.23(C₃₉H₂₈N₂ = 524.67) |
| S-90 | m/z = 665.22(C₄₉H₃₁NS = 665.85) |
| S-91 | m/z = 633.25(C₄₉H₃₁N = 633.79) |
| S-92 | m/z = 775.29(C₅₉H₃₇NO = 775.95) |
| S-93 | m/z = 535.23(C₄₁H₂₉N = 535.69) |
| S-94 | m/z = 623.22(C₄₇H₂₉NO = 623.76) |
| S-95 | m/z = 687.2(C₅₁H₂₉NS = 687.86) |
| S-96 | m/z = 735.29(C₅₇H₃₇N = 735.93) |
| S-97 | m/z = 611.26(C₄₇H₃₃N = 611.79) |
| S-98 | m/z = 679.23(C₅₀H₃₃NS = 679.88) |
| S-99 | m/z = 787.32(C₆₁H₄₁N = 788.01) |
| S-100 | m/z = 743.33(C₅₅H₄₁N₃ = 743.95) |
| S-101 | m/z = 485.21(C₃₇H₂₇N = 485.63) |
| S-102 | m/z = 471.2(C₃₆H₂₅N = 471.6) |
| S-103 | m/z = 571.19(C₄₃H₂₅NO = 571.68) |
| S-104 | m/z = 584.23(C₄₄H₂₈N₂ = 584.72) |
| S-105 | m/z = 539.24(C₄₀H₂₁D₅N₂ = 539.69) |
| S-106 | m/z = 453.15(C₃₂H₁₅NS = 471.6) |
| S-107 | m/z = 563.26(C₄₃H₂₆D₄NO = 563.74) |
| S-108 | m/z = 589.26(C₄₄H₂₃D₅N₂ = 584.72) |
| S-109 | m/z = 587.24(C₄₄H₂₅D₃N₂ = 587.74) |
| S-110 | m/z = 584.23(C₄₄H₂₈N₂ = 584.72) |
| S-111 | m/z = 584.23.(C₄₄H₂₈N₂ = 584.72) |
| S-112 | m/z = 589.26(C₄₄H₂₃D₅N₂ = 589.75) |

### DESCRIPTION OF THE INVENTION

Reorganization energy (hereinafter, RE) refers to the energy lost due to the change in molecular structure arrangement during the movement of charges (electrons, holes). It depends on molecular geometry and has the characteristic that the smaller the structural difference between the neutral state and the charged state, the smaller the value. The RE value can be obtained by the following formula.

$$\begin{matrix}{RE}_{hole} : {λ}^{+} = ( {E}_{NOCE} - {E}_{COCE} ) + ( {E}_{CONE} - {E}_{NONE} ) \\ {RE}_{elec} : {λ}^{-} = ( {E}_{NOAE} - {E}_{AOAE} ) + ( {E}_{AONE} - {E}_{NONE} )\end{matrix}$$

Each factor is described as:
NONE: Neutral geometry of Neutral molecules (hereinafter, NO opt.),
NOAE: Anion geometry of Neutral molecules,
NOCE: Cation geometry of Neutral molecules,
AONE: Neutral geometry of Anion molecules,
AOAE: Anion geometry of Anion molecules (hereinafter, AO opt.),
CONE: Neutral geometry of Cation molecules,
COCE: Cation geometry of Cation molecules (hereinafter, CO opt.)

The value of Reorganization Energy is inversely proportional to mobility, and under the condition that they have the same r and T values, RE value of each material directly affects the mobility. The relation between RE value and mobility is expressed as follows.

$$μ = k \frac{{r}^{2}}{2 {k}_{B} T / e}$$

$$k = ( \frac{4 {π}^{2}}{h} ) \frac{{t}^{2}}{\sqrt{4 πλ {k}_{B} T}} exp [ - \frac{λ}{4 {k}_{B} T} ]$$

Each factor is described as:
λ: Reorganization energy,
μ: mobility,
r: dimer displacement,
t: intermolecular charge transfer matrix element.

From the above equation, it can be seen that the lower RE value, the faster the mobility. In order for exciton formation in the light emitting body, that is, in the dopant, to occur properly, the ratio of electrons and holes transferred from the host compound to the dopant must be balanced (charge balance). Compounds that are useful for charge transport to dopants due to their low RE value and fast mobility provide the possibility of high efficiency by forming more exciton in the dopant, but if one compound has an RE value that is too low compared to other heterogeneous compounds, the charge balance with the dopant is not correct, causing excessive charge injection, which has adverse effects on both lifespan and efficiency.

Reorganization energy value requires a simulation tool that can calculate the potential energy according to the molecular structure, we used Gaussian09 (hereinafter G09) and Jaguar module (hereinafter JG) of Schrodinger Materials Science. Both G09 and JG are tools to analyze the properties of molecules through quantum mechanical (QM) calculations, and have the function of optimizing the molecular structure or calculating the energy for a given molecular structure (single-point energy).

The process of performing QM calculations in molecular structures requires large computational resources, and our company uses 2 cluster servers for these calculations. Each cluster server consists of 4 node workstations and 1 master workstation, each node performed molecular QM calculations by Parallel computing through symmetric multi-processing (SMP) using a CPU with more than 36 cores.

Using G09, the optimized molecular structure and its potential energy (NONE/COCE) in the neutral/charged state required for rearrangement energy were calculated. The charge state potential energy (NOCE) of the structure optimized for the neutral state and the neutral state potential energy (CONE) of the structure optimized for the charge state were calculated by changing only the charges to the 2 optimized structures. After that, the rearrangement energy was calculated according to the following relation.

$${RE}_{charge} : λ = ( {E}_{NOCE} - {E}_{COCE} ) + ( {E}_{CONE} - {E}_{NONE} )$$

Because Schrodinger provides a function to automatically perform such a calculation process, the potential energy according to each state was sequentially calculated through the JG module by providing the molecular structure (NO) of the basic state, and the RE value was calculated.

Meanwhile, the RE values of the present invention calculated according to the above calculation method are shown in Table 4.

TABLE 4

| compound | Reorganization Energy |
|---|---|
| P-1 | 0.29 |
| P-3 | 0.24 |
| P-25 | 0.23 |

Meanwhile, the RE values of the comparative example compounds calculated according to the above calculation method are shown in Table 5.

TABLE 5

| compound | Reorganization Energy |
|---|---|
| Comparative | 0.15 |
| compound A | |
| Comparative | 0.21 |
| compound B | |
| Comparative | 0.22 |
| compound C | |

Among the recently developed dopants, those with the maximum emission wavelength have often shifted to short-wavelengths. This combination of a short-wavelength dopant and a host requires a long-wavelength host. When forming color coordinates, the maximum emission wavelength of the dopant and the maximum emission wavelength of the host have an influence, and by appropriately adjusting the maximum emission wavelength, it can be designed to match the optimal color coordinates: (0.681˜0.684) for the x-color coordinate and (0.316˜0.318) for the y-color coordinate. When designing a material, a common method of increasing the maximum emission wavelength is to narrow the band gap and increase the wavelength, and this method was used in the present invention. In the case of recently developed hosts, a mixture of 2 types of compounds is used, and in this case, both the HOMO energy and the LUMO energy of the different compounds are affected. Among the 2 compounds, the one that plays a large role as an electron donor affects the LUMO energy value of the mixture, and conversely, the one that acts as an electron acceptor affects the HOMO value of the mixture. The length of the covalent bond is determined depending on the type of substituent in the compound, which affects the energy level.

### EXAMPLES

### [Example 1] Red Organic Light Emitting Device (Phosphorescent Host)

An organic electroluminescent device was manufactured according to a conventional method using the compound obtained through synthesis as an emitting host material for the emitting layer. First, a 60 nm thick hole injection layer was formed by vacuum depositing a N1-(naphthalen-2-yl)-N4, N4-bis(4-(naphthalen-2-yl(phenyl)amino)phenyl)-N1-phenylbenzene-1,4-diamine (hereinafter abbreviated as 2-TNATA) film on the ITO layer (anode) formed on the glass substrate, and a hole transport layer was formed by vacuum depositing 4,4-bis[N-(1-naphthyl)-N-phenylamino]biphenyl (hereinafter abbreviated as −NPD) to a thickness of 60 nm as a hole transport compound on the hole injection layer. As a host on the top of the hole transport layer, the compounds of the present invention (P-1) and (S-109) represented by Formula (1) were used at a weight ratio of 5:5, and an emitting layer was deposited to a thickness of 30 nm by doping (D-1) as a dopant material at a weight ratio of 95:5. Next, (1,1′-bisphenyl)-4-oleato)bis(2-methyl-8-quinoline oleato)aluminum (hereinafter abbreviated as BAIq) was vacuum deposited to a thickness of 10 nm as a hole blocking layer, and as an electron transport layer, tris(8-quinolinol) aluminum (hereinafter abbreviated as Alq₃) was formed to a thickness of 40 nm. Afterwards, LiF, an alkali metal halide, was deposited to a thickness of 0.2 nm as an electron injection layer, and then Al was deposited to a thickness of 150 nm and used as a cathode to manufacture an organic electroluminescent device.

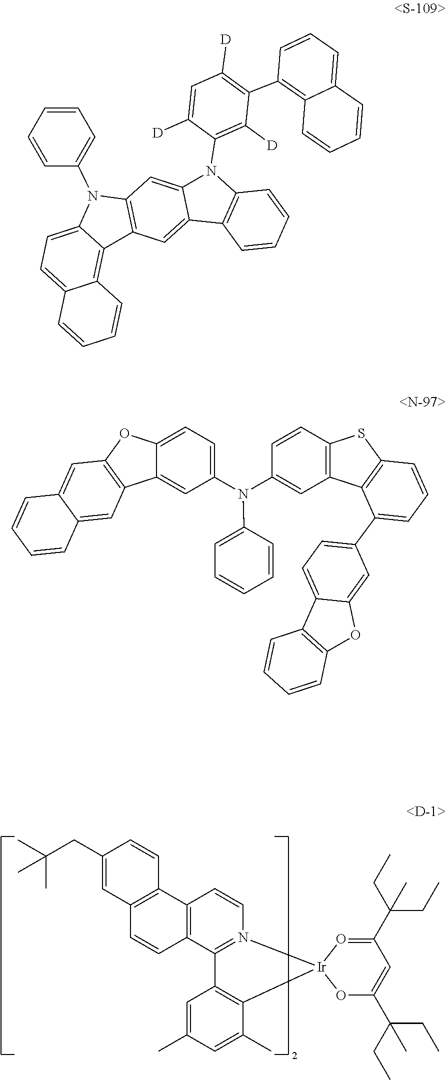

### [Example 2] to [Example 20]

An organic light emitting device was manufactured in the same manner as in Example 1, except that the compound of the present invention shown in Table 6 was used as the host material of the emitting layer instead of the compound (P-1) of the present invention.

### [Comparative Example 1] to [Comparative Example 6]

An organic light emitting device was manufactured in the same manner as in Example 1, except that Comparative Compound A to Comparative Compound C were used as the host material of the emitting layer.

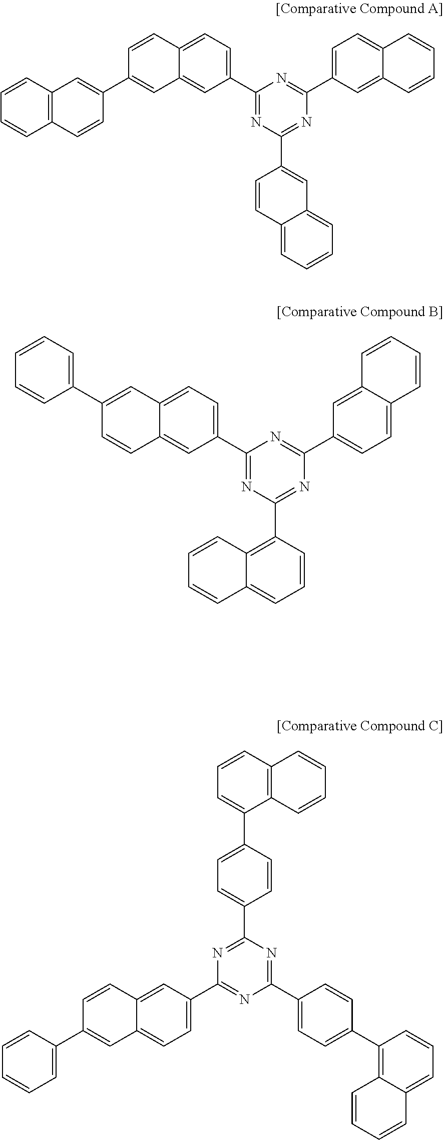 [SMILES: c1ccc2cc(-c3ccc4ccc(-c5nc(-c6ccc7ccccc7c6)nc(-c6ccc7ccccc7c6)n5)cc4c3)ccc2c1 ; c1ccc(-c2ccc3cc(-c4nc(-c5ccc6ccccc6c5)nc(-c5cccc6ccccc56)n4)ccc3c2)cc1 ; c1ccc(-c2ccc3cc(-c4nc(-c5ccc(-c6cccc7ccccc67)cc5)nc(-c5ccc(-c6cccc7ccccc67)cc5)n4)ccc3c2)cc1]

To the organic electroluminescent device manufactured by Examples 1 to 20, Comparative Examples 1 to 6, Electroluminescence (EL) characteristics were measured with a PR-650 of Photoresearch Co., by applying a forward bias DC voltage. As a result of the measurement, T95 life was measured at a standard luminance of 2,500 cd/m² through life measuring apparatus manufactured by McScience. Table 6 shows the results of device fabrication and evaluation according to the example.

TABLE 6

| | | | | Current | | | |
|---|---|---|---|---|---|---|---|
| | Frist | Second | Voltage | Density | Brightness | Efficiency | |
| | host | host | (v) | (mA/cm²) | (cd/m²) | (cd/A) | T(95) |
| Comparative | S- | Comparative | 4.9 | 13.5 | 2500 | 18.5 | 89.7 |
| example (1) | 109 | compound A | | | | | |
| Comparative | | Comparative | 5.0 | 10.9 | 2500 | 22.9 | 110.7 |
| example (2) | | compound B | | | | | |
| Comparative | | Comparative | 5.1 | 10.1 | 2500 | 24.7 | 105.6 |
| example (3) | | compound C | | | | | |
| Comparative | N- | Comparative | 4.8 | 15.2 | 2500 | 16.4 | 77.7 |
| example (4) | 97 | compound A | | | | | |
| Comparative | | Comparative | 4.9 | 11.7 | 2500 | 21.3 | 103.4 |
| example (5) | | compound B | | | | | |
| Comparative | | Comparative | 5.0 | 11.0 | 2500 | 22.8 | 99.2 |
| example (6) | | compound C | | | | | |
| Example (1) | S- | P-1 | 4.7 | 7.4 | 2500 | 33.9 | 147.5 |
| Example (2) | 109 | P-3 | 4.8 | 7.6 | 2500 | 33.1 | 140.2 |
| Example (3) | | P-6 | 4.6 | 7.9 | 2500 | 31.8 | 145.0 |
| Example (4) | | P-9 | 4.8 | 7.6 | 2500 | 32.7 | 146.3 |
| Example (5) | | P-17 | 4.7 | 8.0 | 2500 | 31.1 | 139.1 |
| Example (6) | | P-24 | 4.8 | 7.7 | 2500 | 32.4 | 143.9 |
| Example (7) | | P-25 | 4.9 | 7.8 | 2500 | 32.1 | 141.4 |
| Example (8) | | P-30 | 4.9 | 8.1 | 2500 | 30.9 | 137.8 |
| Example (9) | | P-38 | 4.7 | 7.5 | 2500 | 33.3 | 148.8 |
| Example (10) | | P-39 | 4.9 | 7.9 | 2500 | 31.5 | 142.6 |
| Example (11) | N- | P-1 | 4.6 | 7.5 | 2500 | 33.2 | 134.1 |
| Example (12) | 97 | P-3 | 4.8 | 7.7 | 2500 | 32.5 | 127.9 |
| Example (13) | | P-6 | 4.6 | 8.0 | 2500 | 31.3 | 131.6 |
| Example (14) | | P-9 | 4.7 | 7.7 | 2500 | 32.3 | 132.9 |
| Example (15) | | P-17 | 4.7 | 8.1 | 2500 | 30.7 | 124.7 |
| Example (16) | | P-24 | 4.8 | 7.8 | 2500 | 31.9 | 130.4 |
| Example (17) | | P-25 | 4.7 | 7.9 | 2500 | 31.7 | 128.1 |
| Example (18) | | P-30 | 4.9 | 8.2 | 2500 | 30.4 | 124.4 |
| Example (19) | | P-38 | 4.6 | 7.6 | 2500 | 32.9 | 135.3 |
| Example (20) | | P-39 | 4.8 | 8.1 | 2500 | 31.0 | 129.2 |

As can be seen from the results in Table 6, when the compound of the present invention is used as an emitting layer material, it can be seen that the driving voltage is lowered and the efficiency and lifespan are significantly improved compared to the case where Comparative Compound A to Comparative Compound C are used. In more detail, the compounds of the present invention have higher RE values compared to comparative compounds. These RE values vary greatly depending on the type of component substituted for the triazine, and compounds with high RE values have slower mobility than compounds with low RE values. Generally, faster mobility has the effect of increasing the driving voltage, but, in the case of the present invention, an excessively low RE value results in too fast mobility, resulting in a large difference in hole injection and mobility, and deteriorates the injection characteristics of electrons and holes into the dopant, thereby reducing efficiency and lifespan. As a result, it can be seen that the combination of electrons and holes of the first and second compounds has a significant impact on the overall device result. In addition, in the case of overall luminous efficiency, not only the combination with the two hosts mentioned above, but also the combination of these hosts and dopants is also important. In the present invention, the maximum luminous efficiency was increased by using a dopant shifted to a short-wavelength, but when using the dopant, it is difficult to match the color purity of the color coordinates. In order to control the color coordinates with the highest efficiency, a long-wavelength host must be used, and the compounds of the present invention are compounds that have been shifted to long-wavelengths.

That the compounds of the present invention have moved to long-wavelengths is determined from the HOMO energy level of the first compound and the LUMO energy level of the second compound, as a result of the measurement, it was confirmed that the compound had a narrow band gap compared to the comparative examples. This narrow band gap ultimately shifts the maximum emission wavelength to a longer wavelength, and this long-wavelength host showed maximum emission efficiency in combination with a short-wavelength dopant.

Through comparison of the compounds of the present invention, it was confirmed that the luminous efficiency of the compound with an RE value of 0.29 was higher than that of the compound with a lower RE value, and this may be applied differently depending on the type of the first compound to be combined. In general, compounds substituted with deuterium were used as an advantage in terms of lifespan.

In conclusion, when an emitting layer is composed of multiple mixtures, the characteristics are different depending on the type of the first compound and the second compound, and it can be seen that the driving voltage, efficiency, and lifespan are determined by the injection characteristics of holes and electrons into the dopant.

In the present invention, it can be seen that the relationship between RE value and mobility results in an overall drive reduction effect and an increase in efficiency and lifespan. In addition, it can be seen that the invention of a substituent substituted for the core triazine has a positive effect on the overall mobility when combining specific substituents, acting on the ratio of holes and electrons (e.g. energy balance, stability, etc.), showing overall improved results.

Although exemplary embodiments of the present invention have been described for illustrative purposes, those skilled in the art will appreciate that various modifications, additions and substitutions are possible, without departing from the scope and spirit of the invention as disclosed in the accompanying claims. Therefore, the embodiment disclosed in the present invention is intended to illustrate the scope of the technical idea of the present invention, and the scope of the present invention is not limited by the embodiment. The scope of the present invention shall be construed on the basis of the accompanying claims, and it shall be construed that all of the technical ideas included within the scope equivalent to the claims belong to the present invention.

### INDUSTRIAL APPLICABILITY

According to the present invention, it is possible to manufacture an organic device with excellent device characteristics such as high brightness, high luminescence, and long lifespan, and thus has industrial applicability.

1. A compound represented by Formula (1): 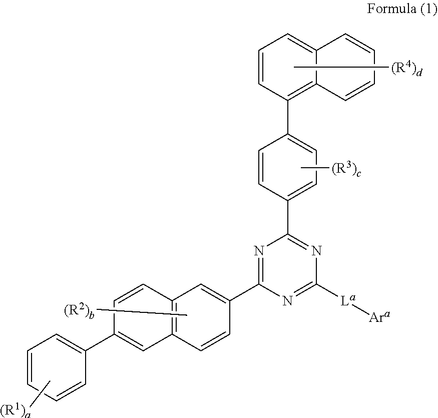
wherein:
1) L^{a} is a direct bond; a phenylene group; a naphthylene group; a biphenylene group; or a phenanthrene group,
2) Ar^{a} is represented by any of Formulas (A-1) to (A-6): 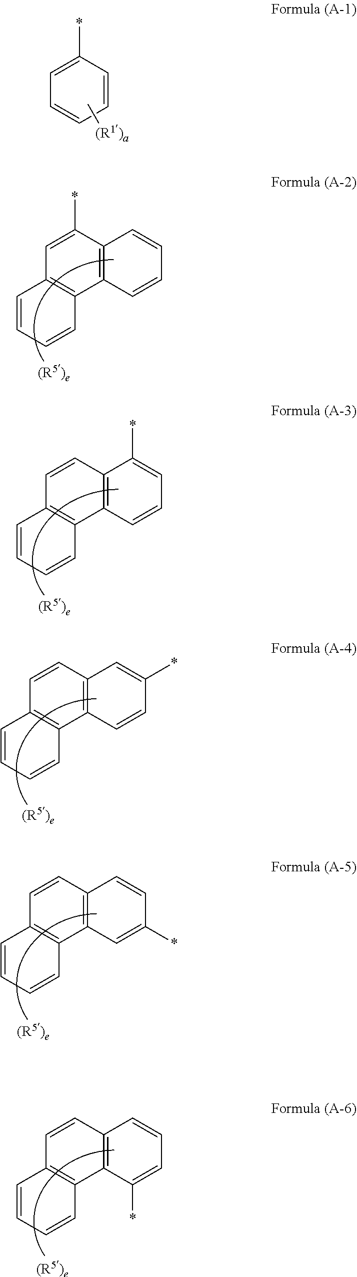
wherein:
* means the position to be bonded,
R^{1′} and R^{5′} are same or different from each other, and each is independently selected from the group consisting of hydrogen; deuterium; a C₆-C₂₀ aryl group; a C₆-C₂₀ aryl group substituted with deuterium, or an adjacent plurality of R¹ or adjacent plurality of R^{5′} may be bonded to each other to form a ring,
a is an integer from 0 to 5, e is an integer from 0 to 9,
3) R¹, R², R³ and R⁴, each being the same or different, are each independently hydrogen or deuterium,
4) a is an integer of 0 to 5, b is an integer of 0 to 6, c is an integer of 0 to 4, d is an integer of 0 to 7,
wherein the phenylene group, naphthylene group, biphenylene group and phenanthrene group may be substituted with one or more substituents selected from the group consisting of deuterium; a C₆-C₂₀ aryl group; and a C₆-C₂₀ aryl group substituted with deuterium.

2. The compound of claim 1, wherein L^{a} is represented by any of Formulas (L-1) to (L-14): 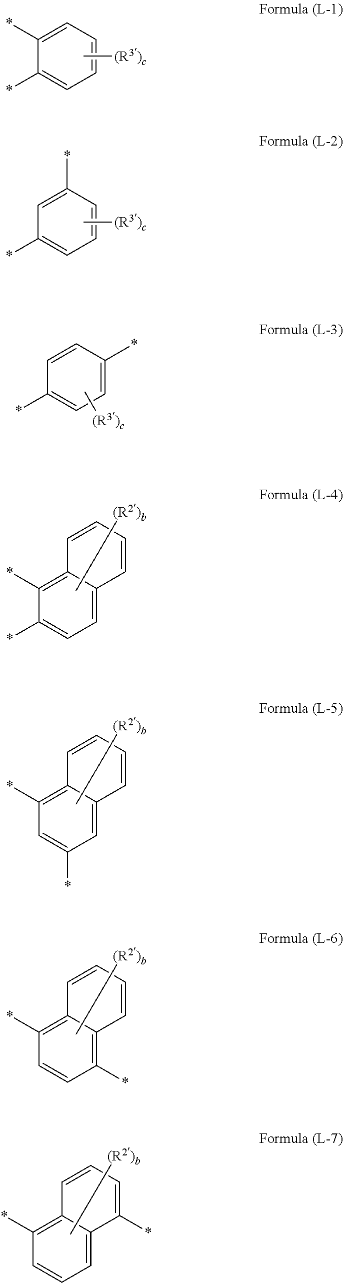 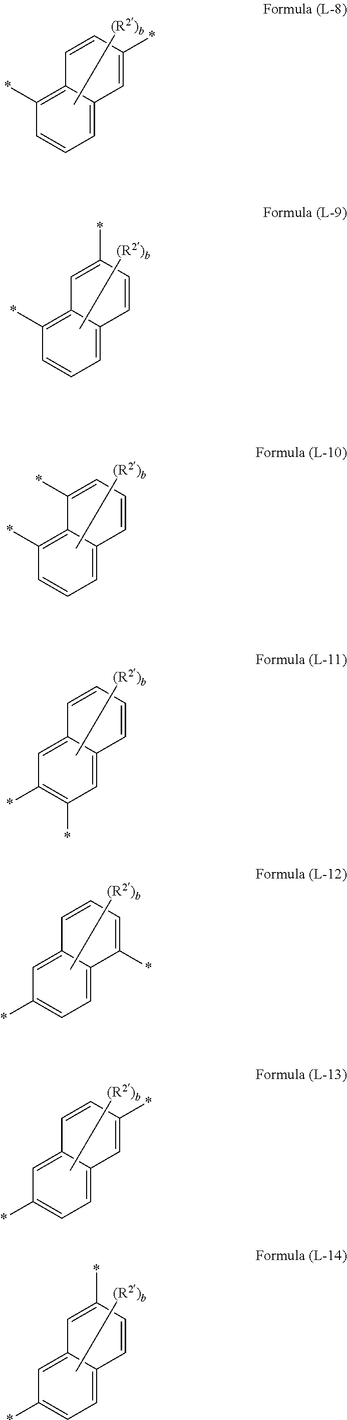
wherein:
1) * means the position to be bonded,
2) R^{2′} and R^{3′} are the same as the definition of R^{1′} in claim 1, and
3) b is an integer of 0 to 6, c is an integer of 0 to 4.

3. The compound of claim 1 having a Reorganization Energy value higher than 0.23.

4. The compound of claim 1 having a Reorganization Energy value higher than 0.23 and lower than 0.29.

5. The compound of claim 1, wherein Formula (1) is selected from the group consisting of Compounds P-1 to P-40:

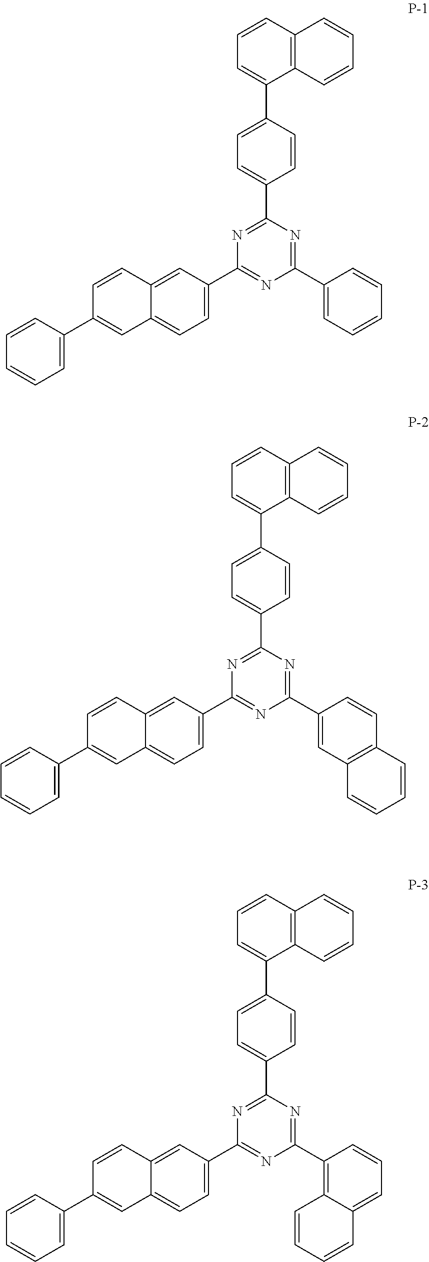 [SMILES: c1ccc(-c2ccc3cc(-c4nc(-c5ccccc5)nc(-c5ccc(-c6cccc7ccccc67)cc5)n4)ccc3c2)cc1 ; c1ccc(-c2ccc3cc(-c4nc(-c5ccc(-c6cccc7ccccc67)cc5)nc(-c5ccc6ccccc6c5)n4)ccc3c2)cc1 ; c1ccc(-c2ccc3cc(-c4nc(-c5ccc(-c6cccc7ccccc67)cc5)nc(-c5cccc6ccccc56)n4)ccc3c2)cc1]

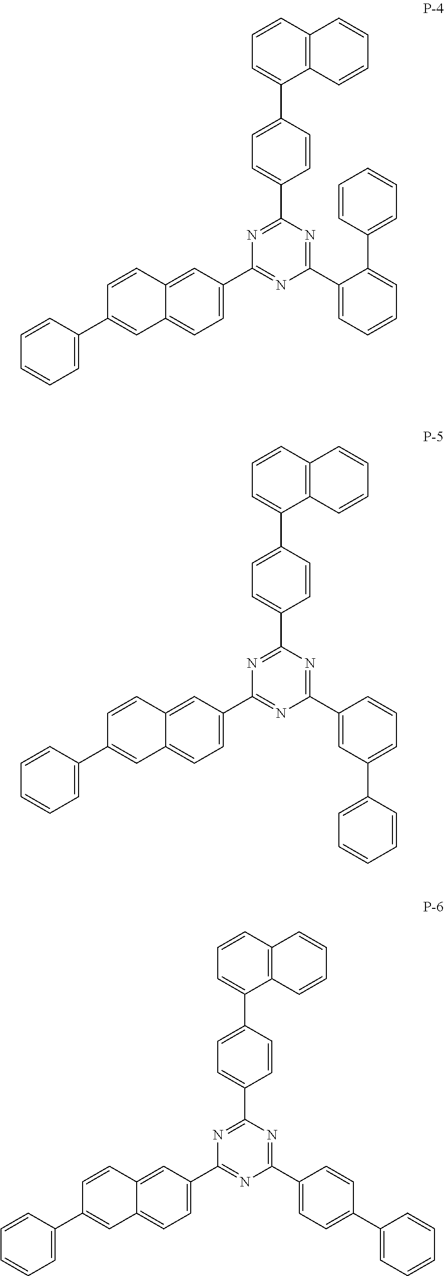 [SMILES: c1ccc(-c2ccc3cc(-c4nc(-c5ccc(-c6cccc7ccccc67)cc5)nc(-c5ccccc5-c5ccccc5)n4)ccc3c2)cc1 ; c1ccc(-c2cccc(-c3nc(-c4ccc(-c5cccc6ccccc56)cc4)nc(-c4ccc5cc(-c6ccccc6)ccc5c4)n3)c2)cc1 ; c1ccc(-c2ccc(-c3nc(-c4ccc(-c5cccc6ccccc56)cc4)nc(-c4ccc5cc(-c6ccccc6)ccc5c4)n3)cc2)cc1]

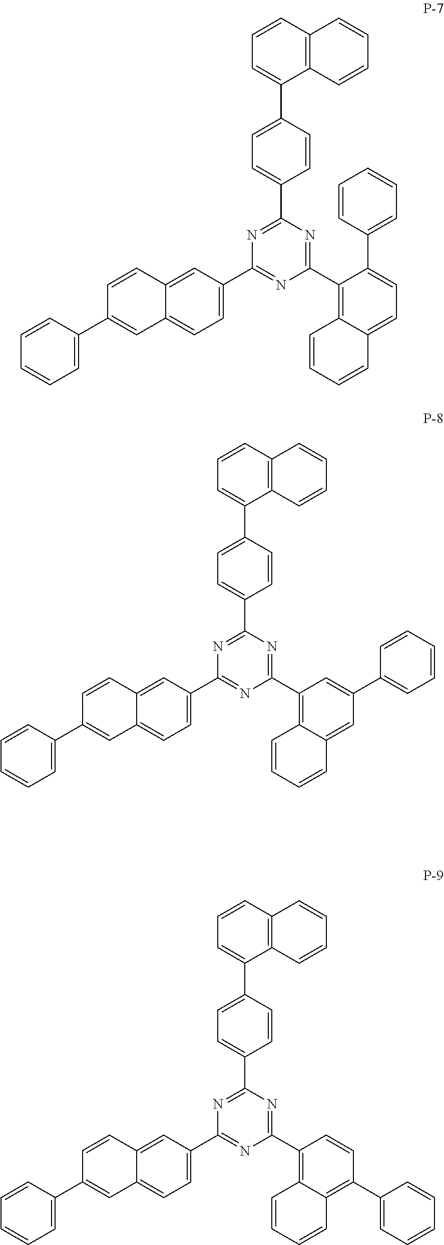 [SMILES: c1ccc(-c2ccc3cc(-c4nc(-c5ccc(-c6cccc7ccccc67)cc5)nc(-c5c(-c6ccccc6)ccc6ccccc56)n4)ccc3c2)cc1 ; c1ccc(-c2ccc3cc(-c4nc(-c5ccc(-c6cccc7ccccc67)cc5)nc(-c5cc(-c6ccccc6)cc6ccccc56)n4)ccc3c2)cc1 ; c1ccc(-c2ccc3cc(-c4nc(-c5ccc(-c6cccc7ccccc67)cc5)nc(-c5ccc(-c6ccccc6)c6ccccc56)n4)ccc3c2)cc1]

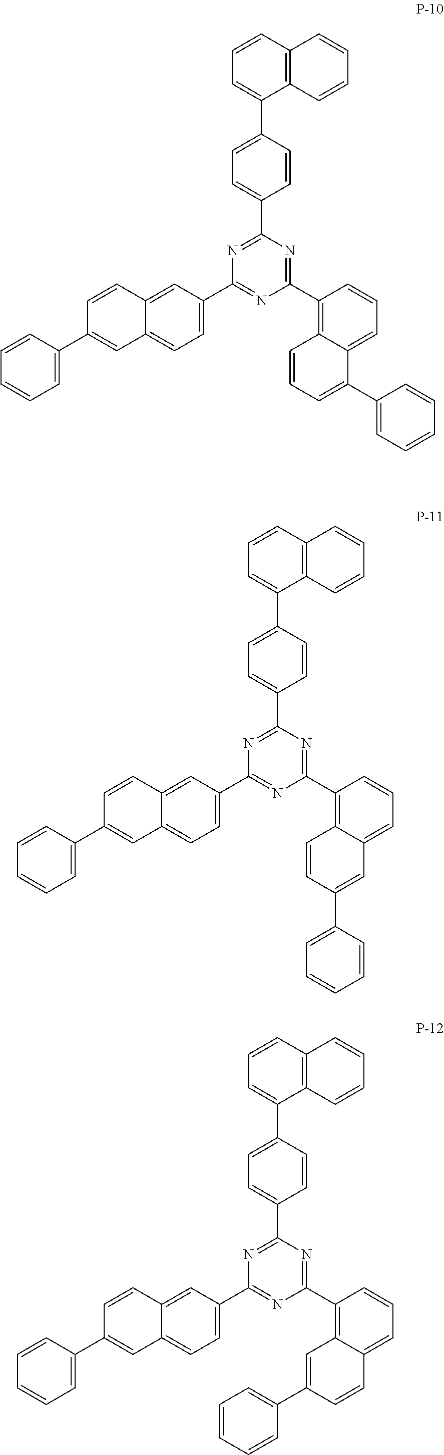

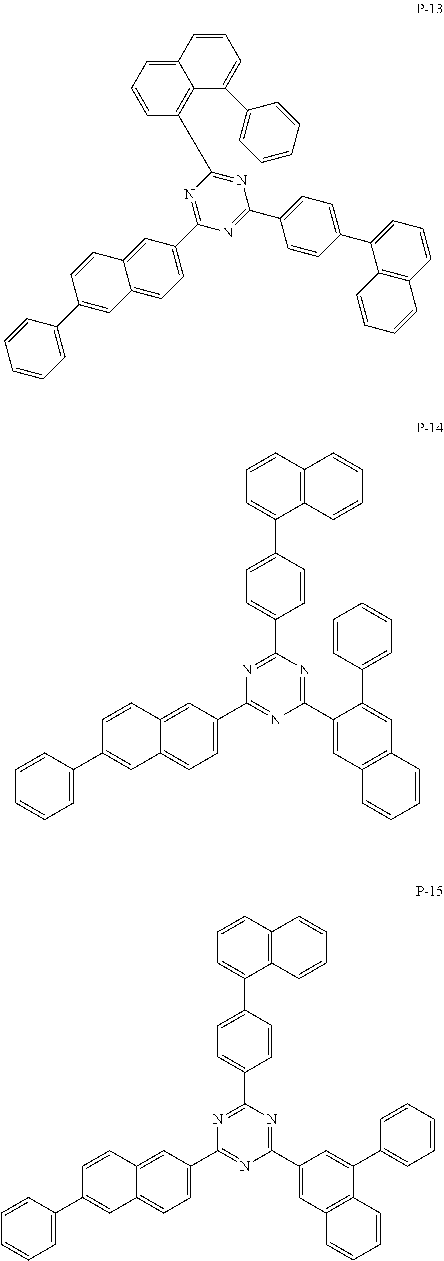 [SMILES: c1ccc(-c2ccc3cc(-c4nc(-c5ccc(-c6cccc7ccccc67)cc5)nc(-c5cccc6cccc(-c7ccccc7)c56)n4)ccc3c2)cc1 ; c1ccc(-c2ccc3cc(-c4nc(-c5ccc(-c6cccc7ccccc67)cc5)nc(-c5cc6ccccc6cc5-c5ccccc5)n4)ccc3c2)cc1 ; c1ccc(-c2ccc3cc(-c4nc(-c5ccc(-c6cccc7ccccc67)cc5)nc(-c5cc(-c6ccccc6)c6ccccc6c5)n4)ccc3c2)cc1]

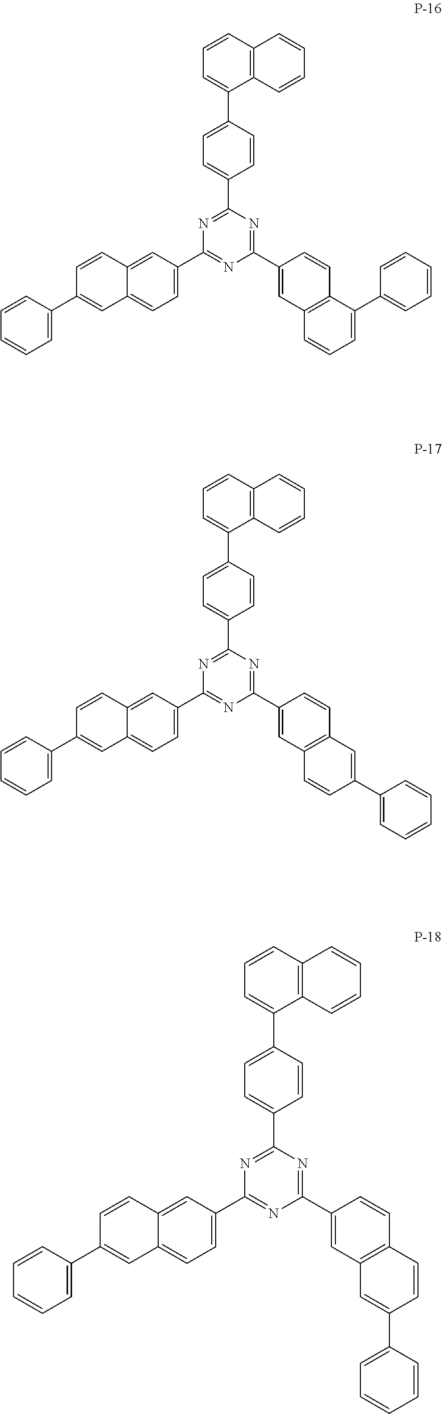 [SMILES: c1ccc(-c2ccc3cc(-c4nc(-c5ccc(-c6cccc7ccccc67)cc5)nc(-c5ccc6c(-c7ccccc7)cccc6c5)n4)ccc3c2)cc1 ; c1ccc(-c2ccc3cc(-c4nc(-c5ccc(-c6cccc7ccccc67)cc5)nc(-c5ccc6cc(-c7ccccc7)ccc6c5)n4)ccc3c2)cc1 ; c1ccc(-c2ccc3cc(-c4nc(-c5ccc(-c6cccc7ccccc67)cc5)nc(-c5ccc6ccc(-c7ccccc7)cc6c5)n4)ccc3c2)cc1]

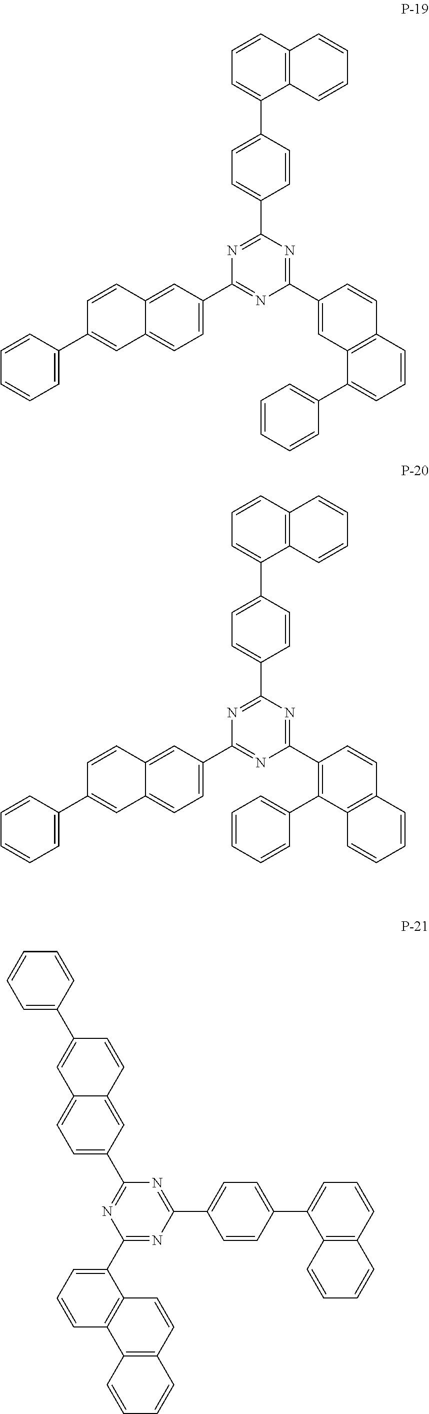 [SMILES: c1ccc(-c2ccc3cc(-c4nc(-c5ccc(-c6cccc7ccccc67)cc5)nc(-c5ccc6cccc(-c7ccccc7)c6c5)n4)ccc3c2)cc1 ; c1ccc(-c2ccc3cc(-c4nc(-c5ccc(-c6cccc7ccccc67)cc5)nc(-c5ccc6ccccc6c5-c5ccccc5)n4)ccc3c2)cc1 ; c1ccc(-c2ccc3cc(-c4nc(-c5ccc(-c6cccc7ccccc67)cc5)nc(-c5cccc6c5ccc5ccccc56)n4)ccc3c2)cc1]

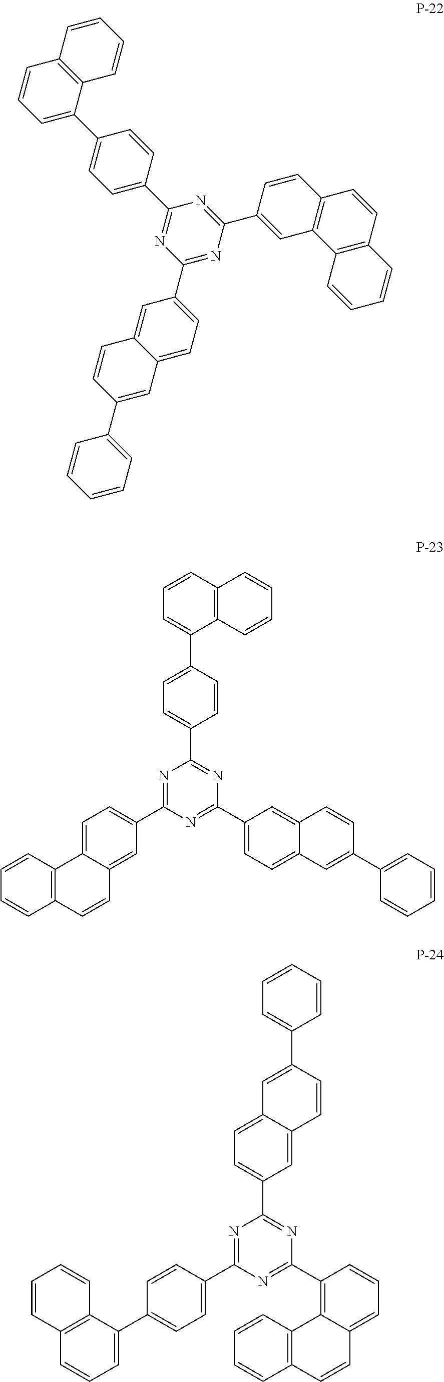 [SMILES: c1ccc(-c2ccc3cc(-c4nc(-c5ccc(-c6cccc7ccccc67)cc5)nc(-c5ccc6ccc7ccccc7c6c5)n4)ccc3c2)cc1 ; c1ccc(-c2ccc3cc(-c4nc(-c5ccc(-c6cccc7ccccc67)cc5)nc(-c5ccc6c(ccc7ccccc76)c5)n4)ccc3c2)cc1 ; c1ccc(-c2ccc3cc(-c4nc(-c5ccc(-c6cccc7ccccc67)cc5)nc(-c5cccc6ccc7ccccc7c56)n4)ccc3c2)cc1]

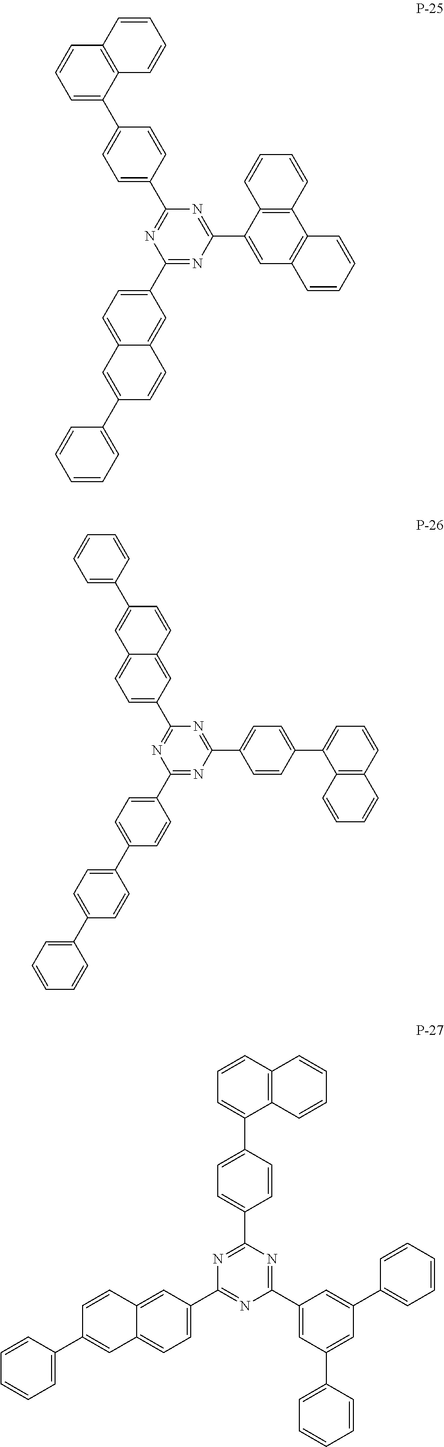 [SMILES: c1ccc(-c2ccc3cc(-c4nc(-c5ccc(-c6cccc7ccccc67)cc5)nc(-c5cc6ccccc6c6ccccc56)n4)ccc3c2)cc1 ; c1ccc(-c2ccc(-c3ccc(-c4nc(-c5ccc(-c6cccc7ccccc67)cc5)nc(-c5ccc6cc(-c7ccccc7)ccc6c5)n4)cc3)cc2)cc1 ; c1ccc(-c2cc(-c3ccccc3)cc(-c3nc(-c4ccc(-c5cccc6ccccc56)cc4)nc(-c4ccc5cc(-c6ccccc6)ccc5c4)n3)c2)cc1]

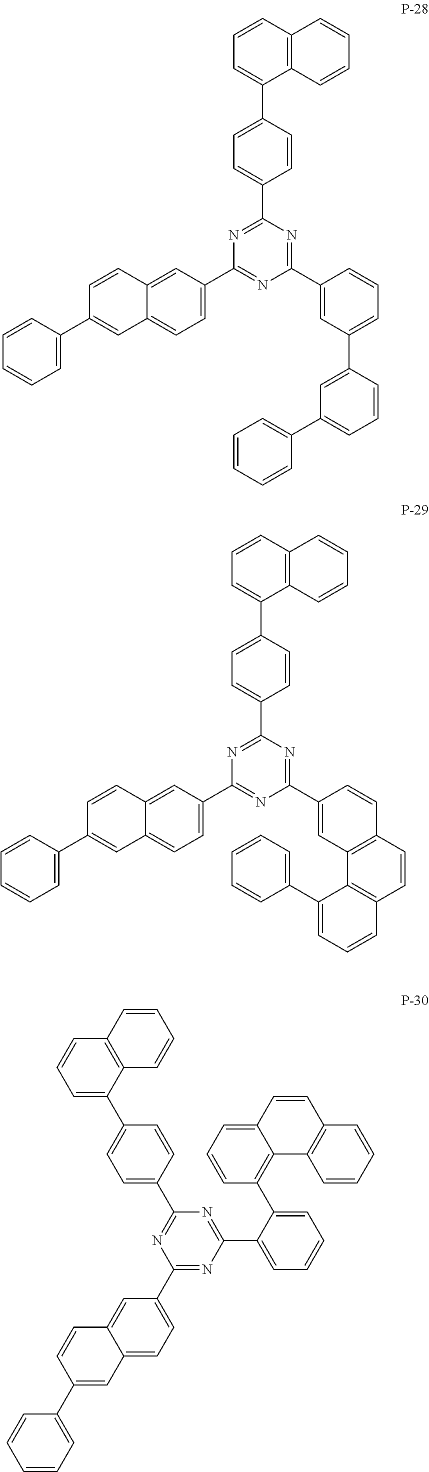 [SMILES: c1ccc(-c2cccc(-c3cccc(-c4nc(-c5ccc(-c6cccc7ccccc67)cc5)nc(-c5ccc6cc(-c7ccccc7)ccc6c5)n4)c3)c2)cc1 ; c1ccc(-c2ccc3cc(-c4nc(-c5ccc(-c6cccc7ccccc67)cc5)nc(-c5ccc6ccc7cccc(-c8ccccc8)c7c6c5)n4)ccc3c2)cc1 ; c1ccc(-c2ccc3cc(-c4nc(-c5ccc(-c6cccc7ccccc67)cc5)nc(-c5ccccc5-c5cccc6ccc7ccccc7c56)n4)ccc3c2)cc1]

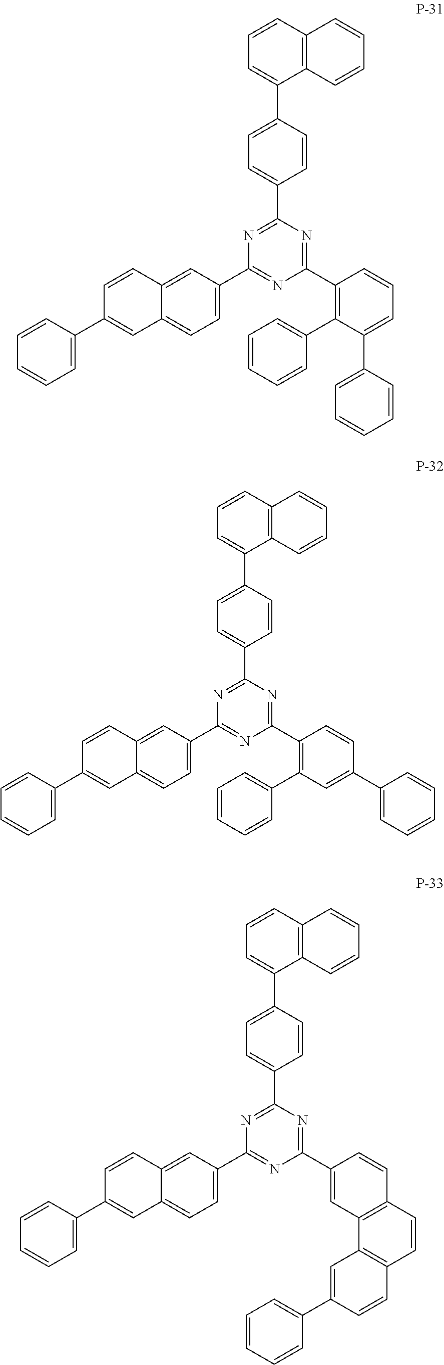 [SMILES: c1ccc(-c2ccc3cc(-c4nc(-c5ccc(-c6cccc7ccccc67)cc5)nc(-c5cccc(-c6ccccc6)c5-c5ccccc5)n4)ccc3c2)cc1 ; c1ccc(-c2ccc(-c3nc(-c4ccc(-c5cccc6ccccc56)cc4)nc(-c4ccc5cc(-c6ccccc6)ccc5c4)n3)c(-c3ccccc3)c2)cc1 ; c1ccc(-c2ccc3cc(-c4nc(-c5ccc(-c6cccc7ccccc67)cc5)nc(-c5ccc6ccc7ccc(-c8ccccc8)cc7c6c5)n4)ccc3c2)cc1]

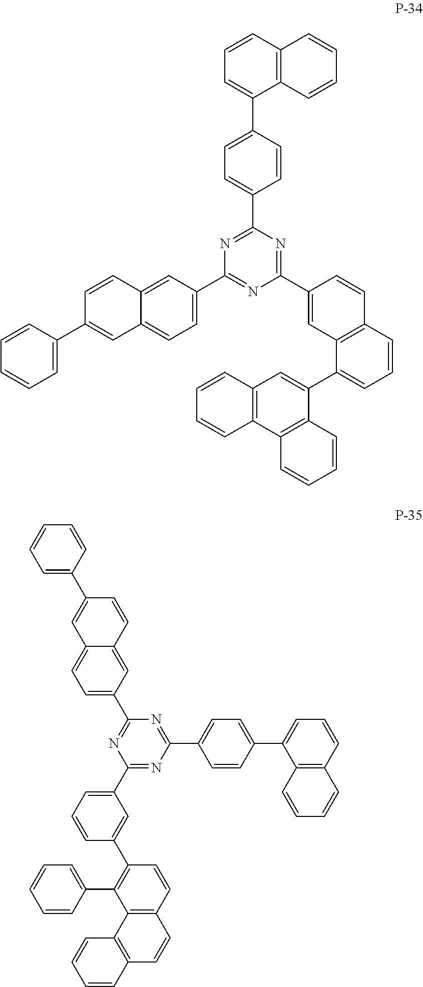 [SMILES: c1ccc(-c2ccc3cc(-c4nc(-c5ccc(-c6cccc7ccccc67)cc5)nc(-c5ccc6cccc(-c7cc8ccccc8c8ccccc78)c6c5)n4)ccc3c2)cc1 ; c1ccc(-c2ccc3cc(-c4nc(-c5ccc(-c6cccc7ccccc67)cc5)nc(-c5cccc(-c6ccc7ccc8ccccc8c7c6-c6ccccc6)c5)n4)ccc3c2)cc1]

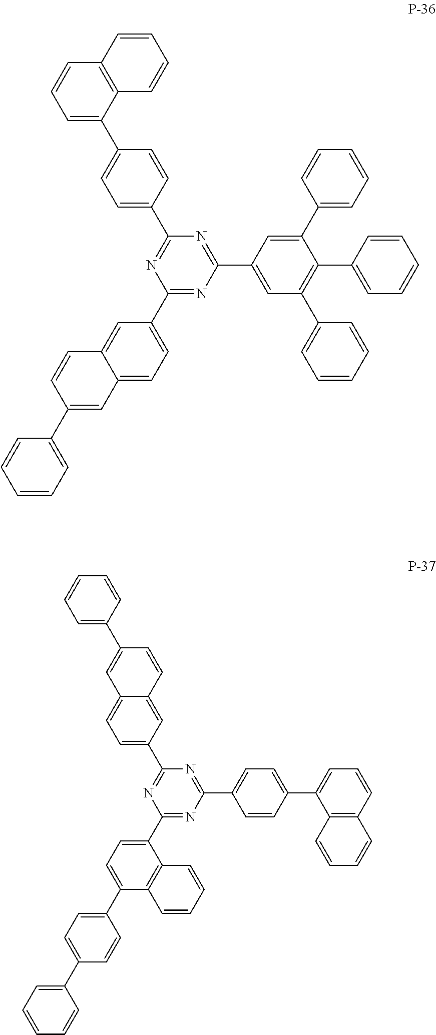 [SMILES: c1ccc(-c2ccc3cc(-c4nc(-c5ccc(-c6cccc7ccccc67)cc5)nc(-c5cc(-c6ccccc6)c(-c6ccccc6)c(-c6ccccc6)c5)n4)ccc3c2)cc1 ; c1ccc(-c2ccc(-c3ccc(-c4nc(-c5ccc(-c6cccc7ccccc67)cc5)nc(-c5ccc6cc(-c7ccccc7)ccc6c5)n4)c4ccccc34)cc2)cc1]

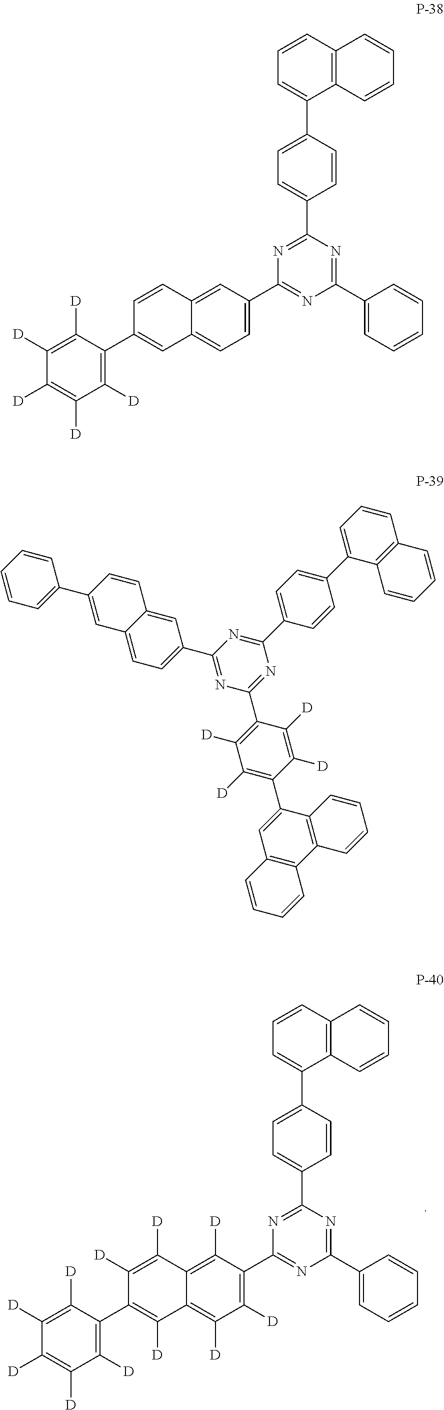 [SMILES: [2H]c1c([2H])c([2H])c(-c2ccc3cc(-c4nc(-c5ccccc5)nc(-c5ccc(-c6cccc7ccccc67)cc5)n4)ccc3c2)c([2H])c1[2H] ; [2H]c1c([2H])c(-c2cc3ccccc3c3ccccc23)c([2H])c([2H])c1-c1nc(-c2ccc(-c3cccc4ccccc34)cc2)nc(-c2ccc3cc(-c4ccccc4)ccc3c2)n1 ; [2H]c1c([2H])c([2H])c(-c2c([2H])c([2H])c3c([2H])c(-c4nc(-c5ccccc5)nc(-c5ccc(-c6cccc7ccccc67)cc5)n4)c([2H])c([2H])c3c2[2H])c([2H])c1[2H]]

6. An organic electronic element comprising an anode; a cathode; and an organic material layer formed between the anode and the cathode; wherein the organic material layer comprises a single compound or 2 or more compounds represented by Formula (1) of claim 1.

7. The organic electronic element of claim 6, wherein the organic material layer comprises at least one of a hole injection layer, a hole transport layer, an emitting-auxiliary layer, an emitting layer, an electron transport-auxiliary layer, an electron transport layer and an electron injection layer.

8. The organic electronic element of claim 7, comprising the compound represented by Formula (1) of claim 1 as a host material in the emitting layer with a dopant material, wherein the dopant material has a maximum emission wavelength of 610 nm to 620 nm.

9. The organic electronic element of claim 6, wherein the organic material layer comprises an emitting layer comprising the compound represented by Formula (1) as a host material with a dopant material, wherein the dopant material has a maximum emission wavelength of 610 nm to 615 nm.

10. The organic electronic element of claim 6, wherein the organic material layer comprises an emitting layer comprising the compound represented by Formula (1) as a host material, and further comprising a compound represented by Formula (2) or (3) as a heterogeneous host material: 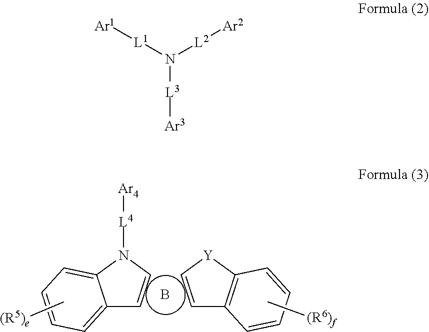
wherein:
1) Y is O, S, CR′R″ or NR^{a},
2) R′ and R″ are each independently selected from the group consisting of a C₆-C₆₀ aryl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₅₀ alkyl group; a C₂-C₂₀ alkenyl group; a C₂-C₂₀ alkynyl group; a C₁-C₃₀ alkoxyl group; and a C₆-C₃₀ aryloxy group; or R′ and R″ may be bonded to each other to form a ring,
3) L¹, L², L³ and L⁴ are each independently selected from the group consisting of a single bond; a C₆-C₆₀ arylene group; and a C₂-C₆₀ heteroarylene group,
4) R^{a}, Ar¹, Ar² and Ar³ are each independently a C₆-C₆₀ aryl group; or a C₂-C₆₀ heteroaryl group,
5) Ar⁴ is selected from the group consisting of a C₆-C₆₀ aryl group; or a C₂-C₆₀ heteroaryl group; and -L′-NR^{b}R^{c},
6) wherein L′ is selected from the group consisting of a single bond; a C₆-C₆₀ arylene group; a fluorenylene group; a C₂-C₆₀ heterocyclic group including at least one heteroatom of O, N, S, Si or P; and a C₃-C₆₀ aliphatic ring, and wherein R^{b} and R^{c} are each independently selected from the group consisting of a C₆-C₆₀ aryl group; a fluorenyl group; a C₂-C₆₀ heterocyclic group including at least one heteroatom of O, N, S, Si or P; and a fused ring group of a C₃-C₆₀ aliphatic ring and a C₆-C₆₀ aromatic ring,
7) Ring B is a C₆-C₂₀ aryl group;
8) R⁵ and R⁶ are each independently same or different, and each independently selected from the group consisting of hydrogen; deuterium; a halogen; a cyano group; a nitro group; a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; and a C₆-C₃₀ aryloxy group, or adjacent plurality of R⁵ or plurality of R⁵ may be bonded to each other to form a ring,
9) e and f are each independently an integer of 0 to 4,
10) wherein the aryl group, arylene group, heterocyclic group, fluorenyl group, fluorenylene group, fused ring group, alkyl group, alkenyl group, alkoxy group and aryloxy group may be substituted with one or more substituents selected from the group consisting of deuterium; a halogen; a silane group; siloxane group; a boron group; a germanium group; a cyano group; a nitro group; a C₁-C₂₀ alkylthio group; a C₁-C₂₀ alkoxy group; a C₁-C₂₀ alkyl group; a C₂-C₂₀ alkenyl group; a C₂-C₂₀ alkynyl group; a C₆-C₂₀ aryl group; a C₆-C₂₀ aryl group substituted with deuterium; a fluorenyl group; a C₂-C₂₀ heterocyclic group; a C₃-C₂₀ cycloalkyl group; C₇-C₂₀ arylalkyl group; a C₈-C₂₀ arylalkenyl group; and -L′-NR^{b}R^{c}, wherein the substituents may be bonded to each other to form a saturated or unsaturated ring, wherein the term ‘ring’ means a C₃-C₆₀ aliphatic ring or a C₆-C₆₀ aromatic ring or a C₂-C₆₀ heterocyclic group or a fused ring formed by the combination thereof.

11. The organic electronic element of claim 10, wherein Formula (2) is represented by any of Formulas (2-1) to (2-3): 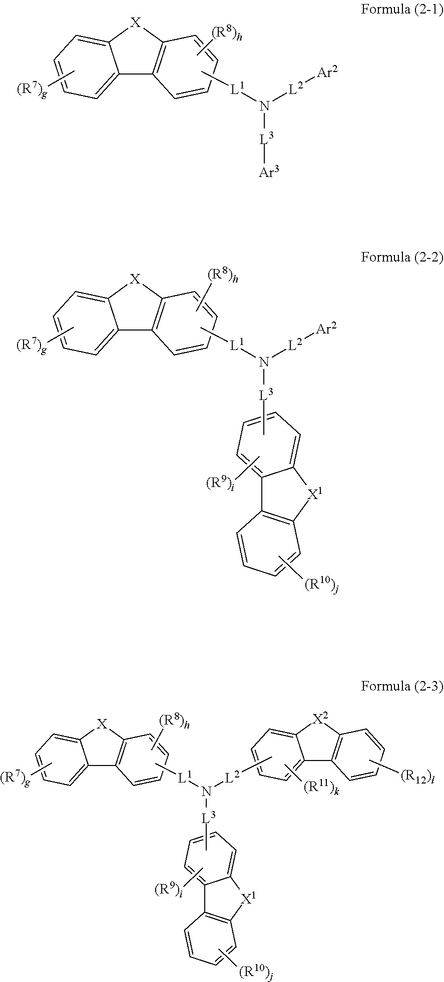
wherein:
1) X, X¹ and X² are the same as definition of Y of claim 10,
2) R⁷, R⁸, R⁹, R¹⁰, R¹¹ and R¹² are each the same or different, and each independently selected from the group consisting of hydrogen; deuterium; a halogen; a cyano group; a nitro group; a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; a C₆-C₆₀ aryloxy group; and -L′-NR^{b}R^{c}, or an adjacent plurality of R⁷ or plurality of R⁸ or plurality of R⁹ or plurality of R¹⁰ or plurality of R¹¹ or plurality of R¹² may be bonded to each other to form a ring,
3) L¹, L², L³, Ar², Ar³, L′, R^{b} and R^{c} are the same as defined in claim 10,
4) g, j and l are each independently an integer of 0 to 4, h, i and k are each independently an integer of 0 to 3.

12. The organic electronic element of claim 10, wherein Formula (3) is represented by any of Formulas (3-1) to (3-6): 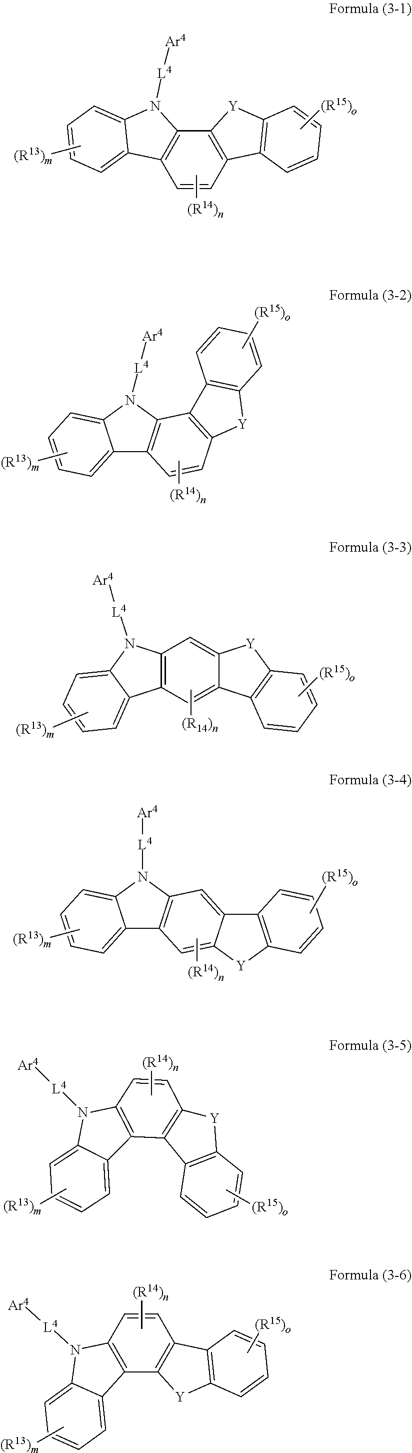
wherein:
1) Y, L⁴ and Ar⁴ are the same as defined in claim 10,
2) R¹³, R¹⁴ and R¹⁵ are the same as definition of R⁵ in claim 10, and
3) m and o are each independently an integer of 0 to 4, n is an integer of 0 to 2.

13. The organic electronic element of claim 10, wherein Formula (3) is represented by any of Formulas (3-7) to (3-9): 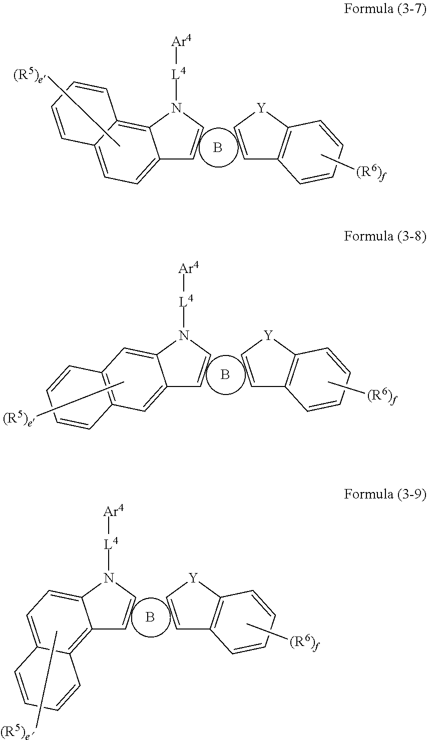
wherein:
1) Ring B, R⁵, R⁶, f, Y, L⁴ and Ar⁴ are the same as defined in claim 10, and
2) e′ is an integer of 0 to 6.

14. The organic electronic element of claim 10, wherein Formula (3) is represented by any of Formulas (3-10) to (3-12): 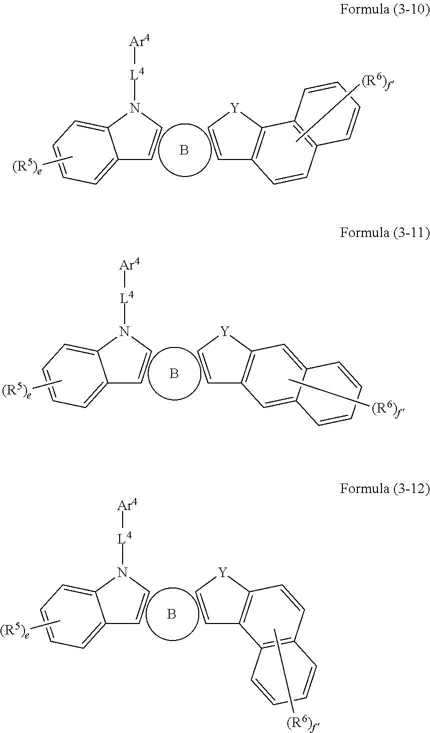
wherein:
1) Ring B, R⁵, R⁶, e, Y, L⁴ and Ar⁴ are the same as defined in claim 10,
2) f′ is an integer of 0 to 6.

15. The organic electronic element of claim 10, wherein Formula (3) is represented by any of Formulas (3-13) to (3-18): 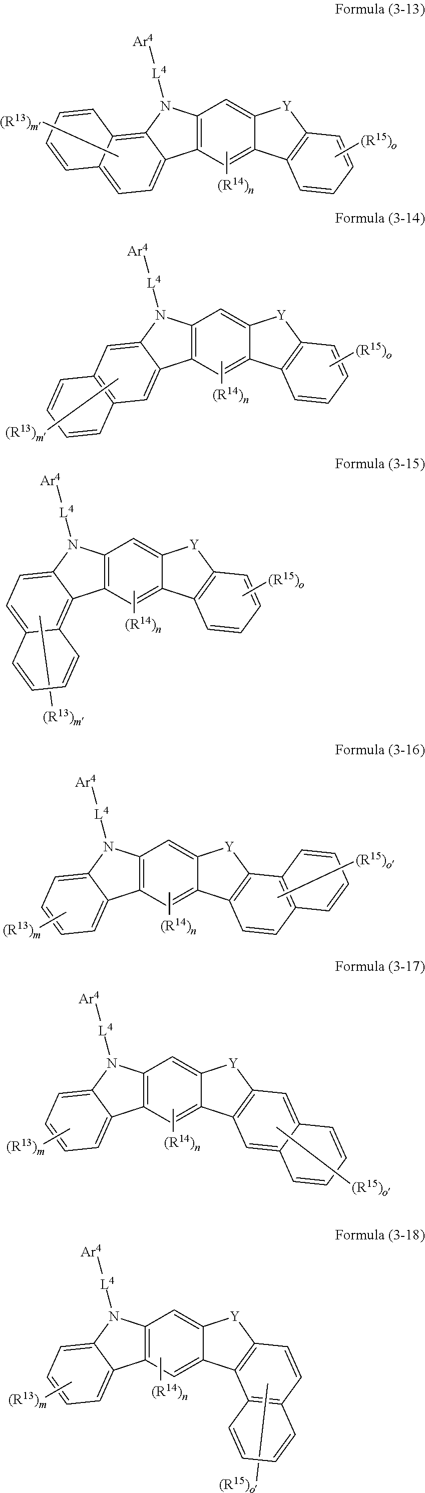
wherein:
1) Y, L⁴ and Ar⁴ are the same as defined in claim 10,
2) R¹³, R¹⁴ and R¹⁵ are each independently same or different, and each independently selected from the group consisting of hydrogen: deuterium: a halogen: a cyano group; a nitro group; a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group; and a C₆-C₃₀ aryloxy group, or an adjacent plurality of R¹³ or plurality of R¹⁴ or plurality of R¹⁵ may be bonded to each other to form a ring, and
3) m and o are each independently an integer of 0 to 4, n is an integer of 0 to 2, and
4) m′ and o′ are each independently an integer of 0 to 6.

16. The organic electronic element of claim 10, wherein 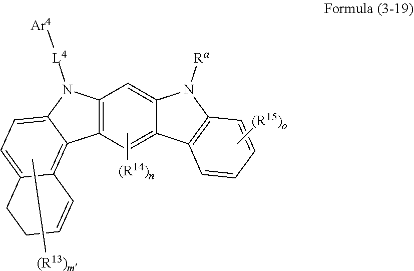
wherein:
1) Ar⁴ and R^{a} are each independently a C₆-C₁₈ aryl group;
2) L⁴ is a single bond or a C₆-C₆₀ arylene group;
3) R¹³, R¹⁴ and R¹⁵ are each independently same or different, and each independently selected from the group consisting of hydrogen; deuterium; a halogen; a cyano group; a nitro group; a C₆-C₆₀ aryl group; a fluorenyl group; a C₂-C₆₀ heterocyclic group including at least one hetero atom of O, N, S, Si or P; a fused ring group of a C₃-C₆₀ aliphatic ring and a C₆-C₆₀ aromatic ring; a C₁-C₆₀ alkyl group; a C₂-C₆₀ alkenyl group; a C₂-C₆₀ alkynyl group; a C₁-C₆₀ alkoxy group;
and a C₆-C₃₀ aryloxy group, or an adjacent plurality of R¹³ or plurality of R¹⁴ or plurality of R¹⁵ may be bonded to each other to form a ring,
4) n is an integer of 0 to 2 and o is an integer of 0 to 4, and
5) m′ is an integer of 0 to 6.

17. The organic electronic element of claim 10, wherein the compound represented by Formula (2) is any of Compounds N-1 to N-100:

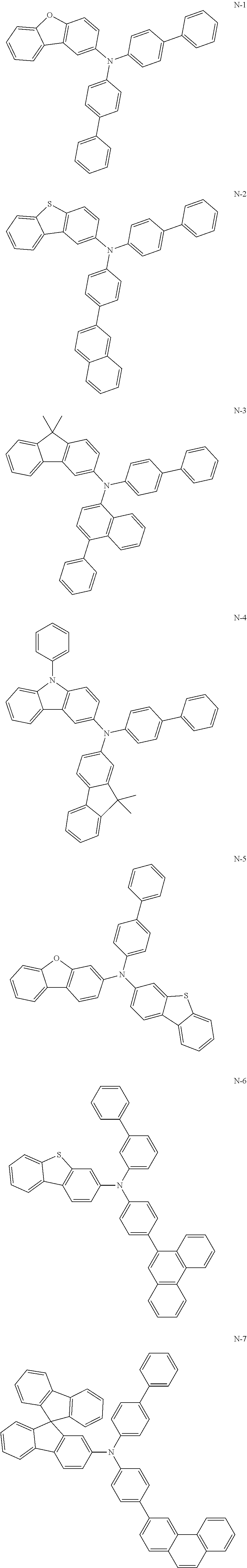

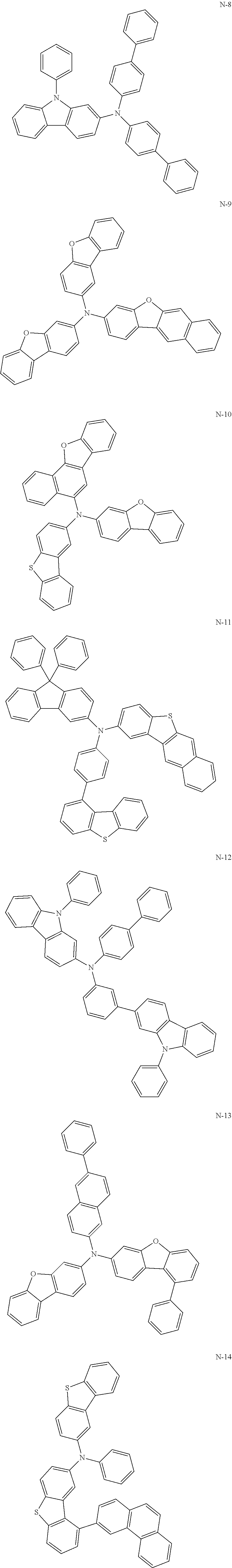

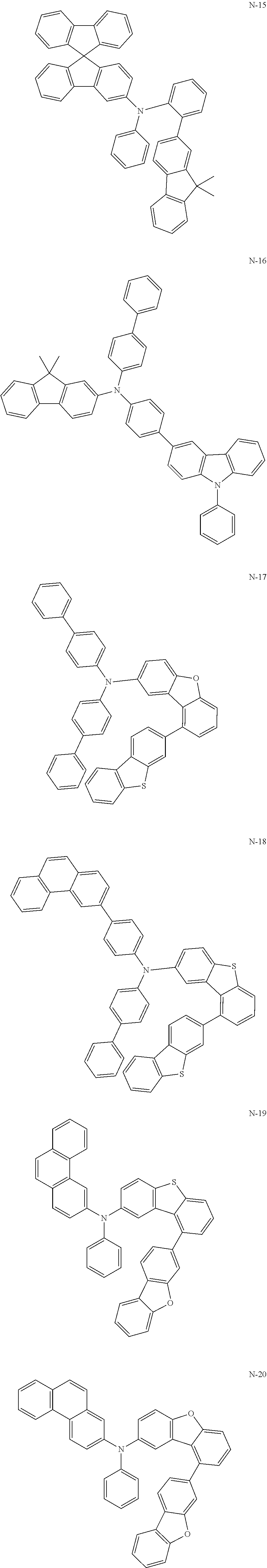

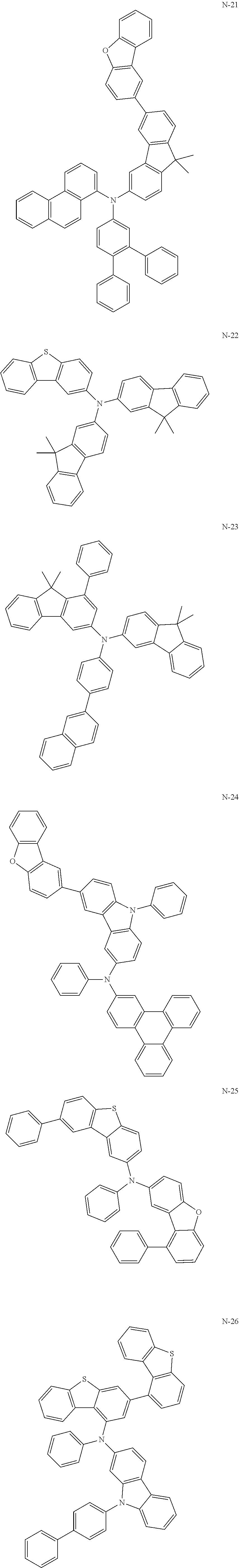

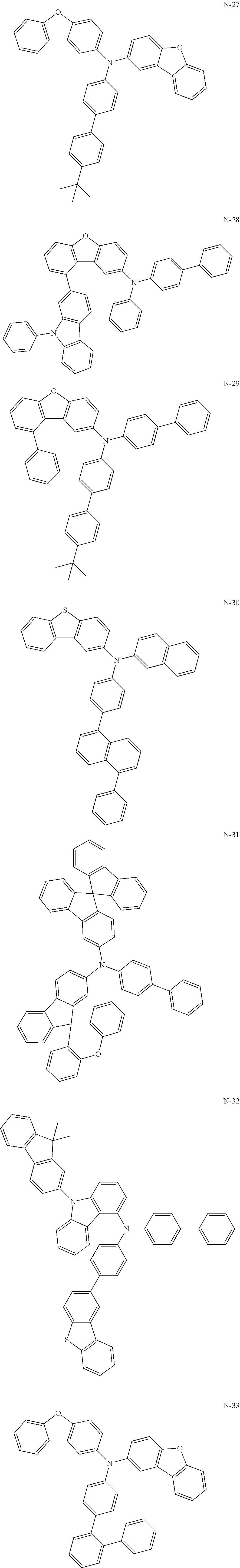

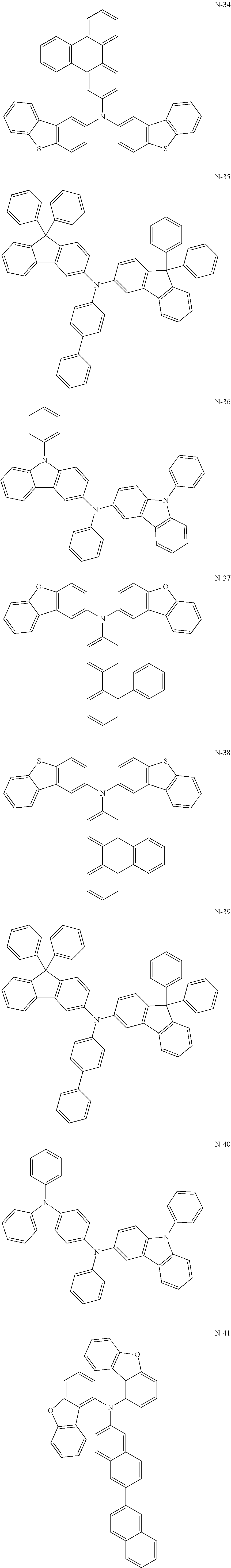

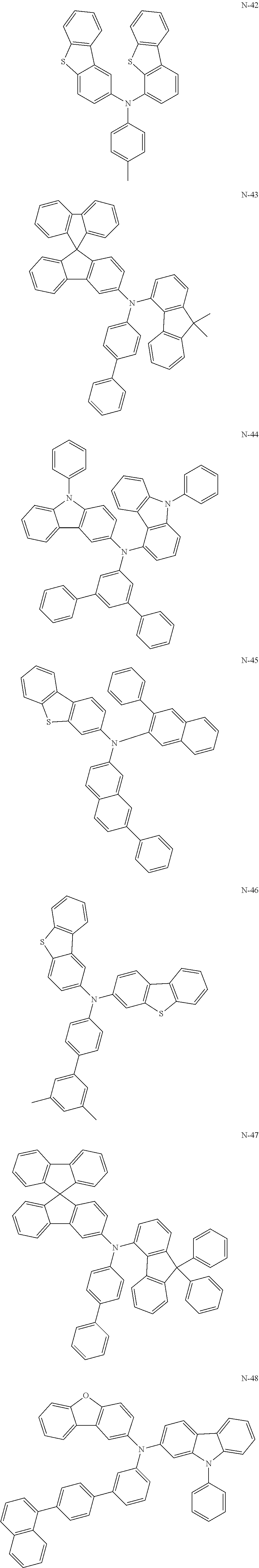

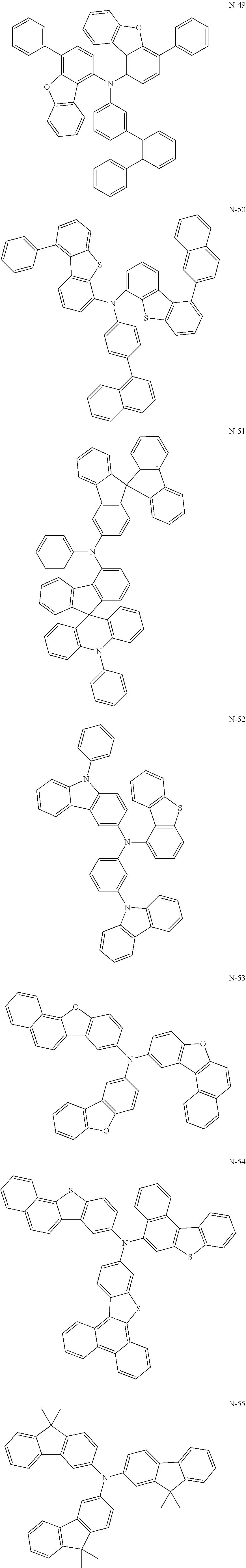

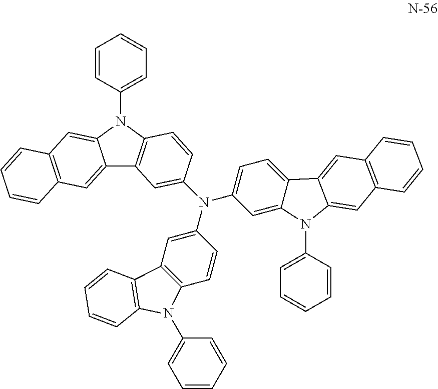 [SMILES: c1ccc(-n2c3ccccc3c3cc(N(c4ccc5c(c4)c4cc6ccccc6cc4n5-c4ccccc4)c4ccc5c6cc7ccccc7cc6n(-c6ccccc6)c5c4)ccc32)cc1]

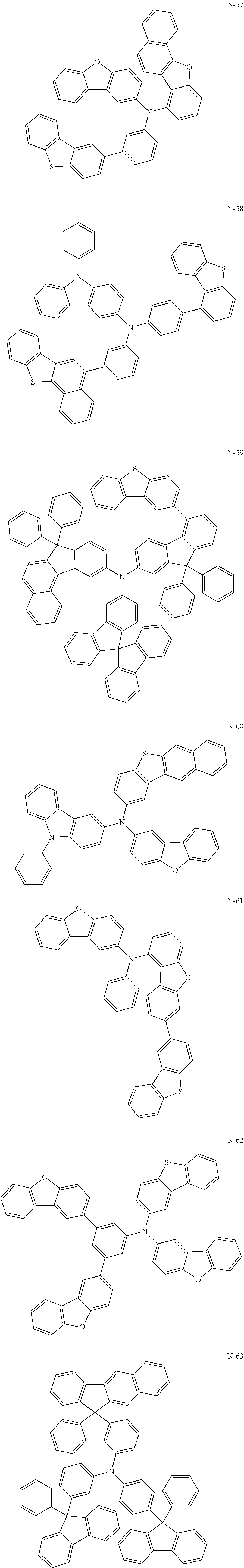

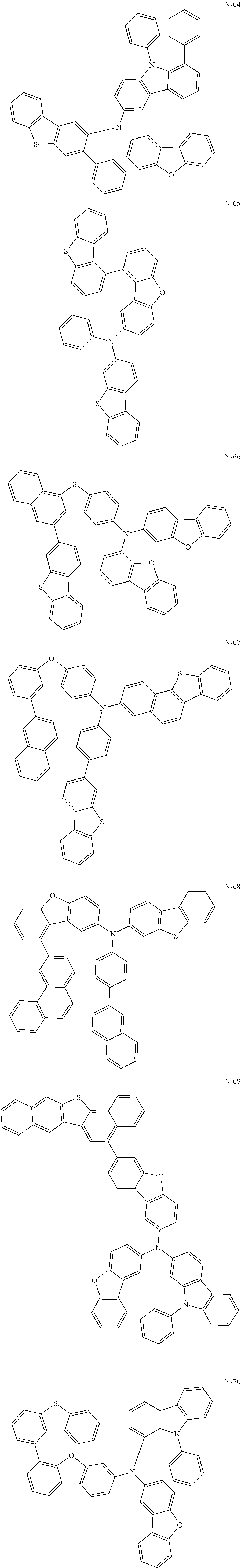

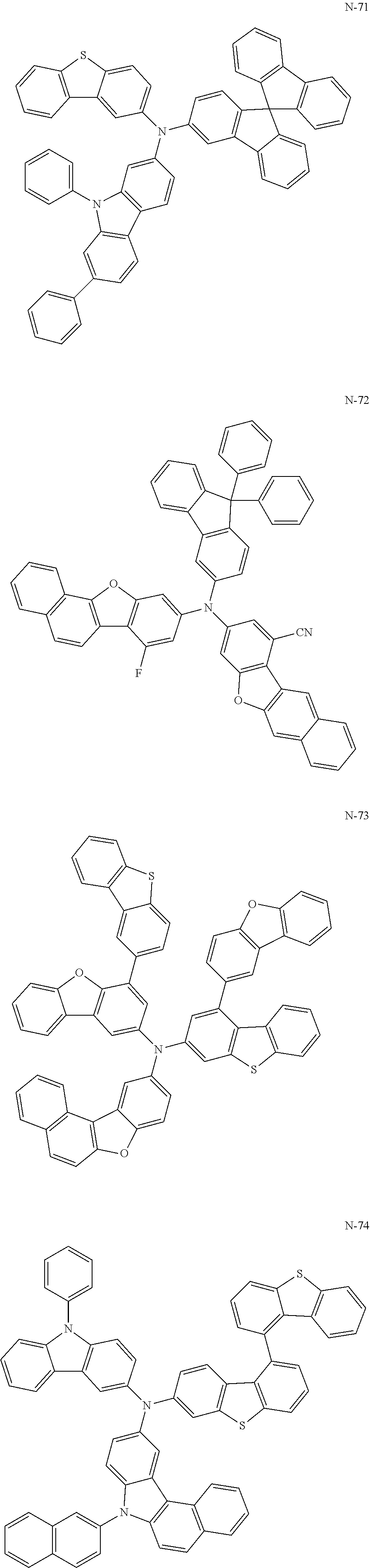 [SMILES: c1ccc(-c2ccc3c4ccc(N(c5ccc6c(c5)-c5ccccc5C65c6ccccc6-c6ccccc65)c5ccc6sc7ccccc7c6c5)cc4n(-c4ccccc4)c3c2)cc1 ; N#Cc1cc(N(c2ccc3c(c2)-c2ccccc2C3(c2ccccc2)c2ccccc2)c2cc(F)c3c(c2)oc2c4ccccc4ccc23)cc2oc3cc4ccccc4cc3c12 ; c1ccc2c(c1)ccc1oc3ccc(N(c4cc(-c5ccc6oc7ccccc7c6c5)c5c(c4)sc4ccccc45)c4cc(-c5ccc6sc7ccccc7c6c5)c5oc6ccccc6c5c4)cc3c12 ; c1ccc(-n2c3ccccc3c3cc(N(c4ccc5c(c4)sc4cccc(-c6cccc7sc8ccccc8c67)c45)c4ccc5c(c4)c4c6ccccc6ccc4n5-c4ccc5ccccc5c4)ccc32)cc1]

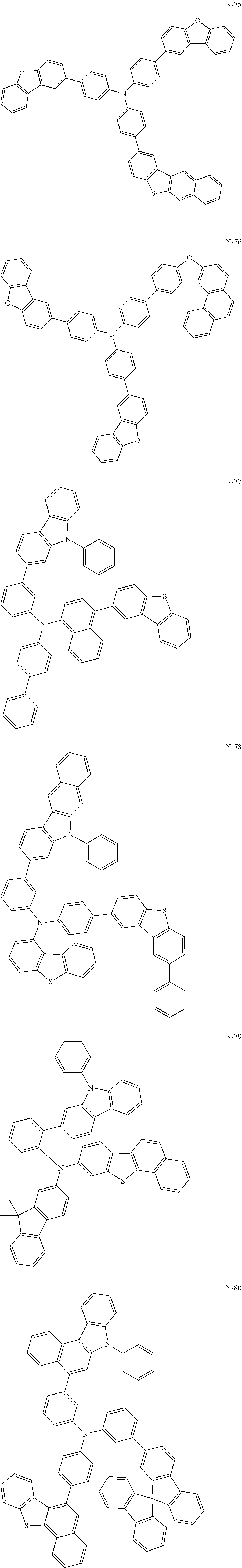

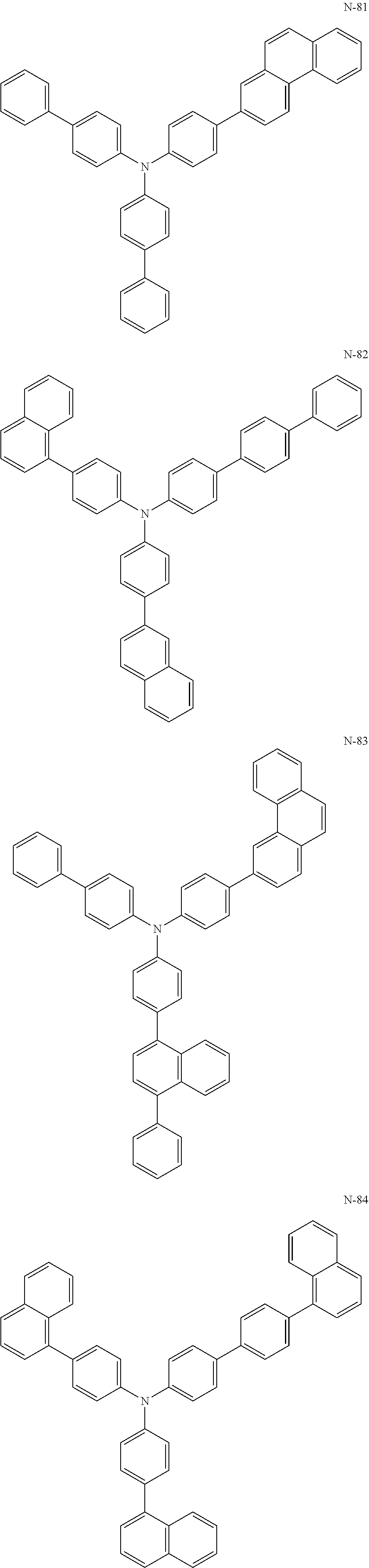 [SMILES: c1ccc(-c2ccc(N(c3ccc(-c4ccccc4)cc3)c3ccc(-c4ccc5c(ccc6ccccc65)c4)cc3)cc2)cc1 ; c1ccc(-c2ccc(-c3ccc(N(c4ccc(-c5ccc6ccccc6c5)cc4)c4ccc(-c5cccc6ccccc56)cc4)cc3)cc2)cc1 ; c1ccc(-c2ccc(N(c3ccc(-c4ccc5ccc6ccccc6c5c4)cc3)c3ccc(-c4ccc(-c5ccccc5)c5ccccc45)cc3)cc2)cc1 ; c1ccc2c(-c3ccc(-c4ccc(N(c5ccc(-c6cccc7ccccc67)cc5)c5ccc(-c6cccc7ccccc67)cc5)cc4)cc3)cccc2c1]

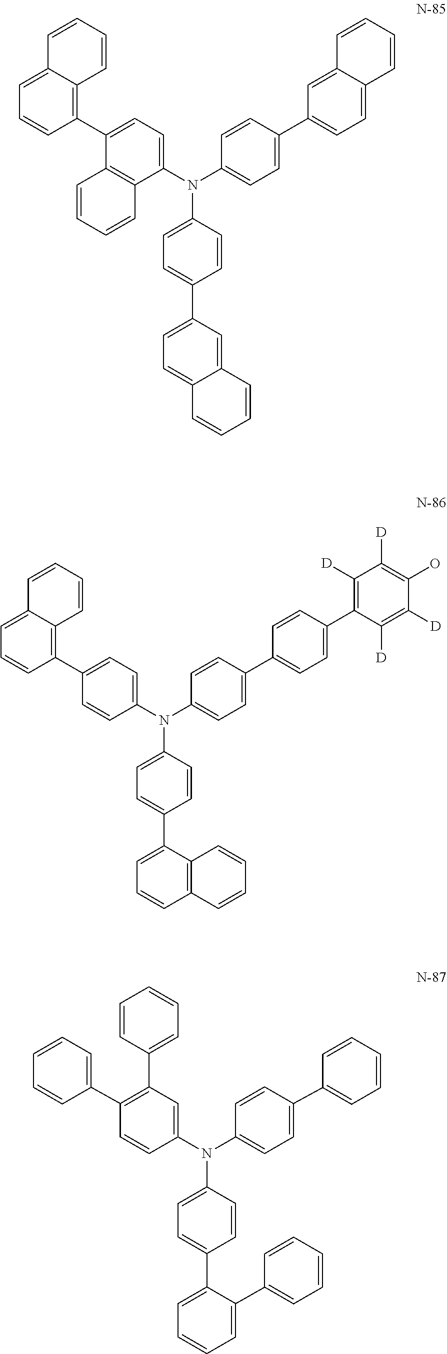

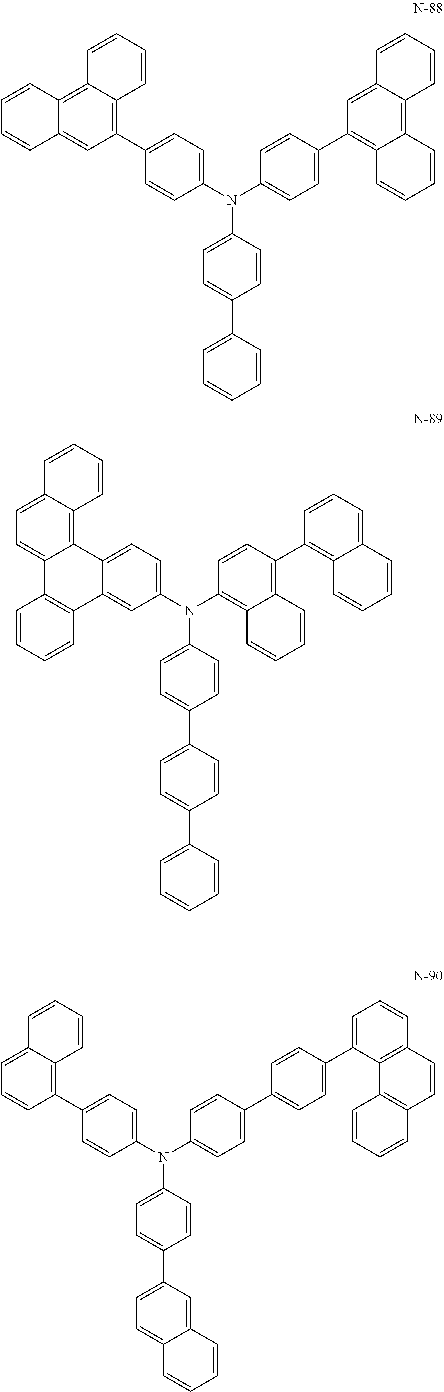 [SMILES: c1ccc(-c2ccc(N(c3ccc(-c4cc5ccccc5c5ccccc45)cc3)c3ccc(-c4cc5ccccc5c5ccccc45)cc3)cc2)cc1 ; c1ccc(-c2ccc(-c3ccc(N(c4ccc5c(c4)c4ccccc4c4ccc6ccccc6c45)c4ccc(-c5cccc6ccccc56)c5ccccc45)cc3)cc2)cc1 ; c1ccc2cc(-c3ccc(N(c4ccc(-c5ccc(-c6cccc7ccc8ccccc8c67)cc5)cc4)c4ccc(-c5cccc6ccccc56)cc4)cc3)ccc2c1]

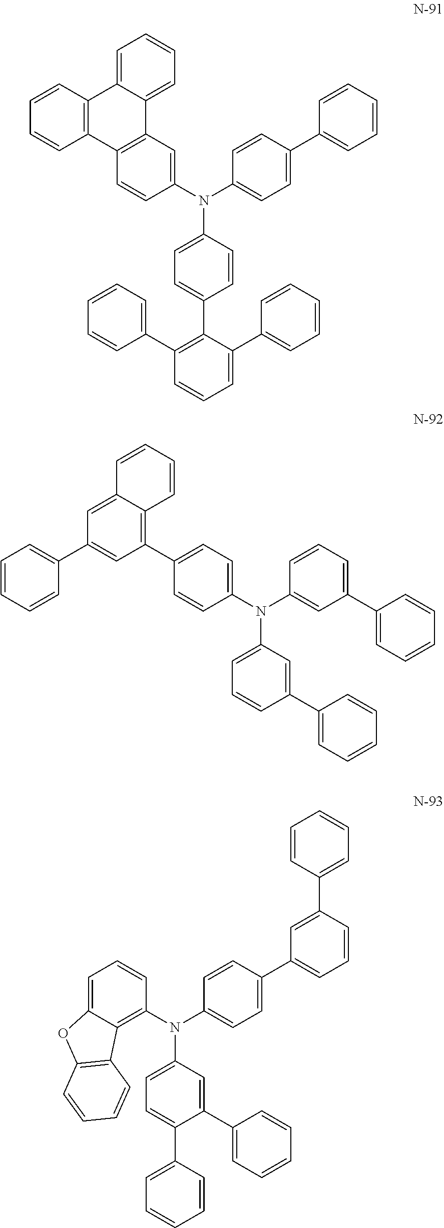 [SMILES: c1ccc(-c2ccc(N(c3ccc(-c4c(-c5ccccc5)cccc4-c4ccccc4)cc3)c3ccc4c5ccccc5c5ccccc5c4c3)cc2)cc1 ; c1ccc(-c2cccc(N(c3ccc(-c4cc(-c5ccccc5)cc5ccccc45)cc3)c3cccc(-c4ccccc4)c3)c2)cc1 ; c1ccc(-c2cccc(-c3ccc(N(c4ccc(-c5ccccc5)c(-c5ccccc5)c4)c4cccc5oc6ccccc6c45)cc3)c2)cc1]

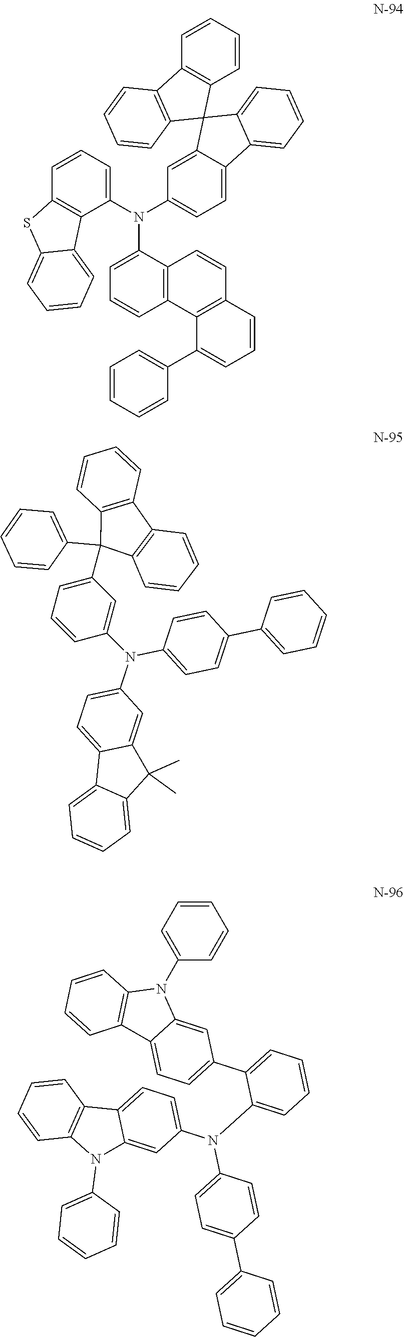

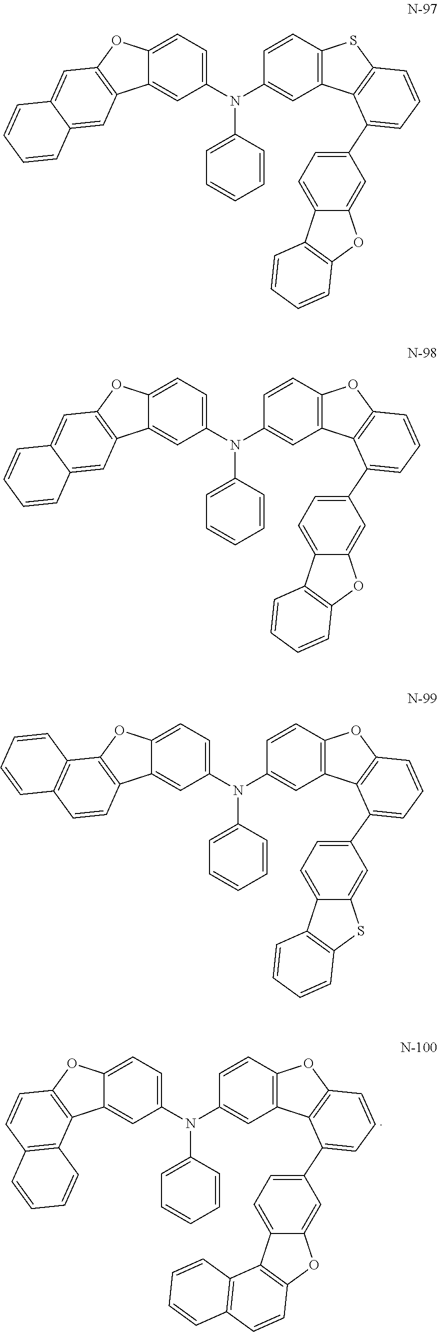 [SMILES: c1ccc(N(c2ccc3oc4cc5ccccc5cc4c3c2)c2ccc3sc4cccc(-c5ccc6c(c5)oc5ccccc56)c4c3c2)cc1 ; c1ccc(N(c2ccc3oc4cc5ccccc5cc4c3c2)c2ccc3oc4cccc(-c5ccc6c(c5)oc5ccccc56)c4c3c2)cc1 ; c1ccc(N(c2ccc3oc4c5ccccc5ccc4c3c2)c2ccc3oc4cccc(-c5ccc6c(c5)sc5ccccc56)c4c3c2)cc1 ; c1ccc(N(c2ccc3oc4cccc(-c5ccc6c(c5)oc5ccc7ccccc7c56)c4c3c2)c2ccc3oc4ccc5ccccc5c4c3c2)cc1]

18. The organic electronic element of claim 10, wherein the compound represented by Formula (3) is any of Compounds S-1 to S-112:

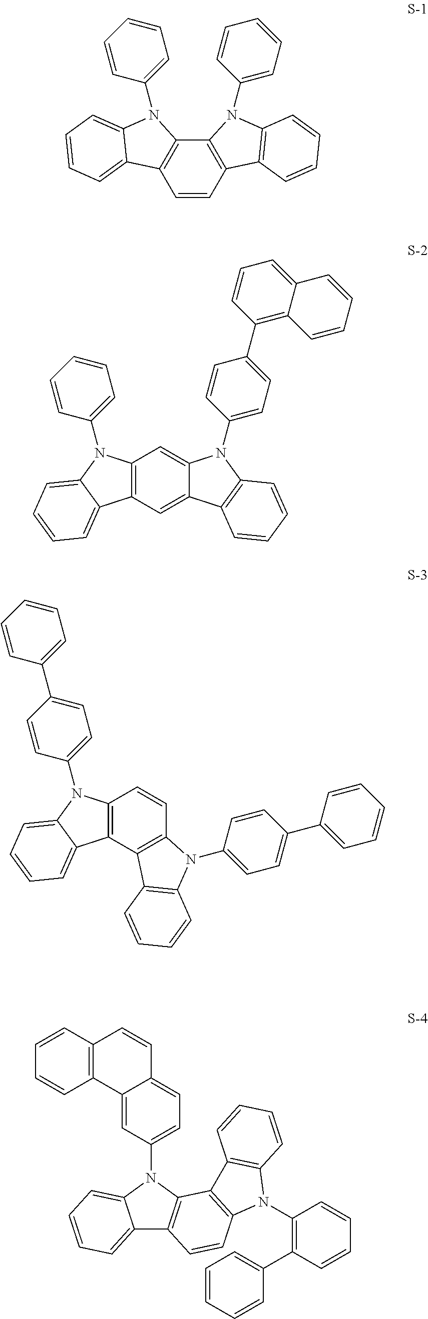 [SMILES: c1ccc(-n2c3ccccc3c3ccc4c5ccccc5n(-c5ccccc5)c4c32)cc1 ; c1ccc(-n2c3ccccc3c3cc4c5ccccc5n(-c5ccc(-c6cccc7ccccc67)cc5)c4cc32)cc1 ; c1ccc(-c2ccc(-n3c4ccccc4c4c5c6ccccc6n(-c6ccc(-c7ccccc7)cc6)c5ccc43)cc2)cc1 ; c1ccc(-c2ccccc2-n2c3ccccc3c3c2ccc2c4ccccc4n(-c4ccc5ccc6ccccc6c5c4)c23)cc1]

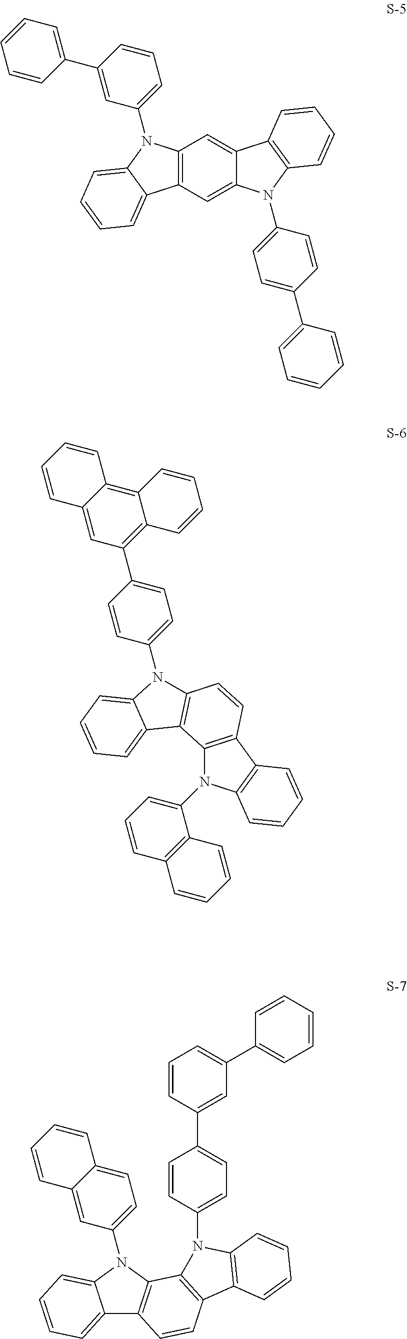 [SMILES: c1ccc(-c2ccc(-n3c4ccccc4c4cc5c(cc43)c3ccccc3n5-c3cccc(-c4ccccc4)c3)cc2)cc1 ; c1ccc2c(-n3c4ccccc4c4ccc5c(c6ccccc6n5-c5ccc(-c6cc7ccccc7c7ccccc67)cc5)c43)cccc2c1 ; c1ccc(-c2cccc(-c3ccc(-n4c5ccccc5c5ccc6c7ccccc7n(-c7ccc8ccccc8c7)c6c54)cc3)c2)cc1]

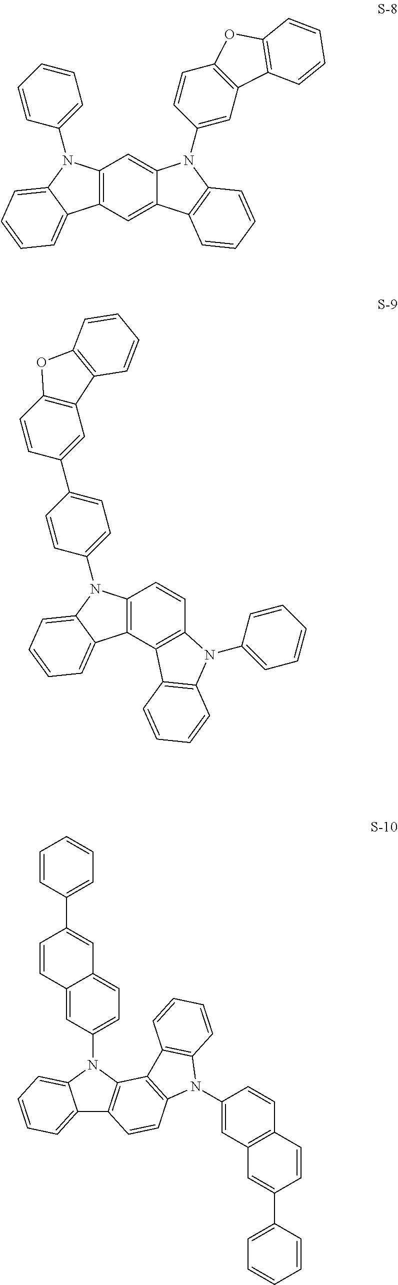 [SMILES: c1ccc(-n2c3ccccc3c3cc4c5ccccc5n(-c5ccc6oc7ccccc7c6c5)c4cc32)cc1 ; c1ccc(-n2c3ccccc3c3c4c5ccccc5n(-c5ccc(-c6ccc7oc8ccccc8c7c6)cc5)c4ccc32)cc1 ; c1ccc(-c2ccc3cc(-n4c5ccccc5c5ccc6c(c7ccccc7n6-c6ccc7ccc(-c8ccccc8)cc7c6)c54)ccc3c2)cc1]

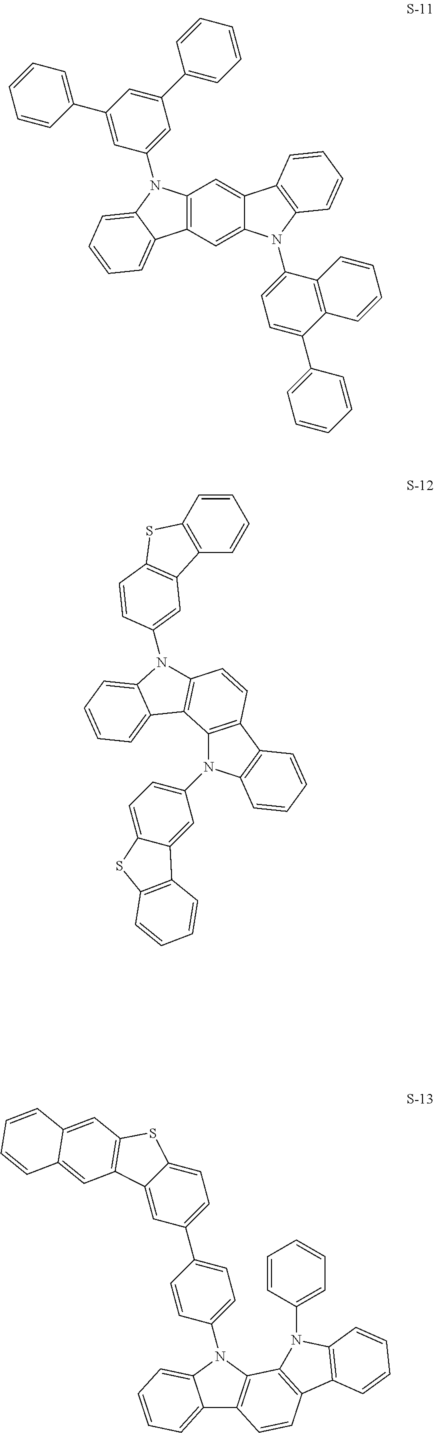 [SMILES: c1ccc(-c2cc(-c3ccccc3)cc(-n3c4ccccc4c4cc5c(cc43)c3ccccc3n5-c3ccc(-c4ccccc4)c4ccccc34)c2)cc1 ; c1ccc2c(c1)sc1ccc(-n3c4ccccc4c4c3ccc3c5ccccc5n(-c5ccc6sc7ccccc7c6c5)c34)cc12 ; c1ccc(-n2c3ccccc3c3ccc4c5ccccc5n(-c5ccc(-c6ccc7sc8cc9ccccc9cc8c7c6)cc5)c4c32)cc1]

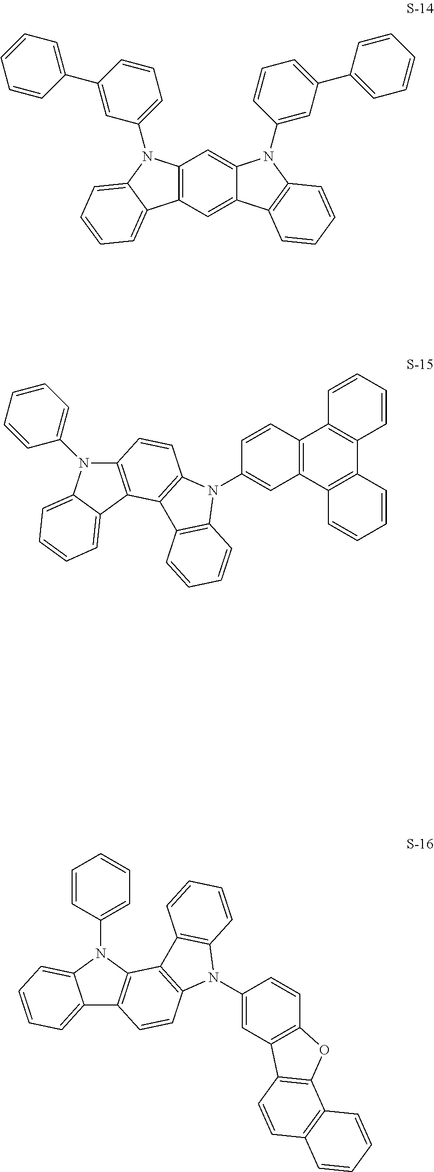 [SMILES: c1ccc(-c2cccc(-n3c4ccccc4c4cc5c6ccccc6n(-c6cccc(-c7ccccc7)c6)c5cc43)c2)cc1 ; c1ccc(-n2c3ccccc3c3c4c5ccccc5n(-c5ccc6c7ccccc7c7ccccc7c6c5)c4ccc32)cc1 ; c1ccc(-n2c3ccccc3c3ccc4c(c5ccccc5n4-c4ccc5oc6c7ccccc7ccc6c5c4)c32)cc1]

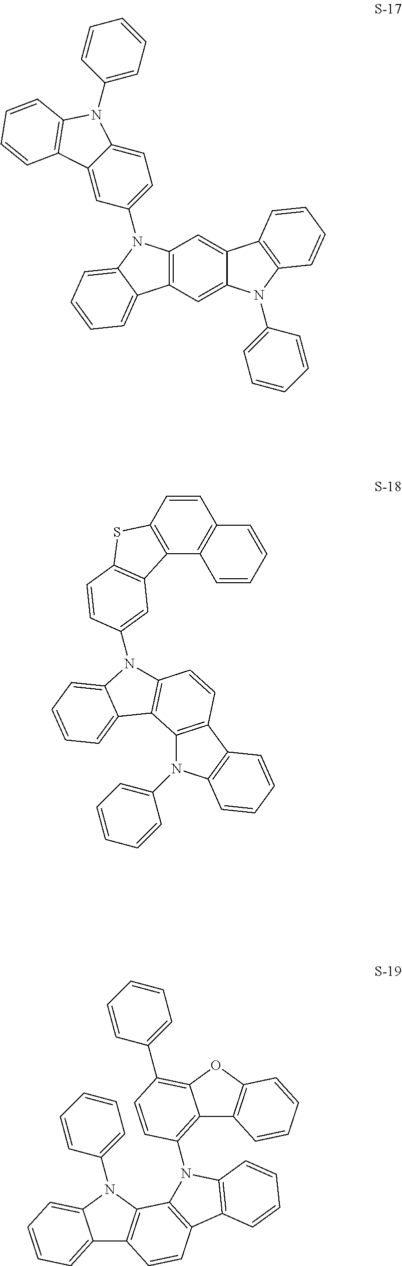 [SMILES: c1ccc(-n2c3ccccc3c3cc(-n4c5ccccc5c5cc6c(cc54)c4ccccc4n6-c4ccccc4)ccc32)cc1 ; c1ccc(-n2c3ccccc3c3ccc4c(c5ccccc5n4-c4ccc5sc6ccc7ccccc7c6c5c4)c32)cc1 ; c1ccc(-c2ccc(-n3c4ccccc4c4ccc5c6ccccc6n(-c6ccccc6)c5c43)c3c2oc2ccccc23)cc1]

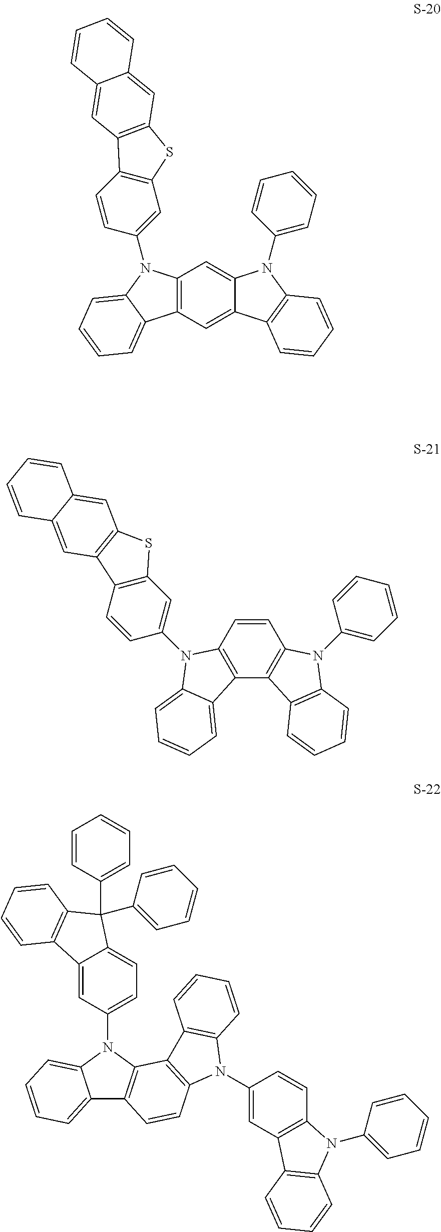 [SMILES: c1ccc(-n2c3ccccc3c3cc4c5ccccc5n(-c5ccc6c(c5)sc5cc7ccccc7cc56)c4cc32)cc1 ; c1ccc(-n2c3ccccc3c3c4c5ccccc5n(-c5ccc6c(c5)sc5cc7ccccc7cc56)c4ccc32)cc1 ; c1ccc(-n2c3ccccc3c3cc(-n4c5ccccc5c5c4ccc4c6ccccc6n(-c6ccc7c(c6)-c6ccccc6C7(c6ccccc6)c6ccccc6)c45)ccc32)cc1]

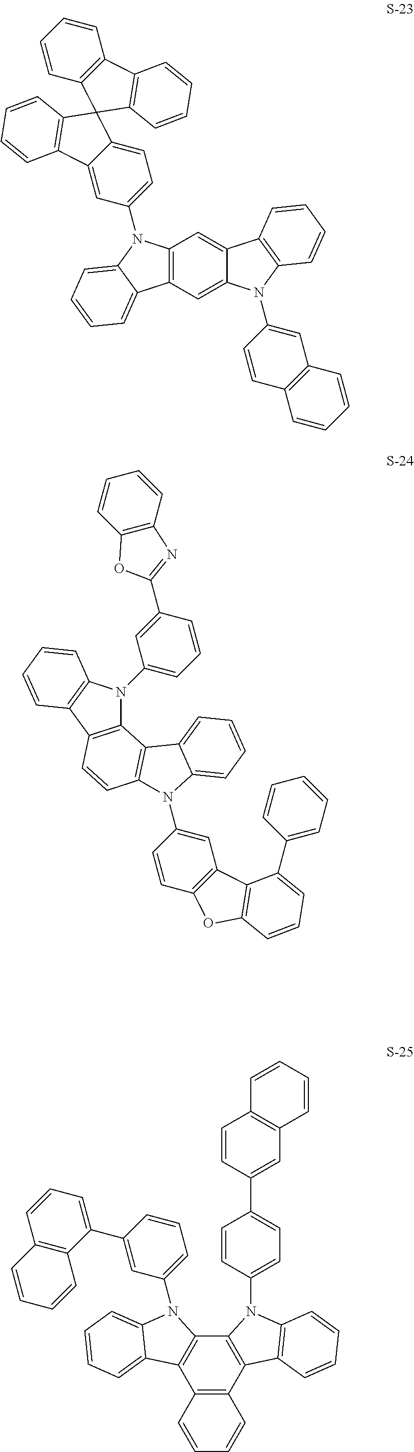 [SMILES: c1ccc2c(c1)-c1ccccc1C21c2ccccc2-c2cc(-n3c4ccccc4c4cc5c(cc43)c3ccccc3n5-c3ccc4ccccc4c3)ccc21 ; c1ccc(-c2cccc3oc4ccc(-n5c6ccccc6c6c5ccc5c7ccccc7n(-c7cccc(-c8nc9ccccc9o8)c7)c56)cc4c23)cc1 ; c1cc(-c2cccc3ccccc23)cc(-n2c3ccccc3c3c4ccccc4c4c5ccccc5n(-c5ccc(-c6ccc7ccccc7c6)cc5)c4c32)c1]

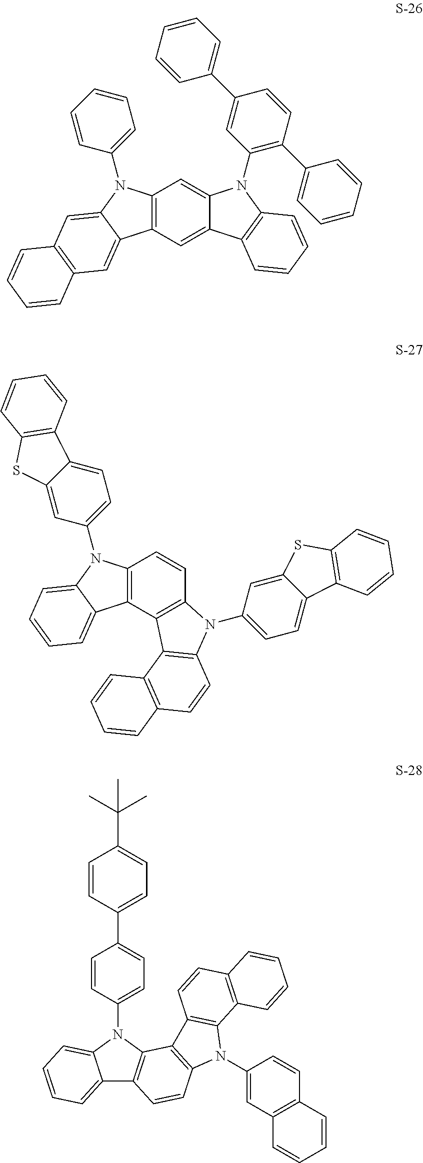 [SMILES: c1ccc(-c2ccc(-c3ccccc3)c(-n3c4ccccc4c4cc5c6cc7ccccc7cc6n(-c6ccccc6)c5cc43)c2)cc1 ; c1ccc2c(c1)ccc1c2c2c3c4ccccc4n(-c4ccc5c(c4)sc4ccccc45)c3ccc2n1-c1ccc2c(c1)sc1ccccc12 ; CC(C)(C)c1ccc(-c2ccc(-n3c4ccccc4c4ccc5c(c6ccc7ccccc7c6n5-c5ccc6ccccc6c5)c43)cc2)cc1]

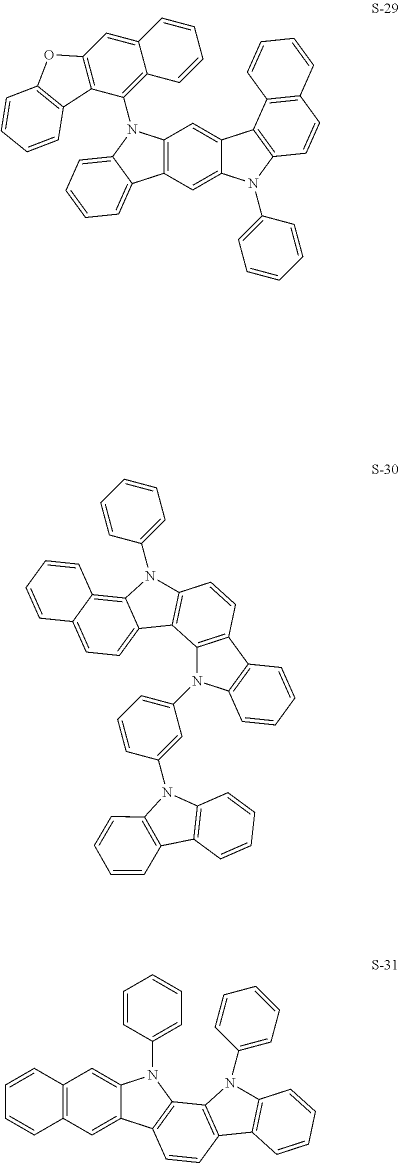 [SMILES: c1ccc(-n2c3cc4c5ccccc5n(-c5c6ccccc6cc6oc7ccccc7c56)c4cc3c3c4ccccc4ccc32)cc1 ; c1ccc(-n2c3ccc4c5ccccc5n(-c5cccc(-n6c7ccccc7c7ccccc76)c5)c4c3c3ccc4ccccc4c32)cc1 ; c1ccc(-n2c3ccccc3c3ccc4c5cc6ccccc6cc5n(-c5ccccc5)c4c32)cc1]

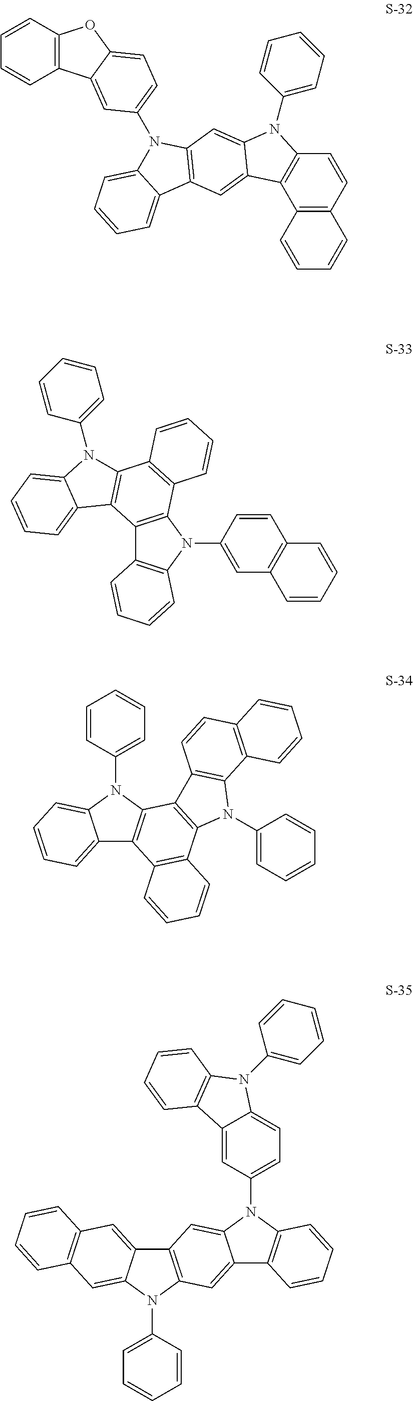 [SMILES: c1ccc(-n2c3cc4c(cc3c3c5ccccc5ccc32)c2ccccc2n4-c2ccc3oc4ccccc4c3c2)cc1 ; c1ccc(-n2c3ccccc3c3c4c5ccccc5n(-c5ccc6ccccc6c5)c4c4ccccc4c32)cc1 ; c1ccc(-n2c3ccccc3c3c4ccccc4c4c(c5ccc6ccccc6c5n4-c4ccccc4)c32)cc1 ; c1ccc(-n2c3ccccc3c3cc(-n4c5ccccc5c5cc6c(cc54)c4cc5ccccc5cc4n6-c4ccccc4)ccc32)cc1]

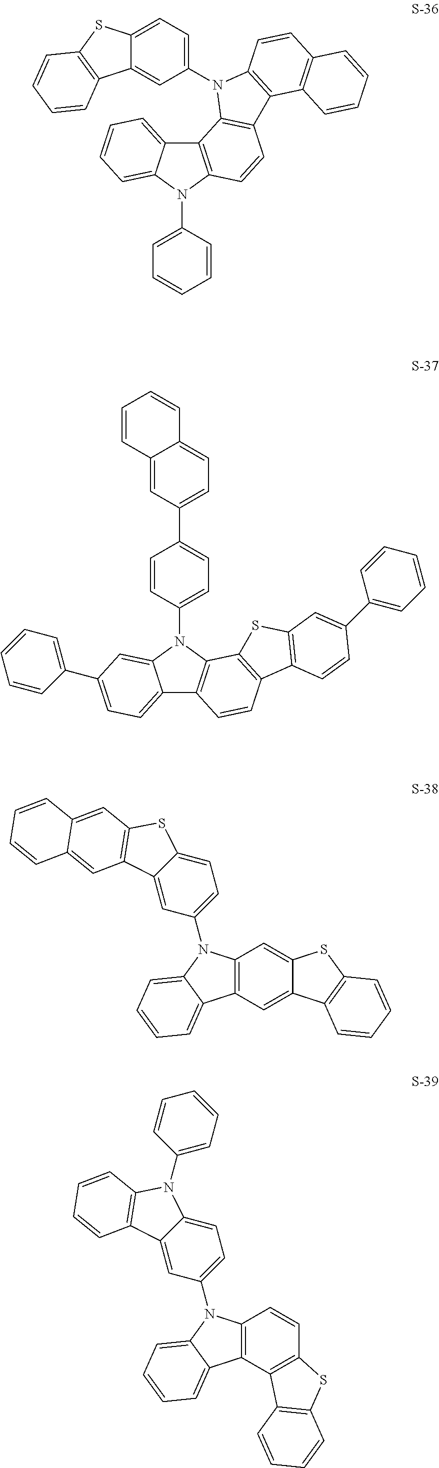 [SMILES: c1ccc(-n2c3ccccc3c3c2ccc2c4c5ccccc5ccc4n(-c4ccc5sc6ccccc6c5c4)c23)cc1 ; c1ccc(-c2ccc3c(c2)sc2c3ccc3c4ccc(-c5ccccc5)cc4n(-c4ccc(-c5ccc6ccccc6c5)cc4)c32)cc1 ; c1ccc2cc3c(cc2c1)sc1ccc(-n2c4ccccc4c4cc5c(cc42)sc2ccccc25)cc13 ; c1ccc(-n2c3ccccc3c3cc(-n4c5ccccc5c5c6c(ccc54)sc4ccccc46)ccc32)cc1]

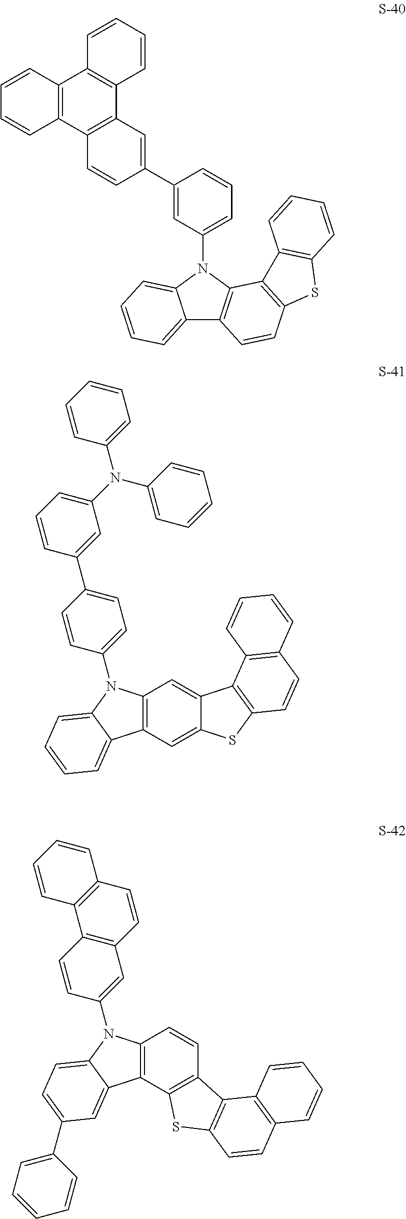 [SMILES: c1cc(-c2ccc3c4ccccc4c4ccccc4c3c2)cc(-n2c3ccccc3c3ccc4sc5ccccc5c4c32)c1 ; c1ccc(N(c2ccccc2)c2cccc(-c3ccc(-n4c5ccccc5c5cc6sc7ccc8ccccc8c7c6cc54)cc3)c2)cc1 ; c1ccc(-c2ccc3c(c2)c2c4sc5ccc6ccccc6c5c4ccc2n3-c2ccc3c(ccc4ccccc43)c2)cc1]

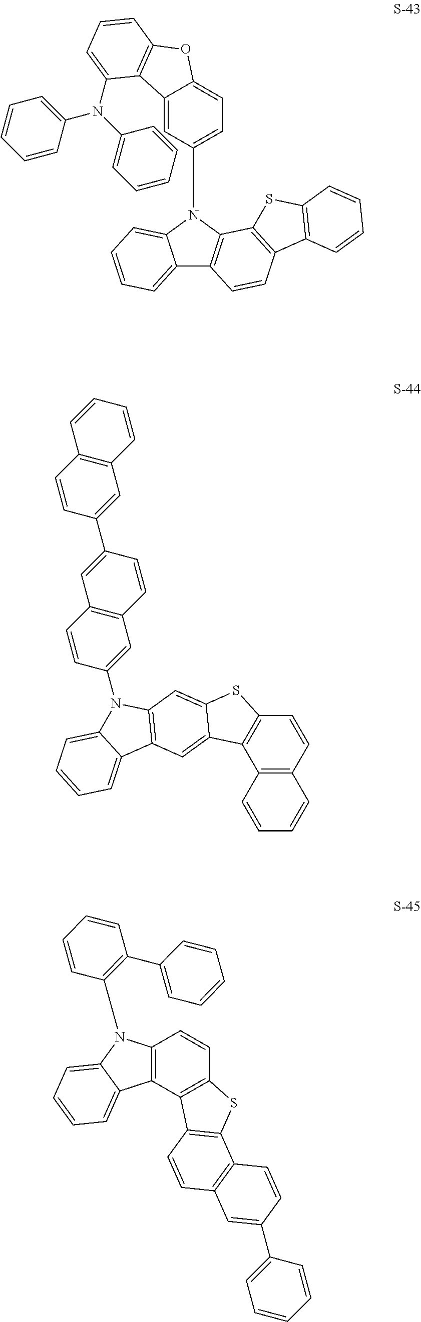 [SMILES: c1ccc(N(c2ccccc2)c2cccc3oc4ccc(-n5c6ccccc6c6ccc7c8ccccc8sc7c65)cc4c23)cc1 ; c1ccc2cc(-c3ccc4cc(-n5c6ccccc6c6cc7c(cc65)sc5ccc6ccccc6c57)ccc4c3)ccc2c1 ; c1ccc(-c2ccc3c(ccc4c3sc3ccc5c(c6ccccc6n5-c5ccccc5-c5ccccc5)c34)c2)cc1]

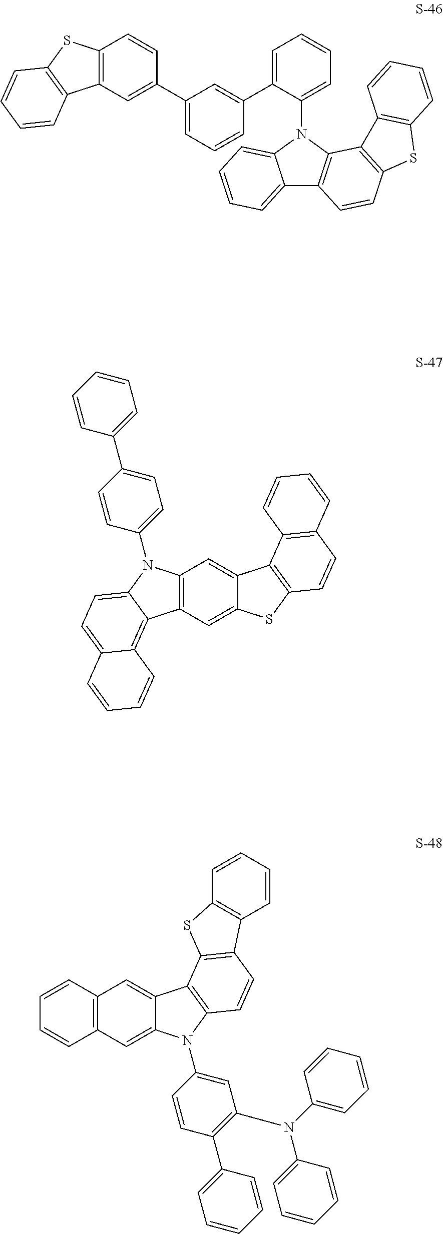 [SMILES: c1cc(-c2ccc3sc4ccccc4c3c2)cc(-c2ccccc2-n2c3ccccc3c3ccc4sc5ccccc5c4c32)c1 ; c1ccc(-c2ccc(-n3c4cc5c(cc4c4c6ccccc6ccc43)sc3ccc4ccccc4c35)cc2)cc1 ; c1ccc(-c2ccc(-n3c4cc5ccccc5cc4c4c5sc6ccccc6c5ccc43)cc2N(c2ccccc2)c2ccccc2)cc1]

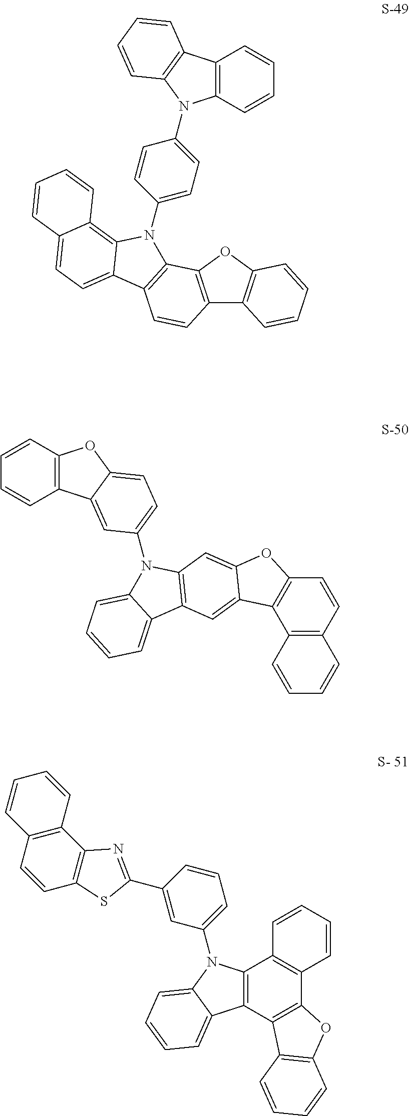 [SMILES: c1ccc2c(c1)ccc1c3ccc4c5ccccc5oc4c3n(-c3ccc(-n4c5ccccc5c5ccccc54)cc3)c21 ; c1ccc2c(c1)ccc1oc3cc4c(cc3c12)c1ccccc1n4-c1ccc2oc3ccccc3c2c1 ; c1cc(-c2nc3c(ccc4ccccc43)s2)cc(-n2c3ccccc3c3c4c5ccccc5oc4c4ccccc4c32)c1]

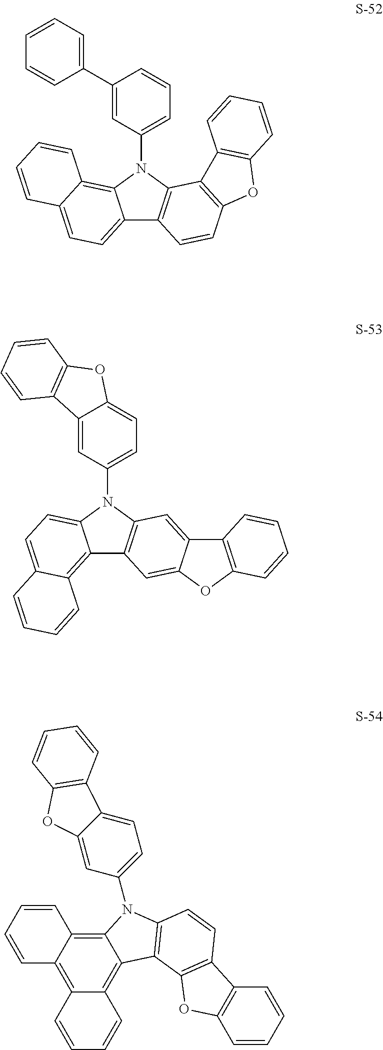 [SMILES: c1ccc(-c2cccc(-n3c4c5ccccc5ccc4c4ccc5oc6ccccc6c5c43)c2)cc1 ; c1ccc2c(c1)ccc1c2c2cc3oc4ccccc4c3cc2n1-c1ccc2oc3ccccc3c2c1 ; c1ccc2c(c1)oc1cc(-n3c4ccc5c6ccccc6oc5c4c4c5ccccc5c5ccccc5c43)ccc12]

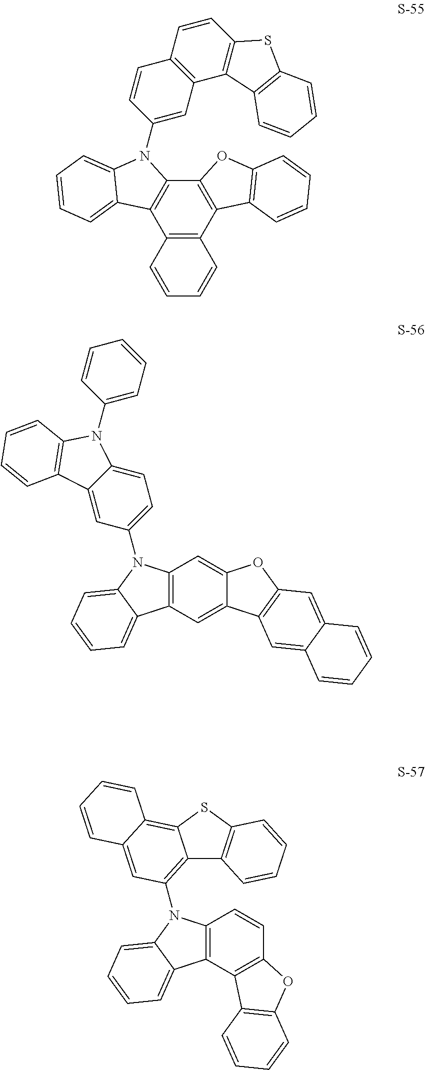 [SMILES: c1ccc2c(c1)oc1c2c2ccccc2c2c3ccccc3n(-c3ccc4ccc5sc6ccccc6c5c4c3)c12 ; c1ccc(-n2c3ccccc3c3cc(-n4c5ccccc5c5cc6c(cc54)oc4cc5ccccc5cc46)ccc32)cc1 ; c1ccc2c(c1)cc(-n1c3ccccc3c3c4c(ccc31)oc1ccccc14)c1c3ccccc3sc21]

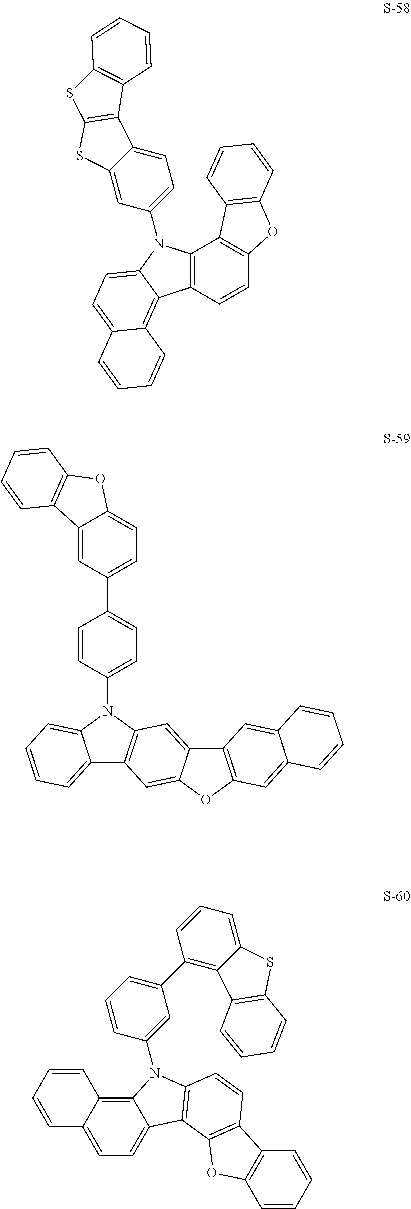 [SMILES: c1ccc2c(c1)ccc1c2c2ccc3oc4ccccc4c3c2n1-c1ccc2c(c1)sc1sc3ccccc3c12 ; c1ccc2cc3c(cc2c1)oc1cc2c4ccccc4n(-c4ccc(-c5ccc6oc7ccccc7c6c5)cc4)c2cc13 ; c1cc(-c2cccc3sc4ccccc4c23)cc(-n2c3ccc4c5ccccc5oc4c3c3ccc4ccccc4c32)c1]

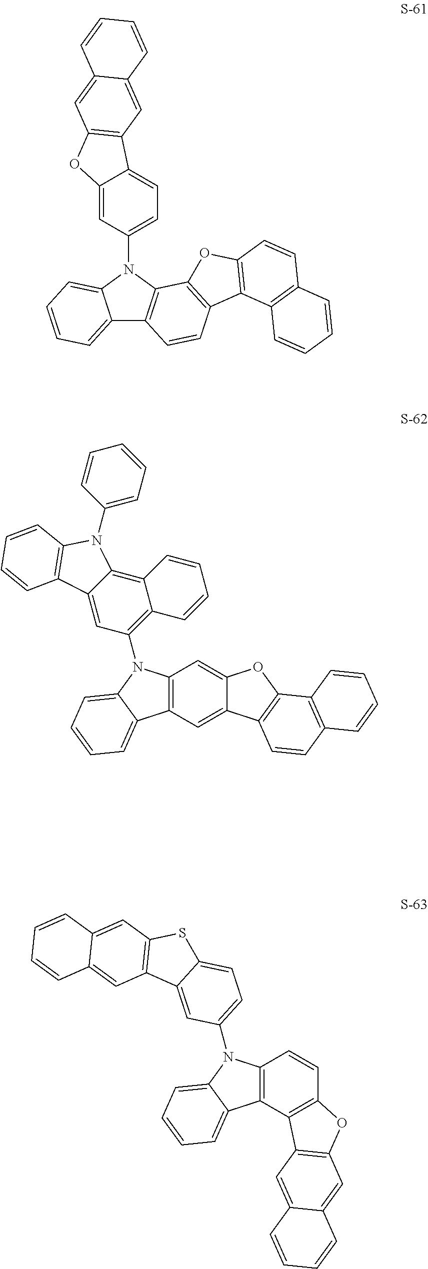 [SMILES: c1ccc2cc3c(cc2c1)oc1cc(-n2c4ccccc4c4ccc5c(oc6ccc7ccccc7c65)c42)ccc13 ; c1ccc(-n2c3ccccc3c3cc(-n4c5ccccc5c5cc6c(cc54)oc4c5ccccc5ccc64)c4ccccc4c32)cc1 ; c1ccc2cc3c(cc2c1)oc1ccc2c(c4ccccc4n2-c2ccc4sc5cc6ccccc6cc5c4c2)c13]

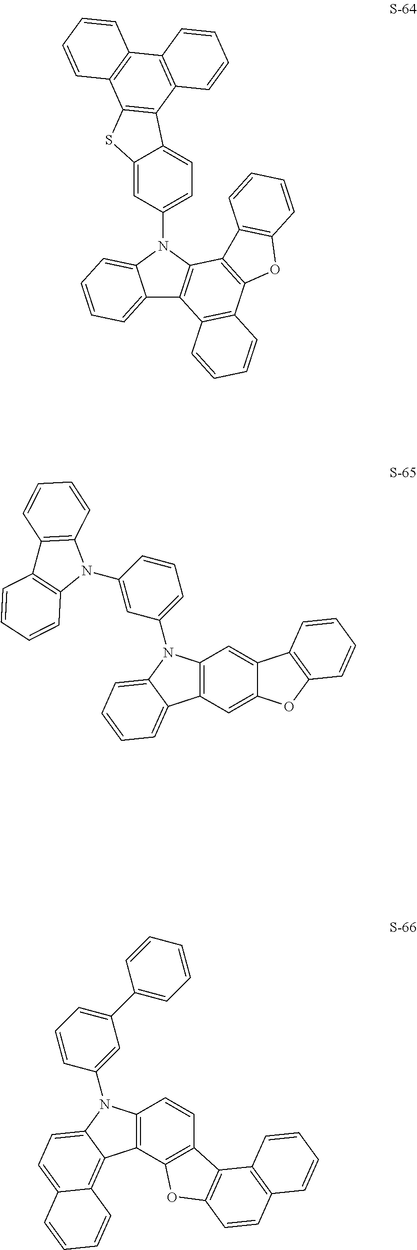 [SMILES: c1ccc2c(c1)oc1c3ccccc3c3c4ccccc4n(-c4ccc5c(c4)sc4c6ccccc6c6ccccc6c54)c3c21 ; c1cc(-n2c3ccccc3c3ccccc32)cc(-n2c3ccccc3c3cc4oc5ccccc5c4cc32)c1 ; c1ccc(-c2cccc(-n3c4ccc5ccccc5c4c4c5oc6ccc7ccccc7c6c5ccc43)c2)cc1]

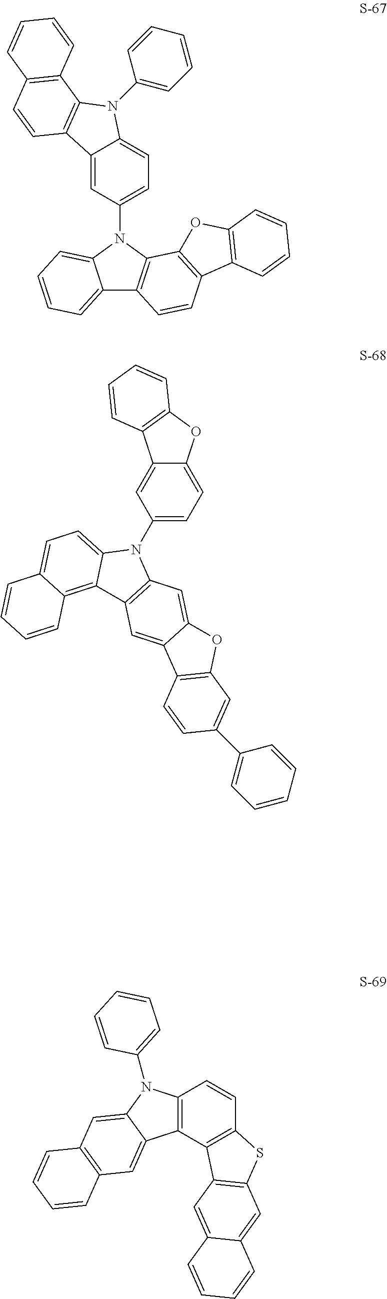 [SMILES: c1ccc(-n2c3ccc(-n4c5ccccc5c5ccc6c7ccccc7oc6c54)cc3c3ccc4ccccc4c32)cc1 ; c1ccc(-c2ccc3c(c2)oc2cc4c(cc23)c2c3ccccc3ccc2n4-c2ccc3oc4ccccc4c3c2)cc1 ; c1ccc(-n2c3cc4ccccc4cc3c3c4c(ccc32)sc2cc3ccccc3cc24)cc1]

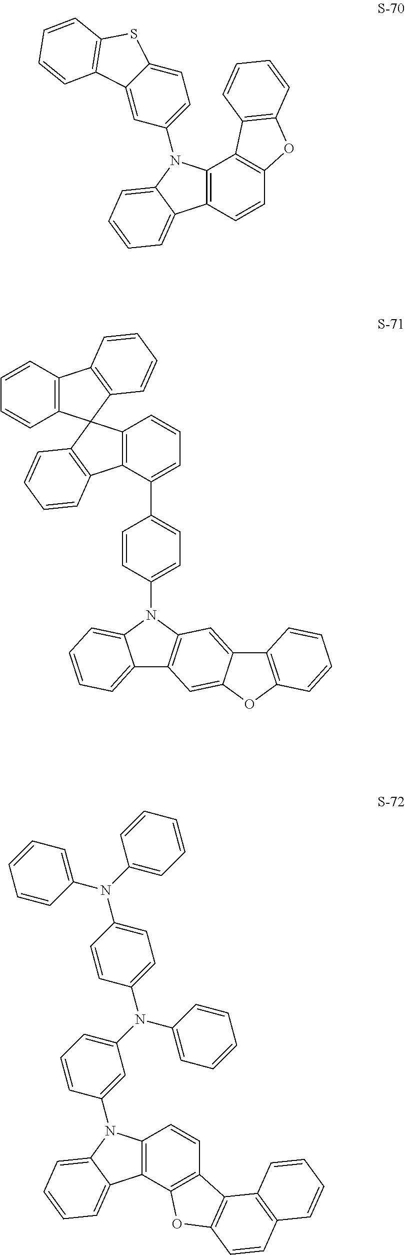 [SMILES: c1ccc2c(c1)oc1ccc3c4ccccc4n(-c4ccc5sc6ccccc6c5c4)c3c12 ; c1ccc2c(c1)-c1ccccc1C21c2ccccc2-c2c(-c3ccc(-n4c5ccccc5c5cc6oc7ccccc7c6cc54)cc3)cccc21 ; c1ccc(N(c2ccccc2)c2ccc(N(c3ccccc3)c3cccc(-n4c5ccccc5c5c6oc7ccc8ccccc8c7c6ccc54)c3)cc2)cc1]

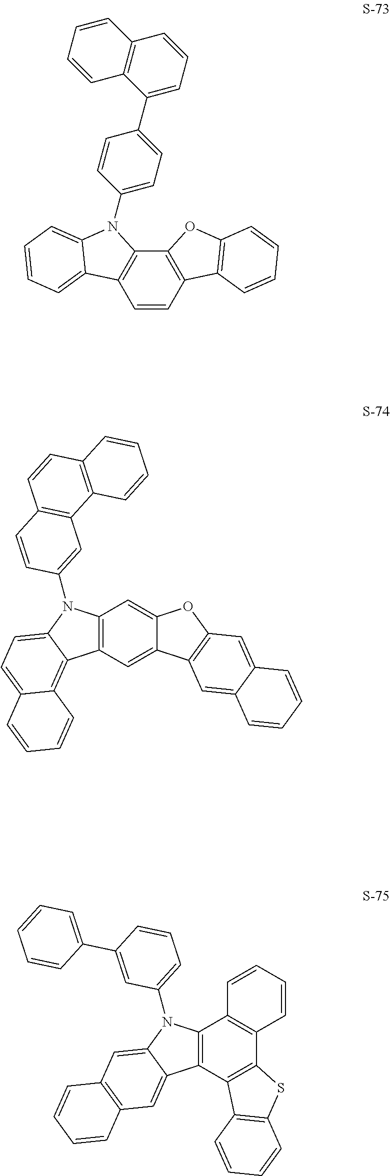 [SMILES: c1ccc2c(-c3ccc(-n4c5ccccc5c5ccc6c7ccccc7oc6c54)cc3)cccc2c1 ; c1ccc2cc3c(cc2c1)oc1cc2c(cc13)c1c3ccccc3ccc1n2-c1ccc2ccc3ccccc3c2c1 ; c1ccc(-c2cccc(-n3c4cc5ccccc5cc4c4c5c6ccccc6sc5c5ccccc5c43)c2)cc1]

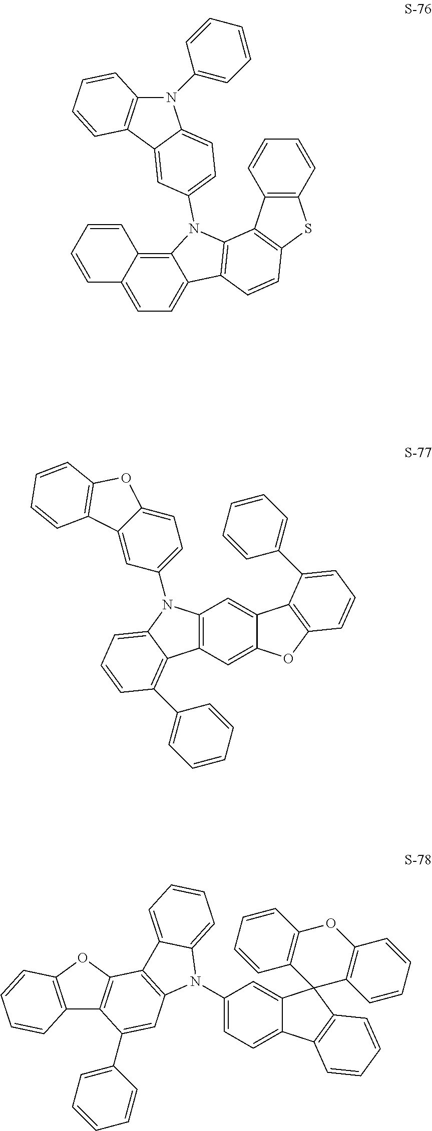 [SMILES: c1ccc(-n2c3ccccc3c3cc(-n4c5c6ccccc6ccc5c5ccc6sc7ccccc7c6c54)ccc32)cc1 ; c1ccc(-c2cccc3oc4cc5c6c(-c7ccccc7)cccc6n(-c6ccc7oc8ccccc8c7c6)c5cc4c23)cc1 ; c1ccc(-c2cc3c(c4ccccc4n3-c3ccc4c(c3)C3(c5ccccc5Oc5ccccc53)c3ccccc3-4)c3oc4ccccc4c23)cc1]

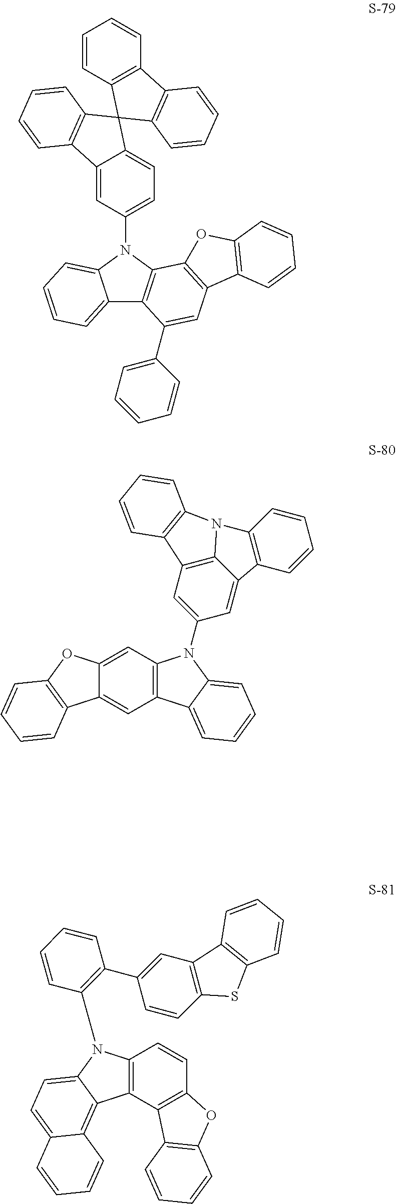 [SMILES: c1ccc(-c2cc3c4ccccc4oc3c3c2c2ccccc2n3-c2ccc3c(c2)-c2ccccc2C32c3ccccc3-c3ccccc32)cc1 ; c1ccc2c(c1)oc1cc3c(cc12)c1ccccc1n3-c1cc2c3ccccc3n3c4ccccc4c(c1)c23 ; c1ccc(-n2c3ccc4ccccc4c3c3c4c(ccc32)oc2ccccc24)c(-c2ccc3sc4ccccc4c3c2)c1]

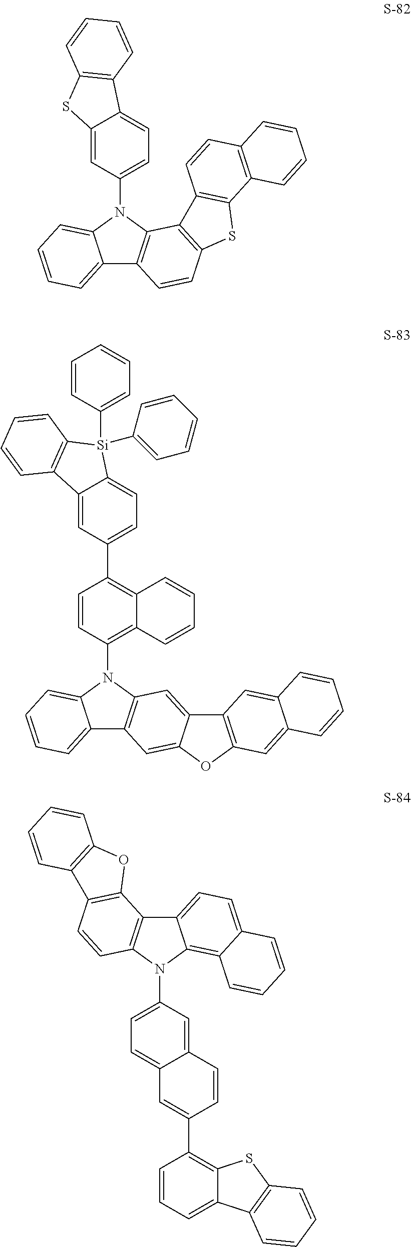 [SMILES: c1ccc2c(c1)ccc1c2sc2ccc3c4ccccc4n(-c4ccc5c(c4)sc4ccccc45)c3c21 ; c1ccc([Si]2(c3ccccc3)c3ccccc3-c3cc(-c4ccc(-n5c6ccccc6c6cc7oc8cc9ccccc9cc8c7cc65)c5ccccc45)ccc32)cc1 ; c1ccc2c(c1)ccc1c3c4oc5ccccc5c4ccc3n(-c3ccc4cc(-c5cccc6c5sc5ccccc56)ccc4c3)c21]

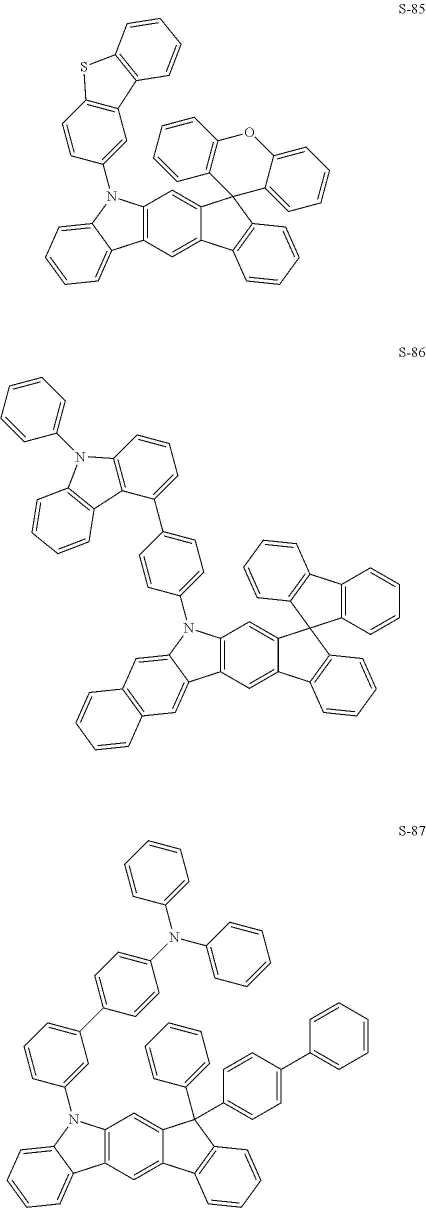 [SMILES: c1ccc2c(c1)Oc1ccccc1C21c2ccccc2-c2cc3c4ccccc4n(-c4ccc5sc6ccccc6c5c4)c3cc21 ; c1ccc(-n2c3ccccc3c3c(-c4ccc(-n5c6cc7c(cc6c6cc8ccccc8cc65)-c5ccccc5C75c6ccccc6-c6ccccc65)cc4)cccc32)cc1 ; c1ccc(-c2ccc(C3(c4ccccc4)c4ccccc4-c4cc5c6ccccc6n(-c6cccc(-c7ccc(N(c8ccccc8)c8ccccc8)cc7)c6)c5cc43)cc2)cc1]

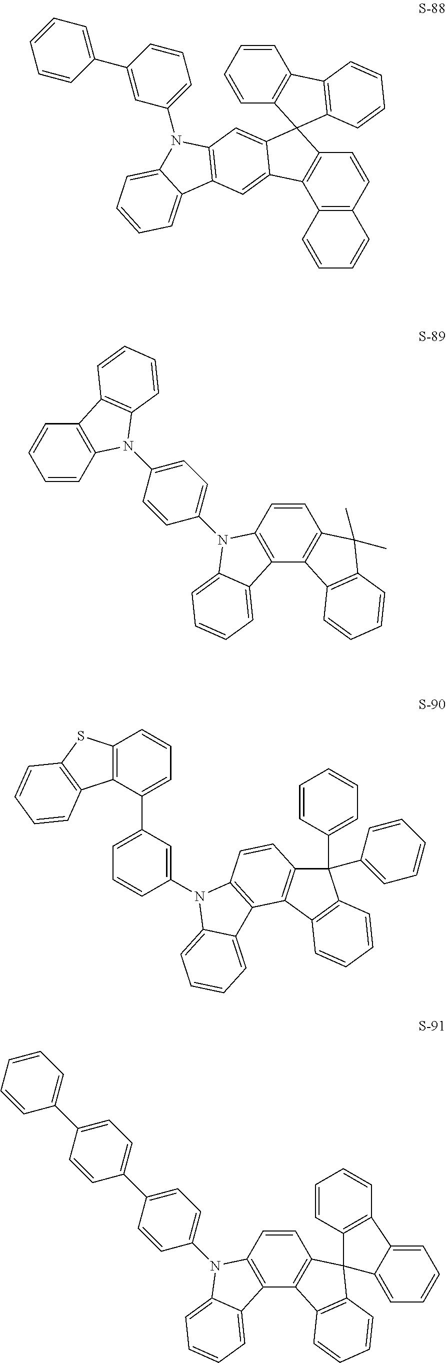 [SMILES: c1ccc(-c2cccc(-n3c4ccccc4c4cc5c(cc43)C3(c4ccccc4-c4ccccc43)c3ccc4ccccc4c3-5)c2)cc1 ; CC1(C)c2ccccc2-c2c1ccc1c2c2ccccc2n1-c1ccc(-n2c3ccccc3c3ccccc32)cc1 ; c1ccc(C2(c3ccccc3)c3ccccc3-c3c2ccc2c3c3ccccc3n2-c2cccc(-c3cccc4sc5ccccc5c34)c2)cc1 ; c1ccc(-c2ccc(-c3ccc(-n4c5ccccc5c5c6c(ccc54)C4(c5ccccc5-c5ccccc54)c4ccccc4-6)cc3)cc2)cc1]

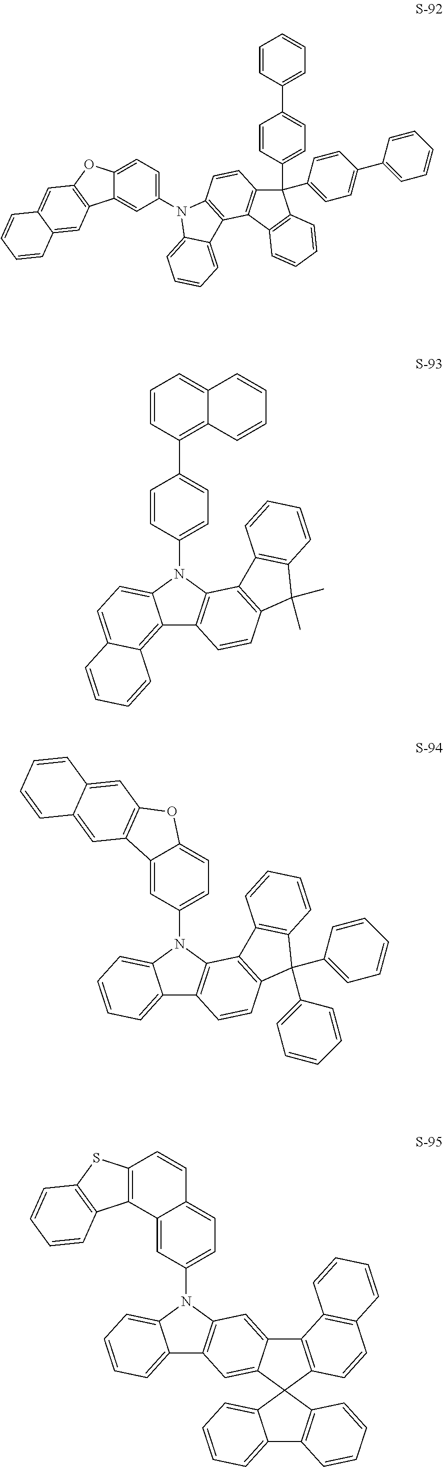 [SMILES: c1ccc(-c2ccc(C3(c4ccc(-c5ccccc5)cc4)c4ccccc4-c4c3ccc3c4c4ccccc4n3-c3ccc4oc5cc6ccccc6cc5c4c3)cc2)cc1 ; CC1(C)c2ccccc2-c2c1ccc1c3c4ccccc4ccc3n(-c3ccc(-c4cccc5ccccc45)cc3)c21 ; c1ccc(C2(c3ccccc3)c3ccccc3-c3c2ccc2c4ccccc4n(-c4ccc5oc6cc7ccccc7cc6c5c4)c32)cc1 ; c1ccc2c(c1)-c1ccccc1C21c2cc3c4ccccc4n(-c4ccc5ccc6sc7ccccc7c6c5c4)c3cc2-c2c1ccc1ccccc21]

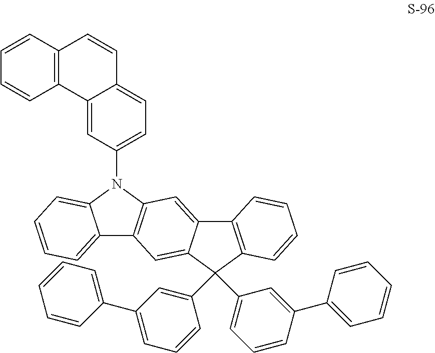 [SMILES: c1ccc(-c2cccc(C3(c4cccc(-c5ccccc5)c4)c4ccccc4-c4cc5c(cc43)c3ccccc3n5-c3ccc4ccc5ccccc5c4c3)c2)cc1]

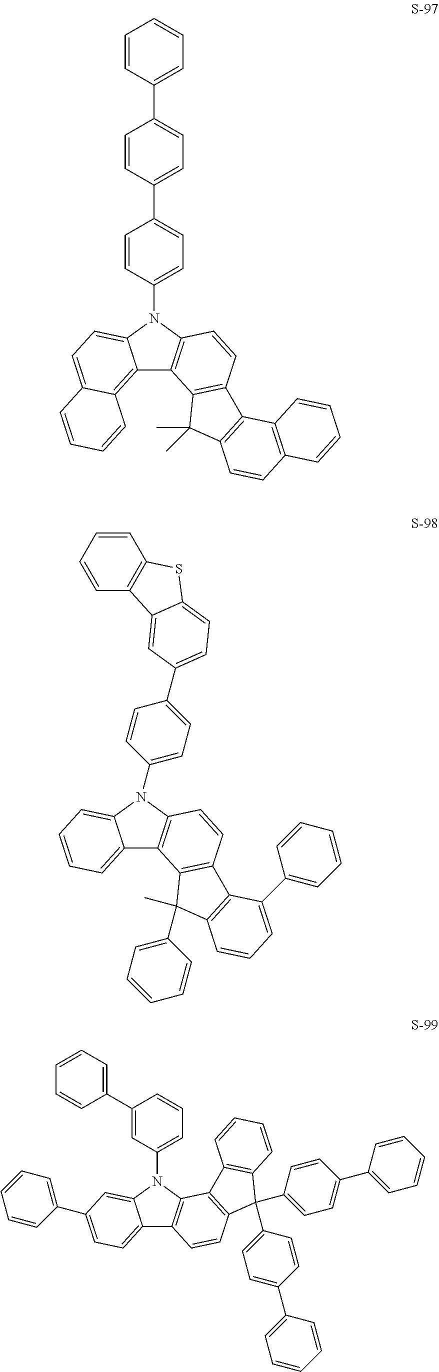 [SMILES: CC1(C)c2ccc3ccccc3c2-c2ccc3c(c21)c1c2ccccc2ccc1n3-c1ccc(-c2ccc(-c3ccccc3)cc2)cc1 ; CC1(c2ccccc2)c2cccc(-c3ccccc3)c2-c2ccc3c(c21)c1ccccc1n3-c1ccc(-c2ccc3sc4ccccc4c3c2)cc1 ; c1ccc(-c2ccc(C3(c4ccc(-c5ccccc5)cc4)c4ccccc4-c4c3ccc3c5ccc(-c6ccccc6)cc5n(-c5cccc(-c6ccccc6)c5)c43)cc2)cc1]

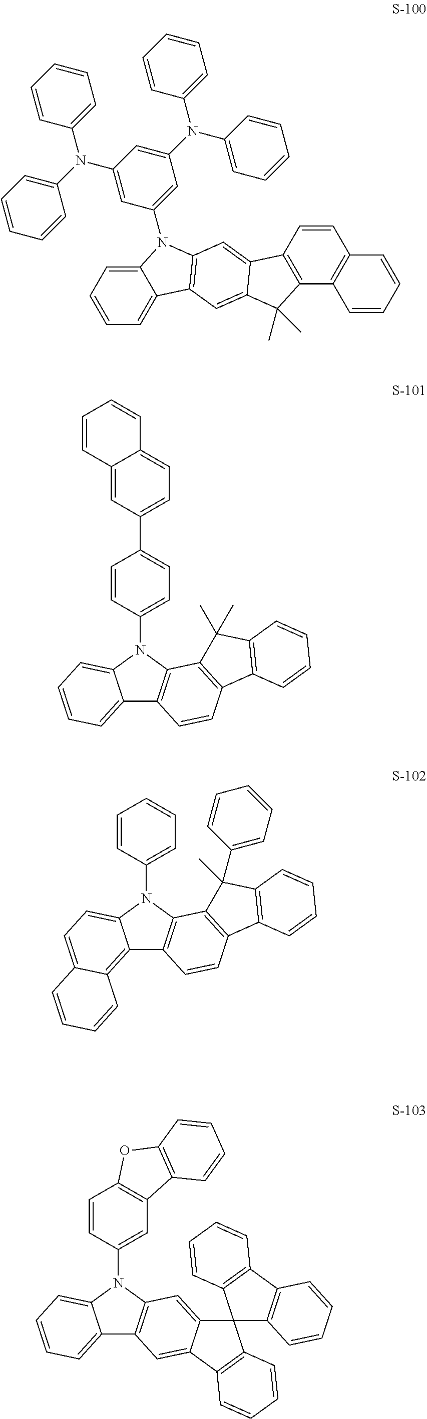 [SMILES: CC1(C)c2cc3c4ccccc4n(-c4cc(N(c5ccccc5)c5ccccc5)cc(N(c5ccccc5)c5ccccc5)c4)c3cc2-c2ccc3ccccc3c21 ; CC1(C)c2ccccc2-c2ccc3c4ccccc4n(-c4ccc(-c5ccc6ccccc6c5)cc4)c3c21 ; CC1(c2ccccc2)c2ccccc2-c2ccc3c4c5ccccc5ccc4n(-c4ccccc4)c3c21 ; c1ccc2c(c1)-c1ccccc1C21c2ccccc2-c2cc3c4ccccc4n(-c4ccc5oc6ccccc6c5c4)c3cc21]

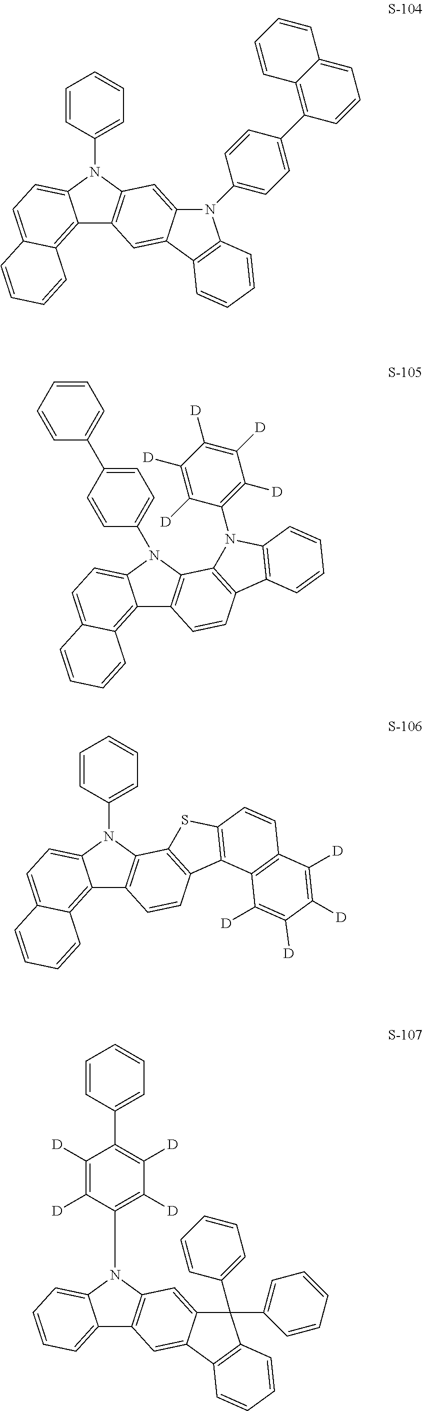 [SMILES: c1ccc(-n2c3cc4c(cc3c3c5ccccc5ccc32)c2ccccc2n4-c2ccc(-c3cccc4ccccc34)cc2)cc1 ; [2H]c1c([2H])c([2H])c(-n2c3ccccc3c3ccc4c5c6ccccc6ccc5n(-c5ccc(-c6ccccc6)cc5)c4c32)c([2H])c1[2H] ; [2H]c1c([2H])c([2H])c2c(ccc3sc4c(ccc5c6c7ccccc7ccc6n(-c6ccccc6)c54)c32)c1[2H] ; [2H]c1c([2H])c(-n2c3ccccc3c3cc4c(cc32)C(c2ccccc2)(c2ccccc2)c2ccccc2-4)c([2H])c([2H])c1-c1ccccc1]

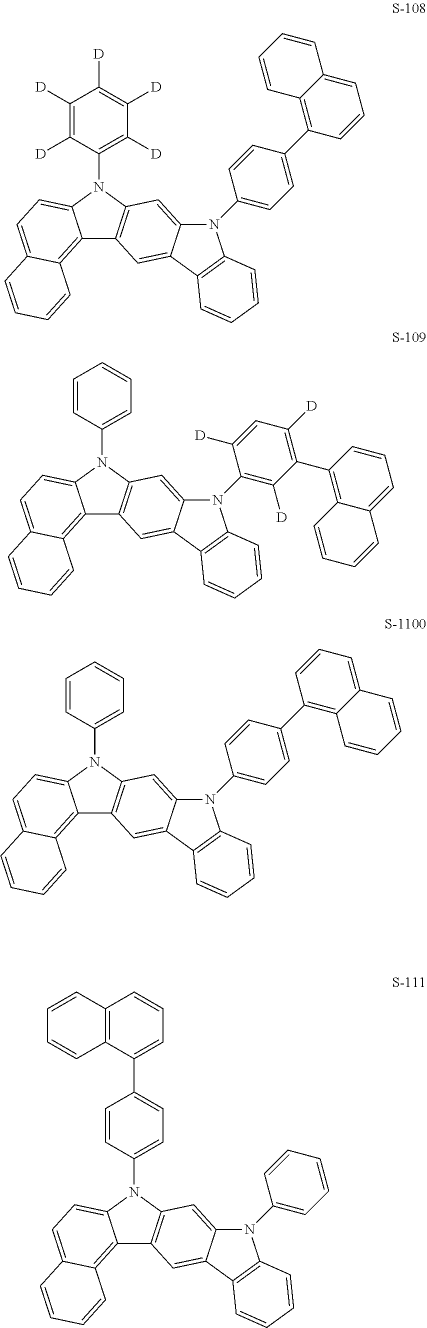 [SMILES: [2H]c1c([2H])c([2H])c(-n2c3cc4c(cc3c3c5ccccc5ccc32)c2ccccc2n4-c2ccc(-c3cccc4ccccc34)cc2)c([2H])c1[2H] ; [2H]c1cc([2H])c(-n2c3ccccc3c3cc4c5c6ccccc6ccc5n(-c5ccccc5)c4cc32)c([2H])c1-c1cccc2ccccc12 ; c1ccc(-n2c3cc4c(cc3c3c5ccccc5ccc32)c2ccccc2n4-c2ccc(-c3cccc4ccccc34)cc2)cc1 ; c1ccc(-n2c3ccccc3c3cc4c5c6ccccc6ccc5n(-c5ccc(-c6cccc7ccccc67)cc5)c4cc32)cc1]

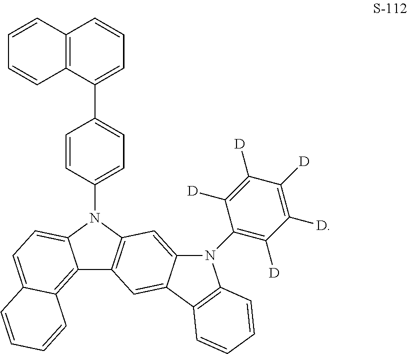

19. The organic electronic element according to claim 6, further comprising a light efficiency enhancing layer formed on at least one surface of the first electrode and the second electrode, the surface being opposite to the organic material layer.

20. The organic electronic element according to claim 6, wherein the organic material layer comprises 2 or more stacks comprising a hole transport layer, an emitting layer and an electron transport layer sequentially formed on the first electrode.

21. The organic electronic element according to claim 20, wherein the organic material layer further comprises a charge generation layer formed between the 2 or more stacks.

22. An electronic device comprising a display device comprising the organic electronic element of claim 6; and a control unit for driving the display device.

23. An electronic device according to claim 22, wherein the organic electronic element is at least one of an OLED, an organic solar cell, an organic photo conductor (OPC), organic transistor (organic TFT) and an element for monochromic or white illumination.
